SEQUENCE LISTING (308 sequences)

SEQ ID NO: 1 - DNA, 360 bp, synthetic construct
  source 1..360: mol_type = other DNA
gaggtgcagctggtggagtctgggggaggcctggtcaagccgggggggtccctgagaatttactgtgtagcctctggattcaccttcagttcctatagcatgaactgggtccgccaggctccagggaaggggctggagtgggtctcatccattagtagcagtagtagttacatacagtacgcagactcagtgaagggccgattcaccatgtccagagacaacgccaagaactcactgtatctgcaaatgaacagcctgagagccgaggacacggctgtgtattattgtgtgagagggggggtagtagtaccagctgctatggtctactggggccagggatccctggtcaccgtctcctca

SEQ ID NO: 2 - protein, 120 aa, synthetic construct
EVQLVESGGGLVKPGGSLRIYCVASGFTFSSYSMNWVRQAPGKGLEWVSSISSSSSYIQYADSVKGRFTMSRDNAKNSLYLQMNSLRAEDTAVYYCVRGGVVVPAAMVYWGQGSLVTVSS

SEQ ID NO: 3 - DNA, 24 bp, synthetic construct
  source 1..24: mol_type = other DNA
ggattcaccttcagttcctatagc

SEQ ID NO: 4 - protein, 8 aa, synthetic construct
GFTFSSYS

SEQ ID NO: 5 - DNA, 24 bp, synthetic construct
  source 1..24: mol_type = other DNA
attagtagcagtagtagttacata

SEQ ID NO: 6 - protein, 8 aa, synthetic construct
ISSSSSYI

SEQ ID NO: 7 - DNA, 39 bp, synthetic construct
  source 1..39: mol_type = other DNA
gtgagagggggggtagtagtaccagctgctatggtctac

SEQ ID NO: 8 - protein, 13 aa, synthetic construct
VRGGVVVPAAMVY

SEQ ID NO: 9 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, synthetic construct
  source 1..18: mol_type = other DNA
cagagcattagcagctat

SEQ ID NO: 12 - protein, 6 aa, synthetic construct
QSISSY

SEQ ID NO: 13-14 - skipped (2 entries)

SEQ ID NO: 15 - DNA, 30 bp, synthetic construct
  source 1..30: mol_type = other DNA
caacagagttacagtacccctccgatcacc

SEQ ID NO: 16 - protein, 10 aa, synthetic construct
QQSYSTPPIT

SEQ ID NO: 17 - DNA, 1341 bp, synthetic construct
  source 1..1341: mol_type = other DNA
gaggtgcagctggtggagtctgggggaggcctggtcaagccgggggggtccctgagaatttactgtgtagcctctggattcaccttcagttcctatagcatgaactgggtccgccaggctccagggaaggggctggagtgggtctcatccattagtagcagtagtagttacatacagtacgcagactcagtgaagggccgattcaccatgtccagagacaacgccaagaactcactgtatctgcaaatgaacagcctgagagccgaggacacggctgtgtattattgtgtgagagggggggtagtagtaccagctgctatggtctactggggccagggatccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 446 aa, synthetic construct
EVQLVESGGGLVKPGGSLRIYCVASGFTFSSYSMNWVRQAPGKGLEWVSSISSSSSYIQYADSVKGRFTMSRDNAKNSLYLQMNSLRAEDTAVYYCVRGGVVVPAAMVYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 360 bp, synthetic construct
  source 1..360: mol_type = other DNA
gaggtgcagctggtggagtctgggggaggcctggtcaaacctggggggtccctgagactctcctgtgcagcctctggattcaccttcagtaaatatagcatgaactgggtccgccaggctccagggaaggggctggagtgggtctcatccattactagtagtagtagttacatacaatacgcagactcactgaagggccgattcaccatctccagagacaacgccaagaactcactgtatctgcaaatgaacagcctgagagccgaggacacggctgtatattattgtgcgagagggggggtagtagtaccagctgctatggcctactggggccagggaaccctggtcaccgtctcctca

SEQ ID NO: 22 - protein, 120 aa, synthetic construct
EVQLVESGGGLVKPGGSLRLSCAASGFTFSKYSMNWVRQAPGKGLEWVSSITSSSSYIQYADSLKGRFTISRDNAKNSLYLQMNSLRAEDTAVYYCARGGVVVPAAMAYWGQGTLVTVSS

SEQ ID NO: 23 - DNA, 24 bp, synthetic construct
  source 1..24: mol_type = other DNA
ggattcaccttcagtaaatatagc

SEQ ID NO: 24 - protein, 8 aa, synthetic construct
GFTFSKYS

SEQ ID NO: 25 - DNA, 24 bp, synthetic construct
  source 1..24: mol_type = other DNA
attactagtagtagtagttacata

SEQ ID NO: 26 - protein, 8 aa, synthetic construct
ITSSSSYI

SEQ ID NO: 27 - DNA, 39 bp, synthetic construct
  source 1..39: mol_type = other DNA
gcgagagggggggtagtagtaccagctgctatggcctac

SEQ ID NO: 28 - protein, 13 aa, synthetic construct
ARGGVVVPAAMAY

SEQ ID NO: 29 - DNA, 1341 bp, synthetic construct
  source 1..1341: mol_type = other DNA
gaggtgcagctggtggagtctgggggaggcctggtcaaacctggggggtccctgagactctcctgtgcagcctctggattcaccttcagtaaatatagcatgaactgggtccgccaggctccagggaaggggctggagtgggtctcatccattactagtagtagtagttacatacaatacgcagactcactgaagggccgattcaccatctccagagacaacgccaagaactcactgtatctgcaaatgaacagcctgagagccgaggacacggctgtatattattgtgcgagagggggggtagtagtaccagctgctatggcctactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 30 - protein, 446 aa, synthetic construct
EVQLVESGGGLVKPGGSLRLSCAASGFTFSKYSMNWVRQAPGKGLEWVSSITSSSSYIQYADSLKGRFTISRDNAKNSLYLQMNSLRAEDTAVYYCARGGVVVPAAMAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 375 bp, synthetic construct
  source 1..375: mol_type = other DNA
caggtgcagctggtgcagtctggggctgaggtgaagaggcctggggcctcagtgaaggtctcctgcaaggcttctggatacattttcaccggctactatattcactggttgcgacaggcccccggacaagggcttgagtggatgggatggatcaaccctaacagtggtgacacaaactatatccagaagtttcagggcagggtcaccatgaccagggacacgtccatcagcacagcctacatggacctgagcaggctgagatctgacgacacggccgtgttttactgtgcgagaatccaggtttattatgatggtggtatttataattaccccctccaccactggggccagggcaccctggtcaccgtctcctca

SEQ ID NO: 32 - protein, 125 aa, synthetic construct
QVQLVQSGAEVKRPGASVKVSCKASGYIFTGYYIHWLRQAPGQGLEWMGWINPNSGDTNYIQKFQGRVTMTRDTSISTAYMDLSRLRSDDTAVFYCARIQVYYDGGIYNYPLHHWGQGTLVTVSS

SEQ ID NO: 33 - DNA, 24 bp, synthetic construct
  source 1..24: mol_type = other DNA
ggatacattttcaccggctactat

SEQ ID NO: 34 - protein, 8 aa, synthetic construct
GYIFTGYY

SEQ ID NO: 35 - DNA, 24 bp, synthetic construct
  source 1..24: mol_type = other DNA
atcaaccctaacagtggtgacaca

SEQ ID NO: 36 - protein, 8 aa, synthetic construct
INPNSGDT

SEQ ID NO: 37 - DNA, 54 bp, synthetic construct
  source 1..54: mol_type = other DNA
gcgagaatccaggtttattatgatggtggtatttataattaccccctccaccac

SEQ ID NO: 38 - protein, 18 aa, synthetic construct
ARIQVYYDGGIYNYPLHH

SEQ ID NO: 39 - DNA, 324 bp, synthetic construct
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 40 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 41 - DNA, 21 bp, synthetic construct
  source 1..21: mol_type = other DNA
cagtcagtctctagctcttat

SEQ ID NO: 42 - protein, 7 aa, synthetic construct
QSVSSSY

SEQ ID NO: 43-44 - skipped (2 entries)

SEQ ID NO: 45 - DNA, 27 bp, synthetic construct
  source 1..27: mol_type = other DNA
caacagtacggaagcagcccgtggacg

SEQ ID NO: 46 - protein, 9 aa, synthetic construct
QQYGSSPWT

SEQ ID NO: 47 - DNA, 1368 bp, synthetic construct
  source 1..1368: mol_type = other DNA
caggtgcagctggtgcagtctggggctgaggtgaagaggcctggggcctcagtgaaggtctcctgcaaggcttctggatacattttcaccggctactatattcactggttgcgacaggcccccggacaagggcttgagtggatgggatggatcaaccctaacagtggtgacacaaactatatccagaagtttcagggcagggtcaccatgaccagggacacgtccatcagcacagcctacatggacctgagcaggctgagatctgacgacacggccgtgttttactgtgcgagaatccaggtttattatgatggtggtatttataattaccccctccaccactggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 48 - protein, 455 aa, synthetic construct
QVQLVQSGAEVKRPGASVKVSCKASGYIFTGYYIHWLRQAPGQGLEWMGWINPNSGDTNYIQKFQGRVTMTRDTSISTAYMDLSRLRSDDTAVFYCARIQVYYDGGIYNYPLHHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 648 bp, synthetic construct
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 50 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - DNA, 372 bp, synthetic construct
  source 1..372: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatccattacagtgggaacacctactacaacccgtccctcaagagtcgaattaccgtatctttagacacgtctaaggaccagttctccctgaggctgagttctgtgactgccgcggacacggccgtgtactactgtgcgagagatgagggacgtggatacagctatggtcggtacttcgatctctggggccgtggcaccctggtcactgtctcctca

SEQ ID NO: 52 - protein, 124 aa, synthetic construct
QVQLQESGPGLVKPSQTLSLTCTVSGGSISSGGYYWSWIRQHPGKGLEWIGYIHYSGNTYYNPSLKSRITVSLDTSKDQFSLRLSSVTAADTAVYYCARDEGRGYSYGRYFDLWGRGTLVTVSS

SEQ ID NO: 53 - DNA, 30 bp, synthetic construct
  source 1..30: mol_type = other DNA
ggtggctccatcagcagtggtggttactac

SEQ ID NO: 54 - protein, 10 aa, synthetic construct
GGSISSGGYY

SEQ ID NO: 55 - DNA, 21 bp, synthetic construct
  source 1..21: mol_type = other DNA
atccattacagtgggaacacc

SEQ ID NO: 56 - protein, 7 aa, synthetic construct
IHYSGNT

SEQ ID NO: 57 - DNA, 48 bp, synthetic construct
  source 1..48: mol_type = other DNA
gcgagagatgagggacgtggatacagctatggtcggtacttcgatctc

SEQ ID NO: 58 - protein, 16 aa, synthetic construct
ARDEGRGYSYGRYFDL

SEQ ID NO: 59 - DNA, 1353 bp, synthetic construct
  source 1..1353: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatccattacagtgggaacacctactacaacccgtccctcaagagtcgaattaccgtatctttagacacgtctaaggaccagttctccctgaggctgagttctgtgactgccgcggacacggccgtgtactactgtgcgagagatgagggacgtggatacagctatggtcggtacttcgatctctggggccgtggcaccctggtcactgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 60 - protein, 450 aa, synthetic construct
QVQLQESGPGLVKPSQTLSLTCTVSGGSISSGGYYWSWIRQHPGKGLEWIGYIHYSGNTYYNPSLKSRITVSLDTSKDQFSLRLSSVTAADTAVYYCARDEGRGYSYGRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - DNA, 369 bp, synthetic construct
  source 1..369: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcacttatatcatatgatggaagtaataaatactatacagactccgtgaagggccgattcaccatctccagagacaattccaagaacacgctgtatctgcaaatgaacagcctgagagctgaggacacggctgtatatttctgtgcgtctctgggagcccctacttactcctacttctccgatatggacgtctggggcctagggaccacggtcaccgtctcctca

SEQ ID NO: 62 - protein, 123 aa, synthetic construct
QVQLVESGGGVVQPGRSLRLSCAASGFTFSSYGMHWVRQAPGKGLEWVALISYDGSNKYYTDSVKGRFTISRDNSKNTLYLQMNSLRAEDTAVYFCASLGAPTYSYFSDMDVWGLGTTVTVSS

SEQ ID NO: 63 - DNA, 24 bp, synthetic construct
  source 1..24: mol_type = other DNA
ggattcaccttcagtagctatggc

SEQ ID NO: 64 - protein, 8 aa, synthetic construct
GFTFSSYG

SEQ ID NO: 65 - DNA, 24 bp, synthetic construct
  source 1..24: mol_type = other DNA
atatcatatgatggaagtaataaa

SEQ ID NO: 66 - protein, 8 aa, synthetic construct
ISYDGSNK

SEQ ID NO: 67 - DNA, 48 bp, synthetic construct
  source 1..48: mol_type = other DNA
gcgtctctgggagcccctacttactcctacttctccgatatggacgtc

SEQ ID NO: 68 - protein, 16 aa, synthetic construct
ASLGAPTYSYFSDMDV

SEQ ID NO: 69 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagttctttaaattggtatcagcagaaaccagggaaagcccctaagttcttgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 70 - protein, 108 aa, synthetic construct
DIQMTQSPSSLSASVGDRVTITCRASQSISSSLNWYQQKPGKAPKFLIYAASSLQSGVPSRFSGSGSGTDFTLTISSLQPEDFATYYCQQSYSTPPITFGQGTRLEIK

SEQ ID NO: 71 - DNA, 18 bp, synthetic construct
  source 1..18: mol_type = other DNA
cagagcattagcagttct

SEQ ID NO: 72 - protein, 6 aa, synthetic construct
QSISSS

SEQ ID NO: 73 - DNA, 1350 bp, synthetic construct
  source 1..1350: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcacttatatcatatgatggaagtaataaatactatacagactccgtgaagggccgattcaccatctccagagacaattccaagaacacgctgtatctgcaaatgaacagcctgagagctgaggacacggctgtatatttctgtgcgtctctgggagcccctacttactcctacttctccgatatggacgtctggggcctagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 74 - protein, 449 aa, synthetic construct
QVQLVESGGGVVQPGRSLRLSCAASGFTFSSYGMHWVRQAPGKGLEWVALISYDGSNKYYTDSVKGRFTISRDNSKNTLYLQMNSLRAEDTAVYFCASLGAPTYSYFSDMDVWGLGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagttctttaaattggtatcagcagaaaccagggaaagcccctaagttcttgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 76 - protein, 215 aa, synthetic construct
DIQMTQSPSSLSASVGDRVTITCRASQSISSSLNWYQQKPGKAPKFLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 375 bp, synthetic construct
  source 1..375: mol_type = other DNA
caggtgcagctggtgcagtctggggctgaggtgaagaagcctggggcctcagtgaaggtctcctgcaaggcttctggatacaccttcaccggctactatatgcactggttgcgacaggcccctggacaagggcttgagtggatgggatggatcaaccctaacactggtgacacaaactatatacagaagtttcagggcagggtcaccatgaccagggacacgtccatcagcacagcctacatggagctgagcaggctgagatctgacgacacggccgtgttttactgtgcgagaatccgggtttattatgaaagtggtatttataactaccccctccaccactggggccagggcaccctggtcaccgtctcctca

SEQ ID NO: 78 - protein, 125 aa, synthetic construct
QVQLVQSGAEVKKPGASVKVSCKASGYTFTGYYMHWLRQAPGQGLEWMGWINPNTGDTNYIQKFQGRVTMTRDTSISTAYMELSRLRSDDTAVFYCARIRVYYESGIYNYPLHHWGQGTLVTVSS

SEQ ID NO: 79 - DNA, 24 bp, synthetic construct
  source 1..24: mol_type = other DNA
ggatacaccttcaccggctactat

SEQ ID NO: 80 - protein, 8 aa, synthetic construct
GYTFTGYY

SEQ ID NO: 81 - DNA, 24 bp, synthetic construct
  source 1..24: mol_type = other DNA
atcaaccctaacactggtgacaca

SEQ ID NO: 82 - protein, 8 aa, synthetic construct
INPNTGDT

SEQ ID NO: 83 - DNA, 54 bp, synthetic construct
  source 1..54: mol_type = other DNA
gcgagaatccgggtttattatgaaagtggtatttataactaccccctccaccac

SEQ ID NO: 84 - protein, 18 aa, synthetic construct
ARIRVYYESGIYNYPLHH

SEQ ID NO: 85 - DNA, 1368 bp, synthetic construct
  source 1..1368: mol_type = other DNA
caggtgcagctggtgcagtctggggctgaggtgaagaagcctggggcctcagtgaaggtctcctgcaaggcttctggatacaccttcaccggctactatatgcactggttgcgacaggcccctggacaagggcttgagtggatgggatggatcaaccctaacactggtgacacaaactatatacagaagtttcagggcagggtcaccatgaccagggacacgtccatcagcacagcctacatggagctgagcaggctgagatctgacgacacggccgtgttttactgtgcgagaatccgggtttattatgaaagtggtatttataactaccccctccaccactggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 86 - protein, 455 aa, synthetic construct
QVQLVQSGAEVKKPGASVKVSCKASGYTFTGYYMHWLRQAPGQGLEWMGWINPNTGDTNYIQKFQGRVTMTRDTSISTAYMELSRLRSDDTAVFYCARIRVYYESGIYNYPLHHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 375 bp, synthetic construct
  source 1..375: mol_type = other DNA
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtttattactgtgcgagtttttgtagtagtaccagctgctactactactactacggtatggacgtctggggccaagggaccacggtcaccgtctcctca

SEQ ID NO: 88 - protein, 125 aa, synthetic construct
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCASFCSSTSCYYYYYGMDVWGQGTTVTVSS

SEQ ID NO: 89 - DNA, 24 bp, synthetic construct
  source 1..24: mol_type = other DNA
ggattcaccttcagtagttatgaa

SEQ ID NO: 90 - protein, 8 aa, synthetic construct
GFTFSSYE

SEQ ID NO: 91 - DNA, 24 bp, synthetic construct
  source 1..24: mol_type = other DNA
attagtagtagtggtagtaccata

SEQ ID NO: 92 - protein, 8 aa, synthetic construct
ISSSGSTI

SEQ ID NO: 93 - DNA, 54 bp, synthetic construct
  source 1..54: mol_type = other DNA
gcgagtttttgtagtagtaccagctgctactactactactacggtatggacgtc

SEQ ID NO: 94 - protein, 18 aa, synthetic construct
ASFCSSTSCYYYYYGMDV

SEQ ID NO: 95 - DNA, 1356 bp, synthetic construct
  source 1..1356: mol_type = other DNA
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtttattactgtgcgagtttttgtagtagtaccagctgctactactactactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 96 - protein, 451 aa, synthetic construct
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCASFCSSTSCYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - DNA, 1356 bp, synthetic construct
  source 1..1356: mol_type = other DNA
caggtgcagctggtgcagtctggggctgaggtgaagaggcctggggcctcagtgaaggtctcctgcaaggcttctggatacattttcaccggctactatattcactggttgcgacaggcccccggacaagggcttgagtggatgggatggatcaaccctaacagtggtgacacaaactatatccagaagtttcagggcagggtcaccatgaccagggacacgtccatcagcacagcctacatggacctgagcaggctgagatctgacgacacggccgtgttttactgtgcgagaatccaggtttattatgatggtggtatttataattaccccctccaccactggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 98 - protein, 451 aa, synthetic construct
QVQLVQSGAEVKRPGASVKVSCKASGYIFTGYYIHWLRQAPGQGLEWMGWINPNSGDTNYIQKFQGRVTMTRDTSISTAYMDLSRLRSDDTAVFYCARIQVYYDGGIYNYPLHHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - DNA, 1362 bp, synthetic construct
  source 1..1362: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcacttatatcatatgatggaagtaataaatactatacagactccgtgaagggccgattcaccatctccagagacaattccaagaacacgctgtatctgcaaatgaacagcctgagagctgaggacacggctgtatatttctgtgcgtctctgggagcccctacttactcctacttctccgatatggacgtctggggcctagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 100 - protein, 453 aa, synthetic construct
QVQLVESGGGVVQPGRSLRLSCAASGFTFSSYGMHWVRQAPGKGLEWVALISYDGSNKYYTDSVKGRFTISRDNSKNTLYLQMNSLRAEDTAVYFCASLGAPTYSYFSDMDVWGLGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1356 bp, synthetic construct
  source 1..1356: mol_type = other DNA
caggtgcagctggtgcagtctggggctgaggtgaagaagcctggggcctcagtgaaggtctcctgcaaggcttctggatacaccttcaccggctactatatgcactggttgcgacaggcccctggacaagggcttgagtggatgggatggatcaaccctaacactggtgacacaaactatatacagaagtttcagggcagggtcaccatgaccagggacacgtccatcagcacagcctacatggagctgagcaggctgagatctgacgacacggccgtgttttactgtgcgagaatccgggtttattatgaaagtggtatttataactaccccctccaccactggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 102 - protein, 451 aa, synthetic construct
QVQLVQSGAEVKKPGASVKVSCKASGYTFTGYYMHWLRQAPGQGLEWMGWINPNTGDTNYIQKFQGRVTMTRDTSISTAYMELSRLRSDDTAVFYCARIRVYYESGIYNYPLHHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - DNA, 342 bp, synthetic construct
  source 1..342: mol_type = other DNA
caggtgcagctggtacagtctggggctgagatgaagaagcctggggactcagtgaaggtctcctgcaaggcttctggatacaccttctccgtctactatatgcattgggtgcgacaggcccctgggcaagggcttgagtggatgggatggatcaaccctaaaagtggtggcacaaactatgcacagaggtttcagggcagggtcgccatgaccagggacacgtccatcaccacagcctacatggaactaaggagactgagatctgacgacatggccgtatattattgtgtaagaagggggattgactactggggccagggaaccctggtcaccgtctcctca

SEQ ID NO: 104 - protein, 114 aa, synthetic construct
QVQLVQSGAEMKKPGDSVKVSCKASGYTFSVYYMHWVRQAPGQGLEWMGWINPKSGGTNYAQRFQGRVAMTRDTSITTAYMELRRLRSDDMAVYYCVRRGIDYWGQGTLVTVSS

SEQ ID NO: 105 - DNA, 24 bp, synthetic construct
  source 1..24: mol_type = other DNA
ggatacaccttctccgtctactat

SEQ ID NO: 106 - protein, 8 aa, synthetic construct
GYTFSVYY

SEQ ID NO: 107 - DNA, 24 bp, synthetic construct
  source 1..24: mol_type = other DNA
atcaaccctaaaagtggtggcaca

SEQ ID NO: 108 - protein, 8 aa, synthetic construct
INPKSGGT

SEQ ID NO: 109 - DNA, 21 bp, synthetic construct
  source 1..21: mol_type = other DNA
gtaagaagggggattgactac

SEQ ID NO: 110 - protein, 7 aa, synthetic construct
VRRGIDY

SEQ ID NO: 111 - DNA, 324 bp, synthetic construct
  source 1..324: mol_type = other DNA
gacatccagatgacccagtctccatcctccctgtctgcatctgtaggagacagagtcaccatcacttgtcgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 112 - protein, 108 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSYSTPPITFGQGTRLEIK

SEQ ID NO: 113 - DNA, 18 bp, synthetic construct
  source 1..18: mol_type = other DNA
cagagcattaacagctat

SEQ ID NO: 114 - protein, 6 aa, synthetic construct
QSINSY

SEQ ID NO: 115 - DNA, 1335 bp, synthetic construct
  source 1..1335: mol_type = other DNA
caggtgcagctggtacagtctggggctgagatgaagaagcctggggactcagtgaaggtctcctgcaaggcttctggatacaccttctccgtctactatatgcattgggtgcgacaggcccctgggcaagggcttgagtggatgggatggatcaaccctaaaagtggtggcacaaactatgcacagaggtttcagggcagggtcgccatgaccagggacacgtccatcaccacagcctacatggaactaaggagactgagatctgacgacatggccgtatattattgtgtaagaagggggattgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 116 - protein, 444 aa, synthetic construct
QVQLVQSGAEMKKPGDSVKVSCKASGYTFSVYYMHWVRQAPGQGLEWMGWINPKSGGTNYAQRFQGRVAMTRDTSITTAYMELRRLRSDDMAVYYCVRRGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 648 bp, synthetic construct
  source 1..648: mol_type = other DNA
gacatccagatgacccagtctccatcctccctgtctgcatctgtaggagacagagtcaccatcacttgtcgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 118 - protein, 215 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - DNA, 384 bp, synthetic construct
  source 1..384: mol_type = other DNA
gaggtgcagctggtggagtctgggggaggcttggtacagccagggcggtccctgagactctcctgtacagcttctggattcacctttggtgattatgctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagagtaccgttttacgatattttgactggcccctactactacggtatggacgtctggggccaagggaccacggtcaccgtctcctca

SEQ ID NO: 120 - protein, 128 aa, synthetic construct
EVQLVESGGGLVQPGRSLRLSCTASGFTFGDYAMSWVRQAPGKGLEWVGFIRSKAYGGTTEYAASVKGRFTISRDDSKSIAYLQMNSLKTEDTAVYYCTRVPFYDILTGPYYYGMDVWGQGTTVTVSS

SEQ ID NO: 121 - DNA, 24 bp, synthetic construct
  source 1..24: mol_type = other DNA
ggattcacctttggtgattatgct

SEQ ID NO: 122 - protein, 8 aa, synthetic construct
GFTFGDYA

SEQ ID NO: 123 - DNA, 30 bp, synthetic construct
  source 1..30: mol_type = other DNA
attagaagcaaagcttatggtgggacaaca

SEQ ID NO: 124 - protein, 10 aa, synthetic construct
IRSKAYGGTT

SEQ ID NO: 125 - DNA, 57 bp, synthetic construct
  source 1..57: mol_type = other DNA
actagagtaccgttttacgatattttgactggcccctactactacggtatggacgtc

SEQ ID NO: 126 - protein, 19 aa, synthetic construct
TRVPFYDILTGPYYYGMDV

SEQ ID NO: 127 - DNA, 1365 bp, synthetic construct
  source 1..1365: mol_type = other DNA
gaggtgcagctggtggagtctgggggaggcttggtacagccagggcggtccctgagactctcctgtacagcttctggattcacctttggtgattatgctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagagtaccgttttacgatattttgactggcccctactactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 128 - protein, 454 aa, synthetic construct
EVQLVESGGGLVQPGRSLRLSCTASGFTFGDYAMSWVRQAPGKGLEWVGFIRSKAYGGTTEYAASVKGRFTISRDDSKSIAYLQMNSLKTEDTAVYYCTRVPFYDILTGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcagcagctactatatacactgggtgcgacaggcccctggacaagggcttgagtggatgggattcatcaaccctagtggtggtggcacaagctacgcccagaggttccagggcagagtctccatgaccagggactcgtccacgcgaacagtctacatggagctgagcagtctgagatctgaggacacggccgtgtattactgtgcgcgatttgggggtcttacgatttaccgatttgactactggggtcagggaaccctggtcaccgtctcctca

SEQ ID NO: 130 - protein, 120 aa, synthetic construct
QVQLVQSGAEVKKPGASVKVSCKASGYTFSSYYIHWVRQAPGQGLEWMGFINPSGGGTSYAQRFQGRVSMTRDSSTRTVYMELSSLRSEDTAVYYCARFGGLTIYRFDYWGQGTLVTVSS

SEQ ID NO: 131 - DNA, 24 bp, synthetic construct
  source 1..24: mol_type = other DNA
ggatacaccttcagcagctactat

SEQ ID NO: 132 - protein, 8 aa, synthetic construct
GYTFSSYY

SEQ ID NO: 133 - DNA, 24 bp, synthetic construct
  source 1..24: mol_type = other DNA
atcaaccctagtggtggtggcaca

SEQ ID NO: 134 - protein, 8 aa, synthetic construct
INPSGGGT

SEQ ID NO: 135 - DNA, 39 bp, synthetic construct
  source 1..39: mol_type = other DNA
gcgcgatttgggggtcttacgatttaccgatttgactac

SEQ ID NO: 136 - protein, 13 aa, synthetic construct
ARFGGLTIYRFDY

SEQ ID NO: 137 - DNA, 1341 bp, synthetic construct
  source 1..1341: mol_type = other DNA
caggtgcagctggtgcagtctggggctgaggtgaagaagcctggggcctcagtgaaggtttcctgcaaggcatctggatacaccttcagcagctactatatacactgggtgcgacaggcccctggacaagggcttgagtggatgggattcatcaaccctagtggtggtggcacaagctacgcccagaggttccagggcagagtctccatgaccagggactcgtccacgcgaacagtctacatggagctgagcagtctgagatctgaggacacggccgtgtattactgtgcgcgatttgggggtcttacgatttaccgatttgactactggggtcagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 138 - protein, 446 aa, synthetic construct
QVQLVQSGAEVKKPGASVKVSCKASGYTFSSYYIHWVRQAPGQGLEWMGFINPSGGGTSYAQRFQGRVSMTRDSSTRTVYMELSSLRSEDTAVYYCARFGGLTIYRFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtattcctccgatcaccttcggccaagggacacgactggagattaaa

SEQ ID NO: 140 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSIPPITFGQGTRLEIK

SEQ ID NO: 141 - DNA, 30 bp, synthetic construct
  source 1..30: mol_type = other DNA
caacagagttacagtattcctccgatcacc

SEQ ID NO: 142 - protein, 10 aa, synthetic construct
QQSYSIPPIT

SEQ ID NO: 143 - DNA, 1353 bp, synthetic construct
  source 1..1353: mol_type = other DNA
gaggtgcagctggtggagtctgggggaggcctggtcaaacctggggggtccctgagactctcctgtgcagcctctggattcaccttcagtaaatatagcatgaactgggtccgccaggctccagggaaggggctggagtgggtctcatccattactagtagtagtagttacatacaatacgcagactcactgaagggccgattcaccatctccagagacaacgccaagaactcactgtatctgcaaatgaacagcctgagagccgaggacacggctgtatattattgtgcgagagggggggtagtagtaccagctgctatggcctactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 144 - protein, 450 aa, synthetic construct
EVQLVESGGGLVKPGGSLRLSCAASGFTFSKYSMNWVRQAPGKGLEWVSSITSSSSYIQYADSLKGRFTISRDNAKNSLYLQMNSLRAEDTAVYYCARGGVVVPAAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtattcctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 146 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSIPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - DNA, 360 bp, synthetic construct
  source 1..360: mol_type = other DNA
gaggtgcagctgttggagtctgggggaggcttggtaccgcctggggagtccctgagactctcctgtgcagcctctggattcacctttagcacctctgccatgagctgggtccgccaggctccagggaaggggctggagtgggtctcaggtataagtagtagtggttttaatacatattatgcagactccgttaagggccggttcatcatttccagagacaattccaagaacacgctgtatctgcaaatgaacagcctgagagccgaggacacggccgtatattactgtgcgaaaggaggggtatttgtgatggcgattgcttacgtctggggcctagggaccacggtcaccgtctcctca

SEQ ID NO: 148 - protein, 120 aa, synthetic construct
EVQLLESGGGLVPPGESLRLSCAASGFTFSTSAMSWVRQAPGKGLEWVSGISSSGFNTYYADSVKGRFIISRDNSKNTLYLQMNSLRAEDTAVYYCAKGGVFVMAIAYVWGLGTTVTVSS

SEQ ID NO: 149 - DNA, 24 bp, synthetic construct
  source 1..24: mol_type = other DNA
ggattcacctttagcacctctgcc

SEQ ID NO: 150 - protein, 8 aa, synthetic construct
GFTFSTSA

SEQ ID NO: 151 - DNA, 24 bp, synthetic construct
  source 1..24: mol_type = other DNA
ataagtagtagtggttttaataca

SEQ ID NO: 152 - protein, 8 aa, synthetic construct
ISSSGFNT

SEQ ID NO: 153 - DNA, 39 bp, synthetic construct
  source 1..39: mol_type = other DNA
gcgaaaggaggggtatttgtgatggcgattgcttacgtc

SEQ ID NO: 154 - protein, 13 aa, synthetic construct
AKGGVFVMAIAYV

SEQ ID NO: 155 - DNA, 1341 bp, synthetic construct
  source 1..1341: mol_type = other DNA
gaggtgcagctgttggagtctgggggaggcttggtaccgcctggggagtccctgagactctcctgtgcagcctctggattcacctttagcacctctgccatgagctgggtccgccaggctccagggaaggggctggagtgggtctcaggtataagtagtagtggttttaatacatattatgcagactccgttaagggccggttcatcatttccagagacaattccaagaacacgctgtatctgcaaatgaacagcctgagagccgaggacacggccgtatattactgtgcgaaaggaggggtatttgtgatggcgattgcttacgtctggggcctagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 156 - protein, 446 aa, synthetic construct
EVQLLESGGGLVPPGESLRLSCAASGFTFSTSAMSWVRQAPGKGLEWVSGISSSGFNTYYADSVKGRFIISRDNSKNTLYLQMNSLRAEDTAVYYCAKGGVFVMAIAYVWGLGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - DNA, 324 bp, synthetic construct
  source 1..324: mol_type = other DNA
gaaagtgtgatgacgcagtctccaggcaccctgtctttgtctccaggggaaagagccaccctctcctgtagggccagtcagagtattagcagcagttatttagcctggtaccaacagagacctggccaggctcccaggctcctcatctatggtgcatccagcagggccactggcatccccgacaggttcagtggcagtgggtctgggacagacttcactctcaccatcagcagactggagcctgaagatattgcagtgtattactgtcagcagtatggtagctcaccttggacgttcggccaagggaccaaggtggaaatcaaa

SEQ ID NO: 158 - protein, 108 aa, synthetic construct
ESVMTQSPGTLSLSPGERATLSCRASQSISSSYLAWYQQRPGQAPRLLIYGASSRATGIPDRFSGSGSGTDFTLTISRLEPEDIAVYYCQQYGSSPWTFGQGTKVEIK

SEQ ID NO: 159 - DNA, 21 bp, synthetic construct
  source 1..21: mol_type = other DNA
cagagtattagcagcagttat

SEQ ID NO: 160 - protein, 7 aa, synthetic construct
QSISSSY

SEQ ID NO: 161 - skipped

SEQ ID NO: 162 - DNA, 27 bp, synthetic construct
  source 1..27: mol_type = other DNA
cagcagtatggtagctcaccttggacg

SEQ ID NO: 163 - DNA, 1353 bp, synthetic construct
  source 1..1353: mol_type = other DNA
gaggtgcagctgttggagtctgggggaggcttggtaccgcctggggagtccctgagactctcctgtgcagcctctggattcacctttagcacctctgccatgagctgggtccgccaggctccagggaaggggctggagtgggtctcaggtataagtagtagtggttttaatacatattatgcagactccgttaagggccggttcatcatttccagagacaattccaagaacacgctgtatctgcaaatgaacagcctgagagccgaggacacggccgtatattactgtgcgaaaggaggggtatttgtgatggcgattgcttacgtctggggcctagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 164 - protein, 450 aa, synthetic construct
EVQLLESGGGLVPPGESLRLSCAASGFTFSTSAMSWVRQAPGKGLEWVSGISSSGFNTYYADSVKGRFIISRDNSKNTLYLQMNSLRAEDTAVYYCAKGGVFVMAIAYVWGLGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - DNA, 648 bp, synthetic construct
  source 1..648: mol_type = other DNA
gaaagtgtgatgacgcagtctccaggcaccctgtctttgtctccaggggaaagagccaccctctcctgtagggccagtcagagtattagcagcagttatttagcctggtaccaacagagacctggccaggctcccaggctcctcatctatggtgcatccagcagggccactggcatccccgacaggttcagtggcagtgggtctgggacagacttcactctcaccatcagcagactggagcctgaagatattgcagtgtattactgtcagcagtatggtagctcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 166 - protein, 215 aa, synthetic construct
ESVMTQSPGTLSLSPGERATLSCRASQSISSSYLAWYQQRPGQAPRLLIYGASSRATGIPDRFSGSGSGTDFTLTISRLEPEDIAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - DNA, 378 bp, synthetic construct
  source 1..378: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgaccacagcctacatggagctgaggagcctgagatctgacgacacggccgtgtattactgtgcgagaggggaattatatagtggctacgatggaacacttggtattctctttgactactggggccagggaaccctggtcaccgtctcctca

SEQ ID NO: 168 - protein, 126 aa, synthetic construct
QVQLVQSGAEVKKPGASVKVSCKASGYTFTSYGISWVRQAPGQGLEWMGWISAYNGNTNYAQKLQGRVTMTTDTSTTTAYMELRSLRSDDTAVYYCARGELYSGYDGTLGILFDYWGQGTLVTVSS

SEQ ID NO: 169 - DNA, 24 bp, synthetic construct
  source 1..24: mol_type = other DNA
ggttacacctttaccagctatggt

SEQ ID NO: 170 - protein, 8 aa, synthetic construct
GYTFTSYG

SEQ ID NO: 171 - DNA, 24 bp, synthetic construct
  source 1..24: mol_type = other DNA
atcagcgcttacaatggtaacaca

SEQ ID NO: 172 - protein, 8 aa, synthetic construct
ISAYNGNT

SEQ ID NO: 173 - DNA, 57 bp, synthetic construct
  source 1..57: mol_type = other DNA
gcgagaggggaattatatagtggctacgatggaacacttggtattctctttgactac

SEQ ID NO: 174 - protein, 19 aa, synthetic construct
ARGELYSGYDGTLGILFDY

SEQ ID NO: 175 - DNA, 1359 bp, synthetic construct
  source 1..1359: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgaccacagcctacatggagctgaggagcctgagatctgacgacacggccgtgtattactgtgcgagaggggaattatatagtggctacgatggaacacttggtattctctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 176 - protein, 452 aa, synthetic construct
QVQLVQSGAEVKKPGASVKVSCKASGYTFTSYGISWVRQAPGQGLEWMGWISAYNGNTNYAQKLQGRVTMTTDTSTTTAYMELRSLRSDDTAVYYCARGELYSGYDGTLGILFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 177 - DNA, 366 bp, synthetic construct
  source 1..366: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggtagcagcagctactacgtggggggctggttcgacccctggggccagggaaccctggtcaccgtctcctca

SEQ ID NO: 178 - protein, 122 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKGSSSYYVGGWFDPWGQGTLVTVSS

SEQ ID NO: 179 - DNA, 24 bp, synthetic construct
  source 1..24: mol_type = other DNA
ggattcacctttgatgattatgcc

SEQ ID NO: 180 - protein, 8 aa, synthetic construct
GFTFDDYA

SEQ ID NO: 181 - DNA, 24 bp, synthetic construct
  source 1..24: mol_type = other DNA
attagttggaatagtggtagcata

SEQ ID NO: 182 - protein, 8 aa, synthetic construct
ISWNSGSI

SEQ ID NO: 183 - DNA, 45 bp, synthetic construct
  source 1..45: mol_type = other DNA
gcaaaaggtagcagcagctactacgtggggggctggttcgacccc

SEQ ID NO: 184 - protein, 15 aa, synthetic construct
AKGSSSYYVGGWFDP

SEQ ID NO: 185 - DNA, 1347 bp, synthetic construct
  source 1..1347: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaaggtagcagcagctactacgtggggggctggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 186 - protein, 448 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKGSSSYYVGGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 187 - DNA, 363 bp, synthetic construct
  source 1..363: mol_type = other DNA
gaggtgcagctgttggagtttgggggaggcttggtacagcctggggggtccctgagactctcctgtgcagcctctggattcacctttagcagctatgccatgacctgggtccgccaggctccagggaaggggctggagtgggtctcagctattagtggtagtggtggtagcacatactacgcagactccgtgaagggccggttcaccatctccagagacaattccaagaacacgttgaatctgcaaatgaacagcctgagagccgaggacacggccgtatattactgtgcgaaagggggttatatcacctcgctcccatacttccagcactggggccagggcaccctggtcaccgtctcctca

SEQ ID NO: 188 - protein, 121 aa, synthetic construct
EVQLLEFGGGLVQPGGSLRLSCAASGFTFSSYAMTWVRQAPGKGLEWVSAISGSGGSTYYADSVKGRFTISRDNSKNTLNLQMNSLRAEDTAVYYCAKGGYITSLPYFQHWGQGTLVTVSS

SEQ ID NO: 189 - DNA, 24 bp, synthetic construct
  source 1..24: mol_type = other DNA
ggattcacctttagcagctatgcc

SEQ ID NO: 190 - protein, 8 aa, synthetic construct
GFTFSSYA

SEQ ID NO: 191 - DNA, 24 bp, synthetic construct
  source 1..24: mol_type = other DNA
attagtggtagtggtggtagcaca

SEQ ID NO: 192 - protein, 8 aa, synthetic construct
ISGSGGST

SEQ ID NO: 193 - DNA, 42 bp, synthetic construct
  source 1..42: mol_type = other DNA
gcgaaagggggttatatcacctcgctcccatacttccagcac

SEQ ID NO: 194 - protein, 14 aa, synthetic construct
AKGGYITSLPYFQH

SEQ ID NO: 195 - DNA, 1344 bp, synthetic construct
  source 1..1344: mol_type = other DNA
gaggtgcagctgttggagtttgggggaggcttggtacagcctggggggtccctgagactctcctgtgcagcctctggattcacctttagcagctatgccatgacctgggtccgccaggctccagggaaggggctggagtgggtctcagctattagtggtagtggtggtagcacatactacgcagactccgtgaagggccggttcaccatctccagagacaattccaagaacacgttgaatctgcaaatgaacagcctgagagccgaggacacggccgtatattactgtgcgaaagggggttatatcacctcgctcccatacttccagcactggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 196 - protein, 447 aa, synthetic construct
EVQLLEFGGGLVQPGGSLRLSCAASGFTFSSYAMTWVRQAPGKGLEWVSAISGSGGSTYYADSVKGRFTISRDNSKNTLNLQMNSLRAEDTAVYYCAKGGYITSLPYFQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 197 - DNA, 357 bp, synthetic construct
  source 1..357: mol_type = other DNA
caggttcagctggtgcagtctggagctgaggtgaagaagcctggggcctcagtgaaggtctcctgcaaggcttctggttacacctttaccagctatggtatcagctggatgcgacaggcccctggacaagggcttgagtggatgggatggatcagcgcttacaatggtaatatatactatgcacagaggctccagggcagagtcaccatgaccacagacacatccacgagcacagcctacatggagctgaggagcctgagatctgacgacacggccgtttattactgtgcgacaacaactggagcgtgggggtggttcgacccctggggccagggaaccctggtcaccgtctcctca

SEQ ID NO: 198 - protein, 119 aa, synthetic construct
QVQLVQSGAEVKKPGASVKVSCKASGYTFTSYGISWMRQAPGQGLEWMGWISAYNGNIYYAQRLQGRVTMTTDTSTSTAYMELRSLRSDDTAVYYCATTTGAWGWFDPWGQGTLVTVSS

SEQ ID NO: 199 - DNA, 24 bp, synthetic construct
  source 1..24: mol_type = other DNA
atcagcgcttacaatggtaatata

SEQ ID NO: 200 - protein, 8 aa, synthetic construct
ISAYNGNI

SEQ ID NO: 201 - DNA, 36 bp, synthetic construct
  source 1..36: mol_type = other DNA
gcgacaacaactggagcgtgggggtggttcgacccc

SEQ ID NO: 202 - protein, 12 aa, synthetic construct
ATTTGAWGWFDP

SEQ ID NO: 203 - DNA, 1338 bp, synthetic construct
  source 1..1338: mol_type = other DNA
caggttcagctggtgcagtctggagctgaggtgaagaagcctggggcctcagtgaaggtctcctgcaaggcttctggttacacctttaccagctatggtatcagctggatgcgacaggcccctggacaagggcttgagtggatgggatggatcagcgcttacaatggtaatatatactatgcacagaggctccagggcagagtcaccatgaccacagacacatccacgagcacagcctacatggagctgaggagcctgagatctgacgacacggccgtttattactgtgcgacaacaactggagcgtgggggtggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 204 - protein, 445 aa, synthetic construct
QVQLVQSGAEVKKPGASVKVSCKASGYTFTSYGISWMRQAPGQGLEWMGWISAYNGNIYYAQRLQGRVTMTTDTSTSTAYMELRSLRSDDTAVYYCATTTGAWGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 205 - DNA, 1350 bp, synthetic construct
  source 1..1350: mol_type = other DNA
caggttcagctggtgcagtctggagctgaggtgaagaagcctggggcctcagtgaaggtctcctgcaaggcttctggttacacctttaccagctatggtatcagctggatgcgacaggcccctggacaagggcttgagtggatgggatggatcagcgcttacaatggtaatatatactatgcacagaggctccagggcagagtcaccatgaccacagacacatccacgagcacagcctacatggagctgaggagcctgagatctgacgacacggccgtttattactgtgcgacaacaactggagcgtgggggtggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 206 - protein, 449 aa, synthetic construct
QVQLVQSGAEVKKPGASVKVSCKASGYTFTSYGISWMRQAPGQGLEWMGWISAYNGNIYYAQRLQGRVTMTTDTSTSTAYMELRSLRSDDTAVYYCATTTGAWGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - DNA, 360 bp, synthetic construct
  source 1..360: mol_type = other DNA
gaggtgcagctggtggagtctgggggaggcctggtcaagccgggggggtccctgagaatttactgtgcagcctctggattcaccttcagaacctatagcatgaactgggtccgccaggctccagggaaggggctggagtgggtctcatccattagtagcagtagtagttacatacagtatgaagactcagtgaagggccgattcaccatgtccagagacaacgccaagaactcactgtatctgcaaatgaacagcctgagagccgaggacacggctgtgtattattgtgtgagagggggggtagtagtaccagctgctatggtctactggggccagggaaccctggtcaccgtctcctca

SEQ ID NO: 208 - protein, 120 aa, synthetic construct
EVQLVESGGGLVKPGGSLRIYCAASGFTFRTYSMNWVRQAPGKGLEWVSSISSSSSYIQYEDSVKGRFTMSRDNAKNSLYLQMNSLRAEDTAVYYCVRGGVVVPAAMVYWGQGTLVTVSS

SEQ ID NO: 209 - DNA, 24 bp, synthetic construct
  source 1..24: mol_type = other DNA
ggattcaccttcagaacctatagc

SEQ ID NO: 210 - protein, 8 aa, synthetic construct
GFTFRTYS

SEQ ID NO: 211 - DNA, 1341 bp, synthetic construct
  source 1..1341: mol_type = other DNA
gaggtgcagctggtggagtctgggggaggcctggtcaagccgggggggtccctgagaatttactgtgcagcctctggattcaccttcagaacctatagcatgaactgggtccgccaggctccagggaaggggctggagtgggtctcatccattagtagcagtagtagttacatacagtatgaagactcagtgaagggccgattcaccatgtccagagacaacgccaagaactcactgtatctgcaaatgaacagcctgagagccgaggacacggctgtgtattattgtgtgagagggggggtagtagtaccagctgctatggtctactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 212 - protein, 446 aa, synthetic construct
EVQLVESGGGLVKPGGSLRIYCAASGFTFRTYSMNWVRQAPGKGLEWVSSISSSSSYIQYEDSVKGRFTMSRDNAKNSLYLQMNSLRAEDTAVYYCVRGGVVVPAAMVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - DNA, 1323 bp, synthetic construct
  source 1..1323: mol_type = other DNA
caggtgcagctggtacagtctggggctgagatgaagaagcctggggactcagtgaaggtctcctgcaaggcttctggatacaccttctccgtctactatatgcattgggtgcgacaggcccctgggcaagggcttgagtggatgggatggatcaaccctaaaagtggtggcacaaactatgcacagaggtttcagggcagggtcgccatgaccagggacacgtccatcaccacagcctacatggaactaaggagactgagatctgacgacatggccgtatattattgtgtaagaagggggattgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 214 - protein, 440 aa, synthetic construct
QVQLVQSGAEMKKPGDSVKVSCKASGYTFSVYYMHWVRQAPGQGLEWMGWINPKSGGTNYAQRFQGRVAMTRDTSITTAYMELRRLRSDDMAVYYCVRRGIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 215 - DNA, 372 bp, synthetic construct
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagc

SEQ ID NO: 216 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 217 - DNA, 24 bp, synthetic construct
  source 1..24: mol_type = other DNA
gggtttacattcgacgattacagc

SEQ ID NO: 218 - protein, 8 aa, synthetic construct
GFTFDDYS

SEQ ID NO: 219 - DNA, 24 bp, synthetic construct
  source 1..24: mol_type = other DNA
atatcatggaactcaggaagcaag

SEQ ID NO: 220 - protein, 8 aa, synthetic construct
ISWNSGSK

SEQ ID NO: 221 - DNA, 51 bp, synthetic construct
  source 1..51: mol_type = other DNA
gcaaaatacggcagtggttatggcaagttttatcattatggactggacgtg

SEQ ID NO: 222 - protein, 17 aa, synthetic construct
AKYGSGYGKFYHYGLDV

SEQ ID NO: 223 - DNA, 372 bp, synthetic construct
  source 1..372: mol_type = other DNA
gaagtgcagctggtggagtctgggggaggcttggtgcagcctggcaggtccctgagactctcctgtgcagcctctggattcacctttgctgattataccatgcactgggtccggcaagctccagggaagggcctggagtgggtctcagatattagttggaatagtggtagtatagcctatgcggactctgtgaagggccgattcaccatctccagagacaacgccaagaactccctctatcttcaaatgaacagtctgagaactgaggacacggccttttattactgtgcaaaagatagtaggggctacggtcactataagtacctcggtttggacgtctggggccaagggaccacggtcaccgtctcctca

SEQ ID NO: 224 - protein, 124 aa, synthetic construct
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSS

SEQ ID NO: 225 - DNA, 24 bp, synthetic construct
  source 1..24: mol_type = other DNA
ggattcacctttgctgattatacc

SEQ ID NO: 226 - protein, 8 aa, synthetic construct
GFTFADYT

SEQ ID NO: 227 - DNA, 24 bp, synthetic construct
  source 1..24: mol_type = other DNA
attagttggaatagtggtagtata

SEQ ID NO: 228 - protein, 8 aa, synthetic construct
ISWNSGSI

SEQ ID NO: 229 - DNA, 51 bp, synthetic construct
  source 1..51: mol_type = other DNA
gcaaaagatagtaggggctacggtcactataagtacctcggtttggacgtc

SEQ ID NO: 230 - protein, 17 aa, synthetic construct
AKDSRGYGHYKYLGLDV

SEQ ID NO: 231 - DNA, 324 bp, synthetic construct; identical to SEQ ID NO: 9
  source 1..324: mol_type = other DNA

SEQ ID NO: 232 - protein, 108 aa, synthetic construct; identical to SEQ ID NO: 10

SEQ ID NO: 233 - DNA, 18 bp, synthetic construct
  source 1..18: mol_type = other DNA
cagagcattagcagctat

SEQ ID NO: 234 - protein, 6 aa, synthetic construct
QSISSY

SEQ ID NO: 235-236 - skipped (2 entries)

SEQ ID NO: 237 - DNA, 30 bp, synthetic construct; identical to SEQ ID NO: 15
  source 1..30: mol_type = other DNA

SEQ ID NO: 238 - protein, 10 aa, synthetic construct
QQSYSTPPIT

SEQ ID NO: 239 - DNA, 1353 bp, synthetic construct
  source 1..1353: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagcgccagcacaaaaggtcctagcgtttttccacttgccccatgttcaaggtcaacctccgaaagtaccgccgctcttggctgtctcgtaaaagattattttcccgaacctgtaactgtctcctggaactccggcgcactcacttccggcgtacataccttccccgctgtcctccaatcttccggtctctactccctgtcttctgttgtcactgttccatcatcctcactcggcacaaaaacatatacctgcaacgttgatcacaagccaagtaataccaaagttgataagcgcgtcgaatccaaatacggtcccccctgccccccatgtcccgctccacctgtggctggtccctctgttttcctttttccccctaaacccaaagataccctcatgatttccagaacccccgaggtcacctgcgtcgtcgttgatgtaagccaagaagatcccgaagtccagttcaattggtatgtagacggtgttgaagtccataatgcaaaaacaaaacccagagaggaacagtttaattcaacctatcgtgtcgttagcgtactcaccgttcttcatcaagactggctcaatggaaaagaatataaatgtaaagttagcaacaaaggtctgcccagttcaatcgaaaaaacaattagcaaagccaaaggccaacctcgcgaaccccaagtctataccttgcccccttctcaggaagaaatgaccaaaaaccaagtttcactcacatgcctcgtaaaaggattctatccatcagacattgcagtagaatgggaatctaacggccaacctgaaaataattacaaaaccactcctcctgtcctcgattctgacggctcttttttcctttactccagattgactgttgataaatcccgctggcaggaaggtaacgttttttcttgttctgtgatgcacgaagccctccataacagattcactcaaaaatctctttctctctcccctggcaaataa

SEQ ID NO: 240 - protein, 450 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 241 - DNA, 1353 bp, synthetic construct
  source 1..1353: mol_type = other DNA
gaagtgcagctggtggagtctgggggaggcttggtgcagcctggcaggtccctgagactctcctgtgcagcctctggattcacctttgctgattataccatgcactgggtccggcaagctccagggaagggcctggagtgggtctcagatattagttggaatagtggtagtatagcctatgcggactctgtgaagggccgattcaccatctccagagacaacgccaagaactccctctatcttcaaatgaacagtctgagaactgaggacacggccttttattactgtgcaaaagatagtaggggctacggtcactataagtacctcggtttggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 242 - protein, 450 aa, synthetic construct
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 243 - DNA, 648 bp, synthetic construct; identical to SEQ ID NO: 19
  source 1..648: mol_type = other DNA

SEQ ID NO: 244 - protein, 215 aa, synthetic construct; identical to SEQ ID NO: 20

SEQ ID NO: 245 - DNA, 648 bp, synthetic construct; identical to SEQ ID NO: 75
  source 1..648: mol_type = other DNA

SEQ ID NO: 246 - protein, 215 aa, synthetic construct; identical to SEQ ID NO: 76

SEQ ID NO: 247 - DNA, 1344 bp, synthetic construct; identical to SEQ ID NO: 195
  source 1..1344: mol_type = other DNA

SEQ ID NO: 248 - protein, 447 aa, synthetic construct; identical to SEQ ID NO: 196

SEQ ID NO: 249 - DNA, 1323 bp, synthetic construct; identical to SEQ ID NO: 213
  source 1..1323: mol_type = other DNA

SEQ ID NO: 250 - protein, 440 aa, synthetic construct; identical to SEQ ID NO: 214

SEQ ID NO: 251 - DNA, 1341 bp, synthetic construct; identical to SEQ ID NO: 211
  source 1..1341: mol_type = other DNA

SEQ ID NO: 252 - protein, 446 aa, synthetic construct; identical to SEQ ID NO: 212

SEQ ID NO: 253 - DNA, 1341 bp, synthetic construct; identical to SEQ ID NO: 29
  source 1..1341: mol_type = other DNA

SEQ ID NO: 254 - protein, 446 aa, synthetic construct; identical to SEQ ID NO: 30

SEQ ID NO: 255 - DNA, 1341 bp, synthetic construct; identical to SEQ ID NO: 17
  source 1..1341: mol_type = other DNA

SEQ ID NO: 256 - protein, 446 aa, synthetic construct; identical to SEQ ID NO: 18

SEQ ID NO: 257 - DNA, 1350 bp, synthetic construct; identical to SEQ ID NO: 73
  source 1..1350: mol_type = other DNA

SEQ ID NO: 258 - protein, 449 aa, synthetic construct; identical to SEQ ID NO: 74

SEQ ID NO: 259 - DNA, 1347 bp, synthetic construct; identical to SEQ ID NO: 185
  source 1..1347: mol_type = other DNA

SEQ ID NO: 260 - protein, 448 aa, synthetic construct; identical to SEQ ID NO: 186

SEQ ID NO: 261 - DNA, 1359 bp, synthetic construct; identical to SEQ ID NO: 175
  source 1..1359: mol_type = other DNA

SEQ ID NO: 262 - protein, 452 aa, synthetic construct; identical to SEQ ID NO: 176

SEQ ID NO: 263 - DNA, 1338 bp, synthetic construct; identical to SEQ ID NO: 203
  source 1..1338: mol_type = other DNA

SEQ ID NO: 264 - protein, 445 aa, synthetic construct; identical to SEQ ID NO: 204

SEQ ID NO: 265 - protein, 457 aa, synthetic construct
HHHHHHEQKLISEEDLGGEQKLISEEDLRAPRCRELPAQKWWHTGALYRIGDLQAFQGHGAGNLAAPISGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 266 - protein, 14 aa, synthetic construct
QGHGAGNLAAPISG

SEQ ID NO: 267 - protein, 675 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKAKFVAAWTLKAAARAPRCRELPAQKWWHTGALYRIGDLQAFQGHGAGNLAAPISGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 268 - DNA, 372 bp, synthetic construct
  source 1..372: mol_type = other DNA
cagatcaccttgaaggagtctggtcctacgctggtgaaacccacacagaccctcacgctgacctgcaccttctctgggttctcactcaccactagtggagtgggtgtgggctggatccgtcagcccccaggaaaggccctggagtggcttgcactcatttattggaatgatggtaaggactacagcccgtctctgaagagcaggctcaccatcaccaaggacacctccaaaaaccaggtggtccttacaatgaccaacatggaccctgttgacacagccacatattactgtgcacacagacgaaaatacgatttttggcgtggttattataaggactactggggccagggaaccctggtcaccgtctcctca

SEQ ID NO: 269 - protein, 124 aa, synthetic construct
QITLKESGPTLVKPTQTLTLTCTFSGFSLTTSGVGVGWIRQPPGKALEWLALIYWNDGKDYSPSLKSRLTITKDTSKNQVVLTMTNMDPVDTATYYCAHRRKYDFWRGYYKDYWGQGTLVTVSS

SEQ ID NO: 270 - DNA, 30 bp, synthetic construct
  source 1..30: mol_type = other DNA
gggttctcactcaccactagtggagtgggt

SEQ ID NO: 271 - protein, 10 aa, synthetic construct
GFSLTTSGVG

SEQ ID NO: 272 - DNA, 21 bp, synthetic construct
  source 1..21: mol_type = other DNA
atttattggaatgatggtaag

SEQ ID NO: 273 - protein, 7 aa, synthetic construct
IYWNDGK

SEQ ID NO: 274 - DNA, 48 bp, synthetic construct
  source 1..48: mol_type = other DNA
gcacacagacgaaaatacgatttttggcgtggttattataaggactac

SEQ ID NO: 275 - protein, 16 aa, synthetic construct
AHRRKYDFWRGYYKDY

SEQ ID NO: 276 - DNA, 324 bp, synthetic construct; identical to SEQ ID NO: 39
  source 1..324: mol_type = other DNA

SEQ ID NO: 277 - protein, 108 aa, synthetic construct; identical to SEQ ID NO: 40

SEQ ID NO: 278 - DNA, 21 bp, synthetic construct
  source 1..21: mol_type = other DNA
cagtcagtctctagctcttat

SEQ ID NO: 279 - protein, 7 aa, synthetic construct
QSVSSSY

SEQ ID NO: 280-281 - skipped (2 entries)

SEQ ID NO: 282 - DNA, 27 bp, synthetic construct
  source 1..27: mol_type = other DNA
caacagtacggaagcagcccgtggacg

SEQ ID NO: 283 - protein, 9 aa, synthetic construct
QQYGSSPWT

SEQ ID NO: 284 - DNA, 1356 bp, synthetic construct; identical to SEQ ID NO: 97
  source 1..1356: mol_type = other DNA

SEQ ID NO: 285 - protein, 451 aa, synthetic construct; identical to SEQ ID NO: 98

SEQ ID NO: 286 - DNA, 1353 bp, synthetic construct
  source 1..1353: mol_type = other DNA
cagatcaccttgaaggagtctggtcctacgctggtgaaacccacacagaccctcacgctgacctgcaccttctctgggttctcactcaccactagtggagtgggtgtgggctggatccgtcagcccccaggaaaggccctggagtggcttgcactcatttattggaatgatggtaaggactacagcccgtctctgaagagcaggctcaccatcaccaaggacacctccaaaaaccaggtggtccttacaatgaccaacatggaccctgttgacacagccacatattactgtgcacacagacgaaaatacgatttttggcgtggttattataaggactactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 287 - protein, 450 aa, synthetic construct
QITLKESGPTLVKPTQTLTLTCTFSGFSLTTSGVGVGWIRQPPGKALEWLALIYWNDGKDYSPSLKSRLTITKDTSKNQVVLTMTNMDPVDTATYYCAHRRKYDFWRGYYKDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 288 - DNA, 648 bp, synthetic construct; identical to SEQ ID NO: 49
  source 1..648: mol_type = other DNA

SEQ ID NO: 289 - protein, 215 aa, synthetic construct; identical to SEQ ID NO: 50

SEQ ID NO: 290 - DNA, 372 bp, synthetic construct
  source 1..372: mol_type = other DNA
cagatcaccttgaaggagtctggtcctacgctggtgaaacccacacagaccctcacgctgacctgcaccttctctggattctcactcagtactagtggagtgggtgtgggctggatccgtcagcccccaggaaaggccctggaatggcttacactcattttttggaatgatgataagcgcttcagtccatctctgaagagcaggattaccatcaacaaggacacctccaaagaccaggtggtccttacaatgaccaacatggaccctgtggacacaggcacatattactgtatatatagacgttcttacgatttttggaggggttattataaggacttctggggccagggaaccctggtcaccgtctcctca

SEQ ID NO: 291 - protein, 124 aa, synthetic construct
QITLKESGPTLVKPTQTLTLTCTFSGFSLSTSGVGVGWIRQPPGKALEWLTLIFWNDDKRFSPSLKSRITINKDTSKDQVVLTMTNMDPVDTGTYYCIYRRSYDFWRGYYKDFWGQGTLVTVSS

SEQ ID NO: 292 - DNA, 30 bp, synthetic construct
  source 1..30: mol_type = other DNA
ggattctcactcagtactagtggagtgggt

SEQ ID NO: 293 - protein, 10 aa, synthetic construct
GFSLSTSGVG

SEQ ID NO: 294 - DNA, 21 bp, synthetic construct
  source 1..21: mol_type = other DNA
attttttggaatgatgataag

SEQ ID NO: 295 - protein, 7 aa, synthetic construct
IFWNDDK

SEQ ID NO: 296 - DNA, 48 bp, synthetic construct
  source 1..48: mol_type = other DNA
atatatagacgttcttacgatttttggaggggttattataaggacttc

SEQ ID NO: 297 - protein, 16 aa, synthetic construct
IYRRSYDFWRGYYKDF

SEQ ID NO: 298 - DNA, 1353 bp, synthetic construct
  source 1..1353: mol_type = other DNA
cagatcaccttgaaggagtctggtcctacgctggtgaaacccacacagaccctcacgctgacctgcaccttctctggattctcactcagtactagtggagtgggtgtgggctggatccgtcagcccccaggaaaggccctggaatggcttacactcattttttggaatgatgataagcgcttcagtccatctctgaagagcaggattaccatcaacaaggacacctccaaagaccaggtggtccttacaatgaccaacatggaccctgtggacacaggcacatattactgtatatatagacgttcttacgatttttggaggggttattataaggacttctggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 299 - protein, 450 aa, synthetic construct
QITLKESGPTLVKPTQTLTLTCTFSGFSLSTSGVGVGWIRQPPGKALEWLTLIFWNDDKRFSPSLKSRITINKDTSKDQVVLTMTNMDPVDTGTYYCIYRRSYDFWRGYYKDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 300 - DNA, 372 bp, synthetic construct
  source 1..372: mol_type = other DNA
cagatcaccttgaaggaatctggtcctacgctggtgaaacccacacagaccctcacgctgacctgcaccttctctgggttctcactcagtactagtggagtgggtgtgggctggatccgtcagcccccaggaaaggccctggaatggcttgcactcctttattggaatgataatgagcggtacagtccatctctgaagaacagactcaccatcaccaaggacacttccaaaaaccaggtggtccttacagtgaccaacatggaccctgtggactcagccacatattactgtatatacaggcgtcaatacgatttttggcgtggttattatcgtgacgactggggccagggaaccctggtcaccgtctcctca

SEQ ID NO: 301 - protein, 124 aa, synthetic construct
QITLKESGPTLVKPTQTLTLTCTFSGFSLSTSGVGVGWIRQPPGKALEWLALLYWNDNERYSPSLKNRLTITKDTSKNQVVLTVTNMDPVDSATYYCIYRRQYDFWRGYYRDDWGQGTLVTVSS

SEQ ID NO: 302 - DNA, 30 bp, synthetic construct
  source 1..30: mol_type = other DNA
gggttctcactcagtactagtggagtgggt

SEQ ID NO: 303 - DNA, 21 bp, synthetic construct
  source 1..21: mol_type = other DNA
ctttattggaatgataatgag

SEQ ID NO: 304 - protein, 7 aa, synthetic construct
LYWNDNE

SEQ ID NO: 305 - DNA, 48 bp, synthetic construct
  source 1..48: mol_type = other DNA
atatacaggcgtcaatacgatttttggcgtggttattatcgtgacgac

SEQ ID NO: 306 - protein, 16 aa, synthetic construct
IYRRQYDFWRGYYRDD

SEQ ID NO: 307 - DNA, 1353 bp, synthetic construct
  source 1..1353: mol_type = other DNA
cagatcaccttgaaggaatctggtcctacgctggtgaaacccacacagaccctcacgctgacctgcaccttctctgggttctcactcagtactagtggagtgggtgtgggctggatccgtcagcccccaggaaaggccctggaatggcttgcactcctttattggaatgataatgagcggtacagtccatctctgaagaacagactcaccatcaccaaggacacttccaaaaaccaggtggtccttacagtgaccaacatggaccctgtggactcagccacatattactgtatatacaggcgtcaatacgatttttggcgtggttattatcgtgacgactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 308 - protein, 450 aa, synthetic construct
QITLKESGPTLVKPTQTLTLTCTFSGFSLSTSGVGVGWIRQPPGKALEWLALLYWNDNERYSPSLKNRLTITKDTSKNQVVLTVTNMDPVDSATYYCIYRRQYDFWRGYYRDDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

(19) United States
(12) Patent Application Publication
(10) Pub. No.: US 2026/0139069 A1
(43) Pub. Date: May 21, 2026

(54) ANTI-SLC3A2-APIS ANTIGEN-BINDING PROTEINS AND METHODS OF USE THEREOF

(71) Applicant: Regeneron Pharmaceuticals, Inc., Tarrytown, NY (US)

(72) Inventors: Michael Van Meter, Somers, NY (US); Frank Kuhnert, Yorktown Heights, NY (US)

(21) Appl. No.: 19/310,327

(22) Filed: Aug. 26, 2025

Related U.S. Application Data
(60) Provisional application No. 63/687,733, filed on Aug. 27, 2024.

(51) Int. Cl. C07K 16/28; A61K 39/00; A61K 45/06; A61P 35/00; C12N 15/85

(52) U.S. Cl. CPC C07K 16/2896; A61K 45/06; A61P 35/00; C07K 16/2809; C12N 15/85; A61K 2039/505; C07K 2317/14; C07K 2317/31; C07K 2317/32; C07K 2317/526; C07K 2317/53; C07K 2317/565; C07K 2317/71; C07K 2317/92

(57) ABSTRACT

The present disclosure provides monospecific antigen-binding proteins that bind human SLC3A2-APIS and bispecific antigen-binding proteins that bind to SLC3A2-APIS and CD3 (SLC3A2-APISxCD3). In certain embodiments, the disclosed bispecific antigen-binding proteins include a first antigen-binding domain that binds SLC3A2-APIS and a second antigen-binding domain that binds CD3. In some embodiments, the disclosed antigen-binding proteins inhibit tumor growth. In some embodiments, the disclosed antigen-binding proteins ameliorate one or more symptoms of a myelodysplastic syndrome.

### FIELD

The present disclosure relates to antigen-binding proteins, antibodies and antigen-binding fragments thereof that bind to SLC3A2-APIS, including monospecific anti-SLC3A2-APIS antibodies and antigen-binding fragments thereof and bispecific anti-SLC3A2-APISxCD3 antibodies and antigen-binding fragments thereof, and methods of use thereof, e.g., for treating or preventing cancer.

### SEQUENCE LISTING

The sequence listing of the present application is submitted electronically as an ST.26 formatted xml file with a file name “SeqList11887.xml,” creation date of Aug. 21, 2025, and a size of 353,833 bytes. This sequence listing submitted is part of the specification and is hereby incorporated by reference in its entirety.

### BACKGROUND

The spliceosome is a large ribonucleoprotein complex that catalyzes the removal of introns from precursor messenger RNA (pre-mRNA) through the process of splicing. Mutations in the genes encoding various components of the spliceosome, therefore, have broad effects on gene expression, and have been found to be related to a wide array of cancers. For example, the gene Splicing Factor 3b Subunit 1 (SF3B1) is frequently mutated in a wide range of solid and hematological tumor types, e.g., breast cancer, chronic lymphocytic leukemia, lung cancer, myelodysplastic syndrome (MDS), pancreatic cancer, prostate cancer, and uveal melanoma. SF3B1 mutations are found in approximately 30% of myelodysplastic syndrome cases and define a distinct disease subtype. Mutations in SF3B1 alter the activity of the tumor spliceosome with resultant broad pattern of tumor-specific aberrant pre-mRNA splicing. One consequence of this pattern of mutant SF3B1-mediated aberrant splicing is the generation of a variant spliceoform of the protein Solute Carrier Family 3 Member 2 (SLC3A2), termed SLC3A2-APIS. SLC3A2-APIS contains a four amino acid insertion-alanine, proline, isoleucine and serine (A-P-I-S) in its extracellular domain resulting from non-canonical 3′ splice site selection of the SF3B1 mutant spliceosome. While multiple SF3B1 mutant alleles have been described across diverse tumor types all major SF3B1 mutations are associated with the expression of SLC3A2-APIS. Because the APIS insertion is localized to SLC3A2 extracellular domain, mutant SF3B1 aberrant splicing of SLC3A2 pre-mRNA results in the expression of a cell-surface neoantigen that can be exploited as a tumor associated antigen (TAA), which can be targeted by SLC3A2-APIS directed immunotherapies. Monoclonal antibodies that specifically recognize this particular variant have great potential to be highly effective in treating the wide array of tumors that express SLC3A2-APIS.

Cluster of Differentiation 3 (CD3) is a homodimeric or heterodimeric antigen expressed on T cells in association with the T cell receptor complex (TCR) and is required for T cell activation. Functional CD3 is formed from the dimeric association of two of four different chains: epsilon, zeta, delta and gamma. The CD3 dimeric arrangements include gamma/epsilon, delta/epsilon and zeta/zeta. Antibodies against CD3 have been shown to cluster CD3 on T cells, thereby causing T cell activation in a manner similar to the engagement of the TCR by peptide-loaded MHC molecules. Thus, anti-CD3 antibodies have been proposed for therapeutic purposes involving the activation of T cells. In addition, bispecific antibodies that are capable of binding CD3 and a tumor associated antigen have been proposed for therapeutic uses involving targeting T cell immune responses to tissues and cells expressing the target antigen.

Accordingly, monospecific anti-SLC3A2-APIS antibodies as well as bispecific antibodies that bind both SLC3A2-APIS and CD3 would be useful in therapeutic settings in which specific targeting and T cell-mediated killing of the wide range of tumor cells that express SLC3A2-APIS is desired. Further, there is a need for additional treatments for cancer, such as immunotherapeutic agents that recognize SLC3A2-APIS.

### SUMMARY

The present disclosure provides antibodies or antigen-binding fragments thereof that selectively bind the APIS neoantigen variant of Solute Carrier Family 3 Member 2 (“SLC3A2-APIS”), including bispecific antibodies that bind both SLC3A2-APIS and CD3 (“SLC3A2-APISxCD3”). The bispecific SLC3A2-APISxCD3 antibodies or antigen-binding fragments thereof of the present disclosure bind to and engage SLC3A2-APIS expressing tumor cells while also targeting T cells expressing CD3 and stimulating T cell activation, e.g., under circumstances where T cell-mediated killing is desired or beneficial. SLC3A2-APIS is expressed on a wide range of different cancer types. As such, the anti-SLC3A2-APIS antibodies and antigen-binding fragments thereof disclosed herein provide an efficacious anti-tumor therapy against many tumor types. Further, the antigen-binding proteins of the present disclosure selectively bind to SLC3A2-APIS which is expressed on tumor cells, but do not bind to SLC3A2 which is expressed in normal tissues. Such preferential targeting of the antigen-binding molecule to tumor cells expressing SLC3A2-APIS may be useful to avoid general/untargeted binding and the consequent adverse side effects or toxicity.

In one aspect, the disclosed technology relates to an isolated antigen-binding protein including an antigen-binding domain that binds specifically to SLC3A2-APIS wherein the antigen-binding protein does not bind to wild-type SLC3A2, as measured by flow cytometry. In some embodiments, the antigen-binding domain interacts with at least one amino acid residue selected from the group consisting of D229, Q231, A232, Q234, G235, H236, G237, A238, G239, N240, L241, A242, A243, P244, S246, G247, K273, P286, N287, F288, Y316, R317, P487, D496, E497, S498, P501, D502, P504, and G505 contained in SLC3A2-APIS, as determined by cryo-electron microscopy. In some embodiments, the antigen-binding domain interacts with at least one amino acid residue selected from the group consisting of Q234, G235, H236, G237, A238, G239, N240, L241, A242, A243, P244, S246 and G247 contained in SLC3A2-APIS, as determined by cryo-electron microscopy. In some embodiments, the antigen-binding domain interacts with A243, P244, S246 contained within SLC3A2-APIS, as determined by cryo-electron microscopy. In some embodiments, the antigen-binding domain interacts withat least one amino acid residue selected from the group consisting of D229, Q231, A232, G235, H236, G237, A238, G239, N240, L241, A242, G247, K273, P286, N287, F288, Y316, R317, P487, D496, E497, S498, P501, D502, P504, and G505 contained in SLC3A2-APIS, as determined by cryo-electron microscopy. In some embodiments, the antigen-binding domain does not interact with A243, P244, or S246 contained within SLC3A2-APIS, as determined by cryo-electron microscopy.

In another aspect, the disclosed technology relates to an isolated antigen-binding protein including an antigen-binding domain that binds specifically to SLC3A2-APIS, wherein the antigen-binding domain (i) does not bind to wild-type SLC3A2; and (ii) interacts with one or more amino acids contained within SEQ ID NO: 265, as determined by cryo-electron microscopy. In some embodiments, the antigen-binding domain interacts with at least one amino acid contained within SEQ ID NO: 266, as determined by cryo-electron microscopy. In some embodiments, the antigen-binding domain interacts with at least ten amino acids contained within SEQ ID NO: 266, as determined by cryo-electron microscopy. In some embodiments, the antigen-binding domain does not interact with A243, P244, or S246 contained within SLC3A2-APIS, as determined by cryo-electron microscopy. In some embodiments, the SLC3A2-APIS is expressed on a tumor cell.

In some embodiments, the antigen-binding domain that binds specifically to SLC3A2-APIS includes: (a) three light chain complementarity determining regions (LCDR1, LCDR2, and LCDR3) contained within a light chain variable region (LCVR) including an amino acid sequence selected from SEQ ID NOs: 10, 40, 70, 112, 140, 158, and 232; and (b) three heavy chain complementarity determining regions (HCDR1, HCDR2, and HCDR3) contained within a heavy chain variable region (HCVR) including an amino acid sequence selected from SEQ ID NOs: 2, 22, 32, 52, 62, 78, 88, 104, 120, 130, 148, 168, 178, 188, 198, and 208. In some embodiments, the antigen-binding domain that binds specifically to SLC3A2-APIS includes three CDRs (LCDR1, LCDR2, and LCDR3) contained within a LCVR and three CDRs (HCDR1, HCDR2 and HCDR3) contained within a HCVR, wherein: LCDR1, LCDR2, and LCDR3 include respective amino acid sequences selected from (i) SEQ ID NO: 12, AAS, and SEQ ID NO: 16; (ii) SEQ ID NO: 42, GAS, and SEQ ID NO: 46; (iii) SEQ ID NO: 72, AAS, and SEQ ID NO: 16; (iv) SEQ ID NO: 114, AAS, and SEQ ID NO: 16; (v) SEQ ID NO: 12, AAS, and SEQ ID NO: 142, (vi) SEQ ID NO: 160, GAS, and SEQ ID NO: 46; and (vii) SEQ ID NO: 234, AAS, and SEQ ID NO: 238; and HCDR1, HCDR2, and HCDR3 include respective amino acid sequences selected from SEQ ID NOs: (i) 4, 6, and 8; (ii) 24, 26, and 28; (iii) 34, 36, and 38; (iv) 54, 56, and 58; (v) 64, 66, and 68; (vi) 80, 82, and 84; (vii) 90, 92, and 94; (viii) 106, 108, and 110; (ix) 122, 124, and 126; (x) 132, 134, and 136; (xi) 150, 152, and 154 (xii) 170, 172, and 174; (xiii) 170, 200, and 202; (xiv) 180, 182, and 184; (xv) 190, 192, and 194; and (xvi) 210, 6, and 8. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 are contained within a LCVR including the amino acid sequence of SEQ ID NO: 10; and HCDR1, HCDR2, and HCDR3 are contained within a HCVR including an amino acid sequence selected from SEQ ID NOs: 2, 22, 32, 52, 62, 78, 88, 104, 120, 130, 148, 168, 178, 188, 198, and 208.

In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 are contained within a LCVR including the amino acid sequence of SEQ ID NO: 40; and HCDR1, HCDR2, and HCDR3 are contained within a HCVR including an amino acid sequence selected from SEQ ID NOs: 2, 22, 32, 52, 62, 78, 88, 104, 120, 130, 148, 168, 178, 188, 198, and 208. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 are contained within a LCVR including the amino acid sequence of SEQ ID NO: 70; and HCDR1, HCDR2, and HCDR3 are contained within a HCVR including an amino acid sequence selected from SEQ ID NOs: 2, 22, 32, 52, 62, 78, 88, 104, 120, 130, 148, 168, 178, 188, 198, and 208. In some embodiments, the antigen-binding domain that binds specifically to SLC3A2-APIS includes three CDRs (LCDR1, LCDR2 and LCDR3) contained within a LCVR and three CDRs (HCDR1, HCDR2 and HCDR3) contained within a HCVR, wherein: LCDR1, LCDR2 and LCDR3 are contained within a LCVR including the amino acid sequence of SEQ ID NO: 112; and HCDR1, HCDR2 and HCDR3 are contained within a HCVR including an amino acid sequence selected from SEQ ID NOs: 2, 22, 32, 52, 62, 78, 88, 104, 120, 130, 148, 168, 178, 188, 198, and 208.

In some embodiments, the antigen-binding domain that binds specifically to SLC3A2-APIS includes three CDRs (LCDR1, LCDR2 and LCDR3) contained within a LCVR and three CDRs (HCDR1, HCDR2, and HCDR3) contained within a HCVR, wherein: LCDR1, LCDR2, and LCDR3 are contained within a LCVR including the amino acid sequence of SEQ ID NO: 140; and HCDR1, HCDR2 and HCDR3 are contained within a HCVR including an amino acid sequence selected from SEQ ID NOs: 2, 22, 32, 52, 62, 78, 88, 104, 120, 130, 148, 168, 178, 188, 198, and 208. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2 and LCDR3 are contained within a LCVR including the amino acid sequence of SEQ ID NO: 158; and HCDR1, HCDR2 and HCDR3 are contained within a HCVR including an amino acid sequence selected from SEQ ID NOs: 2, 22, 32, 52, 62, 78, 88, 104, 120, 130, 148, 168, 178, 188, 198, and 208. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: (a) LCDR1, LCDR2, and LCDR3 are contained within a LCVR including the amino acid sequence of SEQ ID NO: 232; and (b) HCDR1, HCDR2, and HCDR3 are contained within a HCVR including an amino acid sequence selected from SEQ ID NOs: 2, 22, 32, 52, 62, 78, 88, 104, 120, 130, 148, 168, 178, 188, 198, and 208.

In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein LCDR1, LCDR2, and LCDR3 are contained within a LCVR including the amino acid sequence of SEQ ID NO: 10; and HCDR1, HCDR2, and HCDR3 are contained within a HCVR including an amino acid sequence selected from SEQ ID NO: 2, 22, 62, 104, 168, 178, 188, 198, and 208. In some embodiments, the antigen-binding domain that binds specifically to SLC3A2-APIS includes three CDRs (LCDR1, LCDR2, and LCDR3) contained within a LCVR including the amino acid sequence of SEQ ID NO: 40 and three CDRs (HCDR1, HCDR2, and HCDR3) contained within a HCVR contained within a HCVR including an amino acid sequence selected from SEQ ID NO: 32, 52, 78, 88, 120, 130, and 148. In some embodiments, the antigen-binding domain that binds specifically to SLC3A2-APIS includes three CDRs (LCDR1, LCDR2, and LCDR3) contained within a LCVR including the amino acid sequence of SEQ ID NO: 70 and three CDRs (HCDR1, HCDR2, and HCDR3) contained within a HCVR including the amino acid sequence of SEQ ID NO: 62. In some embodiments, the antigen-binding domain that binds specifically to SLC3A2-APIS includes three CDRs (LCDR1, LCDR2, and LCDR3) contained within a LCVR including the amino acid sequence of SEQ ID NO: 112 and three CDRs (HCDR1, HCDR2, and HCDR3) contained within a HCVR including the amino acid sequence of SEQ ID NO: 104. In some embodiments, the antigen-binding domain that binds specifically to SLC3A2-APIS includes three CDRs (LCDR1, LCDR2, and LCDR3) contained within a LCVR including the amino acid sequence of SEQ ID NO: 140 and three CDRs (HCDR1, HCDR2, and HCDR3) contained within a HCVR including the amino acid sequence of SEQ ID NO: 22. In some embodiments, the antigen-binding domain that binds specifically to SLC3A2-APIS includes three CDRs (LCDR1, LCDR2, and LCDR3) contained within a LCVR including the amino acid sequence of SEQ ID NO: 158 and three CDRs (HCDR1, HCDR2, and HCDR3) contained within a HCVR including the amino acid sequence of SEQ ID NO: 148.

In some embodiments, the antigen-binding domain that binds specifically to SLC3A2-APIS includes a LCVR including the amino acid sequence of SEQ ID NO: 10; and a HCVR including an amino acid sequence selected from SEQ ID NOs: 2, 22, 62, 104, 168, 178, 188, 198, and 208. In some embodiments, the antigen-binding domain that binds specifically to SLC3A2-APIS includes a LCVR including the amino acid sequence of SEQ ID NO: 40; and a HCVR including an amino acid sequence selected from SEQ ID NOs: 32, 52, 78, 88, 120, 130, and 148. In some embodiments, the antigen-binding domain that binds specifically to SLC3A2-APIS includes a LCVR including the amino acid sequence of SEQ ID NO: 70 and a HCVR including the amino acid sequence of SEQ ID NO: 62. In some embodiments, the antigen-binding domain that binds specifically to SLC3A2-APIS includes a LCVR including the amino acid sequence of SEQ ID NO: 112 and a HCVR including the amino acid sequence of SEQ ID NO: 104. In some embodiments, the antigen-binding domain that binds specifically to SLC3A2-APIS includes the amino acid sequence of SEQ ID NO: 140 and a HCVR including the amino acid sequence of SEQ ID NO: 22. In some embodiments, the antigen-binding domain that binds specifically to SLC3A2-APIS includes a LCVR including the amino acid sequence of SEQ ID NO: 158 and a HCVR including the amino acid sequence of SEQ ID NO: 148.

In some embodiments, the antigen-binding domain that binds specifically to SLC3A2-APIS includes a LCVR including the amino acid sequence of SEQ ID NO: 232 and a HCVR including an amino acid sequence selected from SEQ ID NOs: 2, 22, 32, 52, 62, 78, 88, 104, 120, 130, 148, 168, 178, 188, 198, and 208. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 include respective amino acid sequences of SEQ ID NO: 12, AAS, and SEQ ID NO: 16; and HCDR1, HCDR2 and HCDR3 include respective amino acid sequences selected from SEQ ID NOs: (i) 4, 6, and 8; (ii) 24, 26, and 28; (iii) 64, 66, and 68; (iv) 106, 108, and 110; (v) 170, 172, and 174; (vi) 170, 200, and 202 (vii) 180, 182, and 184; (viii) 190, 192, and 194; (ix) 170, 200, and 202; and (x) 210, 6, and 8. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 include respective amino acid sequences of SEQ ID NO: 42, GAS, and SEQ ID NO: 46; and HCDR1, HCDR2, and HCDR3 include respective amino acid sequences selected from SEQ ID NOs: (i) 34, 36, and 38; (ii) 54, 56, and 58; (iii) 80, 82, and 84; (iv) 90, 92, and 94; (v) 122, 124, and 126; (vi) 132, 134, and 136; and (vii) 150, 152, and 154.

In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 include respective amino acid sequences of SEQ ID NO: 72, AAS, and SEQ ID NO: 16; and HCDR1, HCDR2, and HCDR3 include respective amino acid sequences of SEQ ID NOs: 64, 66, and 68. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 include respective amino acid sequences of SEQ ID NO: 114, AAS, and SEQ ID NO: 16; and HCDR1, HCDR2, and HCDR3 include respective amino acid sequences of SEQ ID NOs: 106, 108, and 110. In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 include respective amino acid sequences of SEQ ID NO: 12, AAS, and SEQ ID NO: 142; and HCDR1, HCDR2, and HCDR3 include respective amino acid sequences of SEQ ID NOs: 24, 26, and 28.

In some embodiments, the antigen-binding domain that binds specifically to SLC3A2-APIS includes three CDRs (LCDR1, LCDR2, and LCDR3) contained within a LCVR and three CDRs (HCDR1, HCDR2, and HCDR3) contained within a HCVR, wherein: LCDR1, LCDR2, and LCDR3 include respective amino acid sequences of SEQ ID NO: 160, GAS, and SEQ ID NO: 46; and HCDR1, HCDR2, and HCDR3 include respective amino acid sequences of SEQ ID NOs: 150, 152, and 154. In some embodiments, the antigen-binding domain that binds specifically to SLC3A2-APIS includes three CDRs (LCDR1, LCDR2, and LCDR3) contained within a LCVR and three CDRs (HCDR1, HCDR2 and HCDR3) contained within a HCVR, wherein: LCDR1, LCDR2, and LCDR3 include respective amino acid sequences of SEQ ID NO: 234, AAS, and SEQ ID NO: 238; and HCDR1, HCDR2, and HCDR3 include respective amino acid sequences selected from SEQ ID NOs: (i) 4, 6, and 8; (ii) 24, 26, and 28; (iii) 34, 36, and 38; (iv) 54, 56, and 58; (v) 64, 66, and 68; (vi) 80, 82, and 84; (vii) 90, 92, and 94; (viii) 106, 108, and 110; (ix) 122, 124, and 126; (x) 132, 134, and 136; (xi) 150, 152, and 154 (xii) 170, 172, and 174; (xiii) 170, 200, and 202; (xiv) 180, 182, and 184; (xv) 190, 192, and 194; and (xvi) 210, 6, and 8.

In some embodiments, the isolated antigen-binding protein is a monoclonal antibody or antigen-binding fragment thereof. In some embodiments, the monoclonal antibody or antigen-binding fragment thereof includes a heavy chain and a light chain, wherein the heavy chain includes an amino acid sequence selected from SEQ ID NOs: 18, 30, 48, 60, 74, 86, 96, 98, 100, 102, 116, 128, 138, 144, 156, 164, 176, 186, 196, 204, 206, 212, and 214. In some embodiments, monoclonal antibody or antigen-binding fragment thereof includes a heavy chain and a light chain, wherein the light chain includes an amino acid sequence selected from SEQ ID NOs: 20, 50, 76, 118, 146, and 166. In some embodiments, the monoclonal antibody or antigen-binding fragment thereof includes a heavy chain and a light chain, wherein the heavy chain includes an amino acid sequence selected from SEQ ID NOs: 18, 30, 48, 60, 74, 86, 96, 98, 100, 102, 116, 128, 138, 144, 156, 164, 176, 186, 196, 204, 206, 212, and 214; and wherein the light chain includes an amino acid sequence selected from SEQ ID NOs: 20, 50, 76, 118, 146, and 166.

In some embodiments, the isolated antigen-binding protein is a bispecific antibody. In some embodiments, the bispecific antibody further includes an antigen-binding domain that binds specifically to CD3. In some embodiments, the antigen-binding domain of the bispecific antibody that binds specifically to CD3 includes three CDRs (LCDR1, LCDR2, and LCDR3) contained within a LCVR including an amino acid sequence selected from SEQ ID NOs: 70, 232, and 277; and three CDRs (HCDR1, HCDR2, and HCDR3) contained within a HCVR including an amino acid sequence selected from SEQ ID NOs: 216, 224, 269, 291, and 301.

In another aspect, the disclosed technology relates to an isolated bispecific antigen-binding protein including: (a) a first antigen-binding domain that binds specifically to SLC3A2-APIS, wherein the antigen-binding domain (i) does not bind to wild-type SLC3A2; and (ii) interacts with one or more amino acids contained within SEQ ID NO: 266, as determined by cryo-electron microscopy; and (b) a second antigen-binding domain that binds specifically to CD3. In some embodiments, the first antigen-binding domain that binds specifically to SLC3A2-APIS includes: three CDRs (LCDR1, LCDR2, and LCDR3) contained within a LCVR including an amino acid sequence selected from SEQ ID NOs: 10, 40, 70, 112, 140, 158, 232, and 277; and three CDRs (HCDR1, HCDR2, and HCDR3) contained within a HCVR including an amino acid sequence selected from SEQ ID NOs: 2, 22, 32, 52, 62, 78, 88, 104, 120, 130, 148, 168, 178, 188, 198, and 208. In some embodiments, the second antigen-binding domain includes three CDRs (LCDR1, LCDR2, and LCDR3) contained within a LCVR including an amino acid sequence selected from SEQ ID NOs: 70 and 232; and three CDRs (HCDR1, HCDR2, and HCDR3) contained within a HCVR including an amino acid sequence selected from SEQ ID NOs: 216, 224, 269, 291, and 301. In some embodiments, the first antigen-binding domain that binds specifically to SLC3A2-APIS includes: three CDRs (LCDR1, LCDR2, and LCDR3) contained within a LCVR including an amino acid sequence selected from SEQ ID NOs: 70 and 232; and three CDRs (HCDR1, HCDR2, and HCDR3) contained within a HCVR including an amino acid sequence selected from SEQ ID NOs: 2, 22, 62, 104, 168, 178, 188, 198, and 208. In some embodiments, the second antigen-binding domain includes three CDRs (LCDR1, LCDR2, and LCDR3) contained within a LCVR including an amino acid sequence selected from SEQ ID NOs: 70 and 232; and three CDRs (HCDR1, HCDR2, and HCDR3) contained within a HCVR including an amino acid sequence selected from SEQ ID NOs: 216 and 224.

In some embodiments, the first antigen-binding domain that binds specifically to SLC3A2-APIS includes: (a) three CDRs (LCDR1, LCDR2, and LCDR3) contained within a LCVR wherein LCDR1, LCDR2 and LCDR3 include respective amino acid sequences selected from (i) SEQ ID NO: 72, AAS, and SEQ ID NO: 16; and (ii) SEQ ID NO: 234, AAS, and SEQ ID NO: 238; and (b) three CDRs (HCDR1, HCDR2, and HCDR3) contained within a HCVR wherein HCDR1, HCDR2, and HCDR3 include respective amino acid sequences selected from SEQ ID NOs: (i) 4, 6, and 8; (ii) 24, 26, and 28; (iii) 64, 66, and 68; (iv) 106, 108, and 110; (v) 170, 172, and 174; (vi) 170, 200, and 202; (vii) 180, 182, and 184; (viii) 190, 192, and 194; and (ix) 210, 6, and 8. In some embodiments, the second antigen-binding domain includes: (a) three CDRs (LCDR1, LCDR2, and LCDR3) contained within a LCVR wherein LCDR1, LCDR2 and LCDR3 include respective amino acid sequences selected from SEQ ID NO: (i) SEQ ID NO: 72, AAS, and SEQ ID NO: 16; and (ii) SEQ ID NO: 234, AAS, and SEQ ID NO: 238; and (b) three CDRs (HCDR1, HCDR2, and HCDR3) contained within a HCVR wherein HCDR1, HCDR2, and HCDR3 include respective amino acid sequences selected from SEQ ID NOs: (i) 218, 220, and 222; and (ii) 226, 228, and 230. In some embodiments, the first antigen-binding domain that binds specifically to SLC3A2-APIS includes a LCVR including an amino acid sequence selected from SEQ ID NOs: 70 and 232; and a HCVR including an amino acid sequence selected from SEQ ID NOs: 2, 22, 62, 104, 168, 178, 188, 198, and 208. In some embodiments, the second antigen-binding domain includes a LCVR including an amino acid sequence selected from SEQ ID NOs: 70 and 232; and a HCVR including an amino acid sequence selected from SEQ ID NOs: 216 and 224.

In another aspect, the disclosed technology relates to an isolated bispecific antigen-binding protein including: (a) an antigen-binding domain that binds specifically to SLC3A2-APIS including three CDRs (LCDR1, LCDR2, and LCDR3) contained within a LCVR including an amino acid sequence of SEQ ID NO: 70; and three CDRs (HCDR1, HCDR2, and HCDR3) contained within a HCVR including an amino acid sequence of SEQ ID NO: 62; and (b) an antigen-binding domain that binds specifically to CD3 including three CDRs (LCDR1, LCDR2, and LCDR3) contained within a LCVR including an amino acid sequence of SEQ ID NO: 70; and three CDRs (HCDR1, HCDR2, and HCDR3) contained within a HCVR including an amino acid sequence of SEQ ID NO: 216.

In another aspect, the disclosed technology relates to an isolated bispecific antigen-binding protein including: (a) an antigen-binding domain that binds specifically to SLC3A2-APIS including three CDRs (LCDR1, LCDR2, and LCDR3) contained within a LCVR including an amino acid sequence of SEQ ID NO: 70; and three CDRs (HCDR1, HCDR2, and HCDR3) contained within a HCVR including an amino acid sequence of SEQ ID NO: 62; and (b) an antigen-binding domain that binds specifically to CD3 including three CDRs (LCDR1, LCDR2, and LCDR3) contained within a LCVR including an amino acid sequence of SEQ ID NO: 70; and three CDRs (HCDR1, HCDR2, and HCDR3) contained within a HCVR including an amino acid sequence of SEQ ID NO: 224.

In another aspect, the disclosed technology relates to an isolated bispecific antigen-binding protein including: (a) an antigen-binding domain that binds specifically to SLC3A2-APIS including three CDRs (LCDR1, LCDR2, and LCDR3) contained within a LCVR and three CDRs (HCDR1, HCDR2, and HCDR3) contained within a HCVR; wherein LCDR1, LCDR2, and LCDR3 include respective amino acid sequences of SEQ ID NO: 72, AAS, and SEQ ID NO: 16; and HCDR1, HCDR2 and HCDR3 include respective amino acid sequences of SEQ ID NOs: 64, 66, and 68; and (b) an antigen-binding domain that binds specifically to CD3 including three CDRs (LCDR1, LCDR2, and LCDR3) contained within a LCVR and three CDRs (HCDR1, HCDR2, and HCDR3) contained within a HCVR; wherein LCDR1, LCDR2, and LCDR3 include respective amino acid sequences of SEQ ID NO: 72, AAS, and SEQ ID NO: 16; and HCDR1, HCDR2, and HCDR3 include respective amino acid sequences of SEQ ID NOs: 218, 220, and 222.

In another aspect, the disclosed technology relates to an isolated bispecific antigen-binding protein including: (a) an antigen-binding domain that binds specifically to SLC3A2-APIS including three CDRs (LCDR1, LCDR2 and LCDR3) contained within a LCVR and three CDRs (HCDR1, HCDR2 and HCDR3) contained within a HCVR; wherein LCDR1, LCDR2 and LCDR3 include respective amino acid sequences of SEQ ID NO: 72, AAS, and SEQ ID NO: 16; and HCDR1, HCDR2 and HCDR3 include respective amino acid sequences of SEQ ID NOs: 64, 66, and 68; and (b) an antigen-binding domain that binds specifically to CD3 including three CDRs (LCDR1, LCDR2 and LCDR3) contained within a LCVR and three CDRs (HCDR1, HCDR2 and HCDR3) contained within a HCVR; wherein LCDR1, LCDR2 and LCDR3 include respective amino acid sequences of SEQ ID NO: 72, AAS, and SEQ ID NO: 16; and HCDR1, HCDR2 and HCDR3 include respective amino acid sequences of SEQ ID NOs: 226, 228, and 230.

In another aspect, the disclosed technology relates to an isolated bispecific antigen-binding protein including: (a) an antigen-binding domain that binds specifically to SLC3A2-APIS including a LCVR including an amino acid sequence of SEQ ID NO: 70; and a HCVR including an amino acid sequence of SEQ ID NO: 62; and (b) an antigen-binding domain that binds specifically to CD3 including a LCVR including an amino acid sequence of SEQ ID NO: 70; and a HCVR including an amino acid sequence of SEQ ID NO: 216.

In another aspect, the disclosed technology relates to an isolated bispecific antigen-binding protein including: (a) an antigen-binding domain that binds specifically to SLC3A2-APIS including a LCVR including an amino acid sequence of SEQ ID NO: 70; and a HCVR including an amino acid sequence of SEQ ID NO: 62; and (b) an antigen-binding domain that binds specifically to CD3 including a LCVR including an amino acid sequence of SEQ ID NO: 70; and a HCVR including an amino acid sequence of SEQ ID NO: 224.

In some embodiments, the isolated bispecific antigen-binding protein as disclosed herein that is a bispecific antibody includes: a first heavy chain and a paired light chain interconnected by disulfide bonds, wherein the first heavy chain includes a HCVR and a heavy chain constant region including CH1, CH2, and CH3 domains, and the paired light chain includes a LCVR and a light chain constant region, wherein the first heavy chain and paired light chain include the first antigen-binding domain; and a second heavy chain and a paired light chain interconnected by disulfide bonds, wherein the second heavy chain includes a HCVR and a heavy chain constant region including CH1, CH2, and CH3 domains, and the light chain includes a LCVR and a light chain constant region, wherein the second heavy chain and paired light chain include the second antigen-binding domain. In some embodiments, the first heavy chain or the second heavy chain of the bispecific antibody, but not both, includes a CH3 domain including a H435R (EU numbering) modification and a Y436F (EU numbering) modification. In some embodiments, the heavy chain constant region of the first heavy chain of the bispecific antibody and the heavy chain constant region of the second heavy chain of the bispecific antibody are of isotype IgG1. In some embodiments, the heavy chain constant region of the first heavy chain of the bispecific antibody and the heavy chain constant region of the second heavy chain of the bispecific antibody are of isotype IgG4. In some embodiments, the first heavy chain and the second heavy chain of the bispecific antibody include a chimeric hinge that reduces Fcγ receptor binding relative to a wild-type hinge of the same isotype. In some embodiments, the first heavy chain includes an amino acid sequence selected from SEQ ID NOs: 248, 250, 252, 254, 256, 258, 260, 262, and 264; and the second heavy chain includes and amino acid sequence selected from SEQ ID NO: 240 and 242. In some embodiments, the paired light chain includes an amino acid sequence selected from SEQ ID NO: 244 and 246. In some embodiments, the first heavy chain includes the amino acid sequence of SEQ ID NO: 258, the second heavy chain includes the amino acid sequence of SEQ ID NO: 240, and the paired light chain includes the amino acid sequence of SEQ ID NO: 246. In some embodiments, the first heavy chain includes the amino acid sequence of SEQ ID NO: 258, the second heavy chain includes the amino acid sequence of SEQ ID NO: 242, and the paired light chain includes the amino acid sequence of SEQ ID NO: 246.

In another aspect, the disclosed technology relates to a pharmaceutical composition including an isolated antigen-binding protein as disclosed herein, and a pharmaceutically acceptable carrier or diluent.

In another aspect, the disclosed technology relates to a pharmaceutical composition including an isolated bispecific antigen-binding protein as disclosed herein, and a pharmaceutically acceptable carrier or diluent.

In another aspect, the disclosed technology relates to a method for making an isolated antigen-binding protein as disclosed herein including: (a) introducing one or more nucleic acid molecules including polynucleotide sequences that encode the antigen-binding protein into a host cell, and (b) culturing the host cell under conditions favorable to expression of the one or more nucleic acid molecules. In some embodiments, the method, further includes (c) isolating the antigen-binding protein from the host cell and/or medium in which the host cell is grown. In some embodiments, the host cell is a Chinese hamster ovary (CHO) cell.

In another aspect, the disclosed technology relates to a nucleic acid molecule including a nucleotide sequence encoding an antigen-binding protein as described herein, or a group of nucleic acid molecules including nucleotide sequences, encoding the HCVR and the LCVR of an antigen-binding protein as described herein.

In another aspect, the disclosed technology relates to an expression vector including a nucleic acid molecule as disclosed herein, or a group of expression vectors including a group of nucleic acid molecules as disclosed herein.

In another aspect, the disclosed technology relates to a host cell including an antigen-binding protein as described herein, a nucleic acid molecule or group of nucleic acid molecules as described herein, or the expression vector or group of expression vectors as described herein. In some embodiments, the host cell is a CHO cell.

In another aspect, the disclosed technology relates to a method of producing an antigen-binding protein that specifically binds SLC3A2-APIS including: (a) culturing a host cell as described herein under conditions favorable for production of the antigen-binding protein; and (b) optionally, isolating the antigen-binding protein from the host cell and/or medium in which the host cell is grown. In some embodiments, the host is a CHO cell. In some embodiments, the method further includes formulating the antigen-binding protein as a pharmaceutical composition including an acceptable carrier.

In another aspect, the disclosed technology relates to a method for making an isolated bispecific antigen-binding protein as disclosed herein including: (a) introducing one or more nucleic acid molecules including polynucleotide sequences that encode the bispecific antigen-binding protein into a host cell, and (b) culturing the host cell under conditions favorable to expression of the one or more nucleic acid molecules. In some embodiments, the method further includes (c) isolating the bispecific antigen-binding protein from the host cell and/or medium in which the host cell is grown. In some embodiments, the host cell is a CHO cell.

In another aspect, the disclosed technology relates to a nucleic acid molecule including a nucleotide sequence encoding a bispecific antigen-binding protein as disclosed herein, or a group of nucleic acid molecules including nucleotide sequences, respectively, encoding the HCVR of the first antigen-binding domain, the HCVR of the second antigen-binding domain, and the LCVR of the first and second antigen-binding domains of a bispecific antigen-binding protein as disclosed herein.

In another aspect, the disclosed technology relates to an expression vector including a nucleic acid molecule as disclosed herein, or a group of expression vectors including a group of nucleic acid molecules as disclosed herein.

In another aspect, the disclosed technology relates to a host cell including a bispecific antigen-binding protein as disclosed herein, a nucleic acid molecule or group of nucleic acid molecules as disclosed herein, or an expression vector or group of expression vectors as disclosed herein.

In another aspect, the disclosed technology relates to a host cell including: (a) an expression vector including a nucleic acid molecule encoding a first immunoglobulin heavy chain including the amino acid sequence of SEQ ID NO: 258; (b) an expression vector including a nucleic acid molecule encoding a second immunoglobulin heavy chain including the amino acid sequence of SEQ ID NO: 240; and (c) an expression vector including a nucleic acid molecule encoding an immunoglobulin light chain including the amino acid sequence of SEQ ID NO: 246.

In another aspect, the disclosed technology relates to a host cell including: (a) an expression vector including a nucleic acid molecule encoding a first immunoglobulin heavy chain including the amino acid sequence of SEQ ID NO: 258; (b) an expression vector including a nucleic acid molecule encoding a second immunoglobulin heavy chain including the amino acid sequence of SEQ ID NO: 242; and (c) an expression vector including a nucleic acid molecule encoding an immunoglobulin light chain including the amino acid sequence of SEQ ID NO: 246.

In another aspect, the disclosed technology relates to a method of producing a bispecific antigen-binding protein that specifically binds SLC3A2-APIS and CD3 including: (a) culturing a host cell as described herein under conditions favorable for production of the bispecific antigen-binding protein; and (b) optionally, isolating the bispecific antigen-binding protein from the host cell and/or medium in which the host cell is grown. In some embodiments, the host is a CHO cell. In some embodiments, the method further includes formulating the bispecific antigen-binding protein as a pharmaceutical composition including an acceptable carrier.

In another aspect, the disclosed technology relates to a method of inhibiting growth of a tumor in a subject, including administering an isolated antigen-binding protein as described herein, a bispecific antigen-binding protein as described herein, or a pharmaceutical composition as described herein to the subject. In some embodiments, the tumor is anal cancer, angiosarcoma, acute myeloid leukemia, acute lymphocytic leukemia, a B cell cancer, basal cell carcinoma, bladder cancer, bone cancer, brain cancer, breast cancer, cervical cancer, cholangiocarcinoma, chondrosarcoma, chronic lymphocytic leukemia colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, lung cancer, lymphoma, leukemia, Merkel cell carcinoma, melanoma, myeloma, myeloproliferative neoplasm, non-small cell lung cancer, ovarian cancer, pancreatic cancer, prostate cancer, salivary gland cancer, skin cancer, soft tissue sarcoma, stomach cancer, a T cell cancer, testicular cancer, uterine cancer, or uveal melanoma. In some embodiments, the tumor includes SLC3A2-APIS-expressing tumor cells.

In another aspect, the disclosed technology relates to a method of treating or ameliorating one or more symptoms of a myelodysplastic syndrome in a subject, including administering an antigen-binding protein as disclosed herein, a bispecific antigen-binding protein as disclosed herein, or a pharmaceutical composition as disclosed herein to the subject.

In another aspect, the disclosed technology relates to a method of reducing the risk of progression to a cancer, including administering to a subject in need thereof an antigen-binding protein as described herein, or a bispecific antigen-binding protein as described herein, wherein the subject has been diagnosed with a premalignant condition, and wherein the risk of progression is reduced relative to a control subject not administered the antigen-binding protein or bispecific antigen-binding protein. In some embodiments, the premalignant condition is clonal hematopoiesis of indeterminate potential (CHIP), clonal cytopenia of undetermined significance (CCUS), or age-related clonal hematopoiesis (ARCH). In some embodiments, the method further includes administering a second therapeutic agent or therapeutic regimen.

In some embodiments, the second therapeutic agent or therapeutic regimen includes a chemotherapeutic drug, a DNA alkylator, an immunomodulator, a proteasome inhibitor, a histone deacetylase inhibitor, radiotherapy, surgery, a stem cell transplant, leukapheresis, an antibody drug conjugate, a cell containing a chimeric antigen receptor, an oncolytic virus, a bispecific antibody conjugated to an anti-tumor agent, a VEGF inhibitor, a checkpoint inhibitor, a GITR agonist, a CD27 agonist, a CD28 agonist, a 4-1BB activator, a CD38 inhibitor, a PD-1 inhibitor, a PD-L1 inhibitor, a LAG3 inhibitor, a CTLA-4 inhibitor, an EGFR inhibitor, an Ang2 inhibitor, a MUC16 inhibitor, a CD20 inhibitor, a BCMA inhibitor, a bispecific antibody including a CD28-binding arm, a bispecific antibody including a 4-1BB-binding arm, a cancer vaccine, a cytokine, a modified IL2, a modified IL12, an IL1 inhibitor, an IL4 inhibitor, an IL6 inhibitor, a corticosteroid, or combinations thereof.

In another aspect, the disclosed technology relates to an isolated antigen-binding protein that includes an antigen-binding domain that binds specifically to SLC3A2-APIS in a tumor cell for use in a method of treating, inhibiting or preventing the growth of a tumor. In some embodiments, the antigen-binding protein further includes an antigen-binding domain that binds specifically to CD3.

In another aspect, the disclosed technology relates to a kit including an isolated antigen-binding protein that includes an antigen-binding domain that binds specifically to SLC3A2-APIS in a tumor cell in combination with written instructions for use of a therapeutically effective amount of the antigen-binding protin for inhibiting the growth of a tumor in a subject. In some embodiments, the kit includes an isolated antigen-binding protein that includes an antigen-binding domain that binds specifically to SLC3A2-APIS in a tumor cell further includes an antigen-binding domain that binds specifically to CD3.

### BRIEF DESCRIPTION OF THE FIGURES

FIGS. 1A-1E relate to Example 3 and are a set of graphs showing binding to the indicated cell types of the indicated antibodies over a range of concentrations. FIG. 1A shows binding of REGN17192. FIG. 1B shows binding of REGN17193. FIG. 1C shows binding of REGN17195, wherein the left graph shows binding up to 25,000 MFI, and the right graph shows binding up to 4,000 MFI. FIG. 1D shows binding of REGN17198. FIG. 1E shows binding of REGN17201.

FIGS. 2A-2E relate to Example 3 and are a set of graphs showing binding to the indicated cell types of the indicated antibodies over a range of concentrations. FIG. 2A shows binding of REGN17202, wherein the left graph shows binding up to 25,000 MFI, and the right graph shows binding up to 4,000 MFI. FIG. 2B shows binding of REGN17204. FIG. 2C shows binding of REGN17209. FIG. 2D shows binding of REGN17206, wherein the left graph shows binding up to 25,000 MFI, and the right graph shows binding up to 4,000 MFI. FIG. 2E shows binding of REGN17208, wherein the left graph shows binding up to 25,000 MFI, and the right graph shows binding up to 4,000 MFI.

FIGS. 3A-3F relate to Example 3 and are a set of graphs showing binding to the indicated cell types of the indicated antibodies over a range of concentrations. FIG. 3A shows binding of REGN17212. FIG. 3B shows binding of REGN17224. FIG. 3C shows binding of CD3-7221G Control. FIG. 3D shows binding of CD3-7195P Control. FIG. 3E shows binding of Isotype Control. FIG. 3F shows binding of bsAb17225.

FIGS. 4A-4D relate to Example 3 and are a set of graphs showing binding to the indicated cell types of the indicated antibodies over a range of concentrations. FIG. 4A shows binding of bsAb17226, wherein the left graph shows binding up to 10,000 MFI, and the right graph shows binding up to 2,000 MFI. FIG. 4B shows binding of bsAb17227. FIG. 4C shows binding of bsAb17228, wherein the left graph shows binding up to 10,000 MFI, and the right graph shows binding up to 2,000 MFI. FIG. 4D shows binding of bsAb17229, wherein the left graph shows binding up to 10,000 MFI, and the right graph shows binding up to 2,000 MFI.

FIGS. 5A-5F relate to Example 3 and are a set of graphs showing binding to the indicated cell types of the indicated antibodies over a range of concentrations. FIG. 5A shows binding of bsAb17230. FIG. 5B shows binding of bsAb17231. FIG. 5C shows binding of bsAb17232, wherein the left graph shows binding up to 10,000 MFI, and the right graph shows binding up to 2,000 MFI. FIG. 5D shows binding of bsAb17233, wherein the left graph shows binding up to 10,000 MFI, and the right graph shows binding up to 2,000 MFI. FIG. 5E shows binding of bsAb17234. FIG. 5F shows binding of bsAb17235.

FIG. 6 relates to Example 3 and is a set of graphs showing binding to 3T3/hCD20/hSLC3A2-APIS cells (upper graph) and 3T3/hCD20/hSLC3A2-WT cells (lower graph) of the indicated antibodies over a range of concentrations.

FIG. 7 relates to Example 3 and is a set of graphs showing binding to SLC3A2-APIS-positive Mel-2 cells (upper graph) and to SLC3A2-APIS-negative NALM6 cells (lower graph) of the indicated antibodies over a range of concentrations.

FIGS. 8A-8C relate to Examples 4 and 5 are a set of graphs showing responses of 3T3/hCD20/hSLC3A2-APIS cells treated with the indicated antibodies over a range of concentrations. FIG. 8A shows NFAT reporter activity, wherein arrows indicate the EC₅₀ concentration of the indicated antibodies. FIG. 8B shows TNFα release, wherein arrows indicate the EC₅₀ concentration of the indicated antibodies. FIG. 8C shows IFNγ release, wherein the left graph shows release up to 6×10⁴ pg/mL, and the right graph shows release up to 2×10⁴ pg/mL.

FIG. 9 relates to Examples 4 and 5 and is a set of graphs showing the NFAT reporter activity (upper-left graph), IFNγ release (lower-left graph), and TNFα release (lower-right graph) of Mel-202 cells, which endogenously express SLC3A2-APIS, treated with the indicated antibodies over a range of concentrations. Arrows indicate the EC₅₀ concentration of the indicated antibodies.

FIGS. 10A-10C relate to Example 6 and are a set of graphs showing activities for Mel-202 cells (top graphs) and HNT-34 cells (bottom graphs) treated with the indicated antibodies over a range of concentrations. FIG. 10A shows target killing. FIG. 10B shows CD8-T activation. FIG. 10C shows CD4-T activation.

FIGS. 11A-11C relate to Example 6 and are a set of graphs showing activities for 3T3/hCD20/hSLC3A2-APIS cells (top graphs) and 3T3/hCD20/hSLC3A2-WT cells (bottom graphs) treated with the indicated antibodies over a range of concentrations. FIG. 11A shows target killing. FIG. 11B shows CD8-T activation. FIG. 11C shows CD4-T activation.

FIGS. 12A-12B relate to Example 6 and are a set of graphs showing concentration of the indicated cytokines released by Mel-202 cells (top graphs) and HNT-34 cells (bottom graphs) treated with the indicated antibodies over a range of concentrations. FIG. 12A shows IL-2 release (left graphs) and IL-10 release (right graphs). FIG. 12B shows TNF-α release (left graphs) and Granzyme B release (right graphs).

FIGS. 13A-13B relates to Example 6 and are a set of graphs showing concentration of the indicated cytokines released by 3T3/hCD20/hSLC3A2-APIS cells (top graphs) and 3T3/hCD20/hSLC3A2-WT cells treated with the indicated antibodies over a range of concentrations. FIG. 13A shows IL-2 release (left graphs) and IL-10 release (right graphs). FIG. 13B shows TNF-α release (left graphs) and Granzyme B release (right graphs).

FIGS. 14A-14B relate to Example 7 and are images depicting the map and model of the ectodomain of SLC3A2-APIS bound to a Fab of the anti-SLC3A2-APIS monospecific antibody REGN17224. FIG. 14A shows the map, wherein the arrowhead indicates the APIS insertion site. FIG. 14B shows the model, wherein the black portion of the polypeptide shows the portion of the SLC3A2-APIS ectodomain containing the APIS insertion, and the Ca atoms of the APIS insertion residues are shown as black spheres and labeled. Bound Fab is shown as semi-transparent surface. A published structure of wild-type SLC3A2 ectodomain (PDB 8G0M, aligned to the SLC3A2-APIS structure) is shown in light gray.

FIG. 15 relates to Example 8 and is a graph showing average tumor volume in mice engrafted with Mel-202 uveal melanoma tumor cells and administered the indicated antibodies.

FIGS. 16A-16B relate to Example 8 and are a set of graphs showing tumor volume in individual mice engrafted with Mel-202 uveal melanoma tumor cells and administered the indicated antibodies. The fraction of mice that were tumor free is indicated. FIG. 16A shows tumor volumes in mice administered isotype control (4 mg/kg), CD3-7195 Control (4 mg/kg), or bsAb17234 (1 mg/kg). FIG. 16B shows tumor volumes in mice administered bsAb17234 (4 mg/kg), bsAb17235 (1 mg/kg), or bsAb17235 (4 mg/kg).

FIG. 17 relates to Example 8 and is a graph showing average tumor volume in mice engrafted with HNT-34 acute myeloid leukemia tumor cells and administered the indicated antibodies.

FIG. 18 relates to Example 8 and is a set of graphs showing tumor volume in individual mice engrafted with HNT-34 acute myeloid leukemia tumor cells and administered the indicated antibodies. The fraction of mice that were tumor free is indicated.

FIG. 19 relates to Example 8 and is a graph showing average tumor volume in mice engrafted with PANC05.05 pancreatic adenocarcinoma tumor cells and administered the indicated antibodies.

FIG. 20 relates to Example 8 and is a set of graphs showing tumor volume in individual mice engrafted with PANC05.05 pancreatic adenocarcinoma tumor cells and administered the indicated antibodies. The fraction of mice that were tumor free is indicated.

FIGS. 21A-21C relate to Example 8 and are graphs showing average tumor volume in mice engrafted with specific cell types and administered the indicated antibodies. FIG. 21A shows average tumor volume in mice engrafted with NALM6 acute lymphoblastic leukemia cells that were engineered to express mutant SF3B1. FIG. 21B shows average tumor volume in mice engrafted with NALM6 acute lymphoblastic leukemia cells. FIG. 21C shows average tumor volume in mice engrafted with Raji tumor cells.

FIGS. 22A-22C relate to Example 8 and are a set of graphs showing the expression of SLC3A2-APIS and pan-SLC3A2 as evaluated by flow cytometry. FIG. 22A shows expression of SLC3A2-APIS and pan-SLC3A2 for NALM6 acute lymphoblastic leukemia cells that were engineered to express mutant SF3B1. FIG. 22B shows expression of SLC3A2-APIS and pan-SLC3A2 for NALM6 acute lymphoblastic leukemia cells. FIG. 22C shows expression of SLC3A2-APIS and pan-SLC3A2 for Raji tumor cells.

FIG. 23 relates to Example 8 and is a graph showing average tumor volume in mice engrafted with NALM6 acute lymphoblastic leukemia cells that were engineered to express mutant SF3B1 and administered the indicated antibodies.

FIG. 24 relates to Example 8 and is a set of graphs showing tumor volume in individual mice engrafted with NALM6 acute lymphoblastic leukemia cells that were engineered to express mutant SF3B1 and administered the indicated antibodies. The fraction of mice that were tumor free is indicated.

FIG. 25 relates to Example 9 and is a graph showing average tumor volume in mice engrafted with MEL202 cells and administered the indicated antibodies. Symbols represent group means and error bars represent standard error of the mean.

FIGS. 26A-26E relate to relate to Example 9 and are graphs showing tumor volume in individual mice engrafted with MEL202 cells and administered antibodies. The fraction of mice that were tumor free is indicated. FIG. 26A shows tumor growth in mice administered 0.04 mg/kg bsAb17235. FIG. 26B shows tumor growth in mice administered 0.1 mg/kg bsAb17235.

FIG. 26C shows tumor growth in mice administered 1 mg/kg bsAb17235. FIG. 26D shows tumor growth in mice administered 4 mg/kg bsAb17235. FIG. 26E shows tumor growth in mice administered 4 mg/kg non-TAAxCD3.

FIG. 27 relates to Example 9 and is a graph showing average tumor volume in mice engrafted with HNT34 cells and administered the indicated antibodies. Symbols represent group means and error bars represent standard error of the mean.

FIGS. 28A-28D relate to relate to Example 9 and are graphs showing tumor volume in individual mice engrafted with HNT34 cells and administered antibodies. The fraction of mice that were tumor free is indicated. FIG. 28A shows tumor growth in mice administered 0.1 mg/kg bsAb17235. FIG. 28B shows tumor growth in mice administered 1 mg/kg bsAb17235. FIG. 28C shows tumor growth in mice administered 4 mg/kg bsAb17235. FIG. 28D shows tumor growth in mice administered 4 mg/kg non-TAAxCD3.

FIG. 29 relates to Example 10 and are a set of graphs showing flow cytometry data indicating wild-type SLC3A2 expression on the surface of the indicated human blood cell types.

FIG. 30 relates to Example 10 and is a set of graphs showing percent of CD69+ T cells from whole blood from three different donors incubated with the indicated concentrations of the indicated antibodies.

FIG. 31 relates to Example 10 and is a set of graphs showing the concentration of IFNG released from whole blood cells from three different donors incubated with the indicated concentrations of the indicated antibodies.

FIG. 32 relates to Example 10 and is a set of graphs showing the concentration of IL6 released from whole blood cells from three different donors incubated with the indicated concentrations of the indicated antibodies.

FIG. 33 relates to Example 10 and is a set of graphs showing the concentration of TNFA released from whole blood cells from three different donors incubated with the indicated concentrations of the indicated antibodies

FIG. 34 relates to Example 11 and is a cryoEM reconstruction of a complex of the SLC3A2-APIS ectodomain bound to the REGN17212 Fab and to a noncompeting commercial mouse IgG antibody MEM-108, which was added to improve particle alignment.

FIG. 35 relates to Example 11 is a cryoEM reconstruction and shows the structure of the SLC3A2-APIS ectodomain bound to the REGN17212 Fab. The REGN17212 Fab is depicted as a ribbon. The location of the APIS insertion and APIS-adjacent loop are indicated.

FIG. 36 relates to Example 11 and is a cryoEM reconstruction and shows an expanded view of the APIS insertion region of SLC3A2. The wild-type SLC3A2 structure (PDB 8G0M) is shown, with APIS insertion and APIS-adjacent loop residues of SLC3A2-APIS structure overlaid. The movement of residue Leu241 from a buried position in wild type to a surface-exposed position resulting from the APIS insertion is indicated.

FIGS. 37A-37F relate to Example 12 and are graphs showing the mean fluorescence intensity (MFI) of cell lines incubated with the indicated concentrations of the indicated antibodies. FIG. 37A shows the MFI of 3T3/SLC3A2-APIS/hCD20 cells. FIG. 37B shows the MFI of 3T3/SLC3A2 WT/hCD20 cells. FIG. 37C shows the MFI of 3T3/hCD20 cells. FIG. 37D shows the MFI of HNT-34 cells. FIG. 37E shows the MFI of MEL-202 cells. FIG. 37F shows the MFI of NALM-6 cells.

FIGS. 38A-38D relate to Example 12 and are graphs showing MFI of T cells from different donors incubated with the indicated concentrations of the indicated antibodies. FIG. 38A shows the MFI of CD4+ T cells from Donor 1. FIG. 38B shows the MFI of CD8+ T cells from Donor 1. FIG. 38C shows the MFI of CD4+ T cells from Donor 2. FIG. 38D shows the MFI of CD8+ T cells from Donor 2.

FIG. 39 relates to Example 12 and is a graph showing MFI of T cells from the indicated species incubated with the indicated concentrations of the indicated antibodies. Cyno, cynomolgus monkey; Rhesus, rhesus monkey.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

As used herein, “SLC3A2-APIS” refers to the noncanonical spliceoform of the SLC3A2 protein. The amino acid sequence of SLC3A2 protein (also referred to herein as “wild-type SLC3A2” or “SLC3A2-WT”) is provided in the Uniprot accession number P08195. SLC3A2-APIS refers to the SLC3A2 protein comprising the amino acids alanine, proline, isoleucine and serine (APIS) between A242 and G243 of P08195. The term “SLC3A2-APIS” also includes recombinant SLC3A2-APIS or a fragment thereof. The term also encompasses SLC3A2-APIS or a fragment thereof coupled to, for example, histidine tag, or mouse or human Fc. For example, the term includes sequences exemplified by SEQ ID NOs: 265 or 267. Unless specified as being from a non-human species, the term “SLC3A2-APIS” means human SLC3A2-APIS.

As used herein, “CD3” refers to an antigen expressed on T cells as part of the T cell receptor and which consists of a homodimer or heterodimer formed from the association of two of four receptor chains: CD3-epsilon, CD3-delta, CD3-zeta, and CD3-gamma. The term “CD3” means human CD3 unless specified as being from a non-human species.

As used herein, “isolated” antigen-binding molecules (e.g., antibodies or antigen-binding fragments thereof), polypeptides, polynucleotides and vectors, refer to such molecules that are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. The term “isolated” is not intended to refer to a complete absence of such biological molecules or to an absence of water, buffers, or salts or to components of a pharmaceutical formulation that includes the antibodies or antigen-binding fragments thereof.

As used herein, an “antibody” refers to an immunoglobulin molecule comprising four polypeptide chains, two heavy chains (HC) and two light chains (LC) inter-connected by disulfide bonds. Each heavy chain (HC) comprises a heavy chain variable region (abbreviated herein as HCVR or V_{H}) and a heavy chain constant region (e.g., IgG, IgG1 or IgG4). The heavy chain constant region comprises three domains, C_{H}1, C_{H}2 and C_{H}3. Each light chain (LC) comprises a light chain variable region (abbreviated herein as LCVR or V_{L}) and a light chain constant region (e.g., lambda or kappa). The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} includes three CDRs and four FRs, arranged from amino-terminus to carboxy-terminus in the following order: FR1, CDR1, FR2, CDR2, FR3, CDR3, FR4. A heavy chain CDR may be referred to as HCDR and a light chain CDR may be referred to as LCDR. In different embodiments, the FRs of an antibody (or antigen-binding fragment thereof) may be identical to the human germline sequences, or may be naturally or artificially modified.

As used herein, an “antigen-binding arm” or “arm” of a Y-shaped IgG antibody (e.g., a CD3 or SLC3A2-APIS binding arm) refers to a structural portion of the antibody that confers binding specificity to the antigen. For example, an antigen-binding arm of an IgG antibody has a heavy chain (HC) associated with a light chain (LC). An antigen-binding molecule which, for example, is a bispecific antibody includes an antigen-binding arm (or domain) that binds specifically to a first antigen and another antigen-binding arm (or domain) that binds specifically to a second antigen. For example, an SLC3A2-APISxCD3 bispecific antibody includes one antigen-binding arm that binds specifically to SLC3A2-APIS and another antigen-binding arm that binds specifically to CD3.

As used herein, the expression “antigen-binding molecule” or “antigen-binding protein” means a protein, polypeptide, antibody, fragment of an antibody, or other molecular complex comprising or consisting of at least one complementarity determining region (CDR) that alone, or in combination with one or more additional CDRs and/or framework regions (FRs), specifically binds to a particular antigen. The term includes any naturally occurring, enzymatically obtainable, synthetic, or genetically engineered polypeptide or glycoprotein that specifically binds an antigen to form a complex. In certain embodiments, an antigen-binding molecule is an antibody or a fragment of an antibody, including a bispecific antibody or fragment thereof. In the present disclosure, the term “antigen-binding molecule” is used interchangeably with “antigen-binding protein.”

As used herein, the expression “bispecific antigen-binding molecule” or “bispecific antigen-binding molecule” means a protein, polypeptide, antibody, fragment of an antibody, or other molecular complex comprising at least a first antigen-binding arm and a second antigen-binding arm. Each antigen-binding arm within the bispecific antigen-binding molecule comprises at least one CDR that alone, or in combination with one or more additional CDRs and/or FRs, specifically binds to a particular antigen. In the context of the present disclosure, a bispecific antigen-binding molecule specifically binds to two different antigens. In some embodiments, antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments. In the present disclosure, the term “bispecific antigen-binding molecule” is used interchangeably with “bispecific antigen-binding protein.”

In some embodiments, an antigen-binding fragment of an antibody comprises at least one variable domain. The variable domain may be of any size or amino acid composition and generally comprises at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In some embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-CH2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As used herein, “recombinant” antibodies or antigen-binding fragments thereof refer to such molecules created, expressed, isolated or obtained by technologies or methods known in the art as recombinant DNA technology which include, e.g., DNA splicing and transgenic expression. The term includes antibodies or antigen-binding fragments thereof expressed in a non-human mammal (including transgenic non-human mammals, e.g., transgenic mice), or a host cell (e.g., a CHO cell) or cellular expression system or isolated from a recombinant combinatorial human antibody library. The present disclosure includes recombinant antigen-binding proteins.

As used herein, the expression “specifically binds” or “binds specifically” refers to those antibodies or antigen-binding fragments thereof having a binding affinity to an antigen, such as SLC3A2-APIS or CD3 protein, expressed as K_{D}, of less than about 10⁻⁶ M (e.g., 10⁻⁷ M, 10⁻⁸ M, 10⁻⁹ M, 10⁻¹⁰ M, 10⁻¹¹ M or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA.

As used herein, “anti-SLC3A2-APIS” refers to an antigen-binding protein (or other molecule such as an antigen-binding arm), for example an antibody or antigen-binding fragment thereof, that binds specifically to SLC3A2-APIS and “anti-CD3” refers to an antigen-binding protein (or other molecule such as an antigen-binding arm), for example an antibody or antigen-binding fragment thereof, that binds specifically to CD3. The term “anti-CD3” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). “SLC3A2-APISxCD3” refers to an antibody or antigen-binding fragment thereof that binds specifically to SLC3A2-APIS and to CD3 (and, optionally, to one or more other antigens).

As used herein, the term “epitope” refers to an antigenic determinant (e.g., on SLC3A2-APIS or CD3) that interacts with a specific antigen-binding site of an antigen-binding protein, e.g., a variable region of an antibody molecule, known as a paratope. A single antigen may have more than one epitope. Thus, different antibodies or antigen-binding fragments thereof may bind to different areas on an antigen and may have different biological effects. This term may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding molecule or polypeptide include alanine scanning mutational analysis, peptide blot analysis (Reineke (2004) Methods Mol. Biol. 248:443-63), peptide cleavage analysis, crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antigen-binding molecule or polypeptide interacts is hydrogen/deuterium exchange detected by mass spectrometry. See, e.g., Ehring (1999) Analytical Biochemistry 267:252-259; Engen and Smith (2001) Anal. Chem. 73: 256A-265A.

The present disclosure includes antigen-binding molecules that compete for binding to SLC3A2-APIS with another antibody or antigen-binding fragment thereof. As used herein, an antigen-binding molecule that “competes” refers to an antigen-binding protein (e.g., antibody or antigen-binding fragment thereof) that binds to an antigen and inhibits or blocks the binding of another antigen-binding protein (e.g., antibody or antigen-binding fragment thereof) to the antigen. Unless otherwise stated, the term also includes competition between two antigen-binding molecules, in both orientations, i.e., a first antibody that binds antigen and blocks binding by a second antibody and vice versa. In some embodiments, competition occurs in one such orientation. In some embodiments, the first antigen-binding protein and second antigen-binding protein may bind to the same epitope. Alternatively, in some embodiments, the first and second antigen-binding proteins may bind to different but overlapping or non-overlapping epitopes, wherein binding of one inhibits or blocks the binding of the second antibody, e.g., via steric hindrance. Competition between antigen-binding proteins may be measured by known methods, such as a real-time, label-free bio-layer interferometry assay. Also, binding competition between antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment of the disclosure which is modified in some way retains the ability to specifically bind to SLC3A2-APIS and CD3, e.g., retains at least 10% of its SLC3A2-APIS and CD3 binding activity (when compared to the parental antibody) when that activity is expressed on a molar basis. Preferably, an antibody or antigen-binding fragment of the disclosure retains at least 20%, at least 50%, at least 70%, at least 80%, at least 90%, at least 95% or 100% of the SLC3A2-APIS and CD3 binding affinity as the parental antibody. In some embodiments, an antibody or antigen-binding fragment of the disclosure may include conservative or non-conservative amino acid substitutions (referred to as “conservative variants” or “function conserved variants” of the antibody) that do not substantially alter its biologic activity.

As used herein, the singular forms “a,” “an,” and “the” include plural reference unless the context clearly dictates otherwise. As used herein, the terms “including,” “comprising,” “containing,” or “having” and variations thereof are meant to encompass the items listed thereafter and equivalents thereof as well as additional subject matter unless otherwise noted. As used herein, the phrases “in one embodiment,” “in various embodiments,” “in some embodiments,” and the like are used repeatedly. Such phrases do not necessarily refer to the same embodiment, but they may unless the context dictates otherwise. As used herein, the terms “and/or” or “/” means any one of the items, any combination of the items, or all of the items with which this term is associated.

The following references relate to BLAST algorithms often used for sequence analysis: Altschul et al. (2005) FEBS J. 272 (20): 5101-5109; Altschul et al., (1990) J. Mol. Biol. 215:403-410; Gish et al., (1993) Nature Genet. 3:266-272; Madden et al., (1996) Meth. Enzymol. 266:131-141; Altschul et al., (1997) Nucleic Acids Res. 25:3389-3402; Zhang (1997) Genome Res. 7:649-656; Wootton et al., (1993) Comput. Chem. 17:149-163; Hancock et al., (1994) Comput. Appl. Biosci. 10:67-70.

The following references relate to Alignment Scoring Systems: Dayhoff et al., “A Model of Evolutionary Change in Proteins,” ATLAS OF PROTEIN SEQUENCE AND STRUCTURE, (1978) vol. 5, suppl. 3., Dayhoff (ed.), pp. 345-352, Natl. Biomed. Res. Found., Washington, DC; Schwartz et al., “Matrices for Detecting Distant Relationships,” ATLAS OF PROTEIN SEQUENCE AND STRUCTURE, (1978) vol. 5, suppl. 3.” Dayhoff (ed.), pp. 353-358, Natl. Biomed. Res. Found., Washington, DC; Altschul (1991) J. Mol. Biol. 219:555-565; States, (1991) Methods 3:66-70; Henikoff et al., (1992) Proc. Natl. Acad. Sci. USA 89:10915-10919; Altschul et al., (1993) J. Mol. Evol. 36:290-300.

The following references relate to Alignment Statistics: Karlin et al., (1990) Proc. Natl. Acad. Sci. USA 87:2264-2268; Karlin et al., (1993) Proc. Natl. Acad. Sci. USA 90:5873-5877; Dembo et al., (1994) Ann. Prob. 22:2022-2039; Altschul, “Evaluating the Statistical Significance of Multiple Distinct Local Alignments,” THEORETICAL AND COMPUTATIONAL METHODS IN GENOME RESEARCH (Suhai, ed.), (1997) pp. 1-14, Plenum, NY.

### SLC3A2-APIS Antibodies and Antigen-Binding Fragments Thereof

The antibodies and antigen-binding fragments thereof of the present disclosure may be monospecific or bispecific. Bispecific antibodies and antigen-binding fragments thereof may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The antibodies and antigen-binding fragments thereof of the present disclosure can be linked to or co-expressed with another functional molecule, e.g., another peptide, protein, or fragment thereof. For example, an antibody thereof can be functionally linked (e.g., by chemical coupling, genetic fusion, noncovalent association or otherwise) to one or more other molecular entities, such as another antibody to produce a bispecific antibody with a second binding specificity.

As used herein, the expression “anti-SLC3A2-APIS antibody” includes both monospecific anti-SLC3A2-APIS antibodies and bispecific antibodies comprising a SLC3A2-APIS-binding arm and a second arm that binds a different antigen (e.g., CD3). The SLC3A2-APIS-binding arm can comprise any of the HCVR, LCVR, and/or CDR amino acid sequences of the examples set forth in Table 1 or 4, or otherwise disclosed herein. In certain embodiments, the CD3-binding arm binds human CD3 and induces human T-cell proliferation.

In some embodiments, the disclosed antibodies or antigen-binding fragments thereof include a SLC3A2-APIS-binding arm that includes a heavy chain immunoglobulin that comprises a HCVR including the combination of heavy chain CDRs (HCDR1, HCDR2 and HCDR3) and the corresponding light chain immunoglobulin that comprises a LCVR including the combination of light chain CDRs (LCDR1, LCDR2 and LCDR3) as set forth herein or in WO2014/004427.

The present disclosure includes monospecific antibodies and antigen-binding fragments thereof that specifically bind SLC3A2-APIS. Such molecules may be referred to herein as “anti-SLC3A2-APIS” or “SLC3A2-APIS.” The present disclosure also includes bispecific antibodies and antigen-binding fragments thereof that specifically bind SLC3A2-APIS and CD3. Such molecules may be referred to herein as “anti-SLC3A2-APIS/anti-CD3,” “anti-CD3xSLC3A2-APIS,” “CD3xSLC3A2-APIS,” “SLC3A2-APISxCD3,” “anti-SLC3A2-APIS/anti-CD3,” “anti-SLC3A2-APISxCD3,” “anti-SLC3A2-APIS×anti-CD3,” “anti-CD3×anti-SLC3A2-APIS,” or similar terminology.

In some embodiments, the disclosure includes a bispecific antibody that has an effector arm and a targeting arm. The effector arm may be the first antigen-binding arm that binds to CD3 on effector cells (e.g., T cells). The targeting arm may be the second antigen-binding arm that binds to an antigen, e.g., SLC3A2-APIS on target cells (e.g., tumor cells). In some embodiments of the present disclosure, the effector arm binds to CD3 and the targeting arm binds to SLC3A2-APIS.

In some embodiments of the disclosed bispecific antigen-binding molecules, the first antigen-binding arm and the second antigen-binding arm may be directly or indirectly connected to one another. Alternatively, the first antigen-binding arm and the second antigen-binding arm may each be connected to a separate multimerizing domain. The association of one multimerizing domain with another multimerizing domain facilitates the association between the two antigen-binding domains, thereby forming a bispecific antigen-binding molecule.

As used herein, a “multimerizing domain” refers to any macromolecule, protein, polypeptide, peptide, or amino acid that has the ability to associate with a second multimerizing domain of the same or similar structure. For example, a multimerizing domain may be a polypeptide comprising an immunoglobulin CH3 domain. A non-limiting example of a multimerizing domain is an Fc portion of an immunoglobulin (comprising a CH2-CH3 domain), e.g., an Fc domain of an IgG selected from the isotypes IgG1, IgG2, IgG3, and IgG4, as well as any allotype within each isotype group. The Fc domain may comprise wild-type or modified IgG isotype.

In some embodiments, the disclosed bispecific antibodies or antigen-binding fragments thereof comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of 1 to about 200 amino acids in length containing at least one cysteine residues. In other embodiments, the multimerizing domain is a cysteine residue, or a short cysteine containing peptide. Other multimerizing domains include peptides or polypeptides comprising or consisting of a leucine zipper, a helix-loop motif, or a coiled-coil motif.

Any bispecific antibody format or technology may be used to make the bispecific antibodies of the present disclosure. For example, an antibody or antigen-binding fragment thereof having a first antigen-binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (OVO)-Ig, Quadroma, knobs-into-holes, common light chain (e.g., common light chain with knobs-intoholes, etc.), CrossMab, CrossFab, (SEEO) body, leucine zipper, Ouobody, IgG1/IgG2, dual acting Fab (OAF)-IgG, and Mab² bispecific formats (see, e.g., Klein et al. 2012, mAbs 4:6, 1-11, and references cited therein, for a review of the foregoing formats).

The multimerizing domains, e.g., Fc domains, may comprise one or more amino acid changes (e.g., insertions, deletions or substitutions) as compared to the wild-type, naturally occurring version of the Fc domain. For example, the disclosed bispecific antibodies or antigen-binding fragments thereof may comprise one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., LN/FIW or T), 254 (e.g., S or T), and 256 (e.g., S/R/Q/EID or T); or a modification at position 428 and/or 433 (e.g., UR/S/P/Q or K) and/or 434 (e.g., H/F or V); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252,254, and 256 (e.g., 252Y, 254T, and 256E) modification; a 250Q and 428L modification (e.g., T250Q and M428L); and a 307 and/or 308 modification (e.g., 308F or 308P).

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L 18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antibodies set forth herein comprises, from N— to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antibodies set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antibodies of the present disclosure are described in WO2014/022540. Chimeric Fc domains having these general structural arrangements, and variants thereof, can have altered Fc receptor binding, which in turn affects Fc effector function.

Antibodies and antigen-binding fragments of the present disclosure comprise immunoglobulin chains including the amino acid sequences specifically set forth herein (and variants thereof) as well as cellular and in vitro post-translational modifications to the antibody or fragment. For example, the present disclosure includes monospecific antibodies and antigen-binding fragments thereof that specifically bind to SLC3A2-APIS, and bispecific antibodies and antigen-binding fragments thereof that specifically bind to SLC3A2-APIS and CD3 comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

### Polynucleotides and Methods of Making

The present disclosure includes isolated polynucleotide molecules or sets of isolated polynucleotide molecules comprising polynucleotide sequences encoding the immunoglobulin chains of a SLC3A2-APIS monospecific or SLC3A2-APISxCD3 bispecific antigen-binding protein. The present disclosure also includes vectors or sets of vectors comprising the polynucleotide molecules and/or a host cell (e.g., a CHO cell) comprising the polynucleotide molecules, vector(s) or antigen-binding protein(s) set forth herein.

A polynucleotide molecule or sequence refers to DNA or RNA. In some embodiments, the disclosed polynucleotide molecules encode an immunoglobulin V_{H}, V_{L}, CDRs-H, CDRs-L, HC, and/or LC of an SLC3A2-APIS binding arm and/or a CD3 binding arm. Optionally, the disclosed polynucleotide molecule is operably linked to a promoter or other expression control sequence or other polynucleotide sequence. Examples of a polynucleotide molecule or set of polynucleotide molecules (e.g., DNA) of the present disclosure include a nucleotide sequence set forth in Table 2 or 5.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the disclosure. Examples of promoters which may be used to control gene expression include, but are not limited to, cytomegalovirus (CMV) promoter (U.S. Pat. Nos. 5,385,839 and 5,168,062), the SV40 early promoter region (Benoist et al., (1981) Nature 290:304-310), the promoter contained in the 3′ long terminal repeat of Rous sarcoma virus (Yamamoto et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

Eukaryotic and prokaryotic host cells, including mammalian cells, may be used as hosts for expression of a SLC3A2-APIS monospecific antibody or antigen-binding arm thereof or SLC3A2-APISxCD3 bispecific antibody or antigen-binding arm thereof. Such host cells are well known in the art and many are available from the American Type Culture Collection (ATCC). These host cells include, inter alia, CHO cells, NSO, SP2 cells, Hela cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used include insect cell lines (e.g., Spodoptera frugiperda or Trichoplusia ni), amphibian cells, bacterial cells, plant cells and fungal cells. Fungal cells include yeast and filamentous fungus cells. The present disclosure includes an isolated host cell (e.g., a CHO cell or any type of host cell set forth above) comprising a SLC3A2-APIS monospecific antigen-binding protein of the present disclosure such as the antiSLC3A2-APIS antigen-binding proteins shown in Table 1, and an isolated host cell (e.g., a CHO cell or any type of host cell set forth above) comprising an SLC3A2-APISxCD3 bispecific antigen-binding protein of the present disclosure, such as bsAb17234 and bsAb17235 and the SLC3A2-APISxCD3 antigen-binding proteins shown in Table 4, or one or more polynucleotide molecules encoding an immunoglobulin (Ig) heavy and/or light chain thereof; and/or one or more polynucleotides encoding the SLC3A2-APIS binding arm and/or CD3 binding arm of an antigen-binding protein of the present disclosure, such as the SLC3A2-APISxCD3 antibodies shown in Table 6.

The present disclosure also includes a cell which is expressing an SLC3A2-APIS and/or CD3 or an antigenic fragment or fusion thereof (e.g., His₆, Fc and/or myc) which is bound by an SLC3A2-APIS or SLC3A2-APISxCD3 antigen-binding protein of the present disclosure (e.g., an antibody or antigen-binding fragment thereof), for example, bsAb17234 and bsAb17235, as well as any of monospecific or bispecific antibodies disclosed herein.

There are several methods by which to produce recombinant antibodies which are known in the art. See, e.g., U.S. Pat. No. 4,816,567. Transformation can be by any known method for introducing polynucleotides into a host cell. Methods for introduction of heterologous polynucleotides into mammalian cells are known. In addition, nucleic acid molecules may be introduced into mammalian cells by viral vectors. Methods of transforming cells are known. See, e.g. U.S. Pat. Nos. 4,399,216; 4,912,040; 4,740,461; 4,959,455.

The present disclosure includes recombinant methods for making an anti-SLC3A2-APIS and anti-SLC3A2-APIS×anti-CD3 antibody, such as an immunoglobulin chain thereof, comprising: (i) introducing, into a host cell, one or more polynucleotides encoding the light and heavy immunoglobulin chains encoding the SLC3A2-APIS antibody's or SLC3A2-APISxCD3 antibody's antigen-binding arms for example, wherein the polynucleotide is in a vector; and/or integrates into the host cell chromosome and/or is operably linked to a promoter; (ii) culturing the host cell (e.g., a CHO cell or Pichia or Pichia pastoris) under conditions favorable to expression of the polynucleotide, and (iii) optionally, isolating the antibody or chain from the host cell and/or medium in which the host cell is grown. The present disclosure also includes SLC3A2-APIS antibodies or antigen-binding fragments thereof and SLC3A2-APISxCD3 antibodies or antigen-binding fragments thereof, which are the products of production methods and, optionally, the purification methods set forth herein.

In some embodiments, the disclosed SLC3A2-APIS or SLC3A2-APISxCD3 antibody or antigen-binding fragment thereof may be made by a method that includes purifying the antibody or antigen-binding fragment thereof, e.g., by column chromatography, precipitation and/or filtration. The present disclosure also includes the product of such a method.

### Sequence Variants

In some embodiments, the disclosed antibodies comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases.

In some embodiments, the disclosed antigen-binding fragments of antibodies are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In some embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired properties such as improved binding specificity, increased binding affinity, improved or enhanced antagonistic or agonistic biological properties (as the case may be), reduced immunogenicity, etc.

### Antibodies Comprising Fc Variants

According to certain embodiments of the present disclosure, anti-SLC3A2-APIS monospecific antibodies and anti-SLC3A2-APIS×anti-CD3 bispecific antibodies are provided comprising an Fc domain comprising one or more mutations which enhance or diminish antibody binding to the FcRn receptor, e.g., at acidic pH as compared to neutral pH. For example, the present disclosure includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
   or a modification at position:
428 and/or 433 (e.g., H/L/R/S/P/Q or K) and/or
434 (e.g., H/F or Y);
   or a modification at position:
250 and/or 428;
   or a modification at position:
307 or 308 (e.g., 308F, V308F), and/or
434.

In one embodiment, the modification comprises one or more of the following:
428L (e.g., M428L) and 434S (e.g., N434S) modification;
a 428L, 259| (e.g., V2591), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the disclosed SLC3A2-APISxCD3 bispecific antibodies may comprise an Fc domain comprising one or more pairs or groups of mutations selected from:
250Q and 248L (e.g., T250Q and M248L);
252Y, 254T and 256E (e.g., M252Y, S254T and T256E);
428L and 434S (e.g., M428L and N434S); and
433K and 434F (e.g., H433K and N434F).

The present disclosure also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, e.g., U.S. Pat. No. 8,586,713. Further modifications that may be found within the second C_{H}3 include: D16E, L18M, N44S, K52N, V57M, and V821 (by IMGT; D356E, L358M, N384S, K392N, V397M, and V4221 by EU) for IgG1 antibodies; N44S, K52N, and V821 (IMGT; N384S, K392N, and V4221 by EU) for IgG2 antibodies; and Q15R, N44S, K52N, V57M, R69K, E79Q, and V821 (by IMGT; Q355R, N384S, K392N, V397M, R409K, E419Q, and V4221 by EU) for IgG4 antibodies.

All possible combinations of the foregoing Fc domain mutations, and other mutations within the antibody variable domains disclosed herein, are within the scope of the present disclosure.

### Biological Characteristics of the Bispecific Antibodies

In some embodiments, the disclosed bispecific antibodies and antigen-binding fragments thereof bind human SLC3A2-APIS and/or CD3 with high affinity, medium affinity or low affinity, depending on the therapeutic context and particular targeting properties that are desired. For example, in the context of a bispecific SLC3A2-APISxCD3 antigen-binding molecule, it may be desirable for the target antigen-binding arm to bind the target antigen with high affinity while the anti-CD3 arm binds CD3 with only moderate or low affinity. In this manner, preferential targeting of the antigen-binding molecule to cells expressing the target antigen may be achieved while avoiding general/untargeted CD3 binding and the consequent adverse side effects associated therewith.

According to certain embodiments, the present disclosure includes antibodies and antigen-binding fragments of antibodies that bind human SLC3A2-APIS (e.g., at 25° C.) with a K_{D} of less than about 200 nM as measured by surface plasmon resonance, e.g., using an assay format as defined in Example 2 herein. In certain embodiments, the antibodies or antigen-binding fragments of the present disclosure bind SLC3A2-APIS with a K_{D} of less than about 100 nM, less than about 90 nM, less than about 80 nM, less than about 60 nM, less than about 40 nM, less than about 30 nM, less than 20 nM, less than 10 nM, or less than 5 nM as measured by surface plasmon resonance, e.g., using an assay format as defined in Example 2 herein, or a substantially similar assay. In certain embodiments, the antibodies or antigen-binding fragments of the present disclosure bind SLC3A2-APIS with a K_{D} of about 1 nM to about 10 nM.

The present disclosure also includes antibodies and antigen-binding fragments thereof that bind SLC3A2-APIS with a dissociative half-life (t½) of greater than about 2 minutes as measured by surface plasmon resonance at 25° C. or 37° C., e.g., using an assay format as defined in the Examples herein, or a substantially similar assay. In certain embodiments, the antibodies or antigen-binding fragments of the present disclosure bind CD3 with a t½ of greater than about 10 minutes, greater than about 20 minutes, greater than about 30 minutes, greater than about 40 minutes, greater than about 50 minutes, greater than about 60 minutes, or greater than about 80 minutes, as measured by surface plasmon resonance at 25° C. or 37° C., e.g., using an assay format as defined in Example 2 herein, or a substantially similar assay.

The present disclosure includes monospecific antibodies capable of binding to human SLC3A2-APIS, and bispecific antibodies capable of simultaneously binding to human CD3 and human SLC3A2-APIS. According to certain embodiments, the disclosed bispecific antibodies specifically interact with cells that express SLC3A2-APIS and CD3. The extent to which a bispecific antigen-binding molecule binds cells that express CD3 and SLC3A2-APIS can be assessed by fluorescence activated cell sorting (FACS), as illustrated in Example 3 herein.

For example, the present disclosure includes bispecific antibodies which specifically bind human cell lines which express CD3 but not SLC3A2-APIS. In some embodiments, the bispecific antibodies bind to CD3-expressing human or cynomolgus T cells with an EC50 value less than 1×10⁻⁵ M. In some embodiments, the bispecific antibodies bind to CD3-expressing human or cynomolgus T cells with an EC50 value of 1×10⁻¹² M to 1×10⁻⁵ M. In certain embodiments, the bispecific antibodies bind to CD3-expressing human or cynomolgus T cells with an EC50 value of 1×10⁻¹² M to 1×10⁻⁹ M. In certain embodiments, the bispecific antigen-binding molecules bind to the surface of cell lines expressing SLC3A2-APIS with an EC₅₀ of less than about 2.5×10⁻⁸ M. The binding of the bispecific antigen-binding molecules to the surface of cells or cell lines can be measured by an in vitro FACS binding assay as described in the Examples.

The present disclosure includes SLC3A2-APIS monospecific antigen-binding molecules and SLC3A2-APISxCD3 bispecific antigen-binding molecules which are capable of depleting tumor cells in a subject. The depletion or cytotoxic activity can be measured in a assay as described in Example 6.

The present disclosure includes SLC3A2-APIS monospecific antigen-binding molecules and SLC3A2-APISxCD3 bispecific antigen-binding molecules which are capable of binding to SLC3A2-APIS expressed on cell surfaces. A variety of tumor cells express SLC3A2-APIS, including acute myleloid leukemia cells (e.g., HNT-34), pancreatic cancer cells (e.g., PANC05.04), and uveal melanoma tumor cells (e.g., Mel-202). As such, the antigen-binding molecules of the disclosure are useful in treating a multitude of cancer indications.

The present disclosure includes SLC3A2-APIS monospecific antigen-binding molecules and SLC3A2-APISxCD3 bispecific antigen-binding molecules which are capable of activating T cells by engaging SLC3A2-APIS on target cells and, in the case of bispecific antigen-binding molecules, CD3 on T cells. As such, the antigen-binding molecules of the disclosure are useful in promoting a T-cell mediated anti-tumor response.

### Epitope Mapping and Related Technologies

The epitope on SLC3A2-APIS or CD3 to which the antigen-binding molecules of the present disclosure may bind may consist of a single contiguous sequence of 3 or more (e.g., 3, 4, 5, 6, 7, 8, 9, 10, 11, 12, 13, 14, 15, 16, 17, 18, 19, 20 or more) amino acids of a SLC3A2-APIS or CD3 protein. Alternatively, the epitope may consist of a plurality of non-contiguous amino acids (or amino acid sequences) of SLC3A2-APIS or CD3. The antigen-binding molecules of the disclosure may interact with amino acids contained within a SLC3A2-APIS or CD3 monomer, or may interact with amino acids on two different SLC3A2-APIS or CD3 chains of a SLC3A2-APIS or CD3 dimer. The term “epitope,” as used herein, refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in ANTIBODIES, Harlow & Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267 (2): 252-259; Engen and Smith (2001) Anal. Chem. 73: 256A-265A. Alternatively, in certain embodiments, the protein of interest binds to the antibody, followed by hydrogen-deuterium exchange. After dissociation of the antibody, the target protein is subjected to protease cleavage and mass spectrometry analysis, thereby revealing the non-deuterium-labeled residues which correspond to the specific amino acids with which the antibody interacts. X-ray crystal structure analysis can also be used to identify the amino acids within a polypeptide with which an antibody interacts. CryoEM imaging can also be used identify the amino acids within a polypeptide with which an antibody interacts. CryoEM imaging is especially effective at identifying the structure of transmembrane proteins like SLC3A2 (or the neoantigen SLC3A2-APIS).

The present disclosure further includes anti-SLC3A2-APIS and anti-CD3 antibodies that bind to the same epitope as any of the specific exemplary antibodies described herein (e.g., antibodies comprising any of the amino acid sequences as set forth in Tables 1, 4, and 6). Likewise, the present disclosure also includes anti-SLC3A2-APIS antibodies that compete for binding to SLC3A2-APIS with any of the specific exemplary antibodies described herein (e.g., antibodies comprising any of the amino acid sequences as set forth in Tables 1, 4, and 6 herein).

The present disclosure also includes bispecific antigen-binding molecules comprising a first antigen-binding domain that specifically binds human CD3, and a second antigen-binding fragment that specifically binds human SLC3A2-APIS, wherein the first antigen-binding domain binds to the same epitope on CD3 as any of the specific exemplary CD3-specific antigen-binding domains described herein, and/or wherein the second antigen-binding domain binds to the same epitope on SLC3A2-APIS as any of the specific exemplary SLC3A2-APIS-specific antigen-binding domains described herein. Likewise, the present disclosure also includes bispecific antigen-binding molecules comprising a first antigen-binding domain that specifically binds human CD3, and a second antigen-binding fragment that specifically binds human SLC3A2-APIS, wherein the first antigen-binding domain competes for binding to CD3 with any of the specific exemplary SLC3A2-APIS-specific antigen-binding domains described herein, and/or wherein the second antigen-binding domain competes for binding to SLC3A2-APIS with any of the specific exemplary SLC3A2-APIS-specific antigen-binding domains described herein.

One can easily determine whether a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on CD3 (or SLC3A2-APIS) as a reference bispecific antigen-binding molecule of the present disclosure, the reference bispecific molecule is first allowed to bind to a CD3 protein (or SLC3A2-APIS protein). Next, the ability of a test antibody to bind to the CD3 (or SLC3A2-APIS) molecule is assessed. If the test antibody is able to bind to CD3 (or SLC3A2-APIS) following saturation binding with the reference bispecific antigen-binding molecule, it can be concluded that the test antibody does not compete for binding to CD3 (or SLC3A2-APIS) with the reference bispecific antigen-binding molecule and/or that there is steric interference between antibodies that are binding different sites on the antigen. On the other hand, if the test antibody is not able to bind to the CD3 (or SLC3A2-APIS) molecule following saturation binding with the reference bispecific antigen-binding molecule, then the test antibody competes for binding to CD3 (or SLC3A2-APIS) with the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antigen-binding proteins compete for binding to an antigen if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins may bind to the same epitope if essentially all amino acid mutations in the antigen that reduce or eliminate binding of one antigen-binding protein reduce or eliminate binding of the other. Two antigen-binding proteins may have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a CD3 protein (or SLC3A2-APIS protein) under saturating conditions followed by assessment of binding of the test antibody to the CD3 (or SLC3A2-APIS) molecule. In a second orientation, the test antibody is allowed to bind to a CD3 (or SLC3A2-APIS) molecule under saturating conditions followed by assessment of binding of the reference antigen-binding molecule to the CD3 (or SLC3A2-APIS) molecule. If, in both orientations, only the first (saturating) antigen-binding molecule is capable of binding to the CD3 (or SLC3A2-APIS) molecule, then it is concluded that the test antibody and the reference antigen-binding molecule compete for binding to CD3 (or SLC3A2-APIS). As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Bispecific Molecules

Antigen-binding domains specific for particular antigens can be prepared by any antibody generating technology known in the art. Once obtained, two different antigen-binding domains, specific for two different antigens (e.g., SLC3A2-APIS and CD3), can be appropriately arranged relative to one another to produce a bispecific antigen-binding molecule of the present disclosure using routine methods. (A discussion of exemplary bispecific antibody formats that can be used to construct the bispecific antigen-binding molecules of the present disclosure is provided elsewhere herein). In certain embodiments, one or more of the individual components (e.g., heavy and light chains) of the bispecific antigen-binding molecules of the disclosure are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., SLC3A2-APIS or CD3) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present disclosure.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454 for a detailed discussion of such engineered mice and the use thereof to produce bispecific antigen-binding molecules).

### Bioequivalents

The present disclosure encompasses antigen-binding molecules having amino acid sequences that vary from those of the described antibodies but that retain the ability to bind SLC3A2-APIS and CD3. Such variant molecules comprise one or more additions, deletions, or substitutions of amino acids when compared to parent sequence, but exhibit biological activity that is essentially equivalent to that of the described antigen-binding molecules. Likewise, the antigen-binding molecules-encoding DNA sequences of the present disclosure encompass sequences that comprise one or more additions, deletions, or substitutions of nucleotides when compared to the disclosed sequence, but that encode an antigen-binding molecule that is essentially bioequivalent to the described antigen-binding molecules of the disclosure. Examples of such variant amino acid and DNA sequences are discussed above.

The present disclosure includes antigen-binding molecules that are bioequivalent to any of the exemplary antigen-binding molecules set forth herein. Two antigen-binding proteins or antibodies are considered bioequivalent if, for example, they are pharmaceutical equivalents or pharmaceutical alternatives having a rate and extent of absorption that do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a subject can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include the exemplary bispecific antigen-binding molecules set forth herein comprising amino acid changes which modify the glycosylation characteristics of the antibodies, e.g., mutations which eliminate or remove glycosylation.

In some embodiments, the disclosed antibodies and antigen-binding fragments thereof comprise an antigen-binding domain with an HCVR, LCVR, and/or CDR amino acid sequence that has at least 90%, at least 95%, at least 98% or at least 99% (but less than 100%) sequence identity to any of the HCVR, LCVR, and/or CDR amino acid sequences disclosed herein. Sequence identity may be measured by methods known in the art (e.g., GAP, BESTFIT, and BLAST). Antibodies and antigen-binding fragments having an HCVR, LCVR, and/or CDR amino acid sequence with less than 100% identity to a corresponding sequence disclosed herein may have one or more substitutions of amino acids with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment.

Those of skill in the art recognize that, in general, limited (e.g., single) amino acid substitutions in non-essential regions of a polypeptide do not substantially alter biological activity (see, e.g., Watson et al. (1987) MOLECULAR BIOLOGY OF THE GENE, The Benjamin/Cummings Pub. Co., p. 224 (4^{th} Ed.)). Also, substitutions of structurally or functionally similar amino acids are less likely to significantly disrupt biological activity. Examples of groups of amino acids that have side chains with similar chemical properties include 1) aliphatic side chains: glycine, alanine, valine, leucine and isoleucine; 2) aliphatic-hydroxyl side chains: serine and threonine; 3)amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine.

### Immunoconjugates

The disclosure encompasses SLC3A2-APIS antigen-binding proteins, e.g., antibodies or antigen-binding fragments, conjugated to another moiety, e.g., a therapeutic moiety (an “immunoconjugate”). In an embodiment, an SLC3A2-APIS antigen-binding protein, e.g., antibody or antigen-binding fragment, is conjugated to any of the further therapeutic agents set forth herein. As used herein, the term “immunoconjugate” refers to an antigen-binding protein, e.g., an antibody or antigen-binding fragment, which is chemically or biologically linked to another antigen-binding protein, a drug, a radioactive agent, a reporter moiety, an enzyme, a peptide, a protein or a therapeutic agent.

In certain embodiments, the therapeutic moiety may be a cytotoxin, a chemotherapeutic drug, an immunosuppressant or a radioisotope. Cytotoxic agents include any agent that is detrimental to cells. Examples of suitable cytotoxic agents and chemotherapeutic agents for forming immunoconjugates are known in the art. See, e.g., WO 05/103081.

### Therapeutic Uses of the Antigen-Binding Molecules

The bispecific antibodies and antigen-binding molecules of the disclosure (and therapeutic compositions comprising the same) are useful, inter alia, for treating any disease or disorder in which stimulation, activation and/or targeting of an immune response would be beneficial. In particular, the SLC3A2-APISxCD3 bispecific antigen-binding molecules of the present disclosure may be used for the treatment, prevention and/or amelioration of a hyperproliferative disease, for example, cancer. In certain embodiments, the present disclosure provides methods for treating cancer in a subject, comprising administering a therapeutically effective dose of SLC3A2-APISxCD3 antigen-binding protein, e.g., bsAb17234 or bsAb17235 to the subject.

A hyperproliferative disease, for the purposes herein, refers to a disease characterized by abnormal, excessive and/or uncontrolled cell growth, e.g., a cancer, wherein the cells express SLC3A2-APIS. For example, hyperproliferative diseases include cancers. Exemplary cancers include, but are not limited to anal cancer, angiosarcoma, a B cell cancer, basal cell carcinoma, bladder cancer, bone cancer, brain cancer, breast cancer, cervical cancer, cholangiocarcinoma, chondrosarcoma, chronic lymphocytic leukemia, colon cancer, colorectal cancer, cutaneous squamous cell carcinoma, endometrial cancer, esophageal cancer, glioblastoma multiforme, head and neck squamous cell cancer, hepatocellular carcinoma, heme cancer, kidney cancer, liver cancer, lung cancer, lymphoma, leukemia, Merkel cell carcinoma, melanoma, myeloma, myeloproliferative malignancy, non-small cell lung cancer, ovarian cancer, pancreatic cancer, prostate cancer, salivary gland cancer, skin cancer, soft tissue sarcoma, stomach cancer, a T cell cancer, testicular cancer, uterine cancer, and uveal melanoma.

Accordingly, the antibodies and the bispecific antigen-binding molecules of the present disclosure can be used in treating a wide range of cancers.

Cancer characterized by solid tumor cells or cancerous blood cells may be an SLC3A2-APIS-expressing cancer e.g., wherein SLC3A2-APIS expression in the cells of the particular subject to be treated has been confirmed, includes esophageal carcinoma, lung squamous cell carcinoma, lung adenocarcinoma, cervical squamous cell carcinoma, endometrial adenocarcinoma, bladder urothelial carcinoma, lung cancer (e.g., non-small cell lung cancer), colorectal cancer, rectal cancer, endometrial cancer, skin cancer (e.g., head & neck squamous cell carcinoma), brain cancer (e.g., glioblastoma multiforme), breast cancer, gastroesophageal cancer, (e.g., gastroesophageal adenocarcinoma), prostate cancer, ovarian cancer, melanoma, basal cell carcinoma, cervical cancer, diffuse large B cell lymphoma, and/or multiple myeloma. Examples of hematologic cancers are leukemia, including acute and chronic myeloid leukemia (AML and CML, respectively) and acute and chronic lymphoblastic leukemia (ALL and CLL, respectively); lymphoma, including B-cell lymphomas such as non-Hodgkin's lymphoma (NHL), Hodgkin's lymphoma (HL), and myeloma, including multiple myeloma (MM).

The antigen-binding molecules of the present disclosure may also be used to treat, e.g., primary and/or metastatic tumors arising in the colon, lung, breast, ovary, kidney, and bladder (or from any cancer discussed herein).

The antigen-binding proteins of the present disclosure may also be used to treat residual cancer in a subject. As used herein, the term “residual cancer” means the existence or persistence of one or more cancerous cells in a subject following treatment with an anti-cancer therapy.

The antigen-binding proteins of the present disclosure may also be used to prevent a premalignant or pre-cancerous condition from progressing to a cancer in a subject. As used herein, the terms “premalignant condition” or “precancerous condition” refer to a general state associated with a significantly increased risk of cancer in a subject. Precancerous conditions include, for example, cytopenia in peripheral blood cells, fibrosis or presence of clinically relevant mutations. In certain embodiments, the subject has a premalignant condition selected from idiopathic cytopenia of undetermined significance (ICUS), clonal hematopoiesis of indeterminate potential (CHIP), clonal cytopenia of undetermined significance (CCUS), idiopathic dysplasia of unknown significance (IDUS) or age-related clonal hematopoiesis (ARCH). In certain embodiments, an antigen-binding protein of the present disclosure is administered to a subject having a premalignant condition or at an increased risk of developing a cancer, wherein the administration results in preventing the premalignant condition from progressing to a cancer.

As used herein, the term “subject” refers to a mammal (e.g., rat, mouse, cat, dog, cow, sheep, horse, goat, rabbit), preferably a human, for example, in need of prevention and/or treatment of cancer. The subject may have a cancer, be predisposed to developing such a condition, and/or would benefit from administration of a bispecific antibody or antigen-binding fragment thereof of the present disclosure. In one embodiment, the subject may have, or be at risk of developing, a hyperproliferative disease.

Methods for treating or preventing a cancer (e.g., a SLC3A2-APIS-expressing cancer) in a subject in need of said treatment or prevention by administering a therapeutically effective dose amount SLC3A2-APISxCD3 antigen-binding protein, in association with an additional therapeutic agent are part of the present disclosure. Additional therapeutic agents are disclosed elsewhere herein.

An “effective” or “therapeutically effective” amount of SLC3A2-APISxCD3 antigen-binding protein, e.g., antibody or antigen-binding fragment, for treating or preventing a hyperproliferative disease, such as cancer, is the amount of the antigen-binding protein sufficient to alleviate one or more signs and/or symptoms of the disease in the treated subject, whether by inducing the regression or elimination of such signs and/or symptoms or by inhibiting the progression of such signs and/or symptoms. In some embodiments, a therapeutically effective dose of SLC3A2-APISxCD3 antigen-binding protein is 0.1-2000 mg. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses may be separated by 1-8 weeks.

The dose of antigen-binding molecule administered to a subject may vary depending upon the age and the size of the subject, target disease, conditions, route of administration, and the like. In one embodiment, the dose is calculated according to body weight or body surface area. In another embodiment, the dose is flat dose. Depending on the severity of the condition, the frequency and the duration of the treatment can be adjusted. Effective dosages and schedules for administering a bispecific antigen-binding molecule may be determined empirically; for example, subject progress can be monitored by periodic assessment, and the dose adjusted accordingly.

### Combination Therapies

The bispecific antigen-binding molecules of the present disclosure may be used in combination with one or more agents, for example, in treating a cancer in a subject. In certain embodiments, the bispecific antigen-binding molecules may be administered in combination with one or more agents, for example, a corticosteroid, to reduce or ameliorate one or more adverse side effects, e.g., cytokine storm. In certain embodiments, the bispecific antigen-binding molecules may be administered in combination with one or more therapeutic agents or therapies for enhanced efficacy in treating cancer. Exemplary additional therapeutic agents or therapies that may be combined with or administered in combination with an antigen-binding molecule of the present disclosure include, e.g., chemotherapy (e.g., anti-cancer chemotherapy, for example, paclitaxel, docetaxel, vincristine, cisplatin, carboplatin or oxaliplatin), radiation therapy, surgery, a checkpoint inhibitor, a PD-1 inhibitor (e.g., an anti-PD-1 antibody such as pembrolizumab, nivolumab, or cemiplimab), a CTLA-4 inhibitor, LAG3 inhibitor, TIM3 inhibitor, a GITR agonist, OX40 agonist, 4-1BB agonist, CD38 inhibitor, an oncolytic virus, a cancer vaccine, a CAR-T cell, a nucleic acid therapeutic, a stem cell transplant, a modified IL2, modified IL12, IL15, IL1 inhibitor, IL6 inhibitor (e.g., sarilumab or tocilizumab), IL4R inhibitor (e.g., dupilumab), BCMA inhibitor, CD20 inhibitor, CD19 inhibitor, EGFR inhibitor, Ang2 inhibitor, VEGF inhibitor, a corticosteroid, a bispecific antibody or antigen-binding fragment thereof that binds CD3, CD28, or 4-1BB and a tumor associated antigen (TAA) (e.g., CD20, CD19, BCMA, CD38, MUC16 (mucin 16), PSMA, EGFR, STEAP1, or STEAP2). Exemplary bispecific antibodies comprising an antigen-binding domain that binds CD3 include, but are not limited to those described in, e.g., WO2017/053856A1, WO2014/047231A1, WO2018/067331A1 and WO2018/058001A1. SLC3A2-APIS is expressed in a wide range of cancers. Accordingly, the bispecific SLC3A2-APISxCD3 antibodies of the present disclosure can be used in combination with a wide range of bispecific antibodies comprising an antigen-binding domain that binds CD3 in treatments of various cancers.

The additional agents may be administered just prior to, concurrent with, or shortly after the administration of an antigen-binding molecule of the present disclosure; (for purposes of the present disclosure, such an administration regimen is considered the administration of an antigen-binding molecule “in combination with” an additional agent or therapeutic agent or therapy).

### Pharmaceutical Formulations and Administration

The present disclosure provides compositions that include SLC3A2-APISxCD3 antigen-binding proteins and one or more ingredients; as well as methods of use thereof and methods of making such compositions. Pharmaceutical formulations (e.g., aqueous pharmaceutical formulations that include water) comprising an SLC3A2-APISxCD3 antigen-binding protein of the present disclosure and a pharmaceutically acceptable carrier or excipient are part of the present disclosure.

The pharmaceutical compositions of the disclosure can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

To prepare pharmaceutical formulations of the SLC3A2-APISxCD3 antigen-binding proteins, e.g., antibodies and antigen-binding fragments thereof (e.g., bsAb17234 or bsAb17235), the antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In some embodiments, the pharmaceutical formulation is sterile. Such compositions are part of the present disclosure.

Pharmaceutical formulations of the present disclosure include an SLC3A2-APISxCD3 antigen-binding protein and a pharmaceutically acceptable carrier including, for example, water, buffering agents, preservatives and/or detergents.

The scope of the present disclosure includes desiccated, e.g., freeze-dried compositions, comprising an SLC3A2-APISxCD3 antigen-binding protein, e.g., antibody or antigen-binding fragment thereof, or a pharmaceutical formulation thereof that includes a pharmaceutically acceptable carrier but substantially lacks water.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, rectal, intestinal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As discussed herein, the present disclosure provides a vessel (e.g., a plastic or glass vial) or injection device (e.g., syringe, pre-filled syringe or autoinjector) comprising any of the SLC3A2-APISxCD3 antigen-binding proteins herein, e.g., antibodies or antigen-binding fragments thereof, or a pharmaceutical formulation comprising a pharmaceutically acceptable carrier or excipient thereof.

A pharmaceutical composition of the present disclosure can be delivered subcutaneously or intravenously with a standard needle and syringe. With respect to subcutaneous delivery, a pen delivery device, as known in the art, may be used in delivering a pharmaceutical composition of the present disclosure. Such a pen delivery device can be reusable or disposable.

Numerous reusable and disposable pens and autoinjector delivery devices have applications in the subcutaneous delivery of a pharmaceutical composition of the present disclosure. See e.g., AUTOPEN™ (Owen Mumford, Inc., Woodstock, UK) or the HUMIRA™ Pen (Abbott Labs, Abbott Park, IL).

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline and other isotonic solutions which may be used in combination with an appropriate solubilizing agent. Injectable oily mediums are also part of the present disclosure. Such oily mediums may be combined with a solubilizing agent.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 2000 mg per dosage form in a unit dose; especially in the form of injection.

The present disclosure also provides kits comprising an isolated antibody or antigen-binding fragment thereof that comprises an antigen-binding domain that binds specifically to human SLC3A2-APIS in a tumor cell, optionally wherein the antibody or antigen-binding fragment thereof further comprises an antigen-binding domain that binds specifically to human CD3 for therapeutic uses as described herein. Kits typically include a label indicating the intended use of the contents of the kit and instructions for use. As used herein, the term “label” includes any writing, or recorded material supplied on, in or with the kit, or which otherwise accompanies the kit. Accordingly, this disclosure provides a kit for inhibiting a tumor in a subject in need thereof, the kit comprising: (a) a therapeutically effective dosage of the isolated antibody or antigen-binding fragment thereof; and (b) instructions for using the isolated antibody or antigen-binding fragment thereof in any of the methods disclosed herein.

### Diagnostic Uses

The antigen-binding proteins of the present disclosure may also be used to detect and/or measure SLC3A2-APIS, or SLC3A2-APIS-expressing cells in a sample, e.g., for diagnostic purposes. For example, SLC3A2-APIS antibody or antigen-binding fragment thereof, may be used to diagnose a condition or disease characterized by aberrant expression (e.g., over-expression, under-expression, lack of expression, etc.) of SLC3A2-APIS. Exemplary diagnostic assays for SLC3A2-APIS may comprise, e.g., contacting a sample, obtained from a subject, with an antibody of the disclosure, wherein the antibody is labeled with a detectable label or reporter molecule. Alternatively, an unlabeled antibody can be used in diagnostic applications in combination with a secondary antibody which is itself detectably labeled. The detectable label or reporter molecule can be a radioisotope, such as ³H, ¹⁴C, ³²p, ³⁵S, or ¹²⁵I; a fluorescent or chemiluminescent moiety such as fluorescein isothiocyanate or rhodamine; or an enzyme such as alkaline phosphatase, betagalactosidase, horseradish peroxidase, or luciferase. Specific exemplary assays that can be used to detect or measure SLC3A2-APIS in a sample include enzyme-linked immunosorbent assay (ELISA), radioimmunoassay (RIA), and fluorescence-activated cell sorting (FACS). Samples that can be used in SLC3A2-APIS diagnostic assays according to the present disclosure include any tissue or fluid sample obtainable from a subject which contains detectable quantities of SLC3A2-APIS protein, or fragments thereof, under normal or pathological conditions. Generally, levels of SLC3A2-APIS in a particular sample obtained from a healthy subject (e.g., a subject not afflicted with a disease or condition associated with abnormal SLC3A2-APIS levels or activity) will be measured to initially establish a baseline, or standard, level of SLC3A2-APIS. This baseline level of SLC3A2-APIS can then be compared against the levels of SLC3A2-APIS measured in samples obtained from individuals suspected of having a SLC3A2-APIS related disease or condition.

### Examples

The following examples are put forth so as to provide those of ordinary skill in the art with a complete disclosure and description of how to make and use the technology of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 250C, and pressure is at or near atmospheric.

### Example 1: Construction of Anti-SLC3A2-APIS and Bispecific SLC3A2-APISxCD3 Antibodies

### Generation of Anti-SLC3A2-APIS Antibodies

Anti-SLC3A2-APIS antibodies were obtained by immunizing a genetically engineered mouse comprising DNA encoding human immunoglobulin heavy and kappa light chain variable regions with a human SLC3A2 antigen bearing the APIS insertion.

Following immunization, splenocytes were harvested from each mouse and B-cell sorted (as described in US 2007/0280945) using a human SLC3A2-APIS fragment as the sorting agent that binds and identifies reactive antibodies (antigen-positive B-cells). Anti-SLC3A2-APIS antibodies were isolated directly from antigen-positive B cells, as described in U.S. Pat. No. 7,582,298.

The antibodies were characterized and selected for desirable characteristics including selectivity, affinity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Exemplified antibodies were constructed using human IgG1 or human IgG4 isotype. In certain embodiments, the IgG4 Fc domain comprises 2 or more amino acid changes as disclosed in US 2010/0331527 or US 2014/0243504. In certain embodiments, the human IgG4 Fc comprises a serine to proline mutation in the hinge region (S108P) to promote dimer stabilization. Table 1 sets forth the amino acid identifiers of HCVRs, LCVRs, CDRs and full-length heavy chain (HC) and light chain (LC) sequences. The corresponding nucleic acid sequence identifiers are set forth in Table 2.

TABLE 1
Amino Acid Sequence Identifiers for Exemplified anti-SLC3A2-APIS Monoclonal Antibodies

| Antibody | SEQ ID NOs: | | | | | | | | | |
|---|---|---|---|---|---|---|---|---|---|---|
| Designation | HCVR | HCDR1 | HCDR2 | HCDR3 | LCVR | LCDR1 | LCDR2 | LCDR3 | HC | LC |
| REGN17192 | 2 | 4 | 6 | 8 | 10 | 12 | AAS | 16 | 18 | 20 |
| REGN17193 | 22 | 24 | 26 | 28 | 10 | 12 | AAS | 16 | 30 | 20 |
| REGN17213 | 32 | 34 | 36 | 38 | 40 | 42 | GAS | 46 | 48 | 50 |
| REGN17214 | 52 | 54 | 56 | 58 | 40 | 42 | GAS | 46 | 60 | 50 |
| REGN17224 | 62 | 64 | 66 | 68 | 70 | 72 | AAS | 16 | 74 | 76 |
| REGN17210 | 78 | 80 | 82 | 84 | 40 | 42 | GAS | 46 | 86 | 50 |
| REGN17211 | 88 | 90 | 92 | 94 | 40 | 42 | GAS | 46 | 96 | 50 |
| REGN17212 | 32 | 34 | 36 | 38 | 40 | 42 | GAS | 46 | 98 | 50 |
| REGN17207 | 62 | 64 | 66 | 68 | 70 | 72 | AAS | 16 | 100 | 76 |
| REGN17209 | 78 | 80 | 82 | 84 | 40 | 42 | GAS | 46 | 102 | 50 |
| REGN17199 | 104 | 106 | 108 | 110 | 112 | 114 | AAS | 16 | 116 | 118 |
| REGN17200 | 120 | 122 | 124 | 126 | 40 | 42 | GAS | 46 | 128 | 50 |
| REGN17205 | 130 | 132 | 134 | 136 | 40 | 42 | GAS | 46 | 138 | 50 |
| REGN17194 | 22 | 24 | 26 | 28 | 140 | 12 | AAS | 142 | 144 | 146 |
| REGN17196 | 148 | 150 | 152 | 154 | 40 | 42 | GAS | 46 | 156 | 50 |
| REGN17197 | 148 | 150 | 152 | 154 | 158 | 160 | GAS | 46 | 164 | 166 |
| REGN17204 | 168 | 170 | 172 | 174 | 10 | 12 | AAS | 16 | 176 | 20 |
| REGN17206 | 62 | 64 | 66 | 68 | 10 | 12 | AAS | 16 | 74 | 20 |
| REGN17208 | 178 | 180 | 182 | 184 | 10 | 12 | AAS | 16 | 186 | 20 |
| REGN17201 | 188 | 190 | 192 | 194 | 10 | 12 | AAS | 16 | 196 | 20 |
| REGN17202 | 198 | 170 | 200 | 202 | 10 | 12 | AAS | 16 | 204 | 20 |
| REGN17203 | 198 | 170 | 200 | 202 | 10 | 12 | AAS | 16 | 206 | 20 |
| REGN17195 | 208 | 210 | 6 | 8 | 10 | 12 | AAS | 16 | 212 | 20 |
| REGN17198 | 104 | 106 | 108 | 110 | 10 | 12 | AAS | 16 | 214 | 20 |

TABLE 2
Nucleic Acid Sequence Identifiers for Exemplified anti-SLC3A2-APIS Monoclonal Antibodies

| Antibody | SEQ ID NOs: | | | | | | | | | |
|---|---|---|---|---|---|---|---|---|---|---|
| Designation | HCVR | HCDR1 | HCDR2 | HCDR3 | LCVR | LCDR1 | LCDR2 | LCDR3 | HC | LC |
| REGN17192 | 1 | 3 | 5 | 7 | 9 | 11 | gctgcatcc | 15 | 17 | 19 |
| REGN17193 | 21 | 23 | 25 | 27 | 9 | 11 | gctgcatcc | 15 | 29 | 19 |
| REGN17213 | 31 | 33 | 35 | 37 | 39 | 41 | ggggcaagt | 45 | 47 | 49 |
| REGN17214 | 51 | 53 | 55 | 57 | 39 | 41 | ggggcaagt | 45 | 59 | 49 |
| REGN17224 | 61 | 63 | 65 | 67 | 69 | 71 | gctgcatcc | 15 | 73 | 75 |
| REGN17210 | 77 | 79 | 81 | 83 | 39 | 41 | ggggcaagt | 45 | 85 | 49 |
| REGN17211 | 87 | 89 | 91 | 93 | 39 | 41 | ggggcaagt | 45 | 95 | 49 |
| REGN17212 | 31 | 33 | 35 | 37 | 39 | 41 | ggggcaagt | 45 | 97 | 49 |
| REGN17207 | 61 | 63 | 65 | 67 | 69 | 71 | gctgcatcc | 15 | 99 | 75 |
| REGN17209 | 77 | 79 | 81 | 83 | 39 | 41 | ggggcaagt | 45 | 101 | 49 |
| REGN17199 | 103 | 105 | 107 | 109 | 111 | 113 | gctgcatcc | 15 | 115 | 117 |
| REGN17200 | 119 | 121 | 123 | 125 | 39 | 41 | ggggcaagt | 45 | 127 | 49 |
| REGN17205 | 129 | 131 | 133 | 135 | 39 | 41 | ggggcaagt | 45 | 137 | 49 |
| REGN17194 | 21 | 23 | 25 | 27 | 139 | 11 | gctgcatcc | 141 | 143 | 145 |
| REGN17196 | 147 | 149 | 151 | 153 | 39 | 41 | ggggcaagt | 45 | 155 | 49 |
| REGN17197 | 147 | 149 | 151 | 153 | 157 | 159 | ggtgcatcc | 162 | 163 | 165 |
| REGN17204 | 167 | 169 | 171 | 173 | 9 | 11 | gctgcatcc | 15 | 175 | 19 |
| REGN17206 | 61 | 63 | 65 | 67 | 9 | 11 | gctgcatcc | 15 | 73 | 19 |
| REGN17208 | 177 | 179 | 181 | 183 | 9 | 11 | gctgcatcc | 15 | 185 | 19 |
| REGN17201 | 187 | 189 | 191 | 193 | 9 | 11 | gctgcatcc | 15 | 195 | 19 |
| REGN17202 | 197 | 169 | 199 | 201 | 9 | 11 | gctgcatcc | 15 | 203 | 19 |
| REGN17203 | 197 | 169 | 199 | 201 | 9 | 11 | gctgcatcc | 15 | 205 | 19 |
| REGN17195 | 207 | 209 | 5 | 7 | 9 | 11 | gctgcatcc | 15 | 211 | 19 |
| REGN17198 | 103 | 105 | 107 | 109 | 9 | 11 | gctgcatcc | 15 | 213 | 19 |

Generation of Bispecific Antibodies (bsAbs) that Bind SLC3A2-APIS and CD3

The present disclosure also provides bispecific antigen-binding molecules that bind SLC3A2-APIS and CD3; such bispecific antigen-binding molecules are also referred to herein as “anti-SLC3A2-APIS×anti-CD3 or anti-SLC3A2-APIS×CD3 or anti-CD3 ×SLC3A2-APIS or SLC3A2-APISxCD3 bispecific molecules or bispecific antibodies.” The anti-SLA3A2-APIS portion of the anti-SLC3A2-APIS×anti-CD3 bispecific molecules is useful for targeting tumor cells that express SLC3A2-APIS, and the anti-CD3 portion of the bispecific molecules is useful for activating T-cells. The simultaneous binding of SLC3A2-APIS on a tumor cell and CD3 on a T-cell facilitates directed killing (cell lysis) of the targeted tumor cell by the activated T-cell.

Bispecific antibodies comprising an anti-SLC3A2-APIS-specific binding domain and an anti-CD3-specific binding domain were constructed using standard methodologies, wherein the anti-SLC3A2-APIS antigen binding domain and the anti-CD3 antigen binding domain each comprise different, distinct HCVRs paired with a common LCVR. In exemplified bispecific antibodies, the molecules were constructed utilizing a heavy chain from an anti-SLC3A2-APIS antibody, a heavy chain from an anti-CD3 antibody (7221G or 7195P; U.S. Pat. No. 9,657,102), and a common light chain (cognate light chain of the anti-SLC3A2-APIS binder or ULC 1-39).

Table 3 summarizes the component parts (parental antibody designation) of selected bispecific SLC3A2-APISxCD3 antibodies. Tables 4 and 5 set forth the amino acid and nucleic acid sequence identifiers, respectively of the HCVRs, LCVRs, and CDRs of selected bispecific SLC3A2-APISxCD3 antibodies. Table 6 shows the amino acid and nucleic acid sequence identifiers of the heavy and light chain amino acid sequences of selected SLC3A2-APISxCD3 bispecific antibodies.

TABLE 3
Summary of Component Parts of Selected
SLC3A2-APIS xCD3 Bispecific Antibodies

| | Anti-SLC3A2-APIS | Anti-CD3 | |
|---|---|---|---|
| Bispecific | Parental | Parental | Light |
| Antibody | Antibody | Antibody | Chain |
| bsAb17225 | REGN17208 | 7221G | ULC 1-39 |
| bsAb17226 | REGN17206 | 7221G | ULC 1-39 |
| bsAb17227 | REGN17204 | 7221G | ULC 1-39 |
| bsAb17228 | REGN17202 | 7221G | ULC 1-39 |
| bsAb17229 | REGN17201 | 7221G | ULC 1-39 |
| bsAb17230 | REGN17198 | 7221G | ULC 1-39 |
| bsAb17231 | REGN17195 | 7221G | ULC 1-39 |
| bsAb17232 | REGN17193 | 7221G | ULC 1-39 |
| bsAb17233 | REGN17192 | 7221G | ULC 1-39 |
| bsAb17234 | REGN17224 | 7221G | REGN17224 |
| bsAb17235 | REGN17224 | 7195P | REGN17224 |
| bsAb23479 | REGN17212 | PN114997 | ULC 3-20 |
| bsAb23483 | REGN17212 | PN115017 | ULC 3-20 |
| bsAb23477 | REGN17212 | PN101664 | ULC 3-20 |

TABLE 4
Amino Acid Sequence Identifiers of Sequences of Selected SLC3A2-APIS × CD3 Bispecific Antibodies

| | | | | | | | | | Common | | | |
|---|---|---|---|---|---|---|---|---|---|---|---|---|
| | Anti-SLC3A2-APIS | | | | Anti-CD3 | | | | Light Chain Variable | | | |
| Bispecific | Antigen-Binding Domain | | | | Antigen-Binding Domain | | | | Region | | | |
| Antibody | HCVR | HCDR1 | HCDR2 | HCDR3 | HCVR | HCDR1 | HCDR2 | HCDR3 | LCVR | LCDR1 | LCDR2 | LCDR3 |
| bsAb17225 | 178 | 180 | 182 | 184 | 216 | 218 | 220 | 222 | 232 | 234 | AAS | 238 |
| bsAb17226 | 62 | 64 | 66 | 68 | 216 | 218 | 220 | 222 | 232 | 234 | AAS | 238 |
| bsAb17227 | 168 | 170 | 172 | 174 | 216 | 218 | 220 | 222 | 232 | 234 | AAS | 238 |
| bsAb17228 | 198 | 170 | 200 | 202 | 216 | 218 | 220 | 222 | 232 | 234 | AAS | 238 |
| bsAb17229 | 188 | 190 | 192 | 194 | 216 | 218 | 220 | 222 | 232 | 234 | AAS | 238 |
| bsAb17230 | 104 | 106 | 108 | 110 | 216 | 218 | 220 | 222 | 232 | 234 | AAS | 238 |
| bsAb17231 | 208 | 210 | 6 | 8 | 216 | 218 | 220 | 222 | 232 | 234 | AAS | 238 |
| bsAb17232 | 22 | 24 | 26 | 28 | 216 | 218 | 220 | 222 | 232 | 234 | AAS | 238 |
| bsAb17233 | 2 | 4 | 6 | 8 | 216 | 218 | 220 | 222 | 232 | 234 | AAS | 238 |
| bsAb17234 | 62 | 64 | 66 | 68 | 216 | 218 | 220 | 222 | 70 | 72 | AAS | 16 |
| bsAb17235 | 62 | 64 | 66 | 68 | 224 | 226 | 228 | 230 | 70 | 72 | AAS | 16 |
| bsAb23479 | 32 | 34 | 36 | 38 | 269 | 271 | 273 | 275 | 277 | 279 | GAS | 283 |
| bsAb23483 | 32 | 34 | 36 | 38 | 291 | 293 | 295 | 297 | 277 | 279 | GAS | 283 |
| bsAb23477 | 32 | 34 | 36 | 38 | 301 | 293 | 304 | 306 | 277 | 279 | GAS | 283 |

TABLE 5
Nucleic Acid Sequence Identifiers of Sequences of Selected SLC3A2-APIS × CD3 Bispecific Antibodies

| | Anti-SLC3A2-APIS | | | | Anti-CD3 | | | | Common | | | |
|---|---|---|---|---|---|---|---|---|---|---|---|---|
| Bispecific | Antigen-Binding Domain | | | | Antigen-Binding Domain | | | | Light Chain Variable Region | | | |
| Antibody | HCVR | HCDR1 | HCDR2 | HCDR3 | HCVR | HCDR1 | HCDR2 | HCDR3 | LCVR | LCDR1 | LCDR2 | LCDR3 |
| bsAb17225 | 177 | 179 | 181 | 183 | 215 | 217 | 219 | 221 | 231 | 233 | gctgcatcc | 237 |
| bsAb17226 | 61 | 63 | 65 | 67 | 215 | 217 | 219 | 221 | 231 | 233 | gctgcatcc | 237 |
| bsAb17227 | 167 | 169 | 171 | 173 | 215 | 217 | 219 | 221 | 231 | 233 | gctgcatcc | 237 |
| bsAb17228 | 197 | 169 | 199 | 201 | 215 | 217 | 219 | 221 | 231 | 233 | gctgcatcc | 237 |
| bsAb17229 | 187 | 189 | 191 | 193 | 215 | 217 | 219 | 221 | 231 | 233 | gctgcatcc | 237 |
| bsAb17230 | 103 | 105 | 107 | 109 | 215 | 217 | 219 | 221 | 231 | 233 | gctgcatcc | 237 |
| bsAb17231 | 207 | 209 | 5 | 7 | 215 | 217 | 219 | 221 | 231 | 233 | gctgcatcc | 237 |
| bsAb17232 | 21 | 23 | 25 | 27 | 215 | 217 | 219 | 221 | 231 | 233 | gctgcatcc | 237 |
| bsAb17233 | 1 | 3 | 5 | 7 | 215 | 217 | 219 | 221 | 231 | 233 | gctgcatcc | 237 |
| bsAb17234 | 61 | 63 | 65 | 67 | 215 | 217 | 219 | 221 | 69 | 71 | gctgcatcc | 15 |
| bsAb17235 | 61 | 63 | 65 | 67 | 223 | 225 | 227 | 229 | 69 | 71 | gctgcatcc | 15 |
| bsAb23479 | 31 | 33 | 35 | 37 | 268 | 270 | 272 | 274 | 276 | 278 | ggggcaagt | 282 |
| bsAb23483 | 31 | 33 | 35 | 37 | 290 | 292 | 294 | 296 | 276 | 278 | ggggcaagt | 282 |
| bsAb23477 | 31 | 33 | 35 | 37 | 300 | 302 | 303 | 305 | 276 | 278 | ggggcaagt | 282 |

TABLE 6
Amino Acid and Nucleic Acid Sequence Identifiers for Full Length Heavy Chain
and Light Chain Sequences of Selected SLC3A2-APIS × CD3 Bispecific Antibodies

| | Bispecific | SEQ ID NOs | | | | | |
|---|---|---|---|---|---|---|---|
| Antibody | Antibody | HC (SLC3A2-APIS) | | HC (CD3) | | LC (SLC3A2-APIS & CD3) | |
| Identifier | Designation | D | P | D | P | D | P |
| bsAb17229 | REGN17229 | 247 | 248 | 239 | 240 | 243 | 244 |
| bsAb17230 | REGN17230 | 249 | 250 | 239 | 240 | 243 | 244 |
| bsAb17231 | REGN17231 | 251 | 252 | 239 | 240 | 243 | 244 |
| bsAb17232 | REGN17232 | 253 | 254 | 239 | 240 | 243 | 244 |
| bsAb17233 | REGN17233 | 255 | 256 | 239 | 240 | 243 | 244 |
| bsAb17234 | REGN17234 | 257 | 258 | 239 | 240 | 245 | 246 |
| bsAb17235 | REGN17235 | 257 | 258 | 241 | 242 | 245 | 246 |
| bsAb17225 | REGN17225 | 259 | 260 | 239 | 240 | 243 | 244 |
| bsAb17226 | REGN17226 | 257 | 258 | 239 | 240 | 243 | 244 |
| bsAb17227 | REGN17227 | 261 | 262 | 239 | 240 | 243 | 244 |
| bsAb17228 | REGN17228 | 263 | 264 | 239 | 240 | 243 | 244 |
| bsAb23479 | REGN23479 | 284 | 285 | 286 | 287 | 288 | 289 |
| bsAb23483 | REGN23483 | 284 | 285 | 298 | 299 | 288 | 289 |
| bsAb23477 | REGN23477 | 284 | 285 | 307 | 308 | 288 | 289 |

D = Nucleic acid sequence of DNA encoding indicated sequence
P = Amino acid of polypeptide for the indicated sequence

Additional bispecific antibodies comprising one HCVR from a parental SLC3A2-APIS antibody and the other HCVR arm from a parental CD3 antibody may be made using the techniques described herein. The parental SLC3A2-APIS antibodies used to generate these additional SLC3A2-APISxCD3 bispecific antibodies have HCVR sequences described above in Table 1. The CD3 parental antibodies used to generate these additional SLC3A2-APISxCD3 bispecific antibodies have the amino acid sequences of the heavy chain from the anti-CD3 antibody 7195P (U.S. Pat. No. 9,657,102).

Controls: In addition to isotype controls, the following bispecific antibody controls were used in certain experiments described in the Examples below: CD3-7211G Control: a bispecific antibody with one arm binding to CD3 (derived from parental antibody 7211G, which is also a parental antibody for bsAb17234) and the other arm binding to an unrelated (non-tumor associated) antigen; CD3-7195P Control: a bispecific antibody with one arm binding to CD3 (derived from parental antibody 7195P, which is also a parental antibody of bsAb17235) and the other arm binding to an unrelated (non-tumor associated) antigen.

### Example 2: Characterization of SLC3A2-APIS Binding of SLC3A2-APIS Monospecific and SLC3A2-APISxCD3 Bispecific Antibodies by Surface Plasmon Resonance

This example relates to a surface plasmon resonance study performed to demonstrate the specificity of selected monospecific anti-SLC3A2-APIS antibodies and anti-SLC3A2-APIS×anti-CD3 bispecific antibodies to bind to the APIS insertion variant of SLC3A2, “SLC3A2-APIS.” The biacore kinetics of binding to monomeric and dimeric human SLC3A2-APIS proteins in an antibody capture format at 25° C. and 37° C. are presented.

Equilibrium dissociation constants (K_{D} values) of anti-SLC3A2-APIS monospecific and bispecific antibodies binding to monomeric and dimeric human SLC3A2-APIS proteins were determined using real-time surface plasmon resonance biosensor technology on a Biacore T-200 instrument. The monomeric human SLC3A2-APIS was expressed with an N-terminal hexahistidine-myc-myc tag (Hmm.SLC3A2-APIS; SEQ ID NO: 265) and dimeric human SLC3A2-APIS expressed with an N-terminal murine Fc tag (mFc.hSLC3A2-APIS; SEQ ID NO: 267). Briefly, the CM5 Biacore sensor surface was derivatized by amine coupling with a monoclonal mouse anti-human Fc monoclonal antibody. All Biacore binding studies were performed in a buffer composed of 10 mM HEPES at pH 7.4, 150 mM NaCl, 0.05% v/v Surfactant P20 (HBS-EP running buffer). Monospecific SLC3A2-APIS bivalent antibodies or bispecific SLC3A2-APISxCD3 antibodies were captured. Different concentrations of monomeric human SLC3A2-APIS (Hmm.hSLC3A2-APIS) or dimeric human SLC3A2-APIS (mFc.hSLC3A2-APIS), ranging from 10 nM to 90 nM in 3-fold serial dilutions, were injected over the captured anti-SLC3A2-APIS surfaces at a flow rate of 50 μL/minute. Antibody-ligand association was monitored for 5 minutes, and dissociation was monitored for 10 minutes. At the end of each cycle, the anti-hFc capture surface was regenerated using a 12 second injection of 20 mM phosphoric acid. The binding kinetics experiments were performed at 25 and 37° C.

Data analysis: The specific SPR-Biacore sensorgrams were obtained by a double referencing procedure. The double referencing was performed by first subtracting the signal of each injection over a reference surface (anti-hFc) from the signal over the experimental surface (anti-hFc-captured anti-SLC3A2-APIS antibodies) thereby removing contributions from refractive index changes. In addition, running buffer injections were performed to allow subtraction of the signal changes resulting from the dissociation of captured antibodies or antigens from the coupled anti-hFc surface. Kinetic association (kₐ) and dissociation (K_{d}) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = \frac{kd}{ka} , and t \frac{1}{2} ( min ) = \frac{ln ( 2 )}{60 * kd}$$

Results: Biacore analysis showed that 14 of the 24 monospecific anti-SLC3A2-APIS antibodies tested bound SLC3A2-APIS (Tables 7-10). 20 of the 24 monospecific anti-SLC3A2-APIS antibodies tested had unique variable domains (Table 1). Of the 20 monospecific anti-SLC3A2-APIS antibodies having unique variable domains, 10 bound SLC3A2-APIS in this assay (Tables 7-10). At 25° C., Kos for binding to dimeric SLC3A2-APIS ranged from ˜1.7E-9 M to ˜3.7E-7 M, and the values for t½ ranged from 0.5 to 94.2 min (Table 9). Under these conditions, 4 of the monospecific anti-SLC3A2-APIS antibodies having unique variable domains showed particularly strong binding to SLC3A2-APIS (i.e. having a K_{D} in the nanomolar range): REGN17208, REGN17209, REGN17212, and REGN17224; and the t½ values for these 4 antibodies ranged rom 30.6 min to 94.2 min (Table 9).

Biacore analysis showed that SLC3A2-APISxCD3 bispecific antibodies exhibited strong binding to SLC3A2-APIS. 7 out of the 11 SLC3A2-APISxCD3 bispecific antibodies tested bound the dimeric form of SLC3A2-APIS at both 25° C. and 37° C., with K_{D}s ranging from ˜1.6E-9 M to ˜1.8E-7 M (Tables 9 and 10). The 2 SLC3A2-APISxCD3 bispecific antibodies that exhibited the strongest binding to SLC3A2-APIS were bsAb17234 and bsAb17235, with a K_{D} of ˜1.3E-9 M and ˜1.4E-9 M, respectively, for binding to monomeric SLC3A2-APIS 25° C.; and the t½ values for bsAb17234 and bsAb17235 54.1 min and 56.0 min, respectively (Table 7). The anti-SLC3A2-APIS monospecific antibody selected as the parental antibody for both of these bispecific SLC3A2-APIS antibodies was REGN17224, which exhibited the strongest SLC3A2-APIS binding of all of the monospecific anti-SLC3A2-APIS antibodies under these conditions, with a K_{D} of 1.1E-09 M and a t½ of 34.7 min.

Table 7 sets forth the equilibrium binding parameters of monomeric human SLC3A2-APIS to surface-captured anti-SLC3A2-APIS monospecific and bispecific antibodies at 25° C.

TABLE 7
Kinetic and Equilibrium Binding Parameters of Monomeric
Human SLC3A2-APIS to Surface-captured anti-SLC3A2-APIS
Monospecific and Bispecific Antibodies at 25° C.

| | | 90 nM | | | | |
|---|---|---|---|---|---|---|
| Antibody | mAb | Hmm.hSLC3A2- | | | | |
| Identifier or | Capture | APIS Bind | ka | kd | K_{D} | t½ |
| Designation | (RU) | (RU) | (1/M/s) | (1/s) | (M) | (min) |
| REGN17192 | 186.3 ± 0.3 | 0.0 | NB* | NB* | NB* | NB* |
| REGN17193 | 226.7 ± 0.5 | 0.1 | NB* | NB* | NB* | NB* |
| REGN17194 | 78.8 ± 0.1 | 0.0 | NB* | NB* | NB* | NB* |
| REGN17195 | 172.6 ± 0.5 | 0.3 | NB* | NB* | NB* | NB* |
| REGN17196 | 210.2 ± 2.3 | 0.1 | NB* | NB* | NB* | NB* |
| REGN17197 | 206.3 ± 0.4 | −0.1 | NB* | NB* | NB* | NB* |
| REGN17198 | 178.8 ± 1.7 | 0.0 | NB* | NB* | NB* | NB* |
| REGN17199 | 193.7 ± 6.2 | 0.0 | NB* | NB* | NB* | NB* |
| REGN17200 | 192.3 ± 1.2 | 0.0 | NB* | NB* | NB* | NB* |
| REGN17201 | 212.5 ± 0.4 | 2.1 | IC** | IC** | IC** | IC** |
| REGN17202 | 200.9 ± 6.9 | 5.0 | 7.32E+04 | 4.81E−03 | 6.57E−08 | 2.4 |
| REGN17203 | 201.2 ± 0.9 | 4.2 | 7.78E+04 | 4.64E−03 | 5.97E−08 | 2.5 |
| REGN17204 | 114.3 ± 0.5 | 3.7 | 4.02E+04 | 3.97E−02 | 9.88E−07 | 0.3 |
| REGN17205 | 122.4 ± 0.6 | 0.2 | NB* | NB* | NB* | NB* |
| REGN17206 | 195.8 ± 1.3 | 5.0 | 6.67E+03 | 2.50E−03 | 3.74E−07 | 4.6 |
| REGN17207 | 196.1 ± 2.0 | 43.4 | 1.30E+05 | 2.45E−04 | 1.89E−09 | 47.1 |
| REGN17208 | 640.9 ± 1.1 | 25.5 | 1.21E+05 | 2.46E−03 | 2.03E−08 | 4.7 |
| REGN17209 | 202.2 ± 0.5 | 43.3 | 1.11E+05 | 2.58E−04 | 2.33E−09 | 44.7 |
| REGN17210 | 181.9 ± 4.9 | 34.6 | 8.21E+04 | 2.99E−04 | 3.64E−09 | 38.6 |
| REGN17211 | 180.5 ± 0.4 | 0.3 | NB* | NB* | NB* | NB* |
| REGN17212 | 121.2 ± 1.3 | 19.0 | 5.18E+04 | 2.07E−04 | 3.98E−09 | 55.9 |
| REGN17213 | 61.6 ± 0.3 | 8.8 | 4.80E+04 | 3.04E−04 | 6.33E−09 | 38.0 |
| REGN17214 | 174.3 ± 1.2 | 4.5 | 4.86E+03 | 5.51E−03 | 1.13E−06 | 2.1 |
| REGN17224 | 167.5 ± 1.2 | 87.0 | 3.10E+05 | 3.33E−04 | 1.07E−09 | 34.7 |
| bsAb17225 | 175.5 ± 1.1 | 3.9 | 1.78E+05 | 4.82E−03 | 2.71E−08 | 2.4 |
| bsAb17226 | 173.0 ± 0.1 | 2.9 | 9.14E+03 | 2.46E−03 | 2.69E−07 | 4.7 |
| bsAb17227 | 171.0 ± 0.7 | 3.5 | 9.15E+04 | 2.76E−02 | 3.02E−07 | 0.4 |
| bsAb17228 | 182.7 ± 0.7 | 2.7 | 1.02E+05 | 4.96E−03 | 4.85E−08 | 2.3 |
| bsAb17229 | 205.6 ± 3.1 | 1.7 | NB* | NB* | NB* | NB* |
| bsAb17230 | 184.6 ± 1.7 | 0.2 | NB* | NB* | NB* | NB* |
| bsAb17231 | 175.8 ± 1.2 | 0.3 | NB* | NB* | NB* | NB* |
| bsAb17232 | 178.5 ± 1.6 | 0.3 | NB* | NB* | NB* | NB* |
| bsAb17233 | 187.1 ± 0.7 | 0.2 | NB* | NB* | NB* | NB* |
| bsAb17234 | 179.6 ± 0.9 | 24.6 | 1.70E+05 | 2.13E−04 | 1.26E−09 | 54.1 |
| bsAb17235 | 187.7 ± 0.3 | 26.1 | 1.48E+05 | 2.06E−04 | 1.40E−09 | 56.0 |

*NB: Non-binding
**IC: Inconclusive

Table 8 sets forth the equilibrium binding parameters of monomeric human SLC3A2-APIS to surface-captured anti-SLC3A2-APIS monospecific and bispecific antibodies at 37° C.

TABLE 8
Kinetic and Equilibrium Binding Parameters of Monomeric
Human SLC3A2-APIS to Surface-captured anti-SLC3A2-APIS
Monospecific and Bispecific Antibodies at 37° C.

| Antibody | mAb | 90 nM | | | | |
|---|---|---|---|---|---|---|
| Identifier or | Capture | Hmm.hSLC3A2- | ka | kd | K_{D} | t½ |
| Designation | (RU) | APIS Bind (RU) | (1/M/s) | (1/s) | (M) | (min) |
| REGN17192 | 221.5 ± 0.8 | 0.0 | NB* | NB* | NB* | NB* |
| REGN17193 | 259.1 ± 1.3 | −0.1 | NB* | NB* | NB* | NB* |
| REGN17194 | 91.7 ± 1.7 | −0.2 | NB* | NB* | NB* | NB* |
| REGN17195 | 197.2 ± 1.1 | −0.1 | NB* | NB* | NB* | NB* |
| REGN17196 | 259.1 ± 8.9 | 0.1 | NB* | NB* | NB* | NB* |
| REGN17197 | 239.2 ± 1.2 | 0.0 | NB* | NB* | NB* | NB* |
| REGN17198 | 210.3 ± 1.3 | −0.5 | NB* | NB* | NB* | NB* |
| REGN17199 | 220.6 ± 1.2 | 0.1 | NB* | NB* | NB* | NB* |
| REGN17200 | 229.3 ± 0.8 | 0.0 | NB* | NB* | NB* | NB* |
| REGN17201 | 241.6 ± 3.9 | 3.6 | IC** | IC** | IC** | IC** |
| REGN17202 | 228.1 ± 2.6 | 4.2 | 7.64E+04 | 2.24E−02 | 2.93E−07 | 0.5 |
| REGN17203 | 238.3 ± 0.5 | 3.6 | IC** | IC** | IC** | IC** |
| REGN17204 | 129.9 ± 0.7 | 3.7 | IC** | IC** | IC** | IC** |
| REGN17205 | 136.6 ± 1.0 | 0.0 | NB* | NB* | NB* | NB* |
| REGN17206 | 233.0 ± 1.0 | 8.7 | 3.72E+03 | 9.14E−03 | 2.46E−06 | 1.3 |
| REGN17207 | 221.2 ± 0.7 | 68.0 | 1.92E+05 | 1.18E−03 | 6.12E−09 | 9.8 |
| REGN17208 | 767.6 ± 3.7 | 33.5 | 7.93E+05 | 2.14E−03 | 2.70E−09 | 5.4 |
| REGN17209 | 247.2 ± 1.0 | 75.2 | 2.69E+05 | 1.27E−03 | 4.72E−09 | 9.1 |
| REGN17210 | 210.8 ± 1.2 | 59.2 | 2.20E+05 | 1.24E−03 | 5.64E−09 | 9.3 |
| REGN17211 | 216.9 ± 0.2 | 0.2 | NB* | NB* | NB* | NB* |
| REGN17212 | 141.8 ± 0.4 | 44.0 | 1.80E+05 | 9.16E−04 | 5.08E−09 | 12.6 |
| REGN17213 | 63.1 ± 0.6 | 20.7 | 1.42E+05 | 1.07E−03 | 7.52E−09 | 10.8 |
| REGN17214 | 208.8 ± 4.7 | 2.2 | IC** | IC** | IC** | IC** |
| REGN17224 | 191.6 ± 0.5 | 112.1 | 3.87E+05 | 1.23E−03 | 3.17E−09 | 9.4 |
| bsAb17225 | 213.4 ± 2.0 | 3.4 | 2.65E+05 | 6.52E−03 | 2.46E−08 | 1.8 |
| bsAb17226 | 197.1 ± 2.2 | 4.0 | 1.60E+03 | 1.03E−02 | 6.41E−06 | 1.1 |
| bsAb17227 | 196.6 ± 1.0 | 3.0 | IC** | IC** | IC** | IC** |
| bsAb17228 | 218.8 ± 1.0 | 1.9 | NB* | NB* | NB* | NB* |
| bsAb17229 | 245.5 ± 2.5 | 2.1 | NB* | NB* | NB* | NB* |
| bsAb17230 | 209.1 ± 4.7 | 0.1 | NB* | NB* | NB* | NB* |
| bsAb17231 | 202.3 ± 7.1 | 0.0 | NB* | NB* | NB* | NB* |
| bsAb17232 | 200.3 ± 8.3 | −0.5 | NB* | NB* | NB* | NB* |
| bsAb17233 | 213.7 ± 0.8 | 0.3 | NB* | NB* | NB* | NB* |
| bsAb17234 | 211.6 ± 1.3 | 36.1 | 1.85E+05 | 1.17E−03 | 6.34E−09 | 9.9 |
| bsAb17235 | 216.9 ± 1.8 | 38.9 | 2.30E+05 | 1.17E−03 | 5.11E−09 | 9.8 |

*NB: Non-binding
**IC: Inconclusive

Table 9 sets forth the equilibrium binding parameters of dimeric human SLC3A2-APIS to surface-captured anti-SLC3A2-APIS monospecific and bispecific antibodies at 25° C.

TABLE 9
Kinetic and Equilibrium Binding Parameters of Dimeric Human SLC3A2-APIS to Surface-
captured anti-SLC3A2-APIS Monospecific and Bispecific Antibodies at 25° C.

| Antibody | mAb | 30 nM | | | | |
|---|---|---|---|---|---|---|
| Identifier or | Capture | mFc.hSLC3A2- | ka | kd | K_{D} | t½ |
| Designation | (RU) | APIS Bind (RU) | (1/M/s) | (1/s) | (M) | (min) |
| REGN17192 | 187.9 ± 0.9 | 1.4 | NB* | NB* | NB* | NB* |
| REGN17193 | 229.9 ± 0.9 | 1.1 | NB* | NB* | NB* | NB* |
| REGN17194 | 80.0 ± 1.3 | 0.2 | NB* | NB* | NB* | NB* |
| REGN17195 | 171.9 ± 0.9 | 2.9 | 1.53E+05 | 2.33E−02 | 1.52E−07 | 0.5 |
| REGN17196 | 205.3 ± 0.3 | 1.1 | NB* | NB* | NB* | NB* |
| REGN17197 | 206.6 ± 0.3 | 0.7 | NB* | NB* | NB* | NB* |
| REGN17198 | 182.9 ± 0.5 | 0.2 | NB* | NB* | NB* | NB* |
| REGN17199 | 200.1 ± 2.9 | 0.2 | NB* | NB* | NB* | NB* |
| REGN17200 | 193.8 ± 0.1 | −0.1 | NB* | NB* | NB* | NB* |
| REGN17201 | 212.3 ± 0.1 | 17.7 | 3.30E+04 | 1.75E−03 | 5.31E−08 | 6.6 |
| REGN17202 | 191.6 ± 0.3 | 9.5 | 7.17E+03 | 2.67E−03 | 3.73E−07 | 4.3 |
| REGN17203 | 206.7 ± 0.7 | 8.0 | 6.16E+04 | 1.81E−03 | 2.94E−08 | 6.4 |
| REGN17204 | 114.4 ± 0.1 | 18.4 | 7.96E+04 | 3.25E−03 | 4.08E−08 | 3.6 |
| REGN17205 | 123.4 ± 0.7 | 0.6 | NB* | NB* | NB* | NB* |
| REGN17206 | 196.3 ± 0.1 | 7.7 | 4.16E+03 | 1.51E−03 | 3.62E−07 | 7.7 |
| REGN17207 | 197.4 ± 0.9 | 62.2 | 8.53E+04 | 1.56E−04 | 1.82E−09 | 74.2 |
| REGN17208 | 649.7 ± 4.3 | 148.6 | 8.61E+04 | 3.78E−04 | 4.39E−09 | 30.6 |
| REGN17209 | 205.1 ± 0.6 | 65.0 | 8.97E+04 | 1.67E−04 | 1.86E−09 | 69.3 |
| REGN17210 | 187.9 ± 0.4 | 52.6 | 8.80E+04 | 1.87E−04 | 2.13E−09 | 61.7 |
| REGN17211 | 180.1 ± 0.8 | 0.6 | NB* | NB* | NB* | NB* |
| REGN17212 | 122.3 ± 0.2 | 27.4 | 7.22E+04 | 1.23E−04 | 1.70E−09 | 94.2 |
| REGN17213 | 62.2 ± 0.2 | 12.9 | 6.25E+04 | 1.67E−04 | 2.67E−09 | 69.2 |
| REGN17214 | 174.6 ± 1.1 | 9.9 | 1.70E+04 | 2.10E−03 | 1.23E−07 | 5.5 |
| REGN17224 | 170.7 ± 0.5 | 48.1 | 5.55E+04 | 1.81E−04 | 3.27E−09 | 63.8 |
| bsAb17225 | 176.8 ± 0.7 | 19.6 | 1.26E+05 | 2.42E−03 | 1.91E−08 | 4.8 |
| bsAb17226 | 173.3 ± 0.2 | 4.1 | 6.31E+03 | 9.30E−04 | 1.47E−07 | 12.4 |
| bsAb17227 | 171.9 ± 0.3 | 13.1 | 1.92E+05 | 2.24E−03 | 1.17E−08 | 5.1 |
| bsAb17228 | 184.5 ± 0.4 | 4.1 | 2.74E+05 | 1.60E−03 | 5.86E−09 | 7.2 |
| bsAb17229 | 210.0 ± 0.7 | 8.6 | 1.50E+04 | 2.66E−03 | 1.77E−07 | 4.3 |
| bsAb17230 | 188.8 ± 0.7 | 0.6 | NB* | NB* | NB* | NB* |
| bsAb17231 | 177.7 ± 0.5 | 1.4 | NB* | NB* | NB* | NB* |
| bsAb17232 | 180.8 ± 0.7 | 0.9 | NB* | NB* | NB* | NB* |
| bsAb17233 | 188.2 ± 1.6 | 1.2 | NB* | NB* | NB* | NB* |
| bsAb17234 | 180.8 ± 0.1 | 33.3 | 9.05E+04 | 1.41E−04 | 1.56E−09 | 82.0 |
| bsAb17235 | 187.7 ± 0.2 | 35.9 | 7.77E+04 | 1.43E−04 | 1.84E−09 | 80.8 |

*NB: Non-binding

Table 10 sets forth the equilibrium binding parameters of dimeric human SLC3A2-APIS to surface-captured anti-SLC3A2-APIS monospecific and bispecific antibodies at 37° C.

TABLE 10
Kinetic and Equilibrium Binding Parameters of Dimeric Human SLC3A2-APIS to Surface-
captured anti-SLC3A2-APIS Monospecific and Bispecific Antibodies at 37° C.

| Antibody | mAb | 30 nM | | | | |
|---|---|---|---|---|---|---|
| Identifier or | Capture | mFc.hSLC3A2- | ka | kd | K_{D} | t½ |
| Designation | (RU) | APIS Bind (RU) | (1/M/s) | (1/s) | (M) | (min) |
| REGN17192 | 222.9 ± 0.4 | 0.9 | NB* | NB* | NB* | NB* |
| REGN17193 | 263.2 ± 0.6 | 1.0 | NB* | NB* | NB* | NB* |
| REGN17194 | 94.6 ± 5.3 | 0.4 | NB* | NB* | NB* | NB* |
| REGN17195 | 199.5 ± 0.3 | 1.7 | NB* | NB* | NB* | NB* |
| REGN17196 | 247.5 ± 0.5 | 1.0 | NB* | NB* | NB* | NB* |
| REGN17197 | 241.2 ± 0.2 | 0.8 | NB* | NB* | NB* | NB* |
| REGN17198 | 212.1 ± 1.2 | 0.1 | NB* | NB* | NB* | NB* |
| REGN17199 | 221.8 ± 4.7 | 0.5 | NB* | NB* | NB* | NB* |
| REGN17200 | 231.6 ± 0.4 | 0.3 | NB* | NB* | NB* | NB* |
| REGN17201 | 252.2 ± 0.7 | 35.9 | 7.87E+04 | 2.41E−03 | 3.06E−08 | 4.8 |
| REGN17202 | 225.0 ± 0.5 | 10.8 | 9.40E+04 | 3.44E−03 | 3.66E−08 | 3.4 |
| REGN17203 | 240.5 ± 1.7 | 9.4 | 3.05E+04 | 3.97E−03 | 1.30E−07 | 2.9 |
| REGN17204 | 131.2 ± 0.3 | 26.5 | 1.05E+05 | 1.47E−03 | 1.40E−08 | 7.9 |
| REGN17205 | 137.8 ± 0.4 | 0.6 | NB* | NB* | NB* | NB* |
| REGN17206 | 234.8 ± 0.4 | 13.2 | 7.02E+03 | 4.06E−03 | 5.79E−07 | 2.8 |
| REGN17207 | 223.5 ± 0.6 | 90.9 | 9.76E+04 | 7.09E−04 | 7.27E−09 | 16.3 |
| REGN17208 | 772.3 ± 1.5 | 192.0 | 1.03E+05 | 1.58E−03 | 1.53E−08 | 7.3 |
| REGN17209 | 245.0 ± 0.7 | 106.8 | 1.12E+05 | 7.01E−04 | 6.24E−09 | 16.5 |
| REGN17210 | 211.9 ± 6.4 | 86.9 | 1.37E+05 | 8.18E−04 | 5.98E−09 | 14.1 |
| REGN17211 | 217.7 ± 4.0 | 1.3 | NB* | NB* | NB* | NB* |
| REGN17212 | 143.0 ± 0.5 | 62.3 | 9.35E+04 | 4.86E−04 | 5.20E−09 | 23.8 |
| REGN17213 | 64.4 ± 0.6 | 29.8 | 7.17E+04 | 5.09E−04 | 7.10E−09 | 22.7 |
| REGN17214 | 201.0 ± 0.3 | 8.9 | 8.60E+04 | 5.35E−03 | 6.22E−08 | 2.2 |
| REGN17224 | 193.9 ± 1.0 | 71.9 | 7.57E+04 | 7.10E−04 | 9.38E−09 | 16.3 |
| bsAb17225 | 215.2 ± 0.0 | 21.7 | 1.97E+05 | 5.62E−03 | 2.86E−08 | 2.1 |
| bsAb17226 | 203.2 ± 0.4 | 6.6 | 2.38E+05 | 3.26E−03 | 1.37E−08 | 3.5 |
| bsAb17227 | 198.3 ± 0.4 | 16.8 | 8.39E+04 | 2.62E−03 | 3.13E−08 | 4.4 |
| bsAb17228 | 220.6 ± 0.4 | 4.8 | 3.38E+04 | 8.64E−04 | 2.55E−08 | 13.4 |
| bsAb17229 | 248.6 ± 0.8 | 16.8 | 1.93E+05 | 4.76E−03 | 2.47E−08 | 2.4 |
| bsAb17230 | 214.4 ± 1.4 | 0.7 | NB* | NB* | NB* | NB* |
| bsAb17231 | 213.5 ± 1.0 | 1.1 | NB* | NB* | NB* | NB* |
| bsAb17232 | 212.3 ± 2.5 | 0.6 | NB* | NB* | NB* | NB* |
| bsAb17233 | 215.5 ± 1.0 | 1.9 | NB* | NB* | NB* | NB* |
| bsAb17234 | 214.8 ± 0.2 | 50.5 | 9.57E+04 | 6.62E−04 | 6.91E−09 | 17.5 |
| bsAb17235 | 219.8 ± 0.7 | 54.4 | 9.63E+04 | 7.35E−04 | 7.63E−09 | 15.7 |

*NB: Non-binding

### Example 3: Characterization of SLC3A2-APIS Monospecific Antibodies and SLC3A2-APISxCD3 Bispecific Antibodies Binding to Cell Surface Human SLC3A2-APIS Expressing and Wild-Type SLC3A2 Expressing Cells as Measured by Flow Cytometry

This example relates to an in vitro study performed to demonstrate the specificity of selected monospecific anti-SLC3A2-APIS antibodies and SLC3A2-APISxCD3 bispecific antibodies to bind wild-type SLC3A2 (either engineered to be expressed on NIH3T3 cells, or endogenously expressed on NALM6 cells) and SLC3A2-APIS (either engineered to be expressed on NIH3T3 cells or endogenously expressed on Mel-202 cells).

The SLC3A2-APIS cryptic neoantigen emerges due to mutations, for example, in the splicing factor 3B subunit 1 (SF3B1), which lead to the aberrant inclusion of four additional amino acids, A-P-I-S, into the sequence of the human solute carrier family 3 member 2 (hSLC3A2). Flow cytometry was used to evaluate binding of SLC3A2-APISxCD3 bispecific and SLC3A2-APIS monospecific antibodies to cells expressing SLC3A2-APIS. NIH3T3-derived cell lines examined in the experiment include NIH3T3 mouse fibroblast cell lines stably expressing wild-type hSLC3A2 (NIH3T3/hSLC3A2) or hSLC3A2-APIS (NIH3T3/hSLC3A2-APIS). Both cell lines were generated by transduction of lentivirus containing a blasticidin-resistant plasmid, pLVX, encoding full-length hSLC3A2 isoform 2 (pRGN20003, amino acids M1-A529 of accession #NP_001013269.1) or hSLC3A2 isoform 2-APIS (pRGN20004, M1-A533 with an insertion of APIS into the full length hSLC3A2 isoform 2 at amino acids 142-145), respectively. To assess the ability of antibodies to bind hSLC3A2 and hSLC3A2-APIS at endogenous levels, NALM6, a B cell precursor leukemia cell line carrying wild-type SF3B1, and Mel-202, a uveal melanoma cell line with a SF3B1 mutation were included in the experiment. The expression of wild-type hSLC3A2 in NALM6 and the APIS variant in Mel-202 was validated by mass spectrometry. NIH3T3 cells lacking hSLC3A2 expression were included as a negative control.

Experiments were carried out according to the following procedure. NIH3T3/hSLC3A2-APIS, NIH3T3/hSLC3A2, NIH3T3 or Mel-202 cells were rinsed once in phosphate-buffered saline (PBS) without Ca2+/Mg2+ and incubated for 10 minutes at 37° C. with Enzyme Free Cell Dissociation Solution to detach cells from the flask. The dissociated cells or NALM6 suspension cells were washed with PBS and counted with Cellometer™ Auto T4 cell counter. Cells were then resuspended to 1×10⁷ cells/mL in PBS and separately stained with 2.5 uM of CellTrace™ reagents for 20 minutes at room temperature to generate a unique fluorescence signature for each cell line (CellTrace™ Violet for NIH3T3/hSLC3A2 or NALM6 cells, CellTrace™ Yellow for NIH3T3 cells, and NIH3T3/hSLC3A2-APIS or Mel-202 cells were unstained). CellTrace™ labeling reaction was stopped by adding FBS to a final concentration of 25% in PBS followed by a 5 minute incubation at room temperature to quench unbound dye. After washing with PBS, equal numbers of NIH3T3/hSLC3A2-APIS, NIH3T3/hSLC3A2, and NIH3T3 cells were mixed in a ratio of 1:1:1 for multiplexing. Similarly, a separate mixture of equal numbers of Mel-202 and NALM6 cells in a ratio of 1:1 was also prepared. Approximately 2×10⁵ cells/well were seeded into 96-well Corning plates and stained with LIVE/DEAD™ Fixable Near-IR in PBS for 20 minutes at 4° C. to discriminate live and dead cells. Cells were washed with 2% FBS in PBS by centrifugation with benchtop centrifuge. Cells were incubated for 30 minutes at 4° C. with serial dilutions of antibodies over a range of 1.7 pM to 100 nM in 2% FBS in PBS. Antibodies tested include SLC3A2-APISxCD3 bispecific antibodies, SLC3A2-APIS monospecific antibodies, CD3-7195P and CD3-7221G bispecific control antibodies, and isotype control antibodies. After washing, cell-bound antibodies were detected with APC-conjugated goat anti-human IgG secondary antibody specific for the Fcγ fragment for 30 minutes at 4° C. Cells were washed and subsequently fixed with 50% solution of Cytofix™ Fixation Buffer in 2% FBS in PBS for 20 minutes at room temperature. Cells were washed and resuspended in 2% FBS in PBS and stored at 4° C. for downstream flow cytometry analysis.

Fluorescence signals were acquired and recorded on a Bio-Rad ZE5 Cell Analyzer according to the manufacturer's recommended procedure. Flow cytometry data analysis was performed using FlowJo™ software. Multiplexed samples were deconvoluted and individual cell populations were identified based on their unique CellTrace™ fluorescence signature. APC Median Fluorescence Intensity (MFI) was recorded to indicate the binding intensity of each antibody over a range of concentrations.

Maximum binding, defined as the ratio of the MFI at the highest antibody concentration tested (100 nM) to the MFI from the sample with secondary APC-detection antibody alone, was reported as indicative of the ability to bind hSLC3A2-APIS or hSLC3A2. Antibodies with a maximum binding greater than 15 were classified as binders and antibodies with a maximum binding of 15 or less were classified as non-binders. Specific binding, defined as the fold-change in maximum binding on hSLC3A2-APIS or hSLC3A2 expressing cells over maximum binding on negative control NIH3T3 cells, was reported as indicative of binding specificity to hSLC3A2-APIS or wild-type hSLC3A2. Antibodies with a specific binding of greater than 5 were classified as specific binders, and those with a specific binding of 5 or less were classified as non-specific binders. Only a few tested antibodies exhibited saturation of the binding signal at the highest tested concentration of 100 nM, therefore EC₅₀ values, defined as the concentration of antibody that yields 50% of the saturation binding, were not determined.

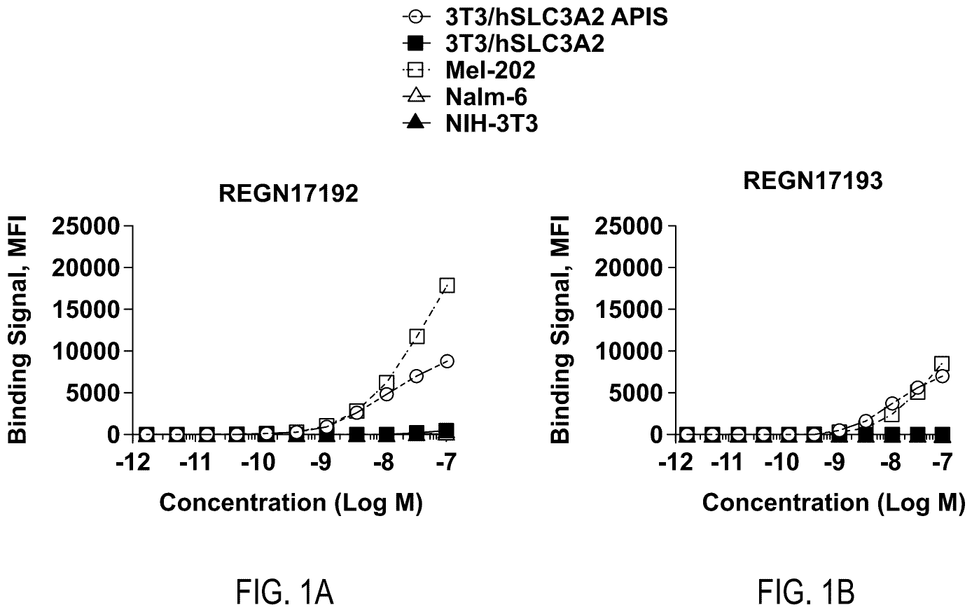

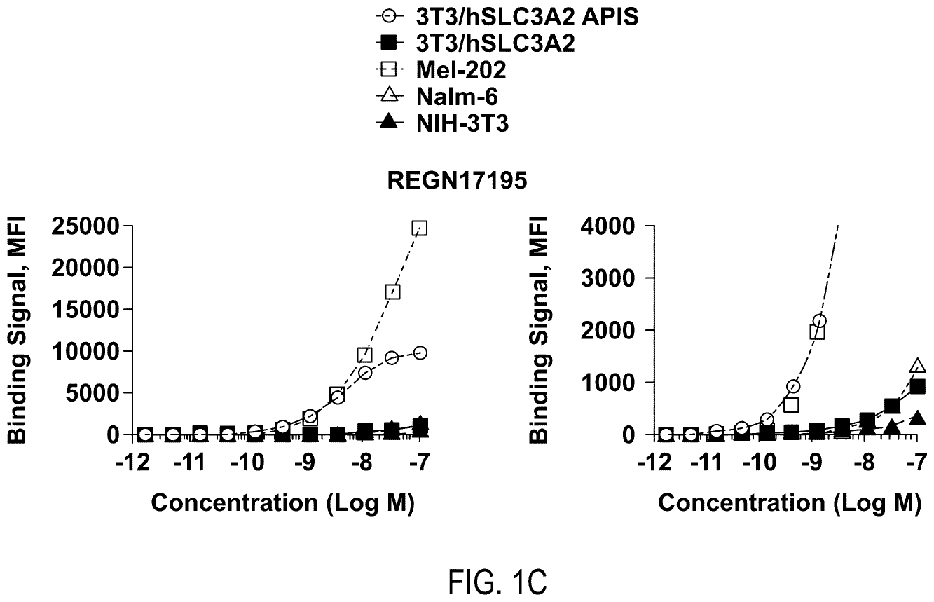

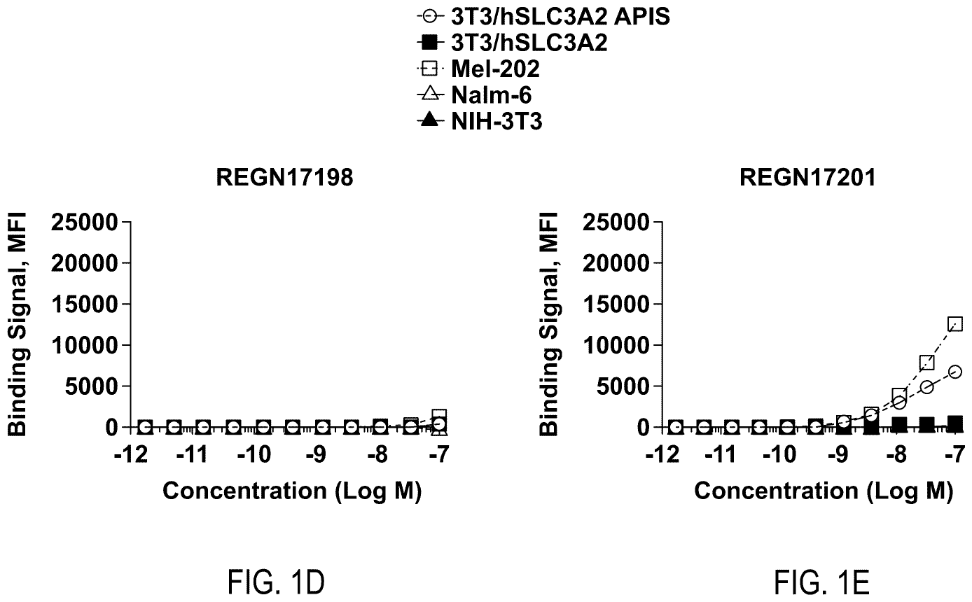

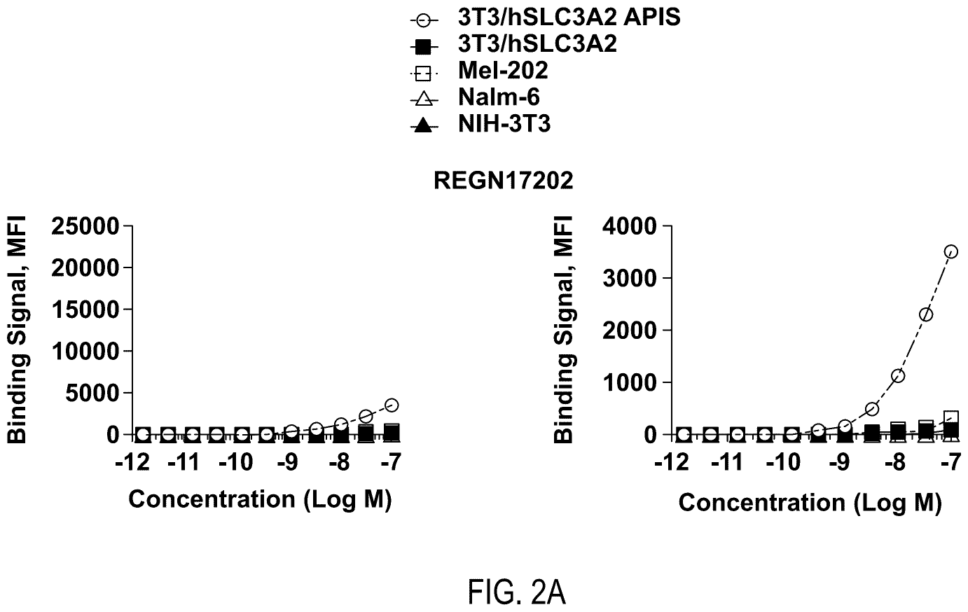

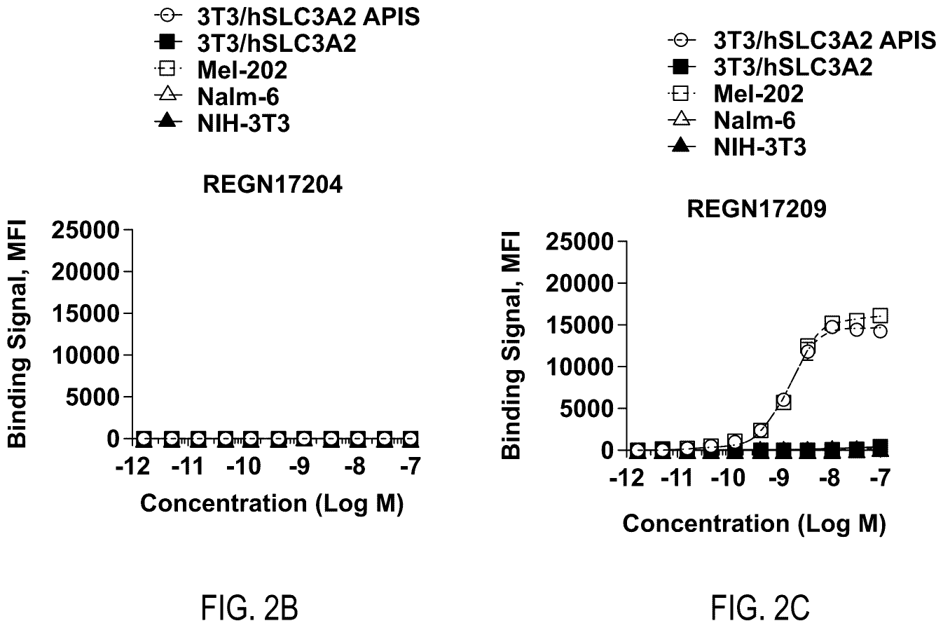

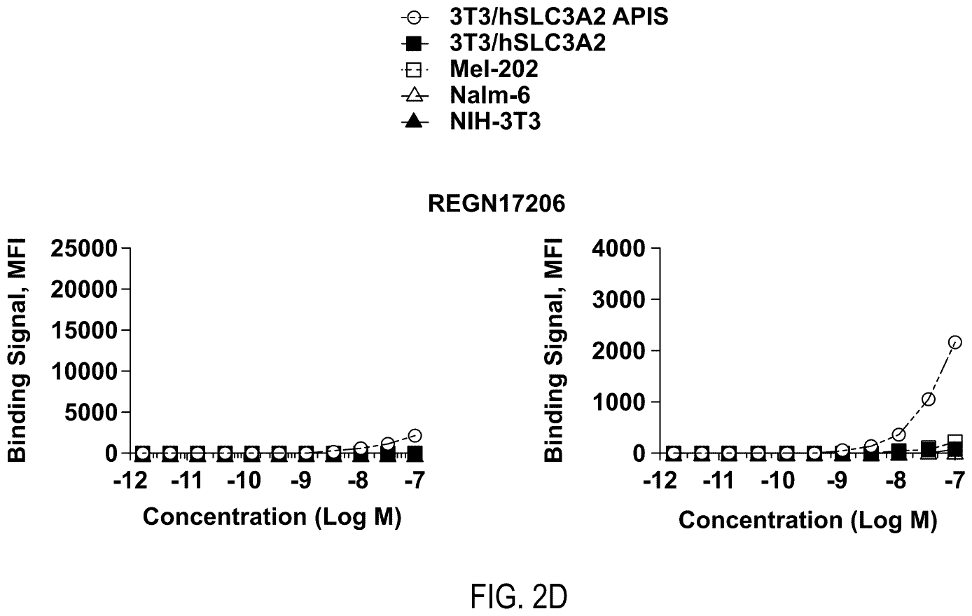

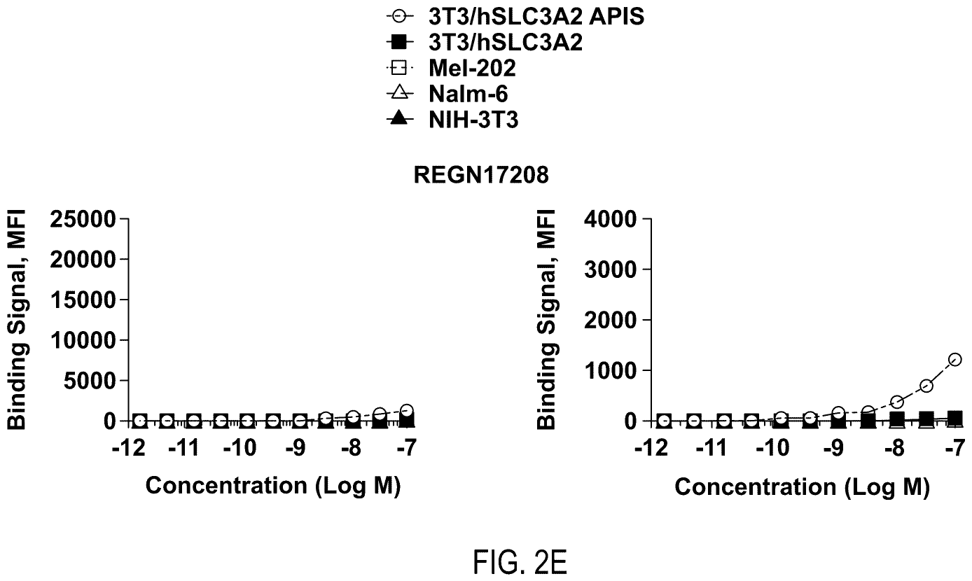

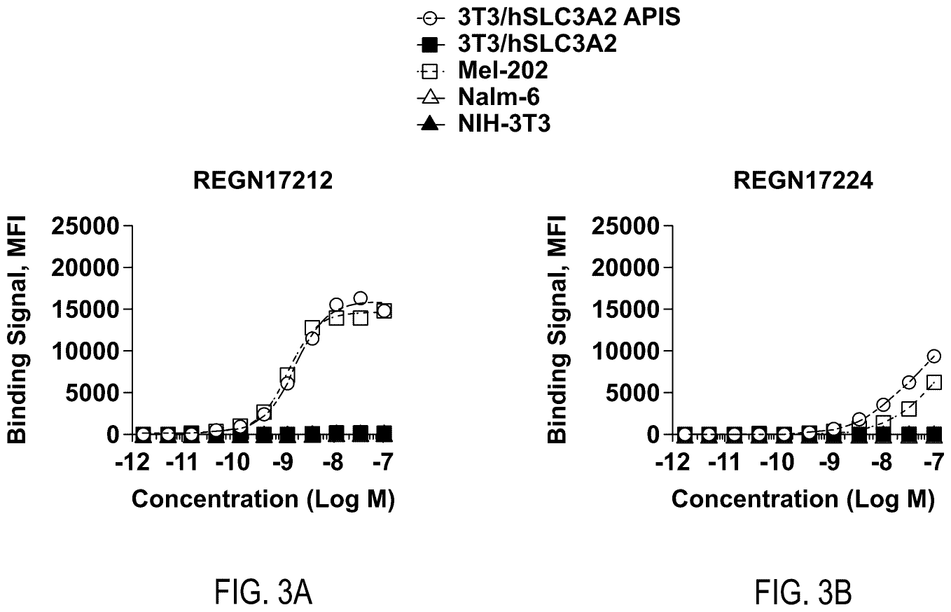

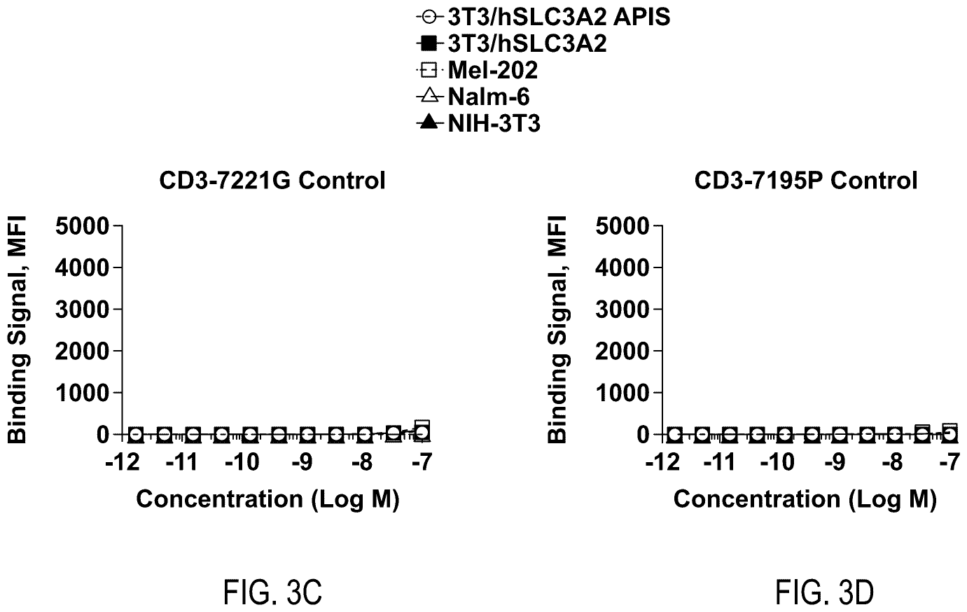

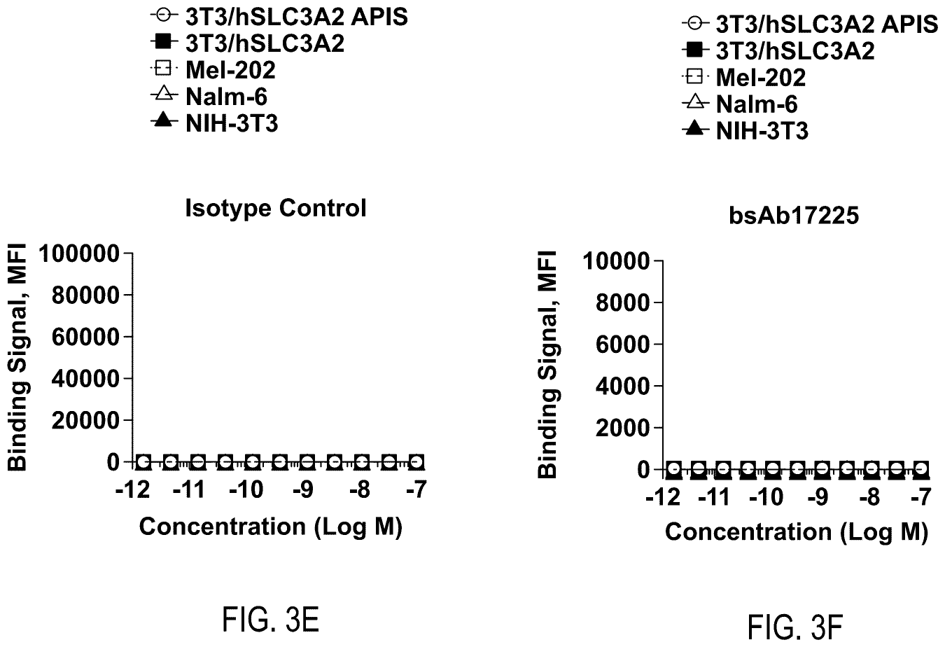

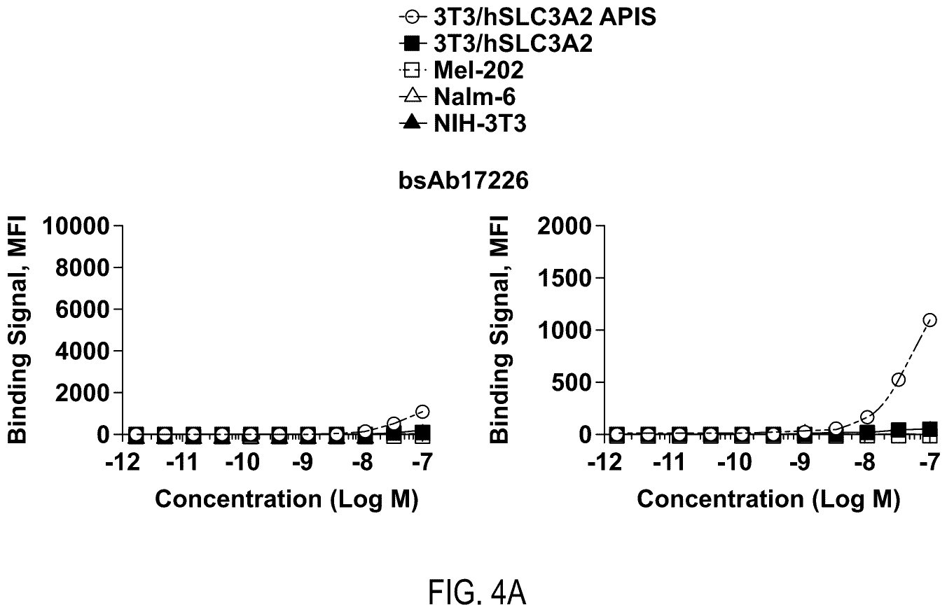

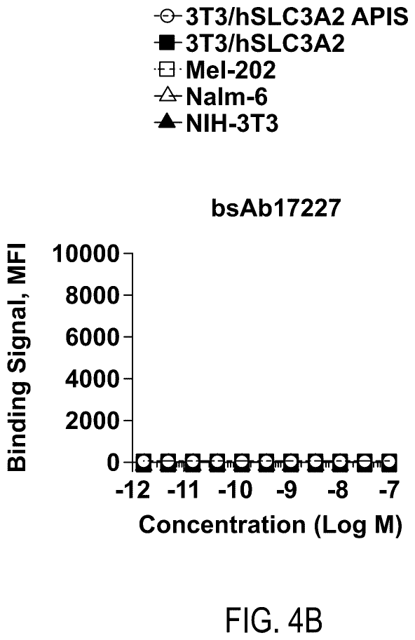

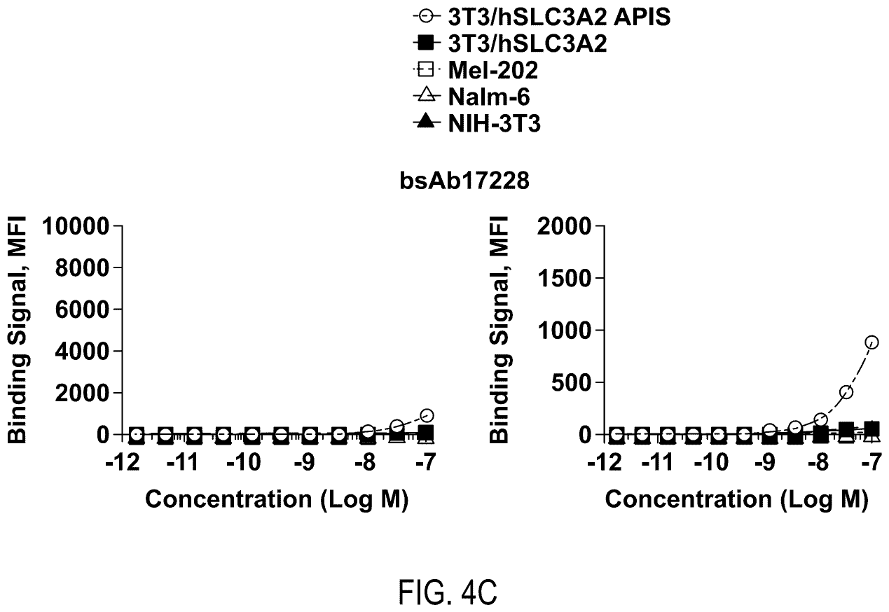

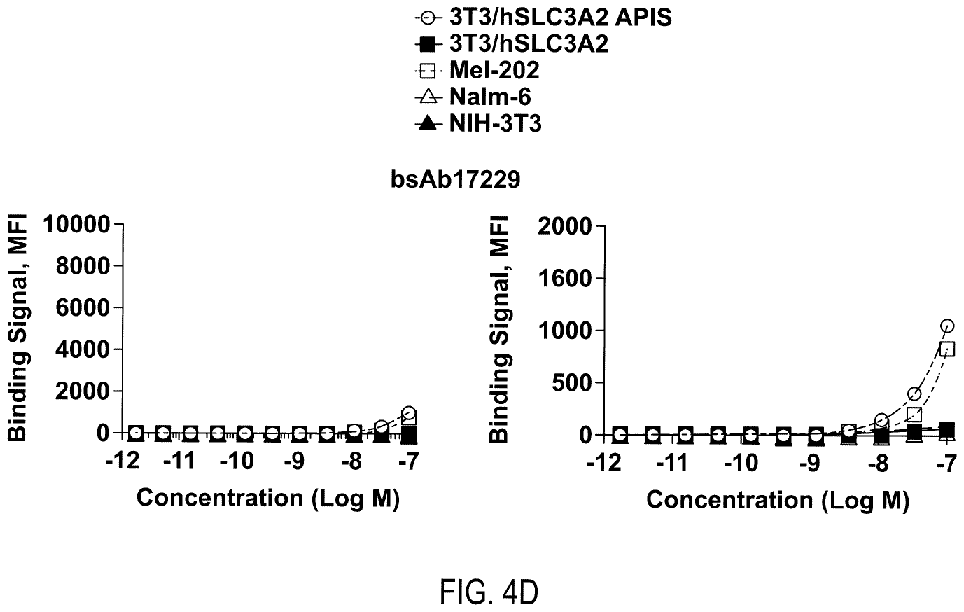

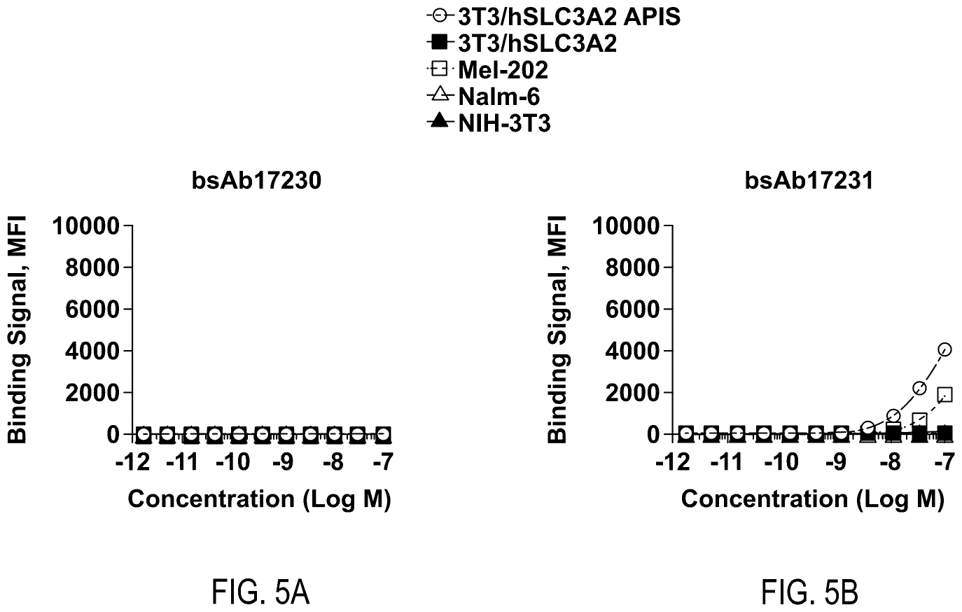

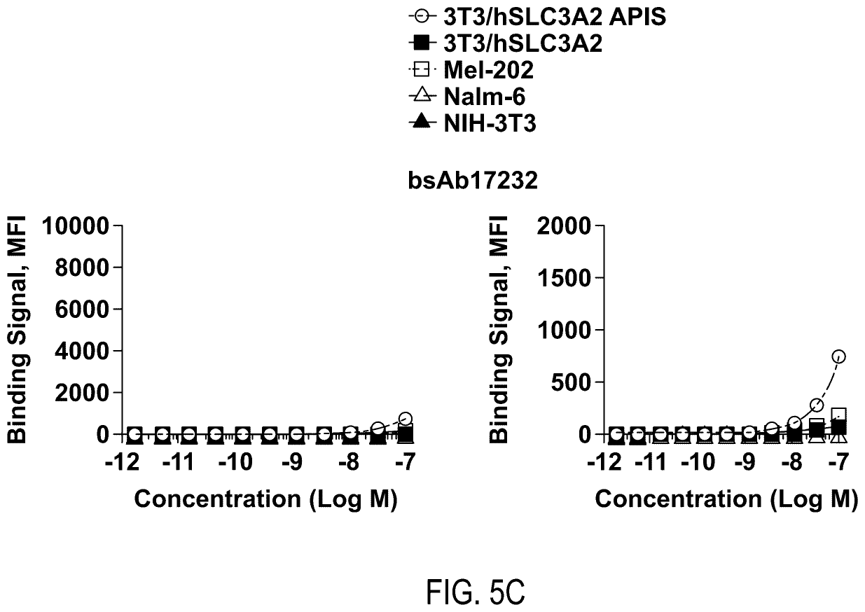

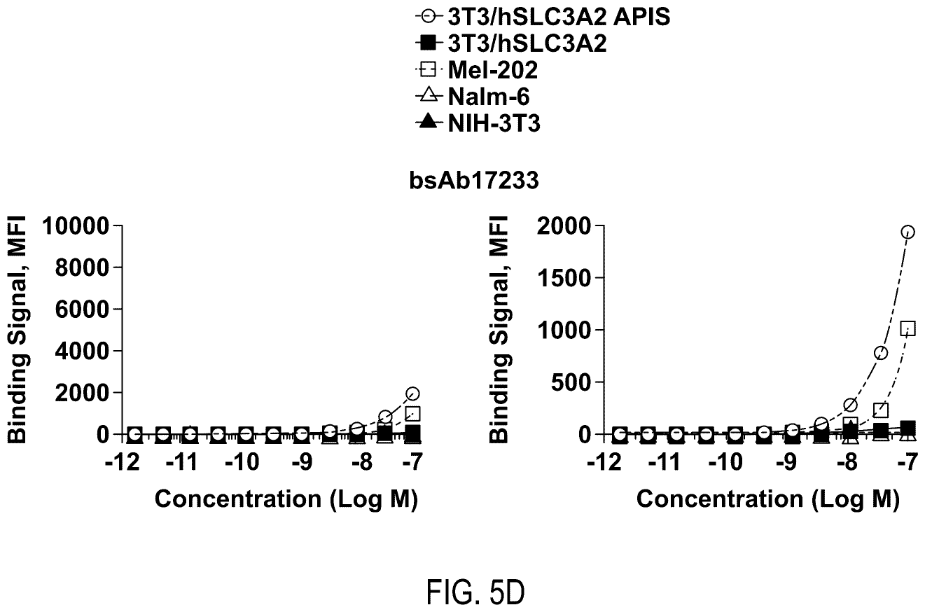

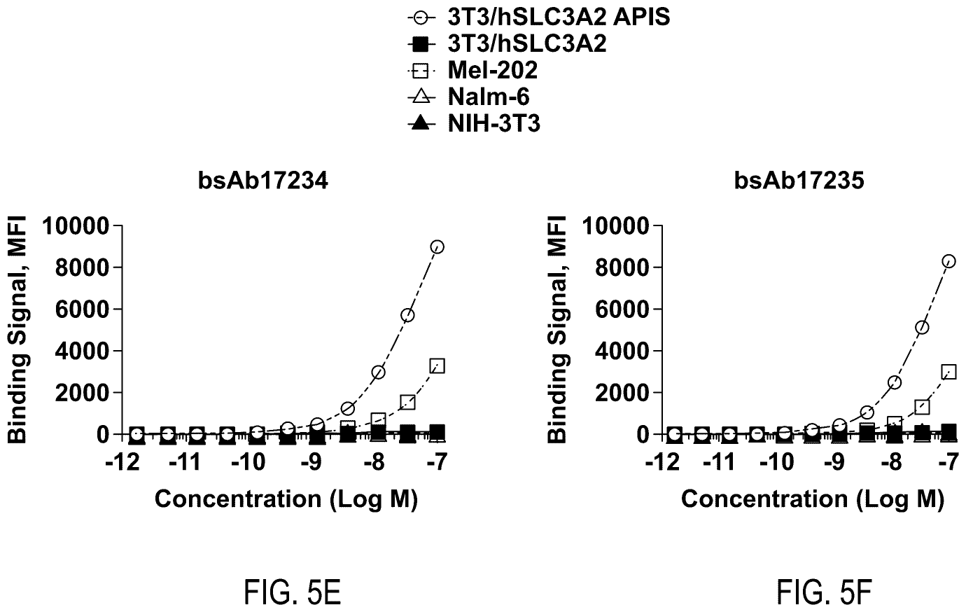

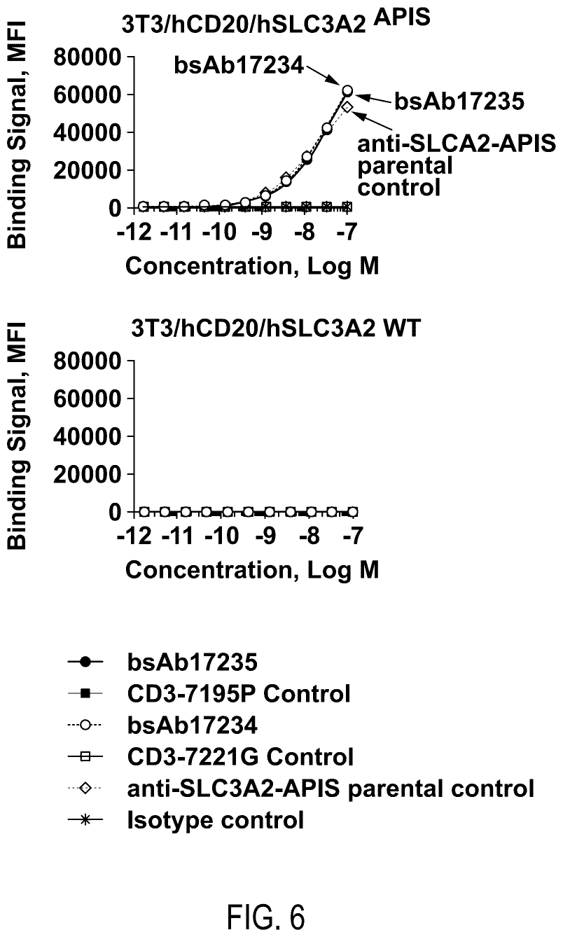

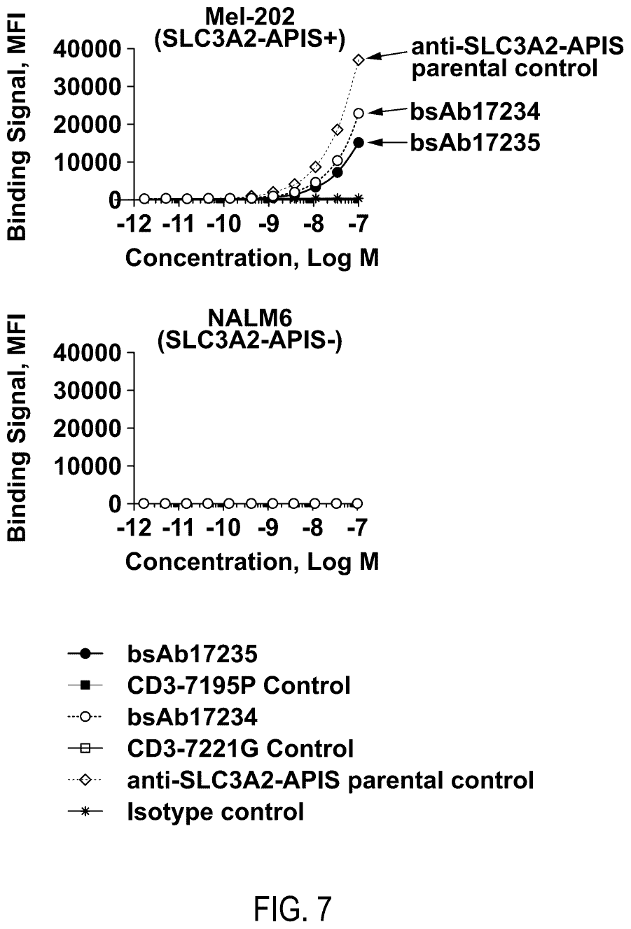

The experimental results for maximum binding (the ratio of antibody binding relative to secondary-antibody alone on the same cell line) and specific binding (the ratio of maximum binding relative to a negative cell line) at the highest antibody concentration tested are summarized in Tables 11 and 12. The binding signal for each antibody over different concentrations of antibody for different cell types is shown in FIGS. 1A-5E. The binding signal for NIH3T3 cells expressing exogenous hSLC3A2-APIS and hSLC3A2-WT over different concentrations of 2 bispecific SLC3A2-APISxCD3 antibodies and their controls are shown in FIG. 6: bispecific SLC3A2-APISxCD3 antibody bsAb17235; bispecific SLC3A2-APISxCD3 antibody bsAb17234; two CD3 bispecific controls (CD3-7195P and CD3-7221G); bivalent SLC3A2-APIS parental control; and Isotype control. The binding signal for Mel-202 cells expressing endogenous hSLC3A2-APIS and NALM6 cells expressing endogenous hSLC3A2-WT over different concentrations of 2 bispecific SLC3A2-APISxCD3 antibodies and their controls are shown in FIG. 7: bispecific SLC3A2-APISxCD3 antibody bsAb17235; bispecific SLC3A2-APISxCD3 antibody bsAb17234; two CD3 bispecific controls (CD3-7195P and CD3-7221G); bivalent SLC3A2-APIS parental control; and Isotype control.

Results—Binding of SLC3A2-APISxCD3 bispecific antibodies to exogenously and endogenously expressed wild-type SLC3A2 or SLC3A2-APIS: Five out of eleven SLC3A2-APISxCD3 bispecific antibodies (bsAb17229, bsAb17231, bsAb17233, bsAb17234, and bsAb17235) exhibited concentration-dependent specific binding to engineered NIH3T3/hSLC3A2-APIS cells. These 5 SLC3A2-APISxCD3 antibodies had a maximum binding ranging from 37.6 to 277.9 and a specific binding ranging from 19.0 to 68.0 at the highest concentration tested (100 nM). These 5 SLC3A2-APISxCD3 antibodies exhibited concentration-dependent specific binding to Mel-202 cells endogeneously expressing hSLC3A2-APIS, with a maximum binding ranging from 33.0 to 144.6 and a specific binding ranging from 15.9 to 35.4. Three out of eleven SLC3A2-APISxCD3 bispecific antibodies (bsAb17226, bsAb17228, and bsAb17232) displayed concentration-dependent specific binding only to engineered NIH3T3/hSLC3A2-APIS cells with a maximum binding ranging from 23.3 to 36.0 and a specific binding ranging from 11.4 to 18.9 at the highest concentration tested (100 nM). These 3 SLC3A2-APISxCD3 bispecific antibodies displayed specific binding values of 1.0 or less to both NIH3T3/hSLC3A2 and NALM6 cells expressing wild-type hSLC3A2, leading to their classification as non-binders to wild-type hSLC3A2. The remaining 3 bispecific antibodies (bsAb17225, bsAb17227, bsAb17230) did not show detectable binding to either hSLC3A2 or hSLC3A2-APIS expressing cells. BsAb17225 and bsAb17227, however, did show significant binding in the Biacore assay of Example 2.

No detectable binding to wild-type hSLC3A2 or hSLC3A2-APIS expressing cells was observed for any of the control antibodies up to the highest antibody concentration tested (100 nM). None of the tested antibodies bound to the negative control NIH3T3 cells.

Binding of anti-SLC3A2-APIS monospecific antibodies to exogenously and endogenously expressed wild-type SLC3A2 or SLC3A2-APIS: Among 12 anti-SLC3A2-APIS monospecific antibodies tested, 1 (REGN17195) demonstrated specific binding to hSLC3A2-APIS expressed on NIH3T3/hSLC3A2-APIS (maximum binding of 293.7 and specific binding of 33.3) and hSLC3A2-APIS expressed on Mel-202 cells (maximum binding of 823.4 and specific binding of 93.4); while also demonstrating specific binding to wild-type hSLC3A2 expressed on NALM6 cells (maximum binding of 58.0 and specific binding of 6.6). Six of the 12 SLC3A2-APIS monospecific antibodies (REGN17192, REGN17193, REGN17201, REGN17209, REGN17212, and REGN17224) displayed specific binding to both NIH3T3/hSLC3A2-APIS (maximum binding ranging from 193.6 to 519.2, specific binding ranging from 34.0 to 85.3) and hSLC3A2-APIS expressed on Mel-202 cells (maximum binding ranging from 245.9 to 750.6, specific binding ranging from 57.0 to 112.3). Three SLC3A2-APIS monospecific antibodies (REGN17202, REGN17206, REGN17208) exhibited specific binding only to NIH3T3/hSLC3A2-APIS cells, (maximum binding ranging from 39.8 to 97.6 and a specific binding ranging from 19.3 to 31.1). Nine monospecific antibodies did not bind to wild-type hSLC3A2 expressed on either NIH3T3/hSLC3A2 or NALM6 cells, as their specific binding was equal to or less than 2.2. Two monospecific antibodies (REGN17198, REGN17204) did not exhibit detectable or specific binding to either wild-type hSLC3A2 or hSLC3A2-APIS expressing cells. Notably, of the 4 monospecific antibodies that did not bind hSLC3A2-APIS in the Biacore assay of Example 2, 3 did exhibit specific binding to hSLC3A2-APIS expressed in cells. Thus, only REGN17198 showed no hSLC3A2-APIS binding in either assay.

Table 11 sets forth the anti-SLC3A2-APIS antibody binding specificity to NIH3T3 cells exogenously expressing human SLC3A2-APIS or wild-type SLC3A2.

TABLE 11
Summary of Anti-SLC3A2-APIS Antibody Binding Specificity to NIH3T3 Cells
Exogenously Expressing Human SLC3A2-APIS or Wild-Type SLC3A2^{a, b}

| | Antibody | | | | | |
|---|---|---|---|---|---|---|
| Antibody | Format | NIH3T3/hSLC3A2-APIS | | NIH3T3/hSLC3A2 | | NIH3T3 |
| Identifier or | (monosp | Max | Specific | Max | Specific | Max |
| Designation | or bisp) | binding | binding | binding | binding | binding |
| bsAb17225 | bi | 4.3 | 3.0 | 1.3 | 0.9 | 1.4 |
| bsAb17226 | bi | 36.0 | 18.9 | 1.8 | 0.9 | 1.9 |
| bsAb17227 | bi | 3.0 | 2.2 | 1.3 | 1.0 | 1.3 |
| bsAb17228 | bi | 26.3 | 13.1 | 1.8 | 0.9 | 2.0 |
| bsAb17229 | bi | 37.6 | 19.0 | 1.9 | 1.0 | 2.0 |
| bsAb17230 | bi | 1.7 | 1.0 | 1.6 | 1.0 | 1.6 |
| bsAb17231 | bi | 135.3 | 36.3 | 3.5 | 0.9 | 3.7 |
| bsAb17232 | bi | 23.3 | 11.4 | 1.9 | 0.9 | 2.0 |
| bsAb17233 | bi | 65.9 | 26.4 | 2.3 | 0.9 | 2.5 |
| bsAb17234 | bi | 277.9 | 68.0 | 3.9 | 1.0 | 4.1 |
| bsAb17235 | bi | 241.0 | 64.7 | 3.5 | 0.9 | 3.7 |
| REGN17192 | mono | 290.9 | 39.5 | 14.0 | 1.9 | 7.4 |
| REGN17193 | mono | 238.6 | 43.0 | 6.3 | 1.1 | 5.5 |
| REGN17195 | mono | 293.7 | 33.3 | 26.3 | 3.0 | 8.8 |
| REGN17198 | mono | 11.2 | 0.8 | 10.9 | 0.8 | 14.0 |
| REGN17201 | mono | 193.6 | 37.9 | 11.2 | 2.2 | 5.1 |
| REGN17202 | mono | 97.6 | 30.6 | 2.9 | 0.9 | 3.2 |
| REGN17204 | mono | 10.6 | 6.9 | 1.3 | 0.9 | 1.5 |
| REGN17206 | mono | 64.7 | 31.1 | 1.9 | 0.9 | 2.1 |
| REGN17208 | mono | 39.8 | 19.3 | 2.0 | 1.0 | 2.1 |
| REGN17209 | mono | 443.6 | 34.0 | 15.0 | 1.2 | 13.0 |
| REGN17212 | mono | 519.2 | 85.3 | 7.4 | 1.2 | 6.1 |
| REGN17224 | mono | 285.1 | 67.3 | 3.9 | 0.9 | 4.2 |
| CD3-7195P | bi | 2.0 | 0.9 | 2.3 | 1.0 | 2.3 |
| CD3-7221G | bi | 1.2 | 1.0 | 1.2 | 1.0 | 1.2 |
| Isotype | — | 1.3 | 0.6 | 1.7 | 0.8 | 2.2 |

^{a}Maximum binding was calculated by dividing the Median Fluorescence Intensity (MFI) at the highest antibody concentration tested (100 nM) by the MFI from the secondary APC detection antibody alone.
^{b}Specific binding was calculated by dividing the maximum binding on NIH3T3/hSLC3A2-APIS, Mel-202, NIH3T3/hSLC3A2 or NALM6 cells by the maximum binding on NIH3T3 cells.

Table 12 sets forth the anti-SLC3A2-APIS antibody binding specificity to Mel-202 cells, which endogenously express human SLC3A2-APIS, and NALM6 cells, which endogenously express wild-type SLC3A2.

TABLE 12
Summary of Anti-SLC3A2-APIS Antibody Binding Specificity
to Mel-202 Cells Expressing Human SLC3A2-APIS and
NALM6 Cells Expressing Wild-Type SLC3A2

| | Antibody | | | | |
|---|---|---|---|---|---|
| Antibody | Format | Mel-202 (hSLC3A2-APIS+) | | NALM6 (hSLC3A2+) | |
| Identifier or | (monosp | Max | Specific | Max | Specific |
| Designation | or bisp) | binding | binding | binding | binding |
| bsAb17225 | bi | 2.5 | 1.8 | 1.0 | 0.7 |
| bsAb17226 | bi | 3.1 | 1.6 | 1.0 | 0.5 |
| bsAb17227 | bi | 1.8 | 1.3 | 1.0 | 0.7 |
| bsAb17228 | bi | 2.8 | 1.4 | 1.0 | 0.5 |
| bsAb17229 | bi | 33.0 | 16.7 | 1.4 | 0.7 |
| bsAb17230 | bi | 2.2 | 1.3 | 1.0 | 0.6 |
| bsAb17231 | bi | 59.0 | 15.9 | 1.4 | 0.4 |
| bsAb17232 | bi | 5.1 | 2.5 | 1.0 | 0.5 |
| bsAb17233 | bi | 44.6 | 17.9 | 1.2 | 0.5 |
| bsAb17234 | bi | 144.6 | 35.4 | 1.7 | 0.4 |
| bsAb17235 | bi | 119.5 | 32.1 | 1.5 | 0.4 |
| REGN17192 | mono | 694.4 | 94.2 | 14.0 | 1.9 |
| REGN17193 | mono | 316.1 | 57.0 | 2.9 | 0.5 |
| REGN17195 | mono | 823.4 | 93.4 | 58.0 | 6.6 |
| REGN17198 | mono | 63.0 | 4.5 | 1.7 | 0.1 |
| REGN17201 | mono | 513.4 | 100.5 | 9.5 | 1.9 |
| REGN17202 | mono | 12.7 | 4.0 | 1.1 | 0.3 |
| REGN17204 | mono | 1.3 | 0.9 | 0.9 | 0.6 |
| REGN17206 | mono | 9.7 | 4.7 | 1.0 | 0.5 |
| REGN17208 | mono | 3.5 | 1.7 | 1.0 | 0.5 |
| REGN17209 | mono | 750.6 | 57.6 | 14.0 | 1.1 |
| REGN17212 | mono | 683.2 | 112.3 | 11.9 | 1.9 |
| REGN17224 | mono | 245.9 | 58.1 | 2.4 | 0.6 |
| CD3-7195P | bi | 7.0 | 3.1 | 1.5 | 0.7 |
| CD3-7221G | bi | 4.0 | 3.3 | 1.2 | 1.0 |
| Isotype | — | 2.4 | 1.1 | 1.6 | 0.7 |

### Example 4: Characterization of SLC3A2-APIS Bispecific Antibody Activity in an Engineered Reporter Assay Using Target Cell Lines Expressing Wild-Type SLC3A2 or SLC3A2-APIS

This example relates to an in vitro study performed to demonstrate the ability of select SLC3A2-APISxCD3 bispecific antibodies to activate CD3 on Jurkat/NFAT-Luc reporter cells in the presence of the following target cell lines expressing either wild-type SLC3A2 or the APIS insertion variant of SLC3A2, SLC3A2-APIS: NIH3T3/hCD20, NIH3T3/hSLC3A2-WT/hCD20, NIH3T3/hSLC3A2-APIS/hCD20, NALM6, Mel-202.

The ability of SLC3A2-APIS bispecific antibodies to specifically activate CD3 in the presence of target cells expressing SLC3A2-APIS, was measured in an engineered reporter assay. In this assay, engineered Jurkat cells express the reporter gene luciferase under the control of the transcription factor NFAT (NFAT-Luc) along with endogenous expression of human CD3. The target cells used in this assay were NIH3T3 cells engineered to express CD20 alone or (1) in combination with wild-type SLC3A2, or (2) in combination with SLC3A2-APIS. NALM6 cells were also used due to their endogenous expression of wild-type SLC3A2, and Mel-202 cells due to their endogenous expression of SLC3A2-APIS. The ability of CD3 bispecific antibodies to stimulate CD3 activity is assessed by combining reporter cells with target cells and a titration of antibody. Activation of CD3 results in NFAT-driven luciferase production, which is then measured via a luminescence readout.

Cell Lines: The following reporter cells and target cells were used in these experiments. Reporter cells were Jurkat/NFAT-luc: an engineered cell line made by stably transducing Jurkat cells, a leukemia T cell line which was isolated from a male with acute T cell leukemia, with a reporter construct expressing luciferase under the control of the Nuclear Factor of Activated T cells (NFAT) promoter. Jurkat cells endogenously express CD3. Target cells were (1) NIH3T3/hCD20: An engineered cell line made by stably transducing NIH3T3 cells, a fibroblast cell line isolated from a mouse NIH/Swiss embryo, with human CD20 (Uniprot accession #: P11836, amino acids M1 to P297); (2) NIH3T3/hSLC3A2-WT/hCD20: An engineered cell line made by stably transducing NIH3T3 cells with wild-type human SLC3A2 (hSLC3A2-amino acids M1 to A529 of accession number P08195) and human CD20; (3) NIH3T3/hSLC3A2-APIS/hCD20: An engineered cell line made by stably transducing NIH3T3 cells with a variant of human SLC3A2 containing an insertion of amino acids, APIS (hSLC3A2-amino acids M1 to A242 of accession number P08195-amino acids APIS-amino acids G243 to A529 of accession number P08195) and human CD20; (4) NALM6: A cell line that endogenously expresses wild-type human SLC3A2, isolated from the peripheral blood of a man with acute lymphoblastic leukemia (ALL); and (5) Mel-202: A cell line that endogenously expresses the APIS insertion variant of human SLC3A2, established from a primary uveal melanoma.

Experimental Procedure: Jurkat/NFAT-luc cells were resuspended in assay media and added to 384-well white plates at a final concentration of 1.5×10⁴ cells/well. Target cells, resuspended in assay media, were then added to wells at a concentration of 5.0×10³ cells/well (NIH3T3/hCD20, NIH3T3/SLC3A2-WT/hCD20, NIH3T3/SLC3A2-APIS/hCD20, NALM6, and Mel-202 cells). SLC3A2-APISxCD3 antibodies and CD3-7221G bispecific control antibodies were prepared in assay media and titrated from 100 nM to 0.38 PM in a 1:4 dilution, with the final point of the 11-point dilution containing no titrated antibody, and added, in duplicate, to the appropriate wells. Plates were incubated at 37° C. and 5% CO2 for 4.5 hours and then ONE-Glo™ luciferase substrate was added to each well according to manufacturer's instructions. The luciferase activity was recorded as a luminescence signal using the ENVISION™ plate reader and expressed as relative light units (RLU). The EC₅₀ values were determined by a 4-parameter logistic equation over an 11-point response curve using GraphPad Prism™ software. Signal recorded for the 11^{th} point on the dilution curve (no titrated antibody) was plotted at 95 fM. Maximal RLU is given as the mean max response detected within the tested dose range.

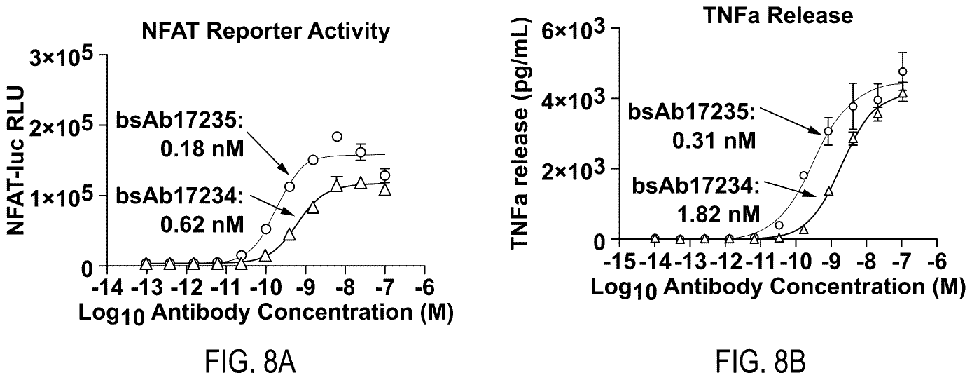

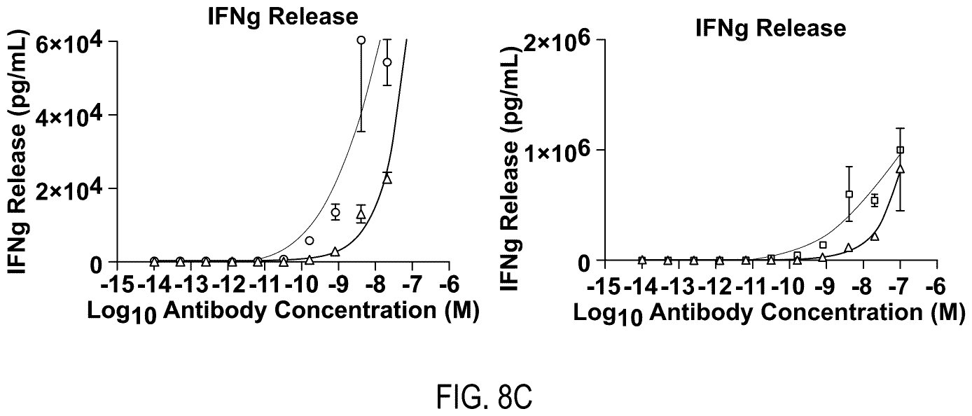

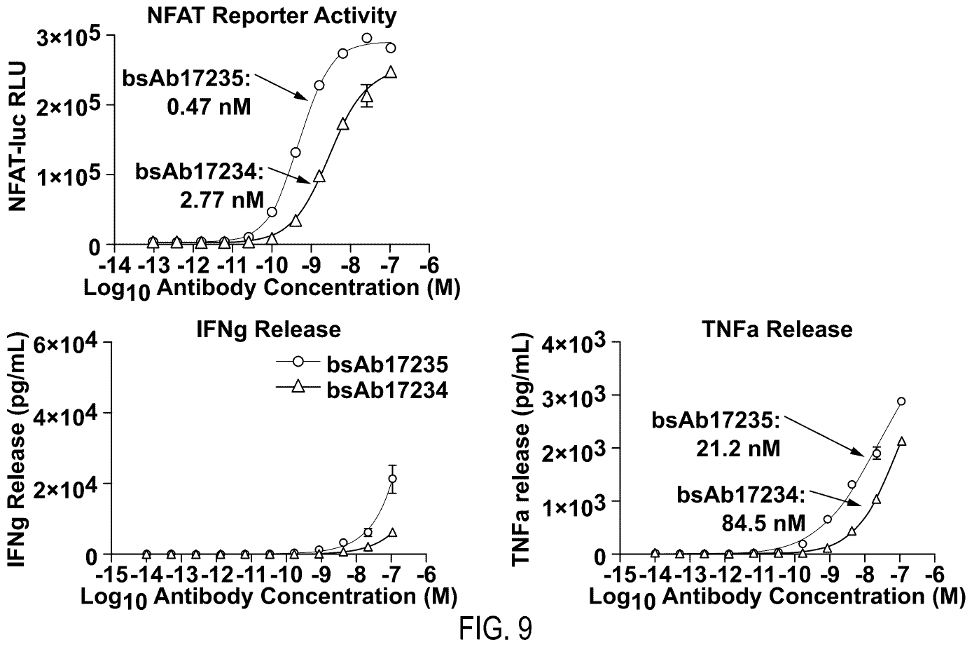

Results: In the presence of Jurkat/NFAT-luc cells and target cells expressing SLC3A2-APIS, the addition of SLC3A2-APISxCD3 antibodies led to a dose dependent increase in NFAT activity. The potency values and maximum reporter activity for the SLC3A2-APISxCD3 antibodies in the different cell types expressing SLC3A2-WT or SLC3A2-APIS are shown in Tables 13 and 14, respectively, below. NFAT activity in NIH3T3/SLC3A2-APIS/hCD20 cells induced by bispecific antibodies bsAb17235 and bsAb17234 is shown in FIG. 8A. NFAT activity in Mel-202 cells induced by bispecific antibodies bsAb17235 and bsAb17234 is shown in FIG. 9, upper-left graph.

In the presence of NIH3T3/hCD20 target cells lacking SLC3A2-APIS, bsAb17229 led to an increase in luciferase activity indicating some activity independent of SLC3A2-APIS expression. The CD3-7221G bispecific control also led to a response at the highest dose tested. In the presence of NIH3T3/hCD20 target cells expressing SLC3A2-APIS, SLC3A2-APISxCD3 bispecific antibodies bsAb17235, bsAb17234, bsAb17226, bsAb17231, bsAb17233, and bsAb17232 led to an increase in luciferase activity, which was specific to target and greater than CD3-7221G bispecific control. bsAb17228, bsAb17227, bsAb17225, and bsAb17230 led to a response similar to CD3-7221G bispecific control. In the presence of NALM6 target cells lacking SLC3A2-APIS, only bsAb17229 led to an increase in luciferase activity, indicating some activity independent of SLC3A2-APIS expression. In the presence of Mel-202 target cells expressing SLC3A2-APIS, SLC3A2-APISxCD3 bispecific antibodies bsAb17235, bsAb17234, and bsAb17226 led to an increase in luciferase activity, which was specific to target and greater than CD3-7221G bispecific control. bsAb17231, bsAb17233, bsAb17232, bsAb17228, bsAb17227, bsAb17225, and bsAb17230 led to a response similar to CD3-7221G bispecific control.

Table 13 sets forth potency values for select SLC3A2-APISxCD3 antibodies, as indicated by EC₅₀ in M.

TABLE 13
Potency values, EC50 [M], for SLC3A2-APISxCD3 antibodies

| Antibody | NIH3T3/ | NIH3T3/hSLC3A2- | NIH3T3/hSLC3A2- | | |
|---|---|---|---|---|---|
| Identifier | hCD20 | WT/hCD20 | APIS/hCD20 | NALM6 | Mel-202 |
| bsAb17225 | ND | ND | 3.80E−09 | ND | ND |
| bsAb17226 | ND | ND | 1.28E−09 | ND | 4.40E−09 |
| bsAb17227 | ND | ND | 3.09E−09 | ND | ND |
| bsAb17228 | ND | ND | 2.75E−09 | ND | ND |
| bsAb17229 | 1.50E−09 | 2.46E−09 | 9.01E−11 | 1.57E−09 | 1.55E−10 |
| bsAb17231 | ND | ND | 4.64E−10 | ND | 1.72E−09 |
| bsAb17232 | ND | ND | 3.19E−09 | ND | 8.86E−09 |
| bsAb17230 | ND | ND | 1.45E−08 | ND | ND |
| bsAb17233 | ND | ND | 1.22E−09† | ND | 2.02E−09† |
| bsAb17234 | ND | ND | 6.19E−10 | ND | 2.77E−09 |
| bsAb17235 | ND | ND | 2.26E−10† | ND | 4.71E−10 |
| CD3-7221G | 1.69E−08 | 1.59E−08 | 2.08E−08 | 1.66E−08 | 8.67E−09 |
| Control | | | | | |

Abbreviations:
ND: Not Determined because no dose dependent response was observed
†While all data points are used to determine Maximum luciferase activity, for EC₅₀ calculation the values for the highest antibody concentration was removed due to a “hook effect”.

Table 14 sets forth Maximum Reporter Activity for select SLC3A2-APISxCD3 antibodies in Max Relative Light Units (RLU).

TABLE 14
Maximum Reporter Activity, Max RLU, for SLC3A2-APISxCD3 antibodies

| Antibody | | NIH3T3/hSLC3A2- | NIH3T3/hSLC3A2- | | |
|---|---|---|---|---|---|
| Identifier | NIH3T3/hCD20 | WT/hCD20 | APIS/hCD20 | NALM6 | Mel-202 |
| bsAb17225 | 7.86E+03 | 7.52E+03 | 1.00E+04 | 6.06E+03 | 5.86E+03 |
| bsAb17226 | 7.70E+03 | 1.02E+04 | 1.06E+05 | 5.94E+03 | 1.17E+05 |
| bsAb17227 | 7.32E+03 | 9.64E+03 | 1.28E+04 | 6.48E+03 | 6.64E+03 |
| bsAb17228 | 8.32E+03 | 8.00E+03 | 1.90E+04 | 6.62E+03 | 7.22E+03 |
| bsAb17229 | 9.56E+04 | 8.57E+04 | 1.26E+05 | 7.29E+04 | 1.91E+05 |
| bsAb17231 | 8.88E+03 | 9.98E+03 | 6.52E+04 | 7.00E+03 | 4.95E+04 |
| bsAb17232 | 7.14E+03 | 7.14E+03 | 4.01E+04 | 5.20E+03 | 1.30E+04 |
| bsAb17230 | 8.26E+03 | 6.74E+03 | 8.24E+03 | 6.32E+03 | 2.45E+04 |
| bsAb17233 | 7.14E+03 | 7.50E+03 | 6.25E+04 | 5.98E+03 | 5.72E+04 |
| bsAb17234 | 6.80E+03 | 7.36E+03 | 1.20E+05 | 6.08E+03 | 2.47E+05 |
| bsAb17235 | 9.44E+03 | 1.00E+04 | 1.84E+05 | 7.68E+03 | 2.94E+05 |
| CD3-7221G | 1.92E+04 | 1.61E+04 | 1.91E+04 | 1.21E+04 | 6.48E+04 |
| Control | | | | | |

Abbreviations:
ND: Not Determined because no dose dependent response was observed
Max (RLU) is the highest mean RLU value within tested dose-range.

### Example 5: Characterization of the Ability SLC3A2-APISxCD3 Bispecific Antibodies to Activate Human Primary CD3+ T Cells Using Cell Lines Expressing Wild-Type SLC3A2 or SLC3A2-APIS as Target Cells

This example relates to an in vitro study performed to demonstrate the ability of select SLC3A2-APIS bispecific antibodies to specifically activate CD3 in human primary T cells in the presence of target cells expressing wild-type SLC3A2 or SLC3A2-APIS, as measured in a primary T cell activation assay. Primary human T cells endogenously express human CD3. The target cells used in this assay were NIH3T3 cells engineered to express CD20 alone or (1) in combination with wild-type SLC3A2, or (2) in combination with SLC3A2-APIS. Mel-202 cells were also used due to their endogenous expression of SLC3A2-APIS. The ability of CD3 bispecific antibodies to stimulate CD3 activity is assessed by combining primary human CD3+ T cells with target cells and a titration of antibody. Activation of CD3 results in cytokine production, indicating T cell activation.

Cell Lines: The following cell lines were used in this study: (1) NIH3T3/hCD20: An engineered cell line made by stably transducing NIH3T3 cells, a fibroblast cell line isolated from a mouse NIH/Swiss embryo, with human CD20 (Uniprot accession #: P11836, amino acids M1 to P297); (2) NIH3T3/hSLC3A2-WT/hCD20: An engineered cell line made by stably transducing NIH3T3 cells with wild-type human SLC3A2 (hSLC3A2-amino acids M1 to A529 of accession number P08195) and human CD20; (3) NIH3T3/hSLC3A2-APIS/hCD20: An engineered cell line made by stably transducing NIH3T3 cells with a variant of human SLC3A2 containing an insertion of amino acids, APIS (hSLC3A2-amino acids M1 to A242 of accession number P08195-amino acids APIS-amino acids G243 to A529 of accession number P08195) and human CD20; (4) Mel-202: A cell line that endogenously expresses the APIS insertion variant of human SLC3A2, established from a primary uveal melanoma.

Isolation of human primary T cells: Human peripheral blood mononuclear cells (PBMCs) were isolated from a healthy donor leukocyte pack from Precision for Medicine using the EasySep™ Direct Human PBMC Isolation Kit following the manufacturers recommended protocol and frozen down. CD3+ T cells were isolated from thawed PBMC's using an EasySep™ Human CD3+ T Cell Isolation Kit from StemCell Technologies following the manufacturer's recommended instructions.

IL2, IFNγ, and TNFα release assay: Enriched CD3+ T cells, resuspended in stimulation media, were added to 384-well plates at a concentration of 3×10⁴ cells/well. Target cells were added to CD3+ T cells at a final concentration of 5×10³ cells/well. Subsequently, test antibodies were titrated from 100 nM to 1.53 pM in a 1:4 dilution and added to wells. The final point of the 10-point dilution contained no titrated antibody. Plates were incubated for 72 hours at 37° C., 5% CO2 and 15 μL of supernatant was removed and used for measuring IL2, IFNγ, and TNFα (5 μL each). The amount of cytokine in assay supernatant was determined using Qbead® kits from Sartorius following the manufacturer's protocol. The cytokine measurements were acquired on Sartorius iQue3® Advanced High Throughput Flow Cytometer and values were reported as pg/mL. All serial dilutions were tested in duplicate. The EC₅₀ values of the antibodies were determined from a four-parameter logistic equation over a 10-point dose-response curve using GraphPad Prism™ software. Maximal IL2, IFNγ, and TNFα is given as the mean max response detected within the tested dose range.

Results: In the presence of T cells and target cells expressing SLC3A2-APIS, several SLC3A2-APISxCD3 bispecific antibodies led to dose dependent increases in IL2, IFNγ, and TNFα. Bispecific antibodies that showed target dependent increase in cytokine included bsAb17226, bsAb17229, bsAb17234, and bsAb17235. The potency values for IL2 release, IFNγ release, and TNFα release for the SLC3A2-APISxCD3 bispecific antibodies are shown in Table 15 below. The maximum cytokine release for IL-2, IFNγ, and TNFα for the SLC3A2-APISxCD3 antibodies are shown in Table 16 below. IFNγ release and TNFα release in NIH3T3/SLC3A2-APIS/hCD20 cells induced by bispecific antibodies bsAb17235 and bsAb17234 is shown in FIGS. 8B-8C. IFNγ release and TNFα release in Mel-202 cells induced by bispecific antibodies bsAb17235 and bsAb17234 is shown in FIG. 9 (two lower graphs).

In the presence of T cells and NIH3T3/hCD20/hSLC3A2-APIS target cells, SLC3A2-APISxCD3 bispecific antibodies, bsAb17226, bsAb17229, bsAb17234, and bsAb17235, led to a dose dependent increase in IL2 release, while in the presence of Mel-202 cells (with endogenous SLC3A2-APIS expression), only bsAb17235 led to a dose dependent increase in IL2. BsAb17235 led to the highest maximum IL-2 release among all antibodies tested in both the Mel-202 and NIH3T3/hCD20/hSLC3A2-APIS assays. In the presence of NIH3T3/hCD20/hSLC3A2-WT cells, no SLC3A2-APISxCD3 bispecific antibody led to a detectable IL2 response.

In the presence of T cells and NIH3T3/hCD20/SLC3A2-APIS, SLC3A2-APISxCD3 bispecific antibodies bsAb17235, bsAb17234, bsAb17229, and bsAb17226 led to a dose dependent increase in IFNγ release. In the presence of Mel-202 cells, bsAb17235 led to the highest maximum IFNγ release with only bsAb17234 and bsAb17226 displaying a weaker response in comparison. bsAb17235 led to the greatest maximum cytokine release and most potent cytokine release in both the Mel-202 and NIH3T3/hCD20/SLC3A2-APIS assays. No antibody led to an IFNγ response in the presence of NIH3T3/hCD20/hSLC3A2-WT cells.

In the presence of T cells and NIH3T3/hCD20/SLC3A2-APIS, the SLC3A-APISxCD3 bispecific antibodies bsAb17235, bsAb17229, bsAb17234, and bsAb17226 led to a dose dependent increase in TNFα release. BsAb17229 led to the highest maximum TNFα release among all molecules tested in the NIH3T3/hCD20/SLC3A2-APIS assay. In the presence of Mel-202, only bsAb17235 and bsAb17234 led to a detectable cytokine response. No antibody led to a TNFα response in the presence of NIH3T3/hCD20/hSLC3A2-WT cells.

Table 15 sets forth potency values for IL-2 release, IFNγ release, and TNFα release in response to SLC3A2-APISxCD3 bispecific antibodies as indicated by EC₅₀ in M.

TABLE 15
Potency values for IL-2 release, IFNγ release, and TNFα release, EC50 [M], for bispecific SLC3A2-APIS × CD3 antibodies

| | | | | NIH3T3/hSLC3A2- | | | NIH3T3/hSLC3A2- | | | | | |
|---|---|---|---|---|---|---|---|---|---|---|---|---|
| Antibody | NIH3T3/hCD20 | | | WT/hCD20 | | | APIS/hCD20 | | | Mel-202 | | |
| Identifier | IL2 | IFNγ | TNFα | IL2 | IFNγ | TNFα | IL2 | IFNγ | TNFα | IL2 | IFNγ | TNFα |
| bsAb17225 | ND | ND | ND | ND | ND | ND | ND | ND | ND | ND | ND | ND |
| bsAb17226 | ND | ND | ND | ND | ND | ND | NC | 1.54E−08 | NC | ND | 2.37E−07 | NC |
| bsAb17227 | ND | ND | ND | ND | ND | ND | ND | ND | ND | ND | ND | ND |
| bsAb17228 | ND | ND | ND | ND | ND | ND | ND | ND | ND | ND | ND | ND |
| bsAb17229 | ND | ND | ND | ND | ND | ND | NC | 8.76E−10 | 1.18E−09 | ND | 1.17E−09 | 2.10E−09 |
| bsAb17231 | ND | ND | ND | ND | ND | ND | ND | ND | ND | ND | ND | ND |
| bsAb17232 | ND | ND | ND | ND | ND | ND | ND | ND | ND | ND | ND | ND |
| bsAb17230 | ND | ND | ND | ND | ND | ND | ND | ND | ND | ND | ND | ND |
| bsAb17233 | ND | ND | ND | ND | ND | ND | ND | ND | ND | ND | ND | ND |
| bsAb17234 | ND | ND | ND | ND | ND | ND | 6.60E−10† | 5.81E−10 | 6.61E−10 | ND | 2.22E−08 | 3.05E−07 |
| bsAb17235 | ND | ND | ND | ND | ND | ND | 5.88E−10†† | 2.47E−10 | 2.64E−10 | ND | 9.05E−09 | 5.60E−09 |
| CD3-7221G | ND | ND | ND | ND | ND | ND | ND | ND | ND | ND | ND | ND |

ND: Not Determined because no dose dependent response was observed
NC: Not calculated because the data did not fit a 4-parameter logistic equation manufacturer's lowest decetable range
†While all data points are used to determine Maximum luciferase activity, for EC50 calculation, the values for the highest antibody concentration were removed due to a “hook effect”.
††While all data points are used to determine Maximum luciferase activity, for EC50 calculation the values for the two highest antibody concentration were removed due to a “hook effect”.

Table 16 sets forth the cytokine release for IL-2, IFNγ, and TNFα in response to SLC3A2-APISxCD3 bispecific antibodies.

TABLE 16
Maximum cytokine release for IL-2, IFNγ, and TNFα for SLC3A2-APIS × CD3 bispecific antibodies

| | | | | NIH3T3/hSLC3A2- | | | NIH3T3/hSLC3A2- | | | | | |
|---|---|---|---|---|---|---|---|---|---|---|---|---|
| Antibody | NIH3T3/hCD20 | | | WT/hCD20 | | | APIS/hCD20 | | | Mel-202 | | |
| Identifier | IL2 | IFNγ | TNFα | IL2 | IFNγ | TNFα | IL2 | IFNγ | TNFα | IL2 | IFNγ | TNFα |
| bsAb17225 | NQ | NQ | NQ | NQ | NQ | NQ | NQ | NQ | NQ | NQ | NQ | NQ |
| bsAb17226 | NQ | NQ | NQ | NQ | NQ | NQ | 29 | 372 | 149 | NQ | 110 | 35 |
| bsAb17227 | NQ | NQ | NQ | NQ | NQ | NQ | NQ | NQ | NQ | NQ | NQ | NQ |
| bsAb17228 | NQ | NQ | NQ | NQ | NQ | NQ | NQ | NQ | NQ | NQ | NQ | NQ |
| bsAb17229 | NQ | NQ | NQ | NQ | NQ | NQ | 36 | 306 | 211 | NQ | 67 | 31 |
| bsAb17231 | NQ | NQ | NQ | NQ | NQ | NQ | NQ | NQ | NQ | NQ | NQ | NQ |
| bsAb17232 | NQ | NQ | NQ | NQ | NQ | NQ | NQ | NQ | NQ | NQ | NQ | NQ |
| bsAb17230 | NQ | NQ | NQ | NQ | NQ | NQ | NQ | NQ | NQ | NQ | NQ | NQ |
| bsAb17233 | NQ | NQ | NQ | NQ | NQ | NQ | NQ | NQ | NQ | NQ | NQ | NQ |
| bsAb17234 | NQ | NQ | NQ | NQ | NQ | NQ | 34 | 289 | 101 | NQ | 253 | 151 |
| bsAb17235 | NQ | NQ | NQ | NQ | NQ | NQ | 62 | 409 | 176 | NQ | 547 | 296 |
| CD3-7221G | NQ | NQ | NQ | NQ | NQ | NQ | NQ | NQ | NQ | NQ | NQ | NQ |

NQ: Not quantifiable because the raw signal was too low and therefore fell below manufacturer's lowest detectable range

### Example 6: Characterization of Selected SLC3A2-APISxCD3 Bispecific Antibodies in Flow-Cytometry Based Cytotoxicity Assays with Human Peripheral Blood Mononuclear Cells

This example relates to an in vitro study performed to demonstrate the ability of select SLC3A2-APISxCD3 bispecific antibodies to redirect killing, by human peripheral blood mononuclear (hPBMC), of SLC3A2-APIS+ tumor cells and cells engineered to exogenously express wild-type SLC3A2 or SLC3A2-APIS. The tumor cell lines were acute myeloid leukemia derived cell line, HNT-34, and uveal melanoma cell line, Mel-202. The 4 anti-SLC3A2-APISxCD3 bispecific antibodies tested were bsAb17226, bsAb17233, bsAb17234, and bsAb17235.

SLC3A2-APISxCD3 mediated killing was evaluated in a 3-day cytotoxicity assay targeting both 3T3 cells engineered to express human CD20 and either (1) wild-type human SLC3A2 or (2) SLC3A2-APIS; and two human cancer cells lines endogenously expressing SLC3A2-APIS (AML cell line HNT-34 and uveal melanoma cell line Mel-202). Briefly, human PBMCs were plated at 1×10⁶ cells/mL and incubated overnight at 37° C. in order to enrich for lymphocytes by depleting adherent macrophages, dendritic cells, and some monocytes. The following day, tumor cells were labeled with 1 μM of the fluorescent tracking dye CFDA-SE, and the adherent cell-depleted naïve PBMC were labeled with 1 μM of the fluorescent tracking dye CellTrace Violet™. Labeled target cells and PBMC (Effector/Target cell 5:1 ratio) were co-incubated with a serial dilution of SLC3A2-APISxCD3 bispecific antibodies or CD3 bispecific control antibody CD3-7221G or CD3-7195P (0.2 pM-67 nM) for 3 days at 37° C. Cells were harvested from the plates and analyzed by flow cytometery. For flow cytometry analysis, cells were stained with a fixable live/dead near IR reactive dye. 20,000 counting beads were added to each well immediately before flow cytometric analysis and 10,000 beads were collected for each sample. For the assessment of specificity of killing, cells were gated on live CFDA-SE labeled populations. Percent of live population was recorded and used for the calculation of survival.

T cell activation was assessed by incubating cells with directly conjugated antibodies to CD2, CD4, CD8, and CD25. The percentage of CD8+ cells and CD4+ T cells expressing CD25 was reported as the measure of T cell activation. EC₅₀ values were calculated using 4-parameter non-linear regression analysis in GraphPad Prism™ software. Supernatants were assessed for cytokine release using the LegendPlex™ kit according to the manufacturer's protocol.

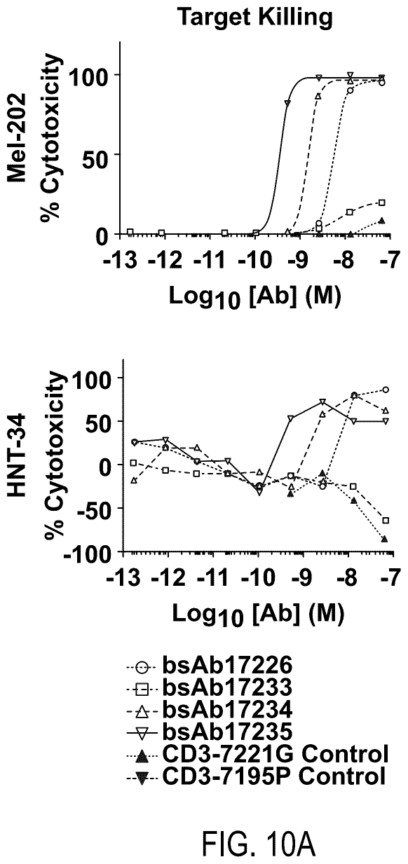

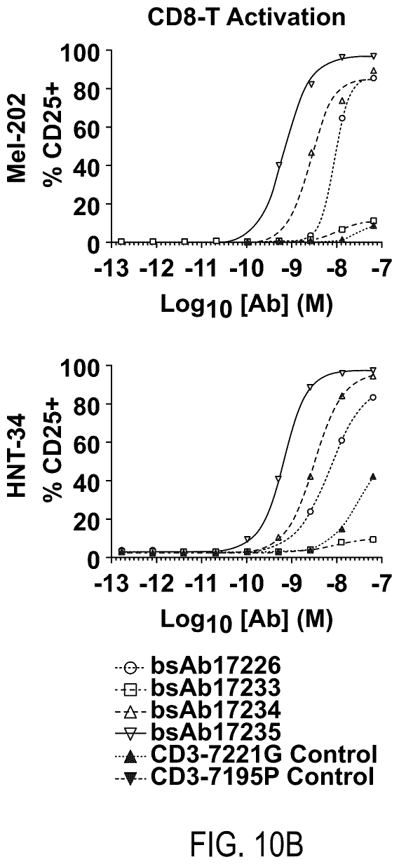

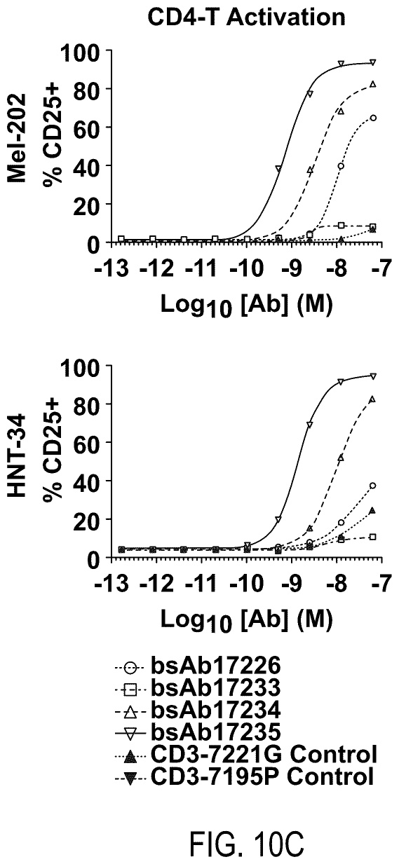

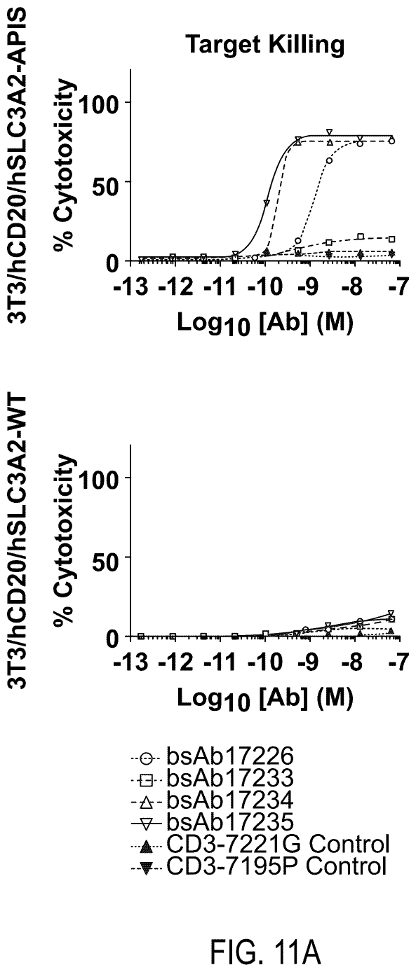

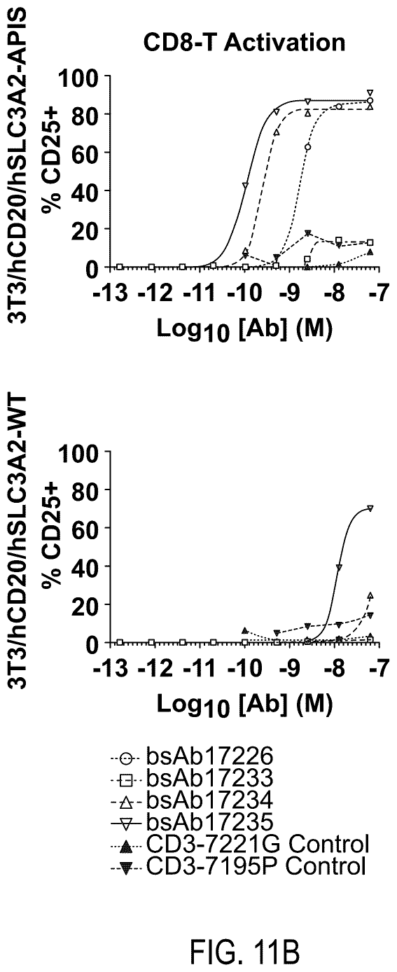

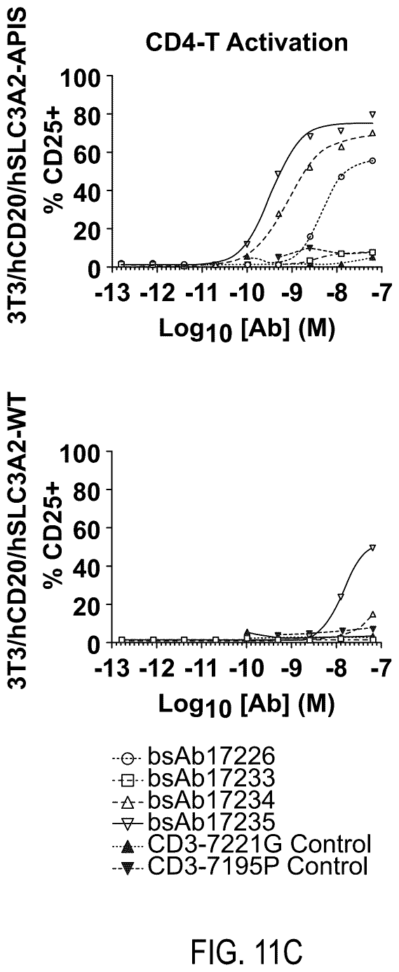

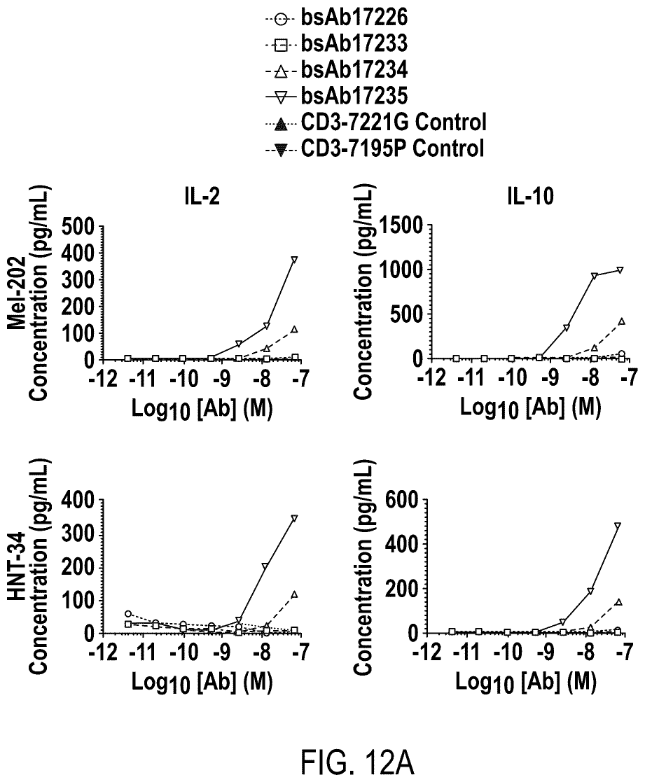

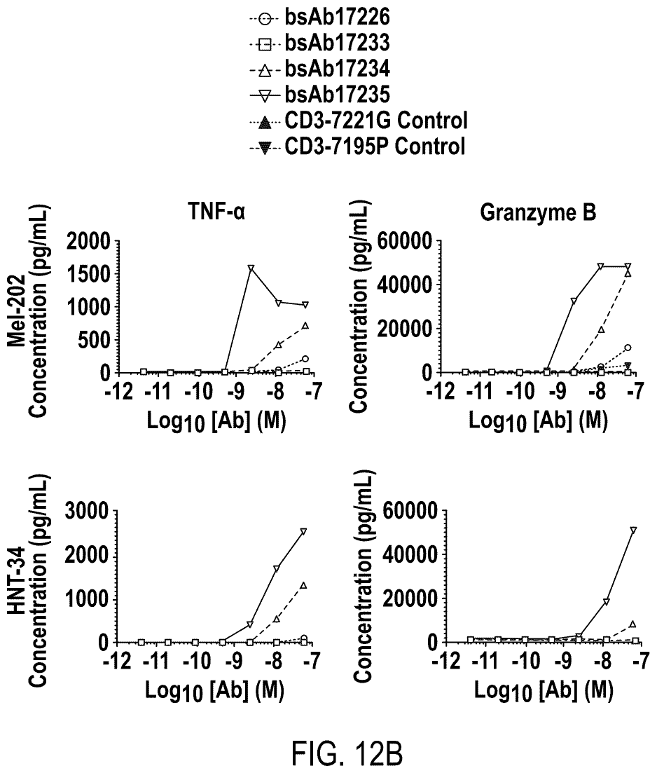

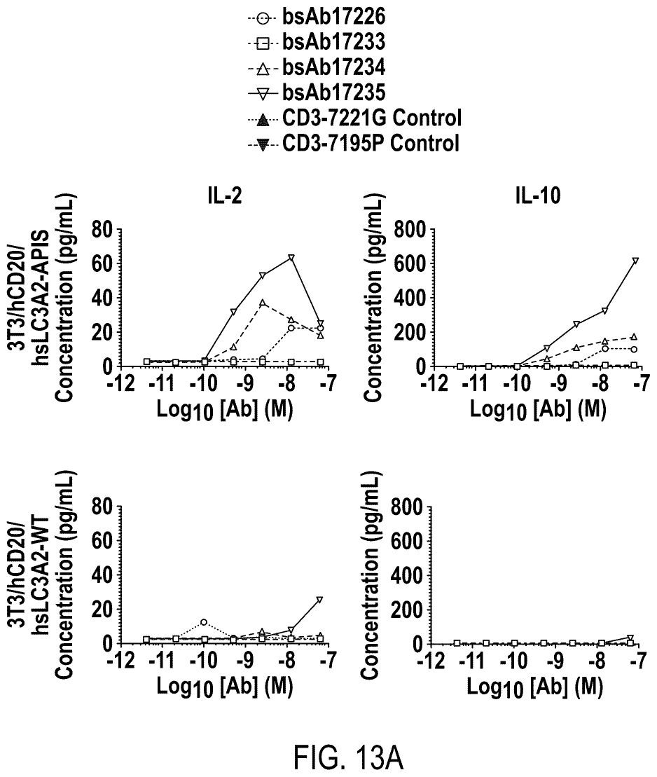

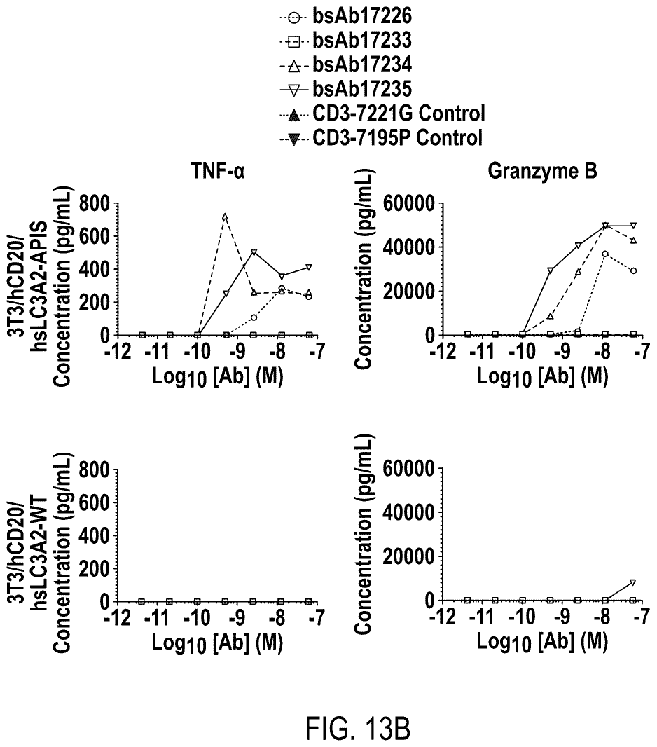

Results: SLC3A2-APISxCD3 bispecific antibodies activated and directed human T cells to deplete SLC3A2-APIS+endogenous (FIG. 10A) and engineered cells (FIG. 11A) in a dose dependent manner with various potency (Table 17). The observed target-cell lysis mediated by the SLC3A2-APISxCD3 bispecific antibodies was associated with T cell activation, as measured by CD25 upregulation on CD8+ and CD4+ T cells (Table 17, FIGS. 10B-10C, 11B-11C). SLC3A2-APISxCD3 bispecific antibodies induced the release of human cytokines in a target dependent manner (Table 18, FIGS. 12A-12B, 13A-13B). Minimal activity was observed with SLC3A2-APISxCD3 antibodies in 3T3 cells engineered to express wild type SLC3A2 and with the CD3 bispecific control antibodies CD3-7221G and CD3-7195P in all cell lines tested (Tables 17 and 18, FIGS. 12 and 13). In summary, SLC3A2-APISxCD3 bispecific antibodies mediated potent and APIS-specific cytotoxic activity in two distinct SLC3A2-APIS+tumor cell lines.

Table 17 sets forth the EC₅₀ values and maximum activity for target cytotoxicity and T cell activation in response to SLC3A2-APISxCD3 bispecific antibodies.

TABLE 17
EC₅₀ Values and Maximum Activity for Target Cytotoxicity and T Cell Activation

| | | bsAb17226 | | bsAb17233 | | bsAb17234 | | bsAb17235 | | CD3-7221G | |
|---|---|---|---|---|---|---|---|---|---|---|---|
| | | EC₅₀ | | EC₅₀ | | EC₅₀ | | EC₅₀ | | EC₅₀ | |
| Cell Line | Parameter | (M) | Max | (M) | Max | (M) | Max | (M) | Max | (M) | Max |
| Mel-202 | Cytotoxicity | 5.7E−09 | 96 | 8.3E−09 | 20 | 1.5E−09 | 98 | 3.5E−10 | 99 | ND | 8.9 |
| | % CD25+ CD8 | 9.0E−09 | 86 | 1.2E−08 | 11 | 2.5E−09 | 89 | 2.0E−11 | 97 | ND | 8.7 |
| | T cells | | | | | | | | | | |
| | % CD25+ CD4 | 1.1E−08 | 65 | 2.9E−09 | 9.1 | 3.4E−09 | 83 | 7.7E−10 | 94 | ND | 7.2 |
| | T cells | | | | | | | | | | |
| HNT-34 | Cytotoxicity | NC | 90 | ND | 5.9 | NC | 81 | NC | 78 | ND | 35 |
| | % CD25+ CD8 | 7.2E−09 | 85 | NC | 10 | 3.4E−09 | 96 | 6.9E−10 | 98 | NC | 43 |
| | T cells | | | | | | | | | | |
| | % CD25+ CD4 | NC | 39 | 7.7E−09 | 12 | 1.1E−08 | 84 | 1.5E−09 | 96 | NC | 25 |
| | T cells | | | | | | | | | | |
| NIH3T3/hSLC3A2- | Cytotoxicity | 1.3E−09 | 76 | NC | 16 | 1.8E−10 | 78 | 1.2E−10 | 81 | ND | 6 |
| APIS/hCD20 | % CD25+ CD8 | 1.8E−09 | 88 | 2.9E−09 | 14 | 2.6E−10 | 85 | 1.1E−10 | 92 | NC | 8.4 |
| | T cells | | | | | | | | | | |
| | % CD25+ CD4 | 4.9E−09 | 56 | 4.0E−09 | 8 | 8.7E−10 | 71 | 3.6E−10 | 80 | ND | 5.9 |
| | T cells | | | | | | | | | | |
| NIH3T3/hSLC3A2- | Cytotoxicity | NC | 11 | ND | 11 | ND | 6 | NC | 15 | ND | 8 |
| WT/hCD20 | % CD25+ CD8 | ND | 2.7 | ND | 2.2 | NC | 25 | 1.2E−08 | 91 | ND | 6.8 |
| | T cells | | | | | | | | | | |
| | % CD25+ CD4 | ND | 2.7 | NC | 2.8 | NC | 15 | 1.5E−08 | 50 | ND | 6.2 |
| | T cells | | | | | | | | | | |

NC: Not Calculated. The fit of the curve does not allow for an accurate reporting of EC₅₀.
ND: Not Determined. A concentration-dependent increase in the parameter evaluated was not observed.

Table 18 sets forth the maximum cytokine release mediated by select SLC3A2-APISxCD3 bispecific antibodies.

TABLE 18
Maximum Cytokine Release Mediated by SLC3A2-APIS × CD3 Bispecific Antibodies

| | Cytokine | bsAb17226 | bsAb17233 | bsAb17234 | bsAb17235 | CD3-7221G |
|---|---|---|---|---|---|---|
| Mel-202 | IL2 (pg/ml) | 11 | 2.7 | 114 | 381 | 256 |
| | IL10 (pg/ml) | 58 | 3.0 | 427 | 1010 | 1.3 |
| | TNFa (pg/ml) | 217 | 9.1 | 728 | 1595 | 9.4 |
| | GranB (pg/ml) | 10865 | 32 | 45665 | 49200* | 41 |
| HNT34 | IL2 (pg/ml) | 67 | 36 | 119 | 349 | 32 |
| | IL10 (pg/ml) | 15 | 9.9 | 141 | 484 | 8.0 |
| | TNFa (pg/ml) | 104 | 0.7 | 1360 | 2588 | 0.7 |
| | GranB (pg/ml) | 1988 | 2036 | 8628 | 52000 | 2075 |
| 3T3/CD20/ | IL2 (pg/ml) | 22 | 3.7 | 37 | 63 | not tested |
| SLC3A2-APIS | IL10 (pg/ml) | 109 | 5.4 | 173 | 608 | not tested |
| | TNFa (pg/ml) | 286 | 1.9 | 713 | 498 | not tested |
| | GranB (pg/ml) | 36619 | 52 | 49200* | 49200* | not tested |
| 3T3/CD20/ | IL2 (pg/ml) | 4.5 | 12 | 7.4 | 25 | not tested |
| SLC3A2-WT | IL10 (pg/ml) | 1.9 | 2.1 | 3.0 | 30 | not tested |
| | TNFa (pg/ml) | 1.3 | 1.3 | 1.3 | 9.0 | not tested |
| | GranB (pg/ml) | 79 | 11 | 119 | 8428 | not tested |

*Values above detection range

### Example 7: CryoEM Structure of SLC3A2-APIS Ectodomain with SLC3A2-APIS Binding Arm of a Selected SLC3A2-APISxCD3 Bispecific Antibody

This example relates to a cryo electron microscopy (CryoEM) study defining residue-level interactions at the epitope/paratope interface of the SLC3A2-APIS binding arm of a selected SLC3A2-APISxCD3 bispecific antibody bound to the noncanonical spliceoform of SLC3A2, SLC3A2-APIS.

REGN17224 Fab production: 5.5 mg of REGN17224 (the parental antibody of bsAb17235) was diluted to 2 mL in PBS. To cleave the IgG below the hinge, 110 μg of IdeS enzyme was added to the sample, followed by incubation at 37° C. for 30 minutes. Hinge cysteines were reduced by beta-mercaptoethanol for 90 minutes at 37° C. The sample was then treated with 0.1 M lodoacetamide to covalently cap free cysteines. The Fc domain was removed by binding to CaptureSelect™ IgG-Fc multispecies resin. A final size exclusion chromatography step using a Superdex 200 increase 10/300 GL column equilibrated to buffer containing 50 mM Tris pH 7.5, 150 mM NaCl was conducted to purify the sample to homogeneity.

CryoEM sample preparation and data collection: REGN17224 Fab and SLC3A2-APIS ectodomain protein (SEQ ID NO: 265) were mixed at a Fab: antigen molar ratio of 1.2:1 and incubated for at least 30 minutes at 4° C. The complex sample was supplemented with 0.2% PMAL-C8 amphipol then immediately applied to UltrAuFoil 1.2/1.3, 300 mesh grids that were freshly plasma cleaned using a Solarus II. The sample was blotted and plunge-frozen into liquid ethane cooled by liquid nitrogen using a Vitrobot Mark IV operated at 4° C. and 100% humidity. CryoEM data were collected on a Glacios microscope equipped with a Falcon 4i camera operating in counted mode. Automated data collection was carried out using EPU software. Movies were collected at a nominal magnification of 165,000×(0.697 Å/pixel).

CryoEM data processing and map generation: CryoEM data were processed using cryoSPARC v4.3.1 software. Movies were motion-corrected and CTF parameters were estimated for the summed micrographs. Particle coordinates were picked using 2D templates and TOPAZ. Particle images corresponding to false positives, contaminants, or broken complexes were removed after multiple rounds of 2D classification. Homogenous subsets of particle images corresponding to the target complex were obtained after multiple rounds of ab initio reconstruction and hetero refinement. The final 2.89 Å resolution map (according to a gold standard FSC=0.143 criterion) was calculated from 300,055 particle images using the Local Refine jobtype.

Model building and refinement: Initial model coordinates were obtained from a published crystal structure of SLC3A2 ectodomain (PDB 8G0M) and a structure of REGN17224 Fab predicted by implementation of AlphaFold2. The Fit-in-map function of UCSF Chimera was used to dock initial models into their corresponding densities. Manual model building was carried out using Coot version 0.8.9, and real space refinements were conducted in Phenix version 1.19. Structures were analyzed in pymol 2.5.4 and PISA as implemented in CCP412.

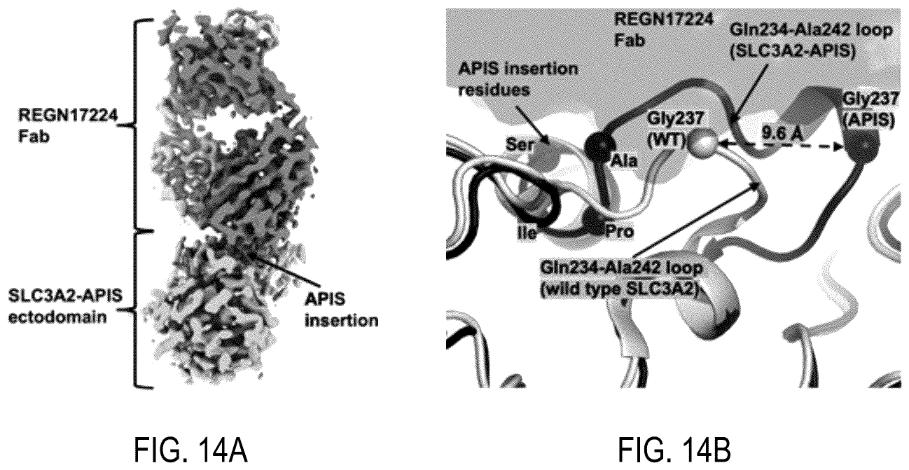

Results: A 2.89 Å resolution structure of REGN17224 Fab-bound SLC3A2-APIS ectodomain was obtained by single particle cryoEM (FIGS. 14A-14B). CryoEM density for most side chains of the antigen and Fab variable region are clearly defined in the map (FIG. 14A), allowing for accurate model building and assessment of residue-level interactions at the epitope/paratope interface (FIG. 14B).

The structural data shows how the APIS insertion impacts the structure of the SLC3A2 ectodomain (FIG. 14B). The overall architecture of the SLC3A2 ectodomain is similar in the APIS mutant and wild type (RMSD=0.92 Å, calculated over 2813 atom pairs in pymol using PDB 8G0M as reference). The APIS residues are inserted between amino acids Ala242 and Gly243 (SLC3A2 numbering matching Uniprot entry P08195), which in the wild-type protein are situated in a partially surface exposed alpha helix formed by residues Leu241-Leu254. In the structure of the SLC3A2-APIS ectodomain/REGN17224 Fab complex, residues Gly243-Leu254 (wild type numbering) remain helical and in a similar position to wild type, and three APIS insertion residues (Pro-Ile-Ser) extend this helix N-terminally. The inserted Ala and preceding residues Gln234-Ala242 form a mostly surface exposed loop that has a substantially altered conformation in the APIS mutant relative to wild type (Table 19). For example, the Ca atom of Gly237 located in the middle of this loop is shifted 9.6 A away from its position in wild type. “Anchoring” of this loop on its N- and C-terminal ends by alpha helices prevents the APIS insertion-induced conformational change from transmitting to the rest of the SLC3A2 protein. In sum, the APIS insertion results in substantial yet localized structural changes at a surface-exposed helix and its preceding loop.

The cryoEM structure of the SLC3A2-APIS ectodomain/REGN17224 complex reveals details of the antibody/antigen interface (summarized in Table 19). The paratope is dominated by the REGN17224 heavy chain, which has an interface area of ˜840 Å2 on the target protein compared to ˜200 Å2 for the light chain. Notably, REGN17224 binds to an epitope that includes the APIS insertion site region; three of the insertion residues (Ala, Pro, Ser) are contacted by the antibody, burying ˜82 Å2. The surface exposed loop (Gln234-Ala242) directly N-terminal to the APIS insertion also contributes prominently to the REGN17224 epitope, burying ˜323 Å². As noted above, this loop has substantially different conformations in the wild-type and SLC3A2-APIS structures. Combined, the APIS insertion residues and Gln234-Ala242 loop comprise ˜39% of the total buried surface area of the REGN17224 epitope. Importantly, this provides a structural explanation for the selective binding of REGN17224 to the APIS-inserted form of SLC3A2.

Table 19 sets forth the epitope residues in SLC3A2-APIS for anti-SLC3A2-APIS monospecific antibody REGN17224

TABLE 19
Summary of Epitope Residues in SLC3A2-APIS for anti-
SLC3A2-APIS Monospecific Antibody REGN17224

| SLC3A2-APIS | Cα(APIS)-Cα(WT) | HC BSA | LC BSA |
|---|---|---|---|
| epitope residue | displacement (Å) | (Å²)** | (Å²) |
| Gln231 | 0.8 | 16 | 0 |
| Gln234* | 1.4 | 2 | 0 |
| Gly237* | 9.6 | 26 | 0 |
| Ala238* | 10.1 | 60 | 36 |
| Gly239* | 11.4 | 16 | 0 |
| Asn240* | 10.1 | 30 | 0 |
| Leu241* | 12.3 | 149 | 0 |
| Ala242* | 6.9 | 39 | 0 |
| Ala243 | N/A | 39 | 0 |
| Pro244 | N/A | 9 | 0 |
| Ser246 | N/A | 34 | 0 |
| Gly247 | 1.4 | 4 | 0 |
| Lys249 | 0.6 | 63 | 0 |
| Gly250 | 0.2 | 53 | 0 |
| Arg251 | 0.4 | 49 | 0 |
| Asp253 | 0.2 | 38 | 30 |
| Tyr254 | 0.1 | 15 | 0 |
| Asn287 | 1.3 | 5 | 0 |
| Gly289 | 1.5 | 5 | 0 |
| Ser290 | 1.1 | 29 | 0 |
| Glu292 | 0.4 | 4 | 0 |
| Asp293 | 0.3 | 24 | 0 |
| Ser296 | 0.5 | 1 | 0 |
| Ser300 | 0.3 | 5 | 0 |
| Lys303 | 0.6 | 17 | 15 |
| Lys304 | 0.2 | 0 | 10 |
| Trp495 | 0.4 | 21 | 0 |
| Asp496 | 0.5 | 8 | 0 |
| Val513 | 0.7 | 10 | 0 |
| Lys514 | 0.6 | 39 | 72 |
| Ser517 | 0.7 | 17 | 6 |
| Glu518 | 0.3 | 0 | 31 |

*Epitope residues that are part of the Gln234-Ala242 loop that adopts a different conformation in APIS-inserted SLC3A2
APIS residues are in bold type (Ile245 not included because REGN17224 does not contact this residue).
**Pairwise Cα-Cα distances of REGN17224 epitope residues in APIS-inserted and wild type SLC3A2 (PDB 8G0M used as reference).
HC = heavy chain; LC = light chain; BSA = buried surface area.
Residue numbering C-terminal to the APIS site accounts for the insertion and differs from the wild type SLC3A2 numbering from Uniprot entry P08195.

### Example 8: Potent Anti-Tumor Efficacy of Selected SLC3A2-APISxCD3 Bispecific Antibodies for a Wide Range of Different Tumor Types

This example relates to a set of in vivo studies demonstrating the efficacy of selected SLC3A2-APISxCD3 bispecific antibodies in treating a wide variety of tumor types related to expression of a mutant spliceosome protein (e.g., SF3B1), which leads to expression of the noncanonical spliceoform of SLC3A2, SLC3A2-APIS. Immunodeficient mice were first engrafted with human immune effector cells, and then engrafted with various tumor cells that either endogenously harbor an SF3B1 hotspot mutation or have been engineered to express mutant SF3B1 and consequently endogenously express SLC3A2-APIS.

Study 1: The anti-tumor activity of SLC3A2-APISxCD3 bispecific antibodies bsAb17235 and bsAb17234 was assessed in comparison to CD3-7195P bispecific control using a MEL-202 uveal melanoma xenograft model. Mel-202 cells endogenously harbor an SF3B1 mutation and consequently endogenously express SLC3A2-APIS. Specifically, NSG mice (NOD·Cg-Prkdc^{scid}II2rg^{tm1Wjl}/SzJ) were first engrafted with 5.0×10⁶ human peripheral blood mononuclear cells (PBMCs) by intraperitoneal (IP) injection, and then seven days later inoculated by subcutaneous (SC) injection with 10×10⁶ Mel-202 uveal melanoma tumor cells that endogenously harbor an SF3B1 hotspot mutation and consequently endogenously express SLC3A2-APIS. Tumors were allowed to grow to an average volume of approximately 65 mm³, at which point mice were randomized to bi-weekly i.p. injection with bsAb17234 at a dose of either 1 mg/kg or 4 mg/kg, bsAb17235 at a dose of either 1 mg/kg or 4 mg/kg, or CD3-7221G or CD3-7195P bispecific control at 4 mg/kg for a total of eight injections. Individual tumor growth was measured approximately twice per week. Tumor volumes (mm³+SD) in each treatment group were calculated as v=ab²/2, where a represents the longest tumor diameter and b is the perpendicular tumor diameter. The number of mice that achieved complete tumor remission in each treatment group was recorded. Mice in which human immune cells or tumor cells did not successfully engraft were excluded from analysis.

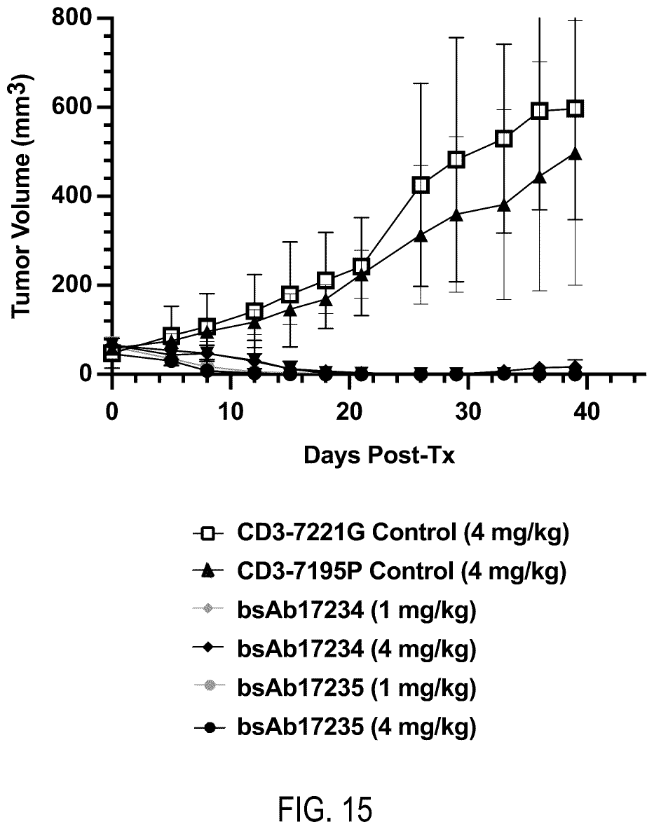

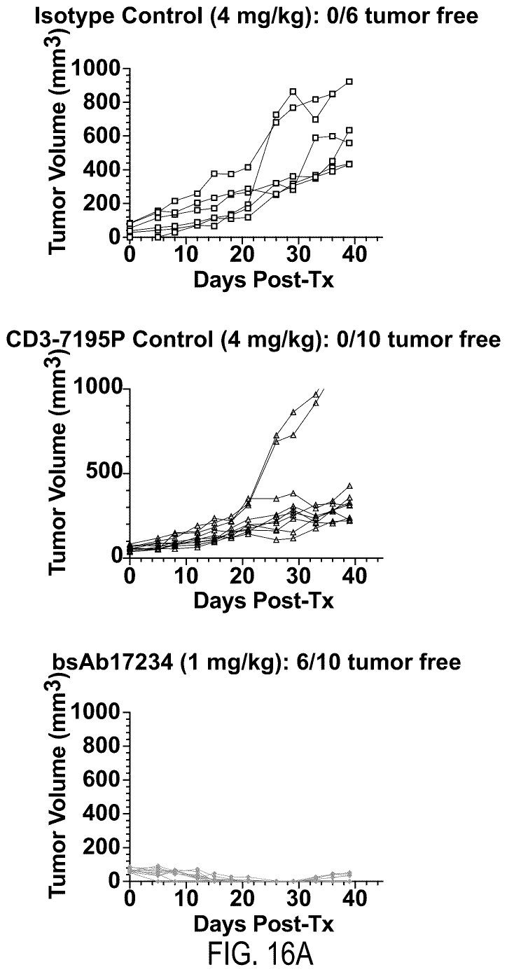

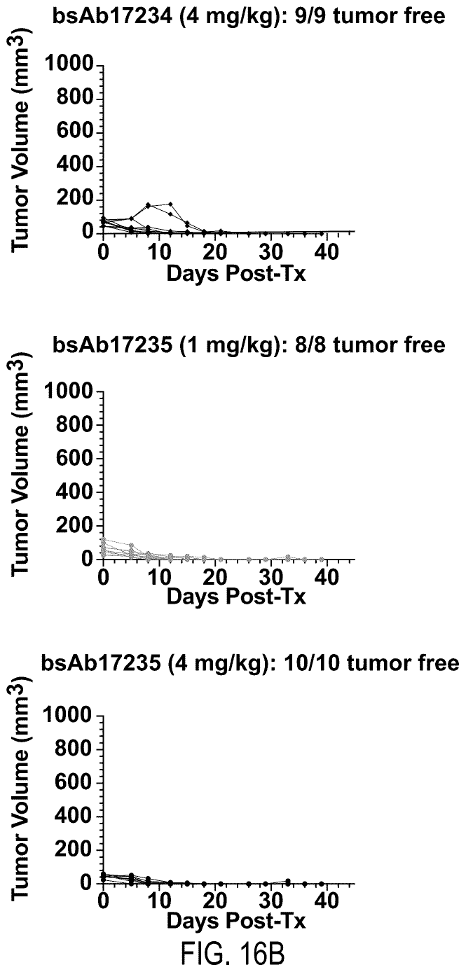

Results: Treatment with bsAb17235 and bsAb17234, at either 1 mg/kg or 4 mg/kg dose levels, demonstrated superior anti-tumor activity compared to either CD3 bispecific control antibody at a dose level of 4 mg/kg (FIGS. 15 and 16). Mice treated with bsAb17235 exhibited more rapid anti-tumor responses and higher rates of complete remissions than mice treated with bsAb17234 (FIGS. 15, 16A-16B).

Study 2: The anti-tumor activity of the SLC3A2-APISxCD3 bispecific antibody, bsAb17235, was assessed in a dose-ranging study in comparison to the CD3-7195P bispecific control using a HNT-34 acute myeloid leukemia xenograft model. HNT-34 cells endogenously harbor an SF3B1 mutation and consequently endogenously express SLC3A2-APIS. Specifically, NSG mice were engrafted with 5.0×10⁶ PBMCs by i.p. injection, and then inoculated s.c. with 20×10⁶ HNT-34 acute myeloid leukemia tumor cells. Tumors were allowed to grow to an average volume of approximately 150 mm³, at which point mice were randomized to bi-weekly i.p. injection of bsAb17234 at either 1 mg/kg, 2 mg/kg, or 4 mg/kg dose levels or CD3-7195P bispecific control at a dose level of 4 mg/kg for a total of eight injections. Individual tumor growth was measured approximately twice per week. Tumor volumes (mm³+SD) in each treatment group were calculated as v=ab²/2, where a represents the longest tumor diameter and b is the perpendicular tumor diameter. The number of mice that achieved complete tumor remission in each treatment group was recorded. Mice in which human immune cells or tumor cells did not successfully engraft were excluded from analysis.

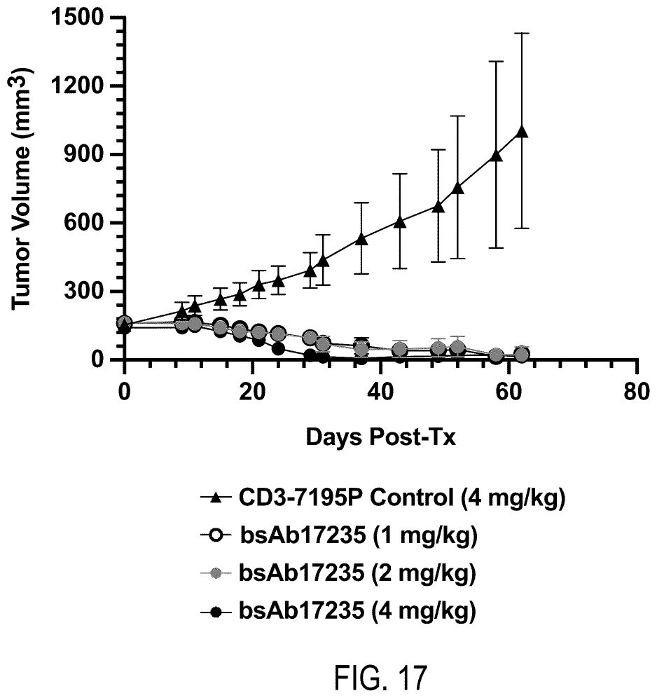

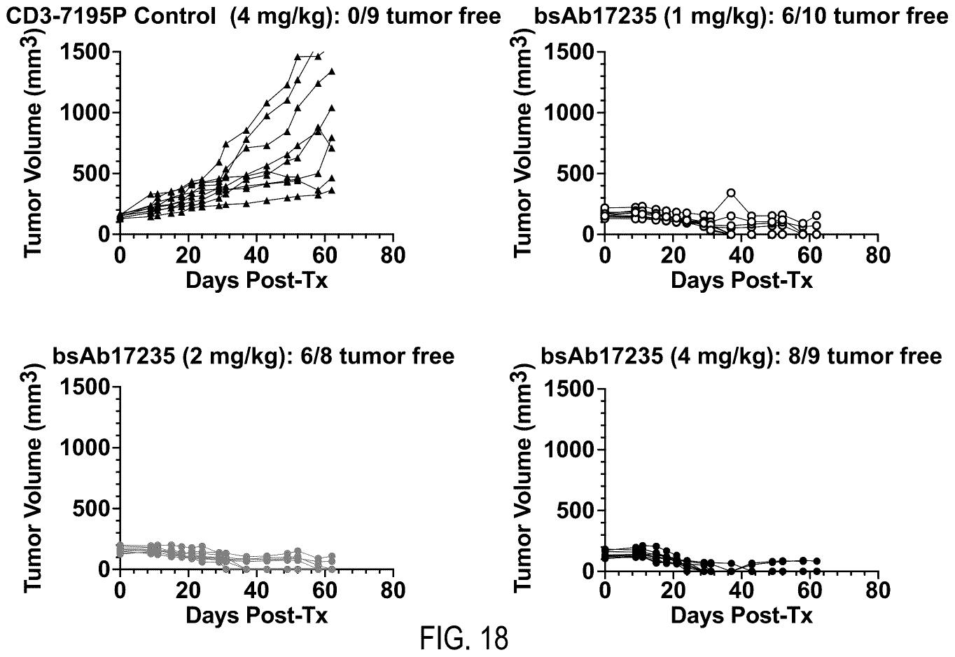

Results: Treatment with bsAb17235 at 1 mg/kg, 2 mg/kg or 4 mg/kg dose levels demonstrated superior anti-tumor activity compared to CD3-7195P bispecific control (FIGS. 17, 18). Mice treated with bsAb17235 exhibited deepening of anti-tumor responses and increases in tumor remissions in a dose-dependent manner (FIGS. 17, 18).

Study 3: The anti-tumor activity of SLC3A2-APISxCD3 bispecific antibodies, bsAb17235 and bsAb17234, was assessed in comparison to CD3-7195P bispecific control using a PANC05.04 pancreatic adenocarcinoma xenograft model. PANC05.04 cells endogenously harbor an SF3B1 mutation and consequently endogenously express SLC3A2-APIS. Specifically, NSG mice were first inoculated by s.c. injection with 10×10⁶ PANC05.05 pancreatic adenocarcinoma tumor cells and then five days later engrafted by i.p. injection with 5.0×10⁶ human PBMCs. Tumors were allowed to grow to an average volume of approximately 250 mm³, at which point mice were randomized to bi-weekly i.p. injection of bsAb17235, bsAb17234, or CD3-7195P bispecific control at a dose level of 4 mg/kg for a total of eight injections. Individual tumor growth was measured approximately twice per week. Tumor volumes (mm³+SD) in each treatment group were calculated as v=ab²/2, where a represents the longest tumor diameter and b is the perpendicular tumor diameter. The number of mice that achieved complete tumor remission in each treatment group was recorded. Mice in which human immune cells or tumor cells did not successfully engraft were excluded from analysis.

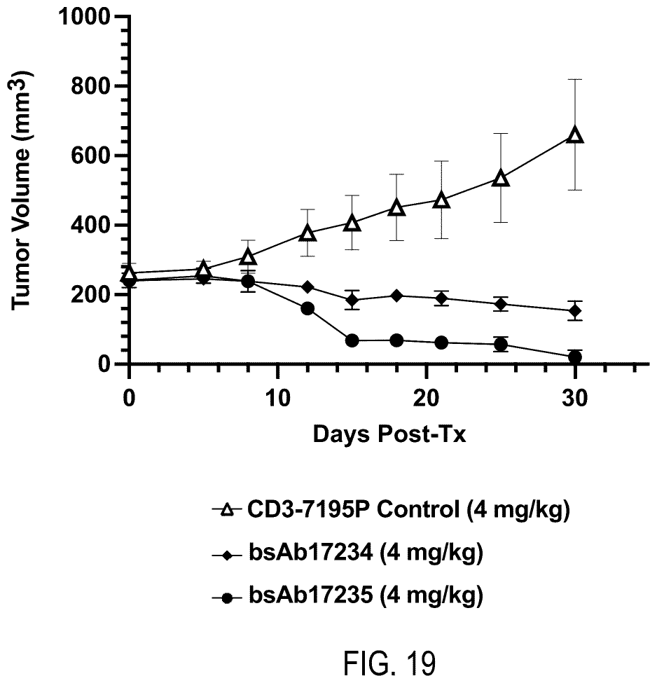

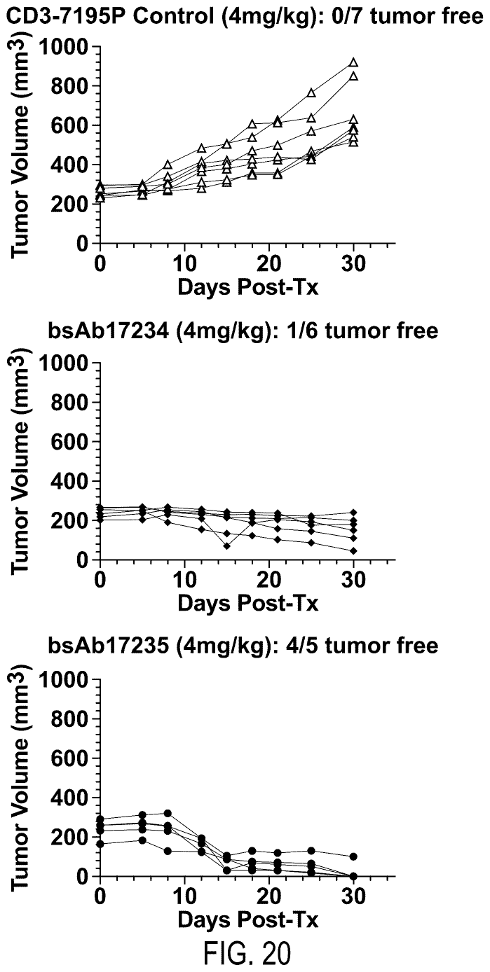

Results: Treatment with bsAb17235 and bsAb17234, at 4 mg/kg dose levels, demonstrated superior anti-tumor activity compared to CD3-7195P bispecific control (FIGS. 19, 20). Mice treated with bsAb17235 exhibited more rapid and deeper anti-tumor responses and higher rates of complete remissions than mice treated with bsAb17234 (FIGS. 19, 20).

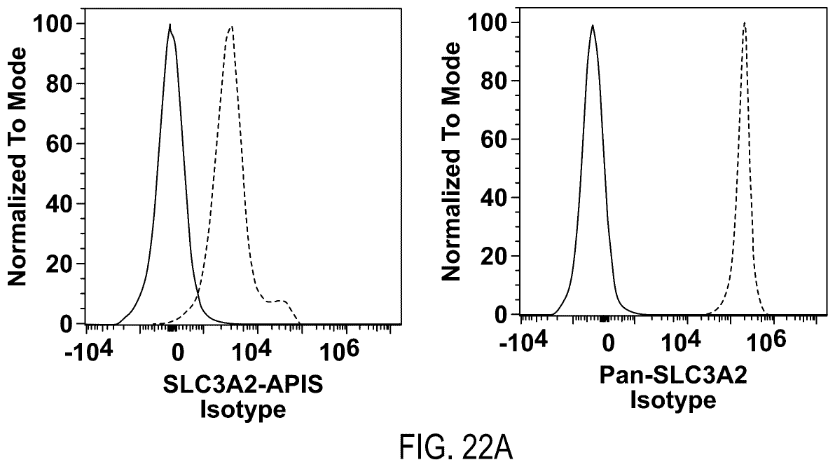

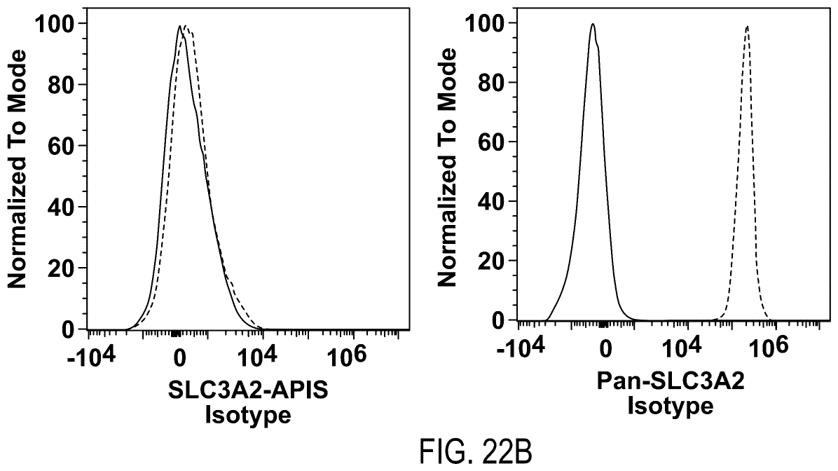

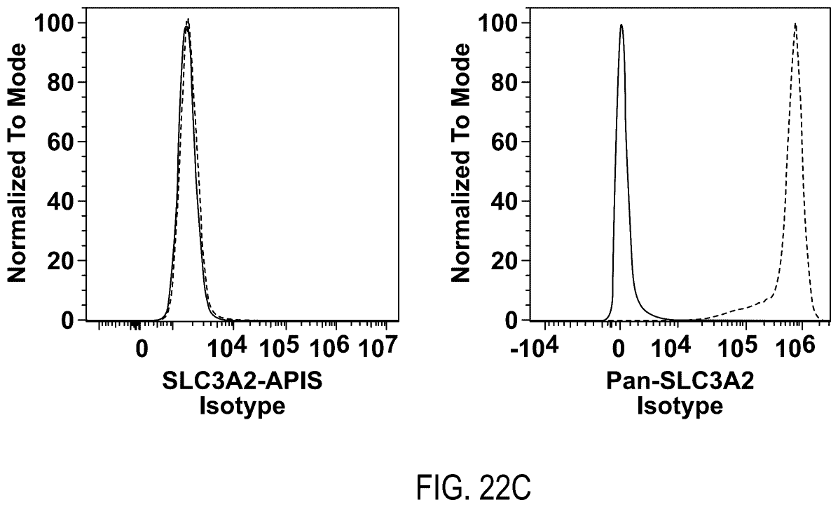

Study 4: A series of tumor xenograft models using human tumor cell lines and NSG mice engrafted with PBMCs were used to test the extent to which the anti-tumor activity of bsAb17235 was dependent on the presence of an SF3B1 hotspot mutation, and consequently the expression of SLC3A2-APIS. First, NALM6 acute lymphoblastic leukemia cells were engineered to express mutant SF3B1 (SF3B1-K700E), and subsequently SLC3A2-APIS. 5×10⁶ SF3B1-K700E NALM6 cells or SF3B1-WT NALM6 cells were injected s.c. into mice that had seven days prior been engrafted with 5×10⁶ PBMCs by i.p. injection. Tumors were allowed to grow to approximately 30 mm³ before initiating bi-weekly i.p. injections of bsAb17235 at 4 mg/kg or CD3-7195P bispecific control at 4 mg/kg (for SF3B1-K700E NALM6 cells) or bsAb17235 at 4 mg/kg or 10 mg/kg or CD3-7195P bispecific control at 4 mg/kg (for SF3B1-WT NALM6 cells) for a total of eight injections. Efficacy of bsAb17235 vs. CD3-7195P bispecific control was evaluated in parallel for each model. In addition, a second SF3B1-WT tumor xenograft model was implemented to assess the dependency of bsAb17235 on the presence of SLC3A2-APIS. 2×10⁶ Raji tumor cells were admixed with 0.5×10⁶ PBMCs and injected s.c. into NSG mice. Tumors were allowed to grow to approximately 100 mm³ before initiating bi-weekly i.p. injections of bsAb17234 at a dose of either 1 mg/kg or 10 mg/kg; bsAb17235 at a dose of either 1 mg/kg or 10 mg/kg; CD3-7195P bispecific control at 10 mg/kg; or a CD20×CD3 bispecific antibody at 1 mg/kg as a positive control, for a total of eight injections. Individual tumor growth was measured approximately twice per week. Tumor volumes (mm³+SD) in each treatment group were calculated as v=ab²/2, where a represents the longest tumor diameter and b is the perpendicular tumor diameter. The number of mice that achieved complete tumor remission in each treatment group was recorded. Mice in which human immune cells or tumor cells did not successfully engraft were excluded from analysis. Expression of SLC3A2-APIS and pan-SLC3A2 was evaluated by flow cytometry (FIGS. 22A-22C).

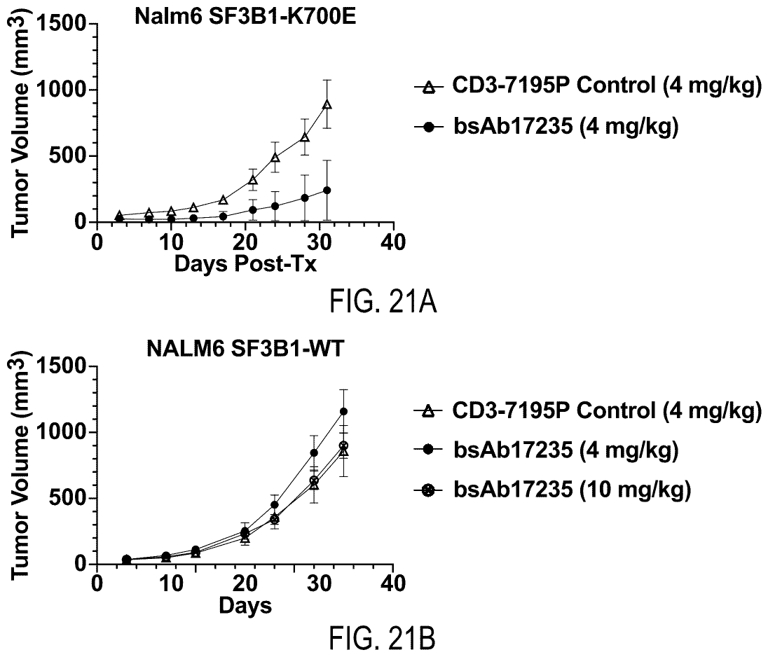

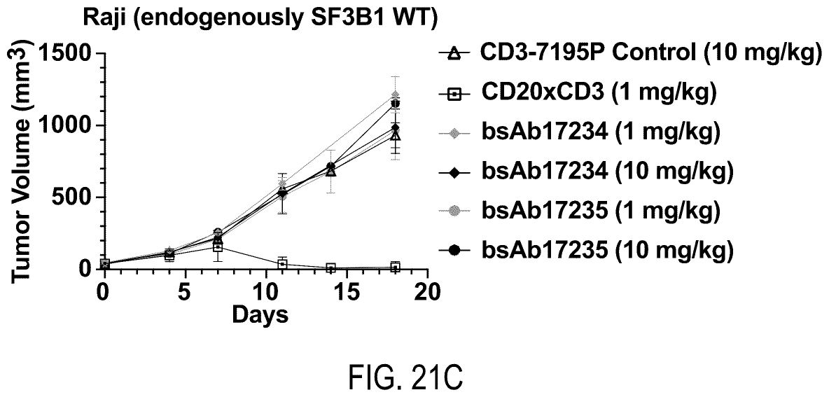

Results: BsAb17235 exhibited no anti-tumor activity against SF3B1-WT NALM6 cells, even at high doses of 10 mg/kg (FIG. 21B). However, when NALM6 cells were engineered to harbor an SF3B1 mutation, and consequently expressed SLC3A2-APIS, the cells were then vulnerable to killing by bsAb17235 in vivo (FIG. 21A). In a second SF3B1-WT tumor model, R17235 exhibited no anti-tumor activity against Raji cells (FIG. 21C). These same tumors, however, were sensitive to killing by a CD20×CD3 bispecific antibody (FIG. 21C, bottom).

Study 5: The ability to augment the monotherapy activity of SLC3A2-APISxCD3 antibodies with CD38×CD28 co-stimulatory antibodies was tested in NSG mice engrafted with PBMCs and SF3B1-K700E NALM6 tumor cells. Specifically, NSG mice were engrafted with 5×10⁶ PBMCs by i.p. injection and inoculated with 5×10⁵ SF3B1-K700E NALM6 tumor cells by s.c. injection. Tumors were allowed to grow to approximately 30 mm³ before initiating bi-weekly i.p. injections of bsAb17235 or a CD38×CD28 antibody at a dose of 4 mg/kg; or once-weekly i.p. injection of a CD38×CD28 antibody or CD3-7195P bispecific control for a total of eight injections. Individual tumor growth was measured approximately twice per week. Tumor volumes (mm³+SD) in each treatment group were calculated as v=ab²/2, where a represents the longest tumor diameter and b is the perpendicular tumor diameter. The number of mice that achieved complete tumor remission in each treatment group was recorded. Mice in which human immune cells or tumor cells did not successfully engraft were excluded from analysis.

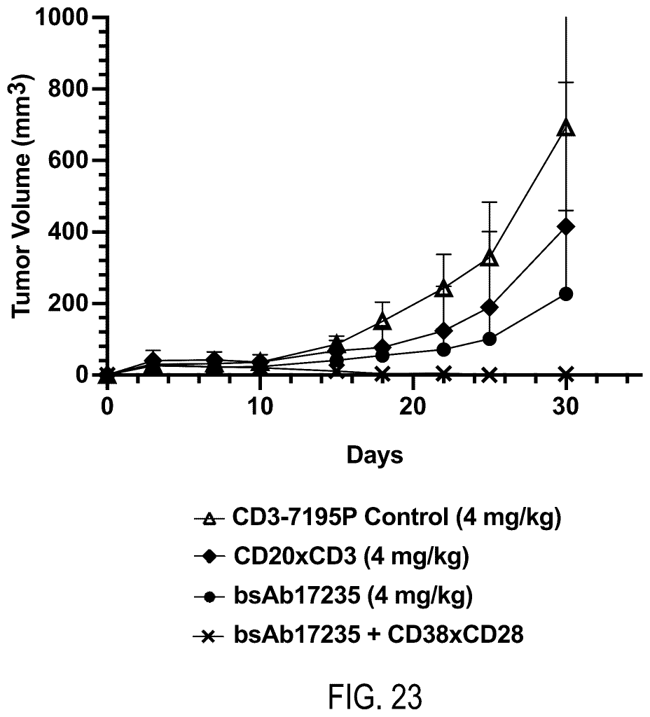

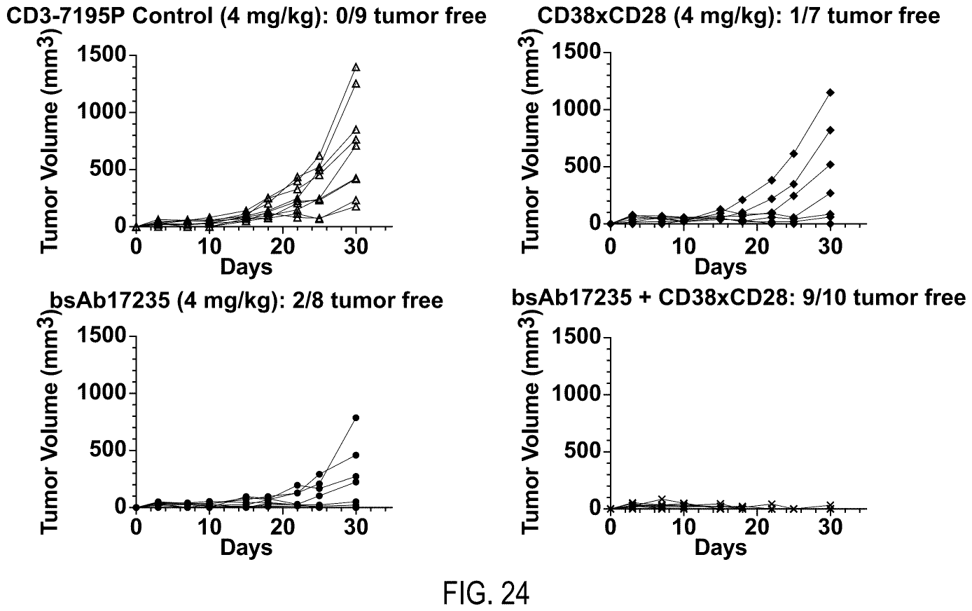

Results: Monotherapy with bsAb17235 drove strong anti-tumor responses in this model (FIGS. 23, 24). Combining bsAb17235 with the CD38×CD28 antibody dramatically enhanced anti-tumor activity in this model well beyond monotherapy levels with either molecule (FIGS. 23, 24). Combination therapy treated mice achieved complete tumor remission at significantly higher rates (FIG. 24).

### Example 9: Robust Anti-Tumor Efficacy of Selected SLC3A2-APISxCD3 Bispecific Antibody in Solid and Hematologic Tumors

This example relates to two additional in vivo studies confirming the efficacy of a selected SLC3A2-APISxCD3 bispecific antibody in treating solid and hematologic tumors related to expression of a mutant spliceosome protein (e.g., SF3B1), which leads to expression of the noncanonical spliceoform of SLC3A2, SLC3A2-APIS. Immunodeficient mice were first engrafted with human immune effector cells, and then engrafted with tumor cells that endogenously harbor an SF3B1 hotspot mutation and consequently endogenously express SLC3A2-APIS.

Study 1: The anti-tumor activity of the SLC3A2-APISxCD3 bispecific antibody bsAb17235 was assessed in comparison to a non-TAAxCD3 bispecific control using a MEL-202 uveal melanoma xenograft model. Mel-202 cells harbor an SF3B1 mutation and consequently endogenously express SLC3A2-APIS. Specifically, 8 to 15-week-old female NSG mice were randomly assigned into 5 dosing groups and implanted IP with 5×10⁶ human PBMC on Day −19. On Day −10 the mice were implanted SC with 10×10⁶ MEL202 cancer cells into the right hind flank. On Day 0, when average tumor size reached approximately 150 mm³, mice were dosed and monitored as indicated in Table 20.

TABLE 20
Study Design

| Antibody | Dose (mg/kg) | n/group^{a} |
|---|---|---|
| bsAb17235 | 0.04 | 9^{b} |
| bsAb17235 | 0.1 | 7^{c} |
| bsAb17235 | 1 | 8^{c} |
| bsAb17235 | 4 | 9^{c} |
| non-TAAxCD3 | 4 | 8^{c} |

^{a}n indicates number of mice per group analyzed; n = 10 mice per group at study onset
^{b}One mouse was excluded on Day 30 of dosing due to graft versus host disease
^{c}Mice were excluded from dosing due to unsuccessful tumor or PBMC engraftment

All groups were dosed on Days 0, 6, 10, 14, 17, 21, 24, and 28 for a total of 8 doses per group. Tumor burden was measured every 3 or 4 days beginning on Day 0 by measuring neoplastic volumes via caliper measurements. Tumor volumes were calculated by the formula: volume=(length×width²)/2. At end of study, at Day 36, tumor-free responses were recorded.

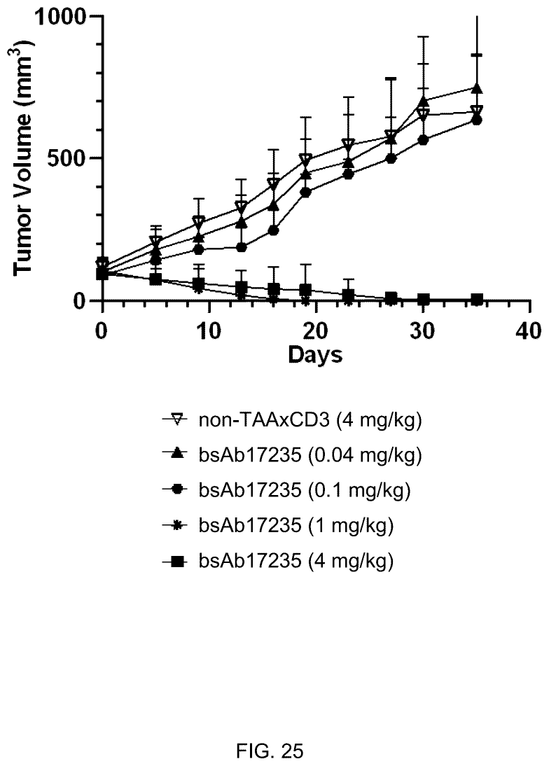

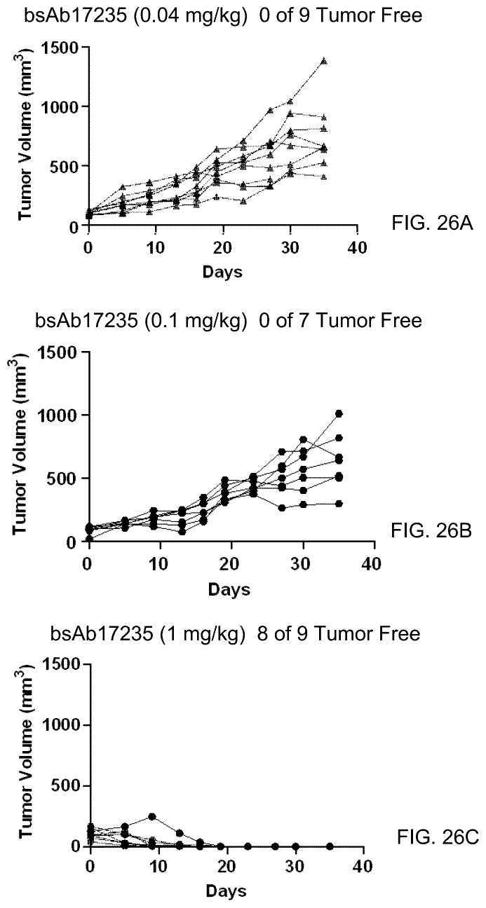

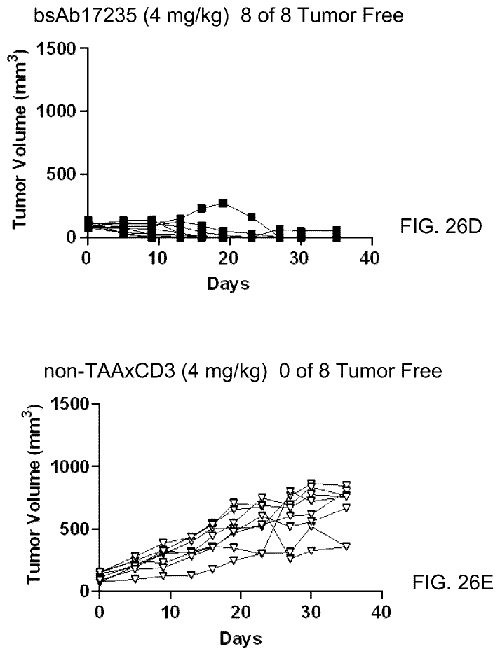

Results: Administration of bsAb17235 resulted in robust tumor growth control. Administration of bsAb17235 at 1 and 4 mg/kg resulted in marked tumor growth control, with 8/9 and 8/8 mice tumor free, respectively. No mice were tumor-free when dosed with 0.04 and 0.1 mg/kg bsAb17235 or non-TAAxCD3 (FIGS. 25 and 26A-E).

Study 2: The anti-tumor activity of SLC3A2-APISxCD3 bispecific antibody bsAb17235 was assessed in comparison to non-TAAxCD3 bispecific control using a HNT-34 acute myeloid leukemia xenograft model. HNT-34 cells endogenously harbor an SF3B1 mutation and consequently endogenously express SLC3A2-APIS. Specifically, 11 to 12-week-old female NSG mice were randomly assigned into 4 groups and implanted SC with 20×10⁶ HNT34 cells into the right hind flank and 5×10⁶ human PBMC IP on Day-16. On Day 0, when average tumor size reached approximately 150 mm³, mice were dosed and measured as indicated in Table 21.

TABLE 21
Study Design

| Antibody | Dose (mg/kg) | n/group^{a} |
|---|---|---|
| bsAb17235 | 0.1 | 9^{b} |
| bsAb17235 | 1 | 10 |
| bsAb17235 | 4 | 9^{c} |
| non-TAAxCD3 | 4 | 10 |

^{a}n indicates number of mice per group analyzed; n = 10 mice per group at study onset
^{b}One mouse was excluded on Day 37 of dosing due to graft versus host disease
^{c}Mice were excluded from dosing due to unsuccessful tumor or PBMC engraftment

All dosing groups were dosed Days 0, 6, 9, 12, 15, 19, 21, 25, 28, and 32 for a total 10 doses per group. Tumor burden was measured every 3 or 4 days beginning on Day 0. At end of study, at Day 60, tumor-free responses were recorded.

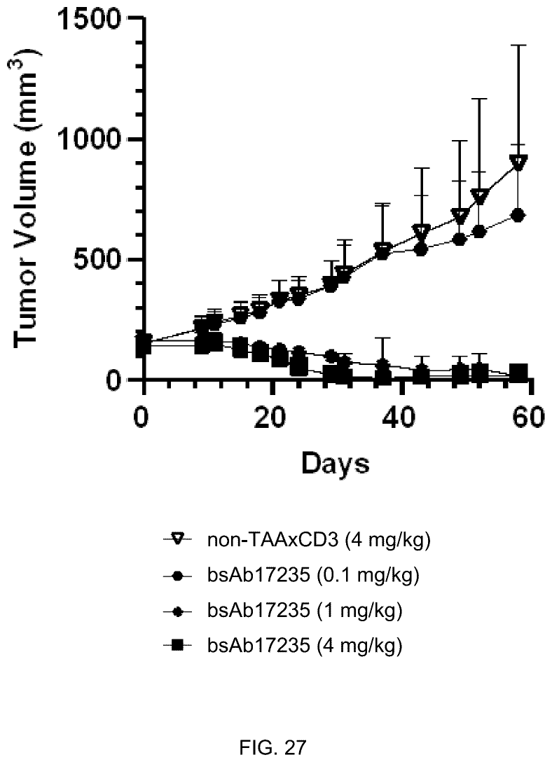

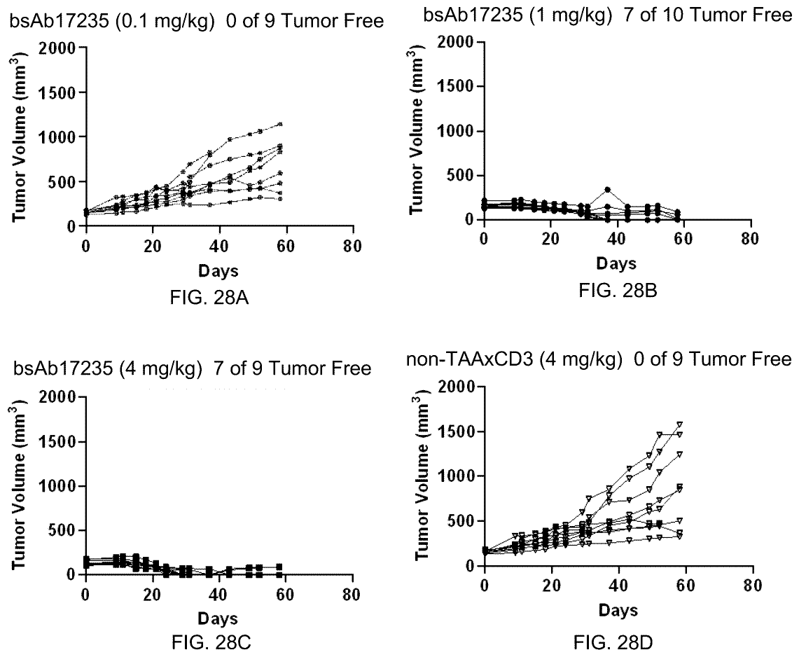

Results: Administration of bsAb17235 resulted in robust tumor growth control. Administration of bsAb17235 at 1 and 4 mg/kg resulted in marked tumor growth control with suppression observed up to day 60. Dosing at 1 and 4 mg/kg led to 7/10 and 7/9 tumor-free mice, respectively, whereas no mice were tumor-free when dosed with 0.1 mg/kg bsAb17235 or non-TAAxCD3 (FIGS. 27 and 28A-28D).

Conclusion: In both solid and hematologic xenograft tumor models that harbor SF3B1 mutations and endogenously express SLC3A2-APIS-MEL-202 (uveal melanoma) and HNT34 (AML)-administration of a SLC3A2-APISxCD3 bispecific antibody resulted in strong dose-dependent tumor growth control, with near complete tumor-free responses at ≥1 mg/kg.

### Example 10: Lack of T-Cell Activation and Cytokine Release Elicited in Whole Blood from Healthy Human Donors by a Selected SLC3A2-APISxCD3 Bispecific Antibody

This example relates to a flow cytometric assay demonstrating the lack of T-cell activation and cytokine release elicited by a selected SLC3A2-APISxCD3 bispecific antibody. The ability of the SLC3A2-APISxCD3 bispecific antibody bsAb17235 to elicit T-cell activation and cytokine release in whole blood from healthy human donors was evaluated relative to the T-cell engaging positive control bispecific antibody CD20×CD3 and a non-TAAxCD3 negative control. This study indicates the specificity of bsAb17235 activity and the extent to which SLC3A2-APIS is a unique epitope associated with SF3B1-mutant cells (and not normal cells).

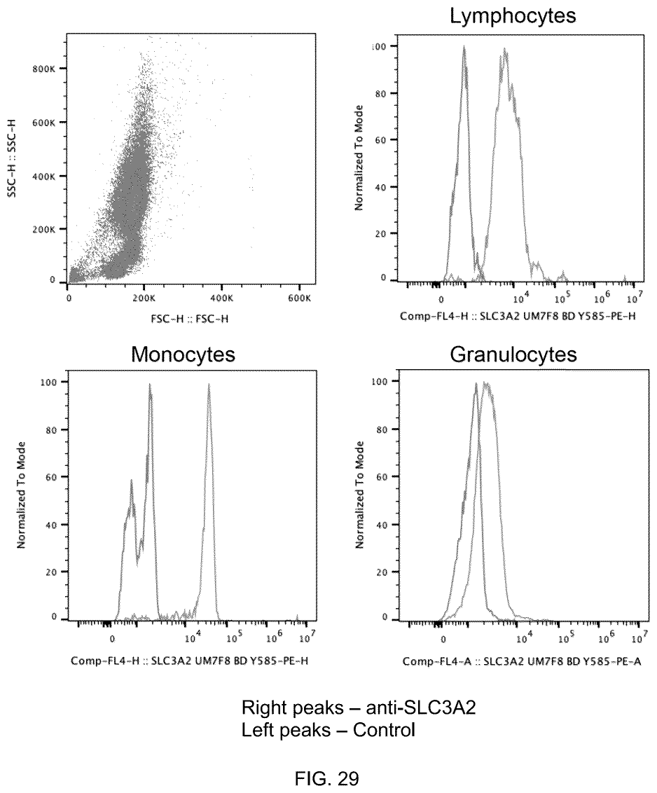

Expression of SLC3A2-WT and SLC3A2-APIS in normal blood was evaluated. SLC3A2-WT is expressed abundantly on various leukocyte populations and SLC3A2-APIS expression was not detectable (FIG. 29). The potential for bsAb17235 to mediate T-cell activation and cytokine release in the presence of whole blood was assessed using whole blood from three healthy human donors. Whole blood was incubated with serial dilutions (4 pM to 269 nM) of bsAb17235, non TAAxCD3, or CD20×CD3. T-cell activation was assessed by flow cytometry using a phenotyping cocktail of fluorophore labeled antibodies to CD2, CD4, CD8, CD16, CD25, CD45, and CD69 to quantify expression of CD69 (a cell-surface marker of early T-cell activation) on T cells. The supernatants of the assay wells were assessed for cytokine (IFNG, IL6, and TNFA) release using the V-PLEX Proinflammatory Panel kit according to the manufacturer's instructions. Samples were analyzed by flow cytometry using a Cytoflex XL flow cytometer. Cytokine concentrations (in pg/mL) were interpolated from the MFI of standards provided in each kit.

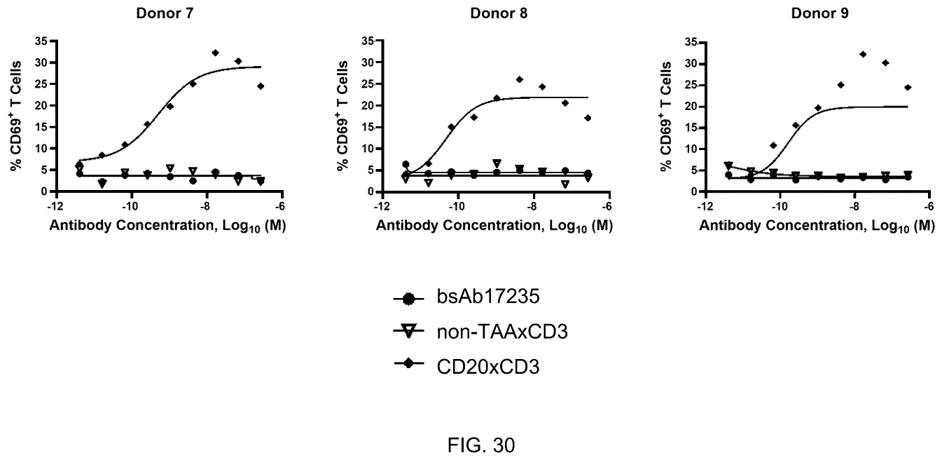

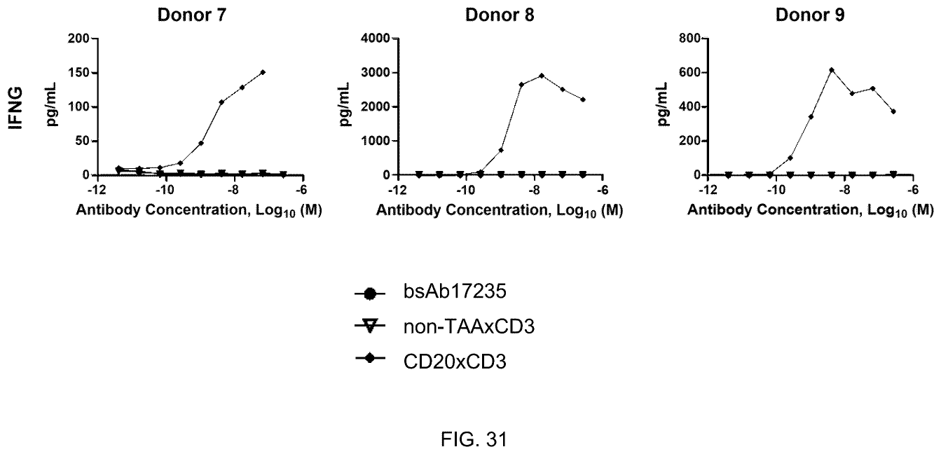

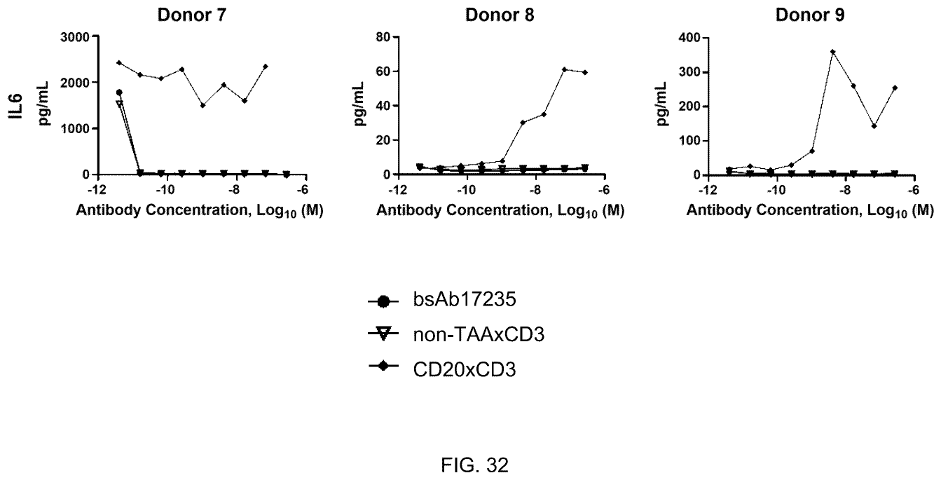

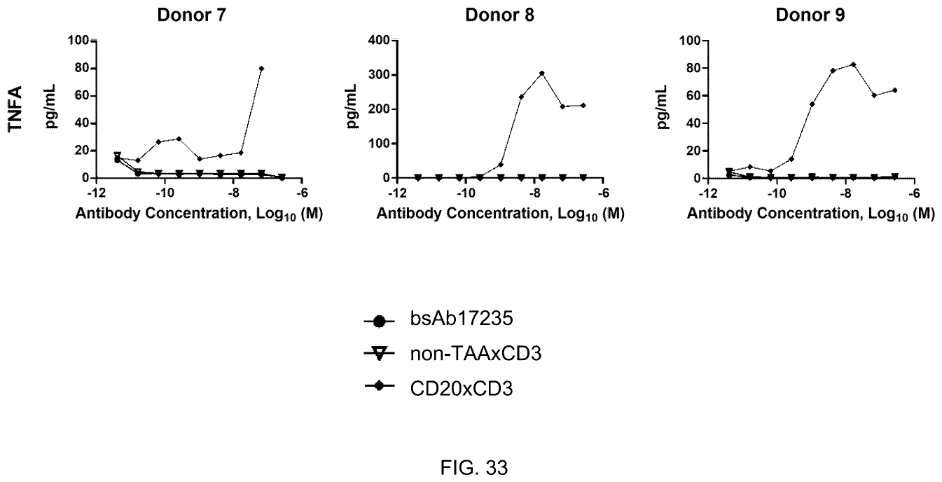

bsAb17235 did not mediate T-cell activation (FIG. 30) or cytokine release (FIGS. 31-33) in the presence of whole blood at any concentration tested. Likewise, non-TAAxCD3 did not mediate T-cell activation and cytokine release was not observed (FIGS. 29-32). By contrast, the positive control for this assay, CD20×CD3, did mediate T-cell activation and release (FIGS. 29-32). This study indicates that bsAb17235 activity is specific to SLC3A2-APIS and that the epitope of SLC3A2-APIS recognized by bsAb17235 is a unique epitope associated with SF3B1-mutant cells (and not normal cells).

### Example 11: CryoEM Structure of SLC3A2-APIS Ectodomain with SLC3A2-APIS Binding Arm of a Selected SLC3A2-APISxCD3 Bispecific Antibody

This example relates to a cryo electron microscopy (CryoEM) study defining residue-level interactions at the epitope/paratope interface of the SLC3A2-APIS binding arm of a selected SLC3A2-APISxCD3 bispecific antibody bound to the noncanonical spliceoform of SLC3A2, SLC3A2-APIS.

REGN17212 Fab production: 5 mg of REGN17212 (the parental antibody of bsAb23479, bsAb23483, and bsAb23477) was diluted to 2 mL in PBS. To cleave the IgG below the hinge, 100 μg of IdeS enzyme was added to the sample, followed by incubation at 37° C. for 30 minutes. Hinge cysteines were reduced by beta-mercaptoethanol for 90 minutes at 37° C. The sample was then treated with 0.1 M lodoacetamide to covalently cap free cysteines. The Fc domain was removed by binding to CaptureSelect™ IgG-Fc multispecies resin. A final size exclusion chromatography step using a Superdex 200 increase 10/300 GL column equilibrated to buffer containing 50 mM Tris pH 7.5, 150 mM NaCl was conducted to purify the sample to homogeneity.

CryoEM sample preparation and data collection: SLC3A2-APIS ectodomain protein (SEQ ID NO: 265), REGN17212 Fab, and noncompeting commercial mouse IgG antibody MEM-108 (added to facilitate 3D reconstruction) were mixed at a 1:1:0.5 molar ratio and incubated for at least 30 minutes at 4° C. The complex sample (˜7 mg/mL concentration) was supplemented with 0.2% A8-35 amphipol then immediately applied to Quantifoil 1.2/1.3, 300 mesh grids (carbon film on copper grid) that were freshly plasma cleaned using a Solarus Il. The sample was blotted and plunge-frozen into liquid ethane cooled by liquid nitrogen using a Vitrobot Mark IV operated at 4° C. and 100% humidity. CryoEM data were collected on a Glacios microscope equipped with a Falcon 4i camera operating in counted mode. Automated data collection was carried out using EPU software. Movies were collected at a nominal magnification of 165,000×(0.697 Å/pixel) with energy filter inserted.

CryoEM data processing and map generation: CryoEM data were processed using cryoSPARC v4.5.3 software. Movies were motion-corrected and CTF parameters were estimated for the summed micrographs. Particle coordinates were picked using 2D templates and TOPAZ. Particle images corresponding to false positives, contaminants, or broken complexes were removed after multiple rounds of 2D classification. Homogenous subsets of particle images corresponding to the target complex were obtained after ab initio reconstruction and hetero refinement. The final 3.45 A resolution map (according to a gold standard FSC=0.143 criterion) was calculated from 130,264 particle images using the Local Refine jobtype.

Model building and refinement: Initial model coordinates were obtained from a previously determined structure of the SLC3A2-APIS ectodomain and a structure of REGN17212 Fab predicted by implementation of AlphaFold2. The Fit-in-map function of UCSF Chimera was used to dock initial models into their corresponding densities. Manual model building was carried out using Coot version 0.8.9, and real space refinements were conducted in Phenix version 1.21. Structures were analyzed in pymol 2.6.2 and PISA as implemented in CCP412.

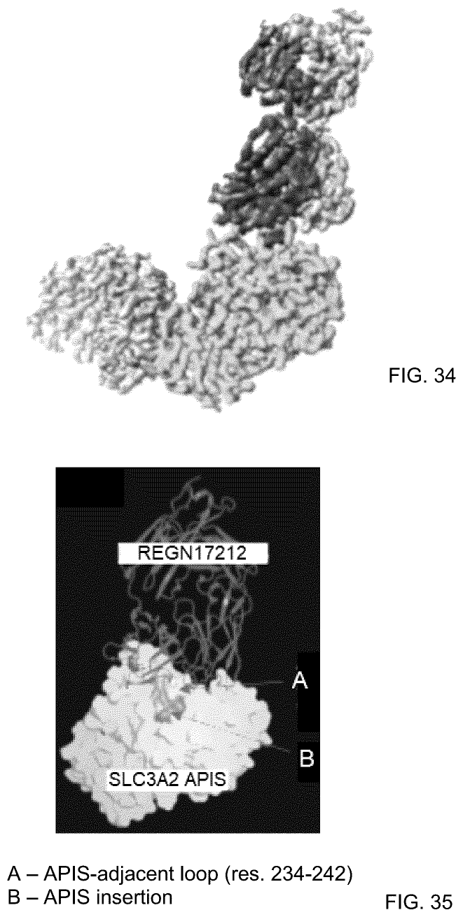

Results: A 3.45 A resolution structure of REGN17212 Fab-bound SLC3A2-APIS ectodomain was obtained by single particle cryoEM (FIGS. 34 and 35). CryoEM density for most side chains of the antigen and Fab variable region are clearly defined in the map, allowing for accurate model building and assessment of residue-level interactions at the epitope/paratope interface.

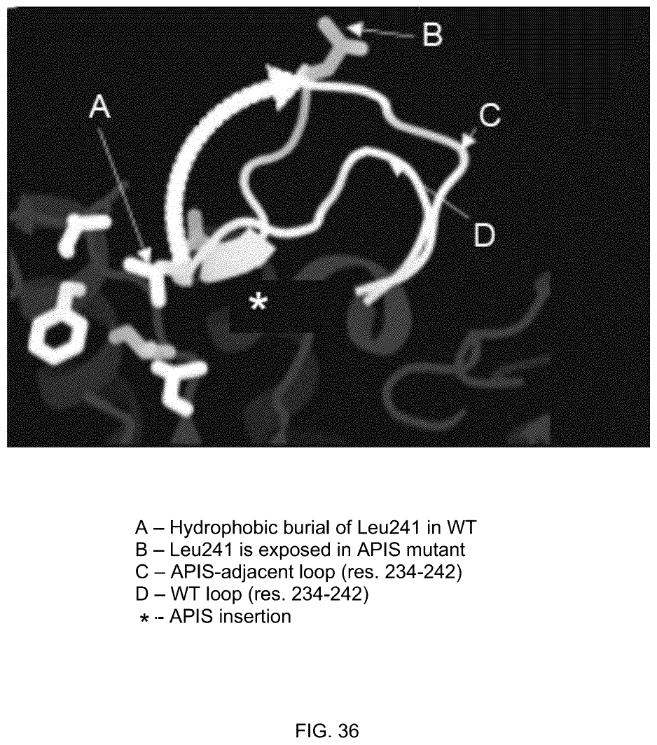

The four residue APIS insertion occurs at a partially surface-exposed a helix in the SLC3A2 ectodomain. The insertion results in a localized structural rearrangement of a loop (residues 234-242) directly N-terminal to the insertion site (FIG. 36). This rearrangement includes a 12.5 A displacement of Leu241, which is buried in the wild type structure (PDB 8G0M), to a surface-exposed position in SLC3A2-APIS. The conformation of the APIS insertion-adjacent loop in the REGN17212 complex structure is distinct from its conformation when bound to a different antibody (REGN17224), indicating its flexibility.

The cryoEM structure shows that REGN17212 buries a total of 1161 A2 surface area on SLC3A2-APIS, with the heavy chain contributing 83% of the paratope surface area. The REGN17212 epitope is summarized in Table 22.

TABLE 22
Summary of Epitope Residues in SLC3A2-APIS for anti-
SLC3A2-APIS Monospecific Antibody REGN17212

| SLC3A2-APIS | Cα(APIS)-Cα(WT) | HC BSA | LC BSA |
|---|---|---|---|
| epitope residue | displacement (Å) | (Å²)** | (Å²) |
| ASP 229 | 0.6 | 26 | 0 |
| GLN 231 | 1.1 | 65 | 0 |
| ALA 232 | 0.7 | 5 | 0 |
| GLY 235* | 0.6 | 2 | 0 |
| HIS 236* | 1 | 89 | 0 |
| GLY 237* | 5.6 | 84 | 0 |
| ALA 238* | 5.4 | 26 | 0 |
| GLY 239* | 8.5 | 54 | 2 |
| ASN 240* | 8.5 | 29 | 78 |
| LEU 241* | 12.5 | 87 | 40 |
| ALA 242* | 7.6 | 2 | 0 |
| LYS 273 | 0.8 | 21 | 0 |
| ASP 285 | 0.4 | 2 | 0 |
| PRO 286 | 1.2 | 0 | 37 |
| ASN 287 | 0.4 | 44 | 40 |
| PHE 288 | 0.7 | 14 | 0 |
| TYR 316 | 0.4 | 6 | 0 |
| ARG 317 | 0.7 | 77 | 0 |
| GLN 486 | 1.5 | 2 | 0 |
| PRO 487 | 1.2 | 42 | 0 |
| ALA 490 | 0.6 | 6 | 0 |
| VAL 492 | 0.5 | 1 | 0 |
| ASP 496 | 0.5 | 15 | 0 |
| GLU 497 | 0.3 | 12 | 0 |
| SER 498 | 0.5 | 67 | 0 |
| PHE 500 | 0.7 | 5 | 0 |
| PRO 501 | 2.1 | 94 | 0 |
| ASP 502 | 4.5 | 38 | 0 |
| ILE 503 | 2.2 | 1 | 0 |
| PRO 504 | 4.2 | 43 | 0 |
| GLY 505 | 4.9 | 7 | 0 |
| ALA 506 | 2.6 | <1 | 0 |
| VAL 507 | 1.4 | <1 | 0 |

Epitope residues and buried surface areas were determined by PISA. Asterisk denotes epitope residues that are part of the Gln234-Ala242 loop that adopts a different conformation in APIS-inserted SLC3A2. The second column from left lists pairwise Ca-Ca distances of REGN17212 epitope residues in APIS-inserted and wild type SLC3A2 (PDB 8G0M used as reference). HC=heavy chain; LC=light chain; BSA=buried surface area. Note that residue numbering C-terminal to the APIS site accounts for the insertion and differs from the wild type SLC3A2 numbering from Uniprot entry P08195.

The REGN17212 epitope covers an area nearby the APIS insertion site but does not contact the inserted APIS residues directly (FIG. 35). The surface exposed loop (residues 234-242) that undergoes a structural rearrangement due to the APIS insertion is centrally located in the epitope; residues in this loop, which have a Ca displacement of >5 Å relative to wild type SLC3A2, contribute 35% of the surface area buried by REGN17212. Leu241 has the largest surface area buried by REGN17212 (127 A2) of any epitope residue, indicating a critical role for REGN17212 binding. As mentioned above, Leu241 is buried in wild type SLC3A2 and not available for antibody interaction but becomes surface exposed in SLC3A2-APIS. REGN17212 may therefore selectively bind SLC3A2-APIS over wild type SLC3A2 by recognizing an epitope adjacent to the APIS insertion that adopts a unique conformation.

### Example 12: Characterization of Binding of a Selected SLC3A2-APISxCD3 Bispecific Antibody to Cell Surface SLC3A2-APIS and CD3 on Primary T Cells

This example relates to an in vitro study demonstrating the ability of a selected SLC3A2-APISxCD3 bispecific antibody to specifically bind SLC3A2-APIS+ cell lines and cell-surface CD3 on unstimulated primary human T cells as determined by flow cytometry.

Binding activity of the SLC3A2-APISxCD3 bispecific antibody bsAb17325 was assessed with the following cell lines: 3T3 cells, which lack expression of SLC3A2; 3T3 cells engineered to express WT SLC3A2; 3T3 cells engineered to express SLC3A2-APIS; NALM-6 cancer cells, which endogenously express WT SLC3A2; and MEL 202 and HNT-34 cancer cells, which endogenously express SLC3A2-APIS. 3T3 cells were also engineered to express human CD20 for reasons unrelated to this study. The ability of bsAb17325 to bind human, cynomolgus monkey, and rhesus monkey T cells was also assessed.

Preparation of Target and Primary T Cells for Cell-Surface Binding Assays: PBMC isolated from 2 human donors were used for cell-surface binding assays. Cells were diluted with PBS and isolated by density gradient centrifugation over Ficoll-Paque PLUS solution followed by removal of supernatants. Cell lines and PBMC were stained with eBioscience Fixable Viability Dye eFluor 780 for a viability test. Cells were incubated with a serial dilution of bsAb17235, IgG4^{P-PVA} isotype control, non-TAAxCD3, or parental SLC3A2-APIS antibody REGN17224. In addition to serially diluted antibodies, PBMC were incubated with fluorophore-labeled CD4 and CD8 antibodies. Titrated antibodies ranged in concentration from 0.3 nM to 15 uM for cell lines and 0.02 nM to 1 M for primary T cells. After primary antibody incubation, cells were incubated with APC AffiniPure F(ab′)₂ Fragment Goat Anti-Human IgG, FcG fragment specific, and subsequently fixed.

Analysis of Cells Using Flow Cytometry: Cells were analyzed by flow cytometry on a ZE5 flow cytometer. Cells were gated such that only viable, single cells were analyzed. For primary T cells, samples were also gated on CD4+ and CD8+. The amount of titrated antibody bound was evaluated by analyzing MFI using the FlowJo software. For EC₅₀ determinations, the measured MFI values for live, single cells were analyzed using a 4-parameter logistic equation over an 11-point concentration-response curve using Prism software. Reported MFI values are the MFI values at the highest tested antibody concentration. MFI fold was calculated as the MFI at the highest tested antibody concentration over the MFI for secondary antibody alone.

Binding to Cell-Surface SLC3A2-APIS and WT SLC3A2: Flow cytometric analysis was used to confirm SLC3A2-APIS expression on 3T3/SLC3A2-APIS/hCD20, MEL-202, and HNT-34 cells, and to confirm WT SLC3A2 expression on 3T3/SLC3A2 WT/hCD20 and NALM-6 cells. The same experimental design as described above, but cells were incubated with a serial dilution of anti-SLC3A2 or IgG1 isotype control (2 pM to 100 nM). After primary antibody incubation, cells were incubated with APC AffiniPure F(ab′) 2 Fragment Goat Anti-Mouse IgG, FcG fragment specific and subsequently fixed.

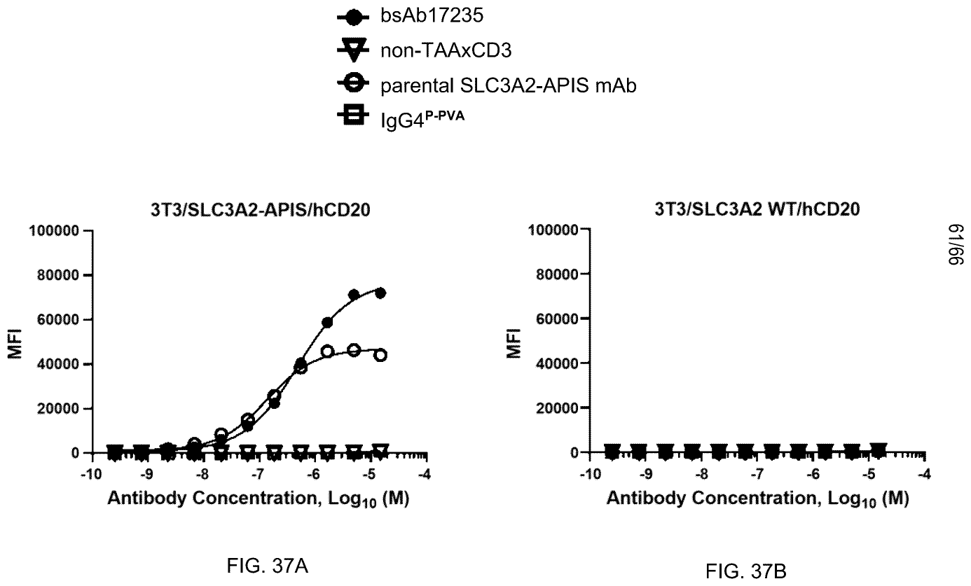

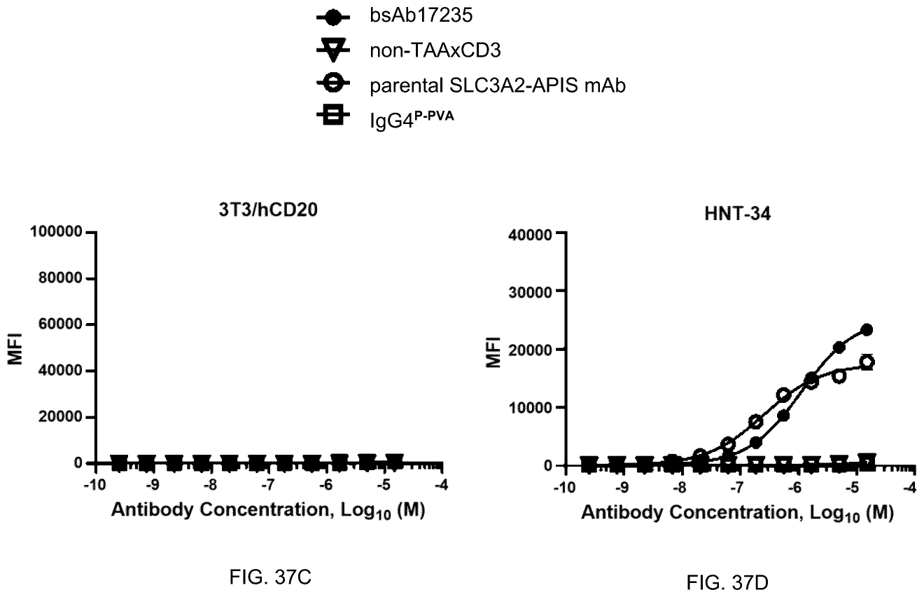

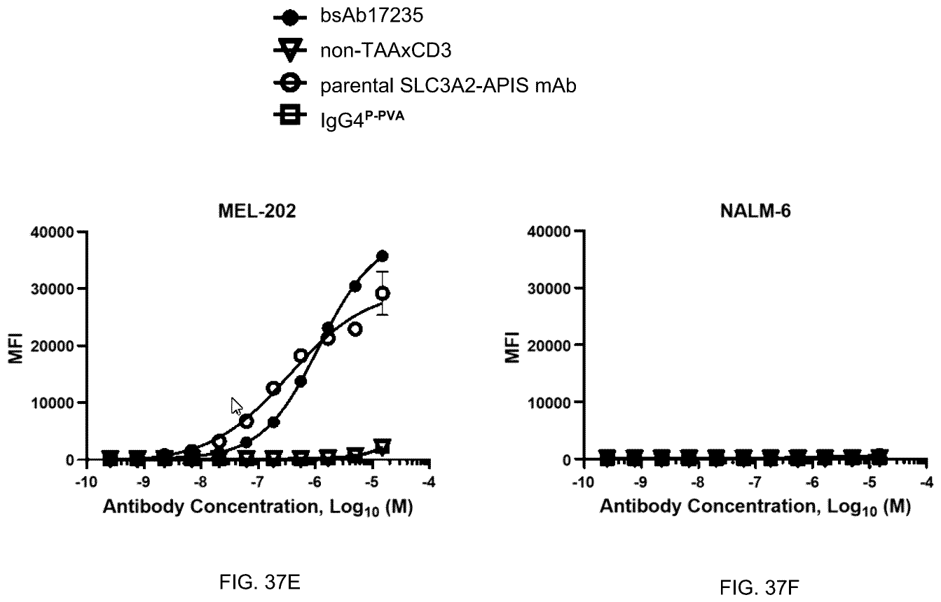

Results: Flow cytometry was used to evaluate the specificity of bsAb17235 to bind SLC3A2-APIS+ cell lines. bsAb17235 displayed concentration-dependent, specific binding to 3T3 cells engineered to express SLC3A2-APIS (3T3/SLC3A2-APIS/hCD20; FIG. 37A) with nanomolar EC₅₀ values (Table 23). bsAb17235 also displayed concentration-dependent, specific binding to HNT 34 and MEL-202 cells, which endogenously express SLC3A2-APIS (FIGS. 37D and 37E), with single-digit micromolar EC₅₀ values (Table 23). bsAb17235 did not bind to 3T3 cells engineered to express WT SLC3A2 (3T3/SLC3A2 WT/hCD20; FIG. 37B) or NALM-6 cells, which endogenously express WT SLC3A2 and not SLC3A2-APIS (FIG. 37F). Table 23 sets forth bsAb17235 binding to cell lines tested in this study.

TABLE 23
Summary of bsAb17235 Binding to Cells Expressing
or Not Expressing SLC3A2-APIS

| | | Antibody | | | |
|---|---|---|---|---|---|
| | | | non- | Parental SLC3A2- | |
| Cell Line | Readout | bsAb17235 | TAA × CD3 | APIS antibody | IgG4^{P-PVA} |
| 3T3/SLC3A2- | EC₅₀ (M) | 4.8E−7 | ND | 1.4E−7 | ND |
| APIS/hCD20 | Fold Binding | 1288 | 10 | 743 | 19 |
| 3T3/SLC3A2 | EC₅₀ (M) | ND | | | |
| WT/hCD20 | Fold Binding | 13 | 13 | 13 | 18 |
| 3T3/hCD20 | EC₅₀ (M) | ND | | | |
| | Fold Binding | 9 | 7 | 8 | 12 |
| HNT-34 | EC₅₀ (M) | 1.1E−6 | ND | 2.6E−7 | ND |
| | Fold Binding | 174 | 5 | 132 | 2 |
| MEL-202 | EC₅₀ (M) | 1.2E−6 | ND | 3.4E−7 | ND |
| | Fold Binding | 234 | 14 | 191 | 15 |
| NALM-6 | EC₅₀ (M) | ND | | | |
| | Fold Binding | 5 | 2 | 7 | 3 |

EC₅₀, Half-maximal effective concentration

The capacity of the anti-SLC3A2-APIS arm to bind cell-surface SLC3A2-APIS was confirmed using the parental SLC3A2-APIS monoclonal antibody, a bivalent antibody comprising 2 Fab arms identical to the single anti-SLC3A2-APIS Fab arm of bsAb17235. The parental SLC3A2-APIS antibody displayed concentration-dependent, specific binding to SLC3A2-APIS+ cell lines with nanomolar EC₅₀ values and exhibited no binding to cells lacking SLC3A2-APIS expression (FIG. 37; summarized in Table 23).

bsAb17235 did not bind to parental 3T3/hCD20 cells that do not express SLC3A2 (FIG. 37C). Binding was not observed for the non TAAxCD3 antibody or IgG4P-PVA isotype control to any of the tested cell lines (FIG. 37; summarized in Table 23). SLC3A2 APIS and WT SLC3A2 expression on cell lines was confirmed using flow cytometry.

Binding to Primary T Cells: Flow cytometric analysis was utilized to determine the ability of bsAb17235 to bind to T cells from cynomolgus monkey and rhesus monkey. Briefly, cells were stained with Zombie Red Fixable Viability Dye and then incubated with a serial dilution of bsAb17235 or IgG4P-PVA isotype control (4.07 pM to 1.07 UM). After primary antibody incubation, cells were incubated with Alexa Fluor 647 AffiniPure Fab Fragment Goat Anti-Human IgG (H+L), and subsequently fixed. Cells were analyzed on a Cytek Aurora flow cytometer.

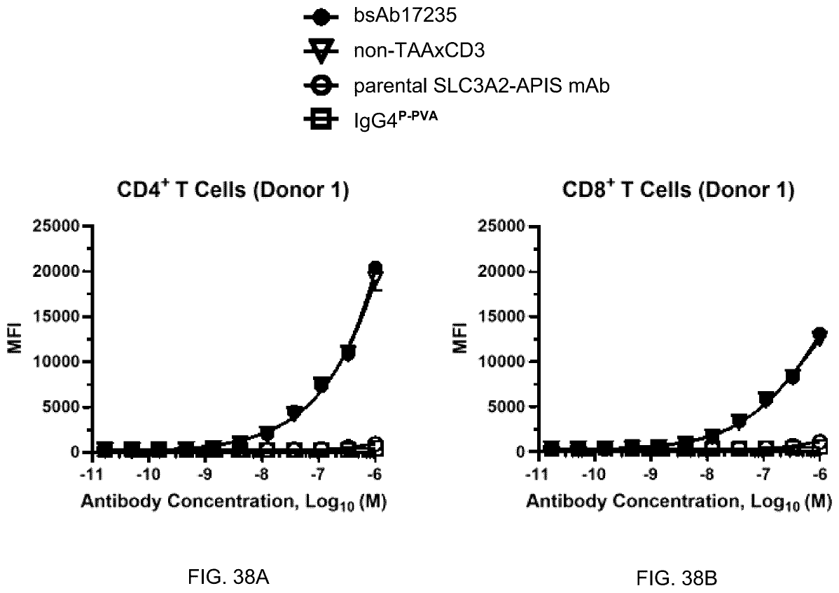

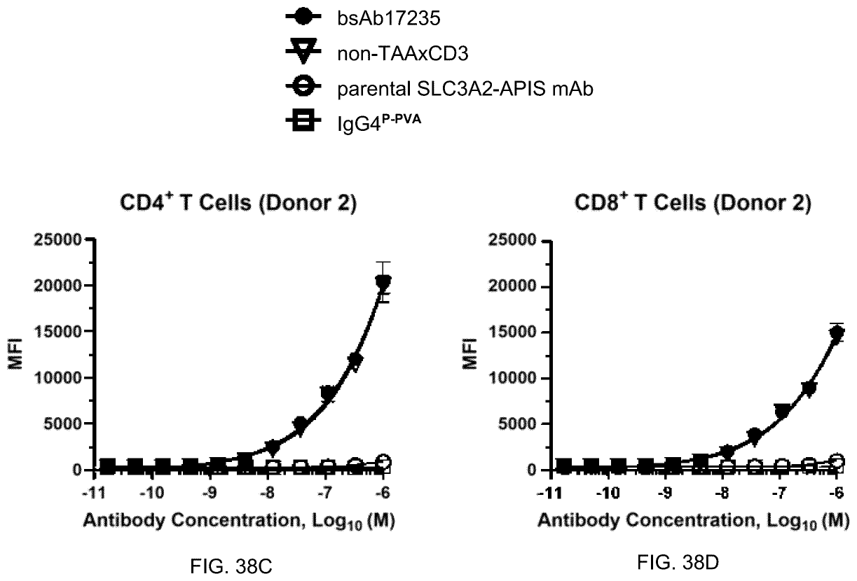

Results: bsAb17235 displayed concentration-dependent binding to primary T cells (unstimulated CD4+ and CD8+ T cells) from 2 human donors (FIG. 38A-D). The capacity of the anti-CD3 arm to bind cell-surface CD3 was confirmed using a non-TAAxCD3 bsAb, which displayed concentration-dependent binding to T cells (FIG. 38A-D). The parental SLC3A2-APIS antibody and IgG4^{P-PVA} control antibody did not display binding to primary T cells (FIG. 38A-D). Table 24 sets forth binding of bsAb17235 and control antibodies to unstimulated human T cells. MFI values are reported instead of EC₅₀ values because a sigmoidal curve fit was not observed, preventing EC₅₀ calculation. The reported MFI values are the average of MFI values from 2 human PBMC donors. MFI fold was obtained by the dividing the MFI at the maximum tested antibody concentration over MFI of the secondary antibody alone.

TABLE 24
Summary of bsAb17235 and control antibody binding to unstimulated human T cells

| | | Antibody | | | |
|---|---|---|---|---|---|
| | | | non- | Parental SLC3A2- | |
| Cell Line | Readout | bsAb17235 | TAAxCD3 | APIS antibody | Ig G4^{P-PVA} |
| CD4⁺ T Cells | MFI | 20555 | 19610 | 922 | 435 |
| | Fold Binding | 63 | 58 | 2 | 1 |
| CD8⁺ T Cells | MFI | 14074 | 13533 | 1037 | 495 |
| | Fold Binding | 36 | 34 | 2 | 1 |

MFI, Median fluorescence intensity

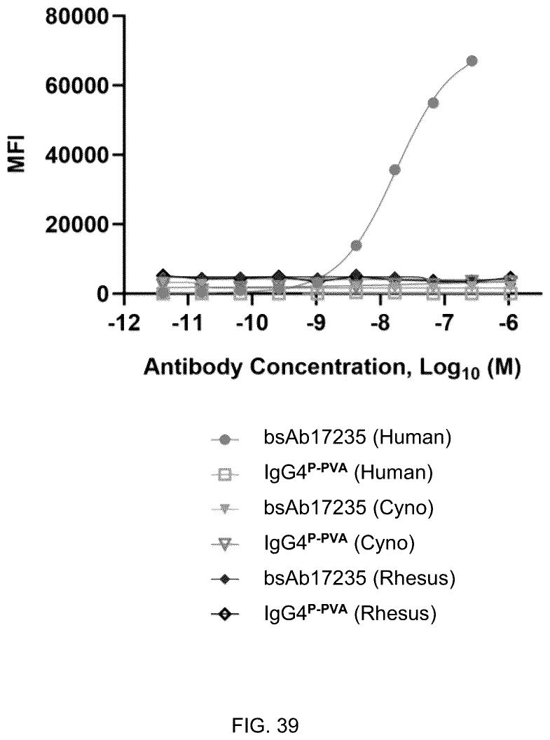

The splicing variant of SLC3A2 resulting in SLC3A2-APIS is unique to humans, therefore, binding of bsAb17235 to cells expressing cynomolgus monkey or rhesus monkey SLC3A2 was not evaluated. However, the ability of bsAb17235 to bind CD3 from cynomolgus monkey and rhesus monkey was evaluated by flow cytometry. bsAb17235 bound to human, but not cynomolgus or rhesus monkey, T cells (FIG. 39).

Conclusion: bsAb17235 displayed concentration-dependent, specific binding to 3T3 cells engineered to express SLC3A2-APIS, and to MEL 202 and HNT-34 cancer cells, which endogenously express SLC3A2-APIS. In contrast, bsAb17235 did not bind to cells lacking SLC3A2 APIS expression (i.e., 3T3 cells lacking expression of SLC3A2, 3T3 cells engineered to express WT SLC3A2, or NALM-6 cancer cells, which endogenously express WT SLC3A2). bsAb17235 displayed concentration-dependent binding to unstimulated primary human T cells, but did not bind to T cells from cynomolgus or rhesus monkeys.

The foregoing merely illustrates the principles of the disclosure. Any examples set forth in this specification are not intended to be limiting and merely set forth some of the many possible embodiments for the appended claims. Those skilled in the art will readily recognize various modifications and changes that may be made without following the example embodiments and applications illustrated and described herein, and without departing from the true spirit and scope of the following claims.

All references cited and/or discussed in this specification are incorporated herein by reference in their entireties and to the same extent as if each reference was individually incorporated by reference.

We claim:

1. An isolated antigen-binding protein comprising an antigen-binding domain that binds specifically to SLC3A2-APIS wherein the antigen-binding protein does not bind to wild-type SLC3A2, as measured by flow cytometry.

2. The isolated antigen-binding protein of claim 1, wherein the antigen-binding domain interacts with at least one amino acid residue selected from the group consisting of D229, Q231, A232, Q234, G235, H236, G237, A238, G239, N240, L241, A242, A243, P244, S246, G247, K273, P286, N287, F288, Y316, R317, P487, D496, E497, S498, P501, D502, P504, and G505 contained in SLC3A2-APIS, as determined by cryo-electron microscopy.

3. The isolated antigen-binding protein of claim 1 or 2, wherein the antigen-binding domain interacts with at least one amino acid residue selected from the group consisting of Q234, G235, H236, G237, A238, G239, N240, L241, A242, A243, P244, S246 and G247 contained in SLC3A2-APIS, as determined by cryo-electron microscopy.

4. The isolated antigen-binding protein of claim 3, wherein the antigen-binding domain interacts with A243, P244, or S246 contained within SLC3A2-APIS, as determined by cryo-electron microscopy.

5. The isolated antigen-binding protein of claim 1 or 2, wherein the antigen-binding domain interacts with at least one amino acid residue selected from the group consisting of D229, Q231, A232, G235, H236, G237, A238, G239, N240, L241, A242, G247, K273, P286, N287, F288, Y316, R317, P487, D496, E497, S498, P501, D502, P504, and G505 contained in SLC3A2-APIS, as determined by cryo-electron microscopy.

6. The isolated antigen-binding protein of any one of claim 1, 2 or 5, wherein the antigen-binding domain does not interact with A243, P244, or S246 contained within SLC3A2-APIS, as determined by cryo-electron microscopy.

7. An isolated antigen-binding protein comprising an antigen-binding domain that binds specifically to SLC3A2-APIS, wherein the antigen-binding domain (i) does not bind to wild-type SLC3A2; and (ii) interacts with one or more amino acids contained within SEQ ID NO: 265, as determined by cryo-electron microscopy.

8. The isolated antigen-binding protein of any one of claims 1-5, wherein the antigen-binding domain interacts with at least one amino acid contained within SEQ ID NO: 266, as determined by cryo-electron microscopy.

9. The isolated antigen-binding protein of claim 8, wherein the antigen-binding domain interacts with at least ten amino acids contained within SEQ ID NO: 266, as determined by cryo-electron microscopy.

10. The isolated antigen-binding protein of claim 7, wherein the antigen-binding domain does not interact with A243, P244, or S246 contained within SLC3A2-APIS, as determined by cryo-electron microscopy.

11. The isolated antigen-binding protein of any one of claims 1-10, wherein the SLC3A2-APIS is expressed on a tumor cell.

12. The isolated antigen-binding protein of any one of claims 1-11, wherein the antigen-binding domain that binds specifically to SLC3A2-APIS comprises:
(a) three light chain complementarity determining regions (LCDR1, LCDR2, and LCDR3) contained within a light chain variable region (LCVR) comprising an amino acid sequence selected from SEQ ID NOs: 10, 40, 70, 112, 140, 158, and 232; and
(b) three heavy chain complementarity determining regions (HCDR1, HCDR2, and HCDR3) contained within a heavy chain variable region (HCVR) comprising an amino acid sequence selected from SEQ ID NOs: 2, 22, 32, 52, 62, 78, 88, 104, 120, 130, 148, 168, 178, 188, 198, and 208.

13. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2 and HCDR3) contained within a HCVR, wherein:
(a) LCDR1, LCDR2, and LCDR3 comprise respective amino acid sequences selected from (i) SEQ ID NO: 12, AAS, and SEQ ID NO: 16; (ii) SEQ ID NO: 42, GAS, and SEQ ID NO: 46; (iii) SEQ ID NO: 72, AAS, and SEQ ID NO: 16; (iv) SEQ ID NO: 114, AAS, and SEQ ID NO: 16; (v) SEQ ID NO: 12, AAS, and SEQ ID NO: 142, (vi) SEQ ID NO: 160, GAS, and SEQ ID NO: 46; and (vii) SEQ ID NO: 234, AAS, and SEQ ID NO: 238; and
(b) HCDR1, HCDR2, and HCDR3 comprise respective amino acid sequences selected from SEQ ID NOs: (i) 4, 6, and 8; (ii) 24, 26, and 28; (iii) 34, 36, and 38; (iv) 54, 56, and 58; (v) 64, 66, and 68; (vi) 80, 82, and 84; (vii) 90, 92, and 94; (viii) 106, 108, and 110; (ix) 122, 124, and 126; (x) 132, 134, and 136; (xi) 150, 152, and 154 (xii) 170, 172, and 174; (xiii) 170, 200, and 202; (xiv) 180, 182, and 184; (xv) 190, 192, and 194; and (xvi) 210, 6, and 8.

14. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
(a) LCDR1, LCDR2, and LCDR3 are contained within a LCVR comprising the amino acid sequence of SEQ ID NO: 10; and
(b) HCDR1, HCDR2, and HCDR3 are contained within a HCVR comprising an amino acid sequence selected from SEQ ID NOs: 2, 22, 32, 52, 62, 78, 88, 104, 120, 130, 148, 168, 178, 188, 198, and 208.

15. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
(a) LCDR1, LCDR2, and LCDR3 are contained within a LCVR comprising the amino acid sequence of SEQ ID NO: 40; and
(b) HCDR1, HCDR2, and HCDR3 are contained within a HCVR comprising an amino acid sequence selected from SEQ ID NOs: 2, 22, 32, 52, 62, 78, 88, 104, 120, 130, 148, 168, 178, 188, 198, and 208.

16. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
(a) LCDR1, LCDR2, and LCDR3 are contained within a LCVR comprising the amino acid sequence of SEQ ID NO: 70; and
(b) HCDR1, HCDR2, and HCDR3 are contained within a HCVR comprising an amino acid sequence selected from SEQ ID NOs: 2, 22, 32, 52, 62, 78, 88, 104, 120, 130, 148, 168, 178, 188, 198, and 208.

17. The isolated antigen-binding protein of any one of claims 1-12, wherein the antigen-binding domain that binds specifically to SLC3A2-APIS comprises three CDRs (LCDR1, LCDR2 and LCDR3) contained within a LCVR and three CDRs (HCDR1, HCDR2 and HCDR3) contained within a HCVR, wherein:
(a) LCDR1, LCDR2 and LCDR3 are contained within a LCVR comprising the amino acid sequence of SEQ ID NO: 112; and
(b) HCDR1, HCDR2 and HCDR3 are contained within a HCVR comprising an amino acid sequence selected from SEQ ID NOs: 2, 22, 32, 52, 62, 78, 88, 104, 120, 130, 148, 168, 178, 188, 198, and 208.

18. The isolated antigen-binding protein of any one of claims 1-12, wherein the antigen-binding domain that binds specifically to SLC3A2-APIS comprises three CDRs (LCDR1, LCDR2 and LCDR3) contained within a LCVR and three CDRs (HCDR1, HCDR2, and HCDR3) contained within a HCVR, wherein:
(a) LCDR1, LCDR2, and LCDR3 are contained within a LCVR comprising the amino acid sequence of SEQ ID NO: 140; and
(b) HCDR1, HCDR2 and HCDR3 are contained within a HCVR comprising an amino acid sequence selected from SEQ ID NOs: 2, 22, 32, 52, 62, 78, 88, 104, 120, 130, 148, 168, 178, 188, 198, and 208.

19. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
(a) LCDR1, LCDR2 and LCDR3 are contained within a LCVR comprising the amino acid sequence of SEQ ID NO: 158; and
(b) HCDR1, HCDR2 and HCDR3 are contained within a HCVR comprising an amino acid sequence selected from SEQ ID NOs: 2, 22, 32, 52, 62, 78, 88, 104, 120, 130, 148, 168, 178, 188, 198, and 208.

20. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
(a) LCDR1, LCDR2, and LCDR3 are contained within a LCVR comprising the amino acid sequence of SEQ ID NO: 232; and
(b) HCDR1, HCDR2, and HCDR3 are contained within a HCVR comprising an amino acid sequence selected from SEQ ID NOs: 2, 22, 32, 52, 62, 78, 88, 104, 120, 130, 148, 168, 178, 188, 198, and 208.

21. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein LCDR1, LCDR2, and LCDR3 are contained within a LCVR comprising the amino acid sequence of SEQ ID NO: 10; and HCDR1, HCDR2, and HCDR3 are contained within a HCVR comprising an amino acid sequence selected from SEQ ID NOs: 2, 22, 62, 104, 168, 178, 188, 198, and 208.

22. The isolated antigen-binding protein of any one of claims 1-12, wherein the antigen-binding domain that binds specifically to SLC3A2-APIS comprises three CDRs (LCDR1, LCDR2, and LCDR3) contained within a LCVR comprising the amino acid sequence of SEQ ID NO: 40 and three CDRs (HCDR1, HCDR2, and HCDR3) contained within a HCVR contained within a HCVR comprising an amino acid sequence selected from SEQ ID NOs: 32, 52, 78, 88, 120, 130, and 148.

23. The isolated antigen-binding protein of any one of claims 1-12, wherein the antigen-binding domain that binds specifically to SLC3A2-APIS comprises three CDRs (LCDR1, LCDR2, and LCDR3) contained within a LCVR comprising the amino acid sequence of SEQ ID NO: 70 and three CDRs (HCDR1, HCDR2, and HCDR3) contained within a HCVR comprising the amino acid sequence of SEQ ID NO: 62.

24. The isolated antigen-binding protein of any one of claims 1-12, wherein the antigen-binding domain that binds specifically to SLC3A2-APIS comprises three CDRs (LCDR1, LCDR2, and LCDR3) contained within a LCVR comprising the amino acid sequence of SEQ ID NO: 112 and three CDRs (HCDR1, HCDR2, and HCDR3) contained within a HCVR comprising the amino acid sequence of SEQ ID NO: 104.

25. The isolated antigen-binding protein of any one of claims 1-12, wherein the antigen-binding domain that binds specifically to SLC3A2-APIS comprises three CDRs (LCDR1, LCDR2, and LCDR3) contained within a LCVR comprising the amino acid sequence of SEQ ID NO: 140 and three CDRs (HCDR1, HCDR2, and HCDR3) contained within a HCVR comprising the amino acid sequence of SEQ ID NO: 22.

26. The isolated antigen-binding protein of any one of claims 1-12, wherein the antigen-binding domain that binds specifically to SLC3A2-APIS comprises three CDRs (LCDR1, LCDR2, and LCDR3) contained within a LCVR comprising the amino acid sequence of SEQ ID NO: 158 and three CDRs (HCDR1, HCDR2, and HCDR3) contained within a HCVR comprising the amino acid sequence of SEQ ID NO: 148.

27. The isolated antigen-binding protein of any one of claims 1-13, wherein the antigen-binding domain that binds specifically to SLC3A2-APIS comprises a LCVR comprising the amino acid sequence of SEQ ID NO: 10; and a HCVR comprising an amino acid sequence selected from SEQ ID NOs: 2, 22, 62, 104, 168, 178, 188, 198, and 208.

28. The isolated antigen-binding protein of any one of claims 1-13, wherein the antigen-binding domain that binds specifically to SLC3A2-APIS comprises a LCVR comprising the amino acid sequence of SEQ ID NO: 40; and a HCVR comprising an amino acid sequence selected from SEQ ID NOs: 32, 52, 78, 88, 120, 130, and 148.

29. The isolated antigen-binding protein of any one of claims 1-13, wherein the antigen-binding domain that binds specifically to SLC3A2-APIS comprises a LCVR comprising the amino acid sequence of SEQ ID NO: 70 and a HCVR comprising the amino acid sequence of SEQ ID NO: 62.

30. The isolated antigen-binding protein of any one of claims 1-13, wherein the antigen-binding domain that binds specifically to SLC3A2-APIS comprises a LCVR comprising the amino acid sequence of SEQ ID NO: 112 and a HCVR comprising the amino acid sequence of SEQ ID NO: 104.

31. The isolated antigen-binding protein of any one of claims 1-13, wherein the antigen-binding domain that binds specifically to SLC3A2-APIS comprises the amino acid sequence of SEQ ID NO: 140 and a HCVR comprising the amino acid sequence of SEQ ID NO: 22.

32. The isolated antigen-binding protein of any one of claims 1-13, wherein the antigen-binding domain that binds specifically to SLC3A2-APIS comprises a LCVR comprising the amino acid sequence of SEQ ID NO: 158 and a HCVR comprising the amino acid sequence of SEQ ID NO: 148.

33. The isolated antigen-binding protein of any one of claims 1-13, wherein the antigen-binding domain that binds specifically to SLC3A2-APIS comprises a LCVR comprising the amino acid sequence of SEQ ID NO: 232 and a HCVR comprising an amino acid sequence selected from SEQ ID NOs: 2, 22, 32, 52, 62, 78, 88, 104, 120, 130, 148, 168, 178, 188, 198, and 208.

34. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
(a) LCDR1, LCDR2, and LCDR3 comprise respective amino acid sequences of SEQ ID NO: 12, AAS, and SEQ ID NO: 16; and
(b) HCDR1, HCDR2 and HCDR3 comprise respective amino acid sequences selected from SEQ ID NOs: (i) 4, 6, and 8; (ii) 24, 26, and 28; (iii) 64, 66, and 68; (iv) 106, 108, and 110; (v) 170, 172, and 174; (vi) 170, 200, and 202 (vii) 180, 182, and 184; (viii) 190, 192, and 194; (ix) 170, 200, and 202; and (x) 210, 6, and 8.

35. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
(a) LCDR1, LCDR2, and LCDR3 comprise respective amino acid sequences of SEQ ID NO: 42, GAS, and SEQ ID NO: 46; and
(b) HCDR1, HCDR2, and HCDR3 comprise respective amino acid sequences selected from SEQ ID NOs: (i) 34, 36, and 38; (ii) 54, 56, and 58; (iii) 80, 82, and 84; (iv) 90, 92, and 94; (v) 122, 124, and 126; (vi) 132, 134, and 136; and (vii) 150, 152, and 154.

36. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
LCDR1, LCDR2, and LCDR3 comprise respective amino acid sequences of SEQ ID NO: 72, AAS, and SEQ ID NO: 16; and HCDR1, HCDR2, and HCDR3 comprise respective amino acid sequences of SEQ ID NOs: 64, 66, and 68.

37. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
LCDR1, LCDR2, and LCDR3 comprise respective amino acid sequences of SEQ ID NO: 114, AAS, and SEQ ID NO: 16; and HCDR1, HCDR2, and HCDR3 comprise respective amino acid sequences of SEQ ID NOs: 106, 108, and 110.

38. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
LCDR1, LCDR2, and LCDR3 comprise respective amino acid sequences of SEQ ID NO: 12, AAS, and SEQ ID NO: 142; and HCDR1, HCDR2, and HCDR3 comprise respective amino acid sequences of SEQ ID NOs: 24, 26, and 28.

39. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2, and HCDR3) contained within a HCVR, wherein:
LCDR1, LCDR2, and LCDR3 comprise respective amino acid sequences of SEQ ID NO: 160, GAS, and SEQ ID NO: 46; and HCDR1, HCDR2, and HCDR3 comprise respective amino acid sequences of SEQ ID NOs: 150, 152, and 154.

40. The isolated antigen-binding protein of any one of claims 1-12, wherein the antigen-binding domain that binds specifically to SLC3A2-APIS comprises three CDRs (LCDR1, LCDR2, and LCDR3) contained within a LCVR and three CDRs (HCDR1, HCDR2 and HCDR3) contained within a HCVR, wherein:
(a) LCDR1, LCDR2, and LCDR3 comprise respective amino acid sequences of SEQ ID NO: 234, AAS, and SEQ ID NO: 238; and
(b) HCDR1, HCDR2, and HCDR3 comprise respective amino acid sequences selected from SEQ ID NOs: (i) 4, 6, and 8; (ii) 24, 26, and 28; (iii) 34, 36, and 38; (iv) 54, 56, and 58; (v) 64, 66, and 68; (vi) 80, 82, and 84; (vii) 90, 92, and 94; (viii) 106, 108, and 110; (ix) 122, 124, and 126; (x) 132, 134, and 136; (xi) 150, 152, and 154 (xii) 170, 172, and 174; (xiii) 170, 200, and 202; (xiv) 180, 182, and 184; (xv) 190, 192, and 194; and (xvi) 210, 6, and 8.

41. The isolated antigen-binding protein of any one of claims 1-40, wherein the antigen-binding protein is a monoclonal antibody or antigen-binding fragment thereof.

42. The isolated antigen-binding protein of claim 41 comprising a heavy chain and a light chain, wherein the heavy chain comprises an amino acid sequence selected from SEQ ID NOs: 18, 30, 48, 60, 74, 86, 96, 98, 100, 102, 116, 128, 138, 144, 156, 164, 176, 186, 196, 204, 206, 212, and 214.

43. The isolated antigen-binding protein of claim 41 comprising a heavy chain and a light chain, wherein the light chain comprises an amino acid sequence selected from SEQ ID NOs: 20, 50, 76, 118, 146, and 166.

44. The isolated antigen-binding protein of claim 41 comprising a heavy chain and a light chain, wherein the heavy chain comprises an amino acid sequence selected from SEQ ID NOs: 18, 30, 48, 60, 74, 86, 96, 98, 100, 102, 116, 128, 138, 144, 156, 164, 176, 186, 196, 204, 206, 212, and 214; and wherein the light chain comprises an amino acid sequence selected from SEQ ID NOs: 20, 50, 76, 118, 146, and 166.

45. The isolated antigen-binding protein of any one of claims 1-41, wherein the antigen-binding protein is a bispecific antibody.

46. The isolated antigen-binding protein of claim 45, wherein the bispecific antibody further comprises an antigen-binding domain that binds specifically to CD3.

47. The isolated antigen-binding protein of claim 46, wherein the antigen-binding domain that binds specifically to CD3 comprises three CDRs (LCDR1, LCDR2, and LCDR3) contained within a LCVR comprising an amino acid sequence selected from SEQ ID NOs: 70, 232, and 277; and three CDRs (HCDR1, HCDR2, and HCDR3) contained within a HCVR comprising an amino acid sequence selected from SEQ ID NOs: 216, 224, 269, 291, and 301.

48. An isolated bispecific antigen-binding protein comprising:
(a) a first antigen-binding domain that binds specifically to SLC3A2-APIS, wherein the antigen-binding domain (i) does not bind to wild-type SLC3A2; and (ii) interacts with one or more amino acids contained within SEQ ID NO: 265, as determined by cryo-electron microscopy; and
(b) a second antigen-binding domain that binds specifically to CD3.

49. The isolated bispecific antigen-binding protein of claim 48, wherein the first antigen-binding domain that binds specifically to SLC3A2-APIS comprises: three CDRs (LCDR1, LCDR2, and LCDR3) contained within a LCVR comprising an amino acid sequence selected from SEQ ID NOs: 10, 40, 70, 112, 140, 158, 232, and 277; and three CDRs (HCDR1, HCDR2, and HCDR3) contained within a HCVR comprising an amino acid sequence selected from SEQ ID NOs: 2, 22, 32, 52, 62, 78, 88, 104, 120, 130, 148, 168, 178, 188, 198, 208 and 269.

50. The isolated bispecific antigen-binding protein of claim 48 or 49, wherein the second antigen-binding domain comprises three CDRs (LCDR1, LCDR2, and LCDR3) contained within a LCVR comprising an amino acid sequence selected from SEQ ID NOs: 70, 232 and 285; and three CDRs (HCDR1, HCDR2, and HCDR3) contained within a HCVR comprising an amino acid sequence selected from SEQ ID NOs: 216, 224, 269, 291, and 301.

51. The isolated bispecific antigen-binding protein of any one of claims 40-42, wherein the first antigen-binding domain that binds specifically to SLC3A2-APIS comprises: three CDRs (LCDR1, LCDR2, and LCDR3) contained within a LCVR comprising an amino acid sequence selected from SEQ ID NOs: 70, 232 and 285; and three CDRs (HCDR1, HCDR2, and HCDR3) contained within a HCVR comprising an amino acid sequence selected from SEQ ID NOs: 2, 22, 62, 104, 168, 178, 188, 198, 208, and 269.

52. The isolated bispecific antigen-binding protein of claim 51, wherein the second antigen-binding domain comprises three CDRs (LCDR1, LCDR2, and LCDR3) contained within a LCVR comprising an amino acid sequence selected from SEQ ID NOs: 70 and 232; and three CDRs (HCDR1, HCDR2, and HCDR3) contained within a HCVR comprising an amino acid sequence selected from SEQ ID NOs: 216 and 224.

53. The isolated bispecific antigen-binding protein of any one of claims 48-52, wherein the first antigen-binding domain that binds specifically to SLC3A2-APIS comprises:
(a) three CDRs (LCDR1, LCDR2, and LCDR3) contained within a LCVR wherein LCDR1, LCDR2 and LCDR3 comprise respective amino acid sequences selected from (i) SEQ ID NO: 72, AAS, and SEQ ID NO: 16; and (ii) SEQ ID NO: 234, AAS, SEQ ID NO: 238; and
(b) three CDRs (HCDR1, HCDR2, and HCDR3) contained within a HCVR wherein HCDR1, HCDR2, and HCDR3 comprise respective amino acid sequences selected from SEQ ID NOs: (i) 4, 6, and 8; (ii) 24, 26, and 28; (iii) 64, 66, and 68; (iv) 106, 108, and 110; (v) 170, 172, and 174; (vi) 170, 200, and 202; (vii) 180, 182, and 184; (viii) 190, 192, and 194; and (ix) 210, 6, and 8.

54. The isolated bispecific antigen-binding protein of any one of claims 48-53, wherein the second antigen-binding domain comprises:
(a) three CDRs (LCDR1, LCDR2, and LCDR3) contained within a LCVR wherein LCDR1, LCDR2 and LCDR3 comprise respective amino acid sequences selected from (i) SEQ ID NO: 72, AAS, SEQ ID NO: 16; and (ii) SEQ ID NO: 234, AAS, and SEQ ID NO: 238; and
(b) three CDRs (HCDR1, HCDR2, and HCDR3) contained within a HCVR wherein HCDR1, HCDR2, and HCDR3 comprise respective amino acid sequences selected from SEQ ID NOs: (i) 218, 220, and 222; and (ii) 226, 228, and 230.

55. The isolated bispecific antigen-binding protein of any one of claims 48-54, wherein the first antigen-binding domain that binds specifically to SLC3A2-APIS comprises a LCVR comprising an amino acid sequence selected from SEQ ID NOs: 70 and 232; and a HCVR comprising an amino acid sequence selected from SEQ ID NOs: 2, 22, 62, 104, 168, 178, 188, 198, and 208.

56. The isolated bispecific antigen-binding protein of any one of claims 48-55, wherein the second antigen-binding domain comprises a LCVR comprising an amino acid sequence selected from SEQ ID NOs: 70 and 232; and a HCVR comprising an amino acid sequence selected from SEQ ID NOs: 216 and 224.

57. An isolated bispecific antigen-binding protein comprising:
(a) an antigen-binding domain that binds specifically to SLC3A2-APIS comprising three CDRs (LCDR1, LCDR2, and LCDR3) contained within a LCVR comprising an amino acid sequence of SEQ ID NO: 70; and three CDRs (HCDR1, HCDR2, and HCDR3) contained within a HCVR comprising an amino acid sequence of SEQ ID NO: 62; and
(b) an antigen-binding domain that binds specifically to CD3 comprising three CDRs (LCDR1, LCDR2, and LCDR3) contained within a LCVR comprising an amino acid sequence of SEQ ID NO: 70; and three CDRs (HCDR1, HCDR2, and HCDR3) contained within a HCVR comprising an amino acid sequence of SEQ ID NO: 216.

58. An isolated bispecific antigen-binding protein comprising:
(a) an antigen-binding domain that binds specifically to SLC3A2-APIS comprising three CDRs (LCDR1, LCDR2, and LCDR3) contained within a LCVR comprising an amino acid sequence of SEQ ID NO: 70; and three CDRs (HCDR1, HCDR2, and HCDR3) contained within a HCVR comprising an amino acid sequence of SEQ ID NO: 62; and
(b) an antigen-binding domain that binds specifically to CD3 comprising three CDRs (LCDR1, LCDR2, and LCDR3) contained within a LCVR comprising an amino acid sequence of SEQ ID NO: 70; and three CDRs (HCDR1, HCDR2, and HCDR3) contained within a HCVR comprising an amino acid sequence of SEQ ID NO: 224.

59. An isolated bispecific antigen-binding protein comprising:
(a) an antigen-binding domain that binds specifically to SLC3A2-APIS comprising three CDRs (LCDR1, LCDR2, and LCDR3) contained within a LCVR and three CDRs (HCDR1, HCDR2, and HCDR3) contained within a HCVR; wherein LCDR1, LCDR2, and LCDR3 comprise respective amino acid sequences of SEQ ID NO: 72, AAS, and SEQ ID NO: 16; and HCDR1, HCDR2 and HCDR3 comprise respective amino acid sequences of SEQ ID NOs: 64, 66, and 68; and
(b) an antigen-binding domain that binds specifically to CD3 comprising three CDRs (LCDR1, LCDR2, and LCDR3) contained within a LCVR and three CDRs (HCDR1, HCDR2, and HCDR3) contained within a HCVR; wherein LCDR1, LCDR2, and LCDR3 comprise respective amino acid sequences of SEQ ID NO: 72, AAS, and SEQ ID NO: 16; and HCDR1, HCDR2, and HCDR3 comprise respective amino acid sequences of SEQ ID NOs: 218, 220, and 222.

60. An isolated bispecific antigen-binding protein comprising:
(a) an antigen-binding domain that binds specifically to SLC3A2-APIS comprising three CDRs (LCDR1, LCDR2 and LCDR3) contained within a LCVR and three CDRs (HCDR1, HCDR2 and HCDR3) contained within a HCVR; wherein LCDR1, LCDR2 and LCDR3 comprise respective amino acid sequences of SEQ ID NO: 72, AAS, and SEQ ID NO: 16; and HCDR1, HCDR2 and HCDR3 comprise respective amino acid sequences of SEQ ID NOs: 64, 66, and 68; and
(b) an antigen-binding domain that binds specifically to CD3 comprising three CDRs (LCDR1, LCDR2 and LCDR3) contained within a LCVR and three CDRs (HCDR1, HCDR2 and HCDR3) contained within a HCVR; wherein LCDR1, LCDR2 and LCDR3 comprise respective amino acid sequences of SEQ ID NO: 72, AAS, and SEQ ID NO: 16; and HCDR1, HCDR2 and HCDR3 comprise respective amino acid sequences of SEQ ID NOs: 226, 228, and 230.

61. An isolated bispecific antigen-binding protein comprising:
(a) an antigen-binding domain that binds specifically to SLC3A2-APIS comprising a LCVR comprising an amino acid sequence of SEQ ID NO: 70; and a HCVR comprising an amino acid sequence of SEQ ID NO: 62; and
(b) an antigen-binding domain that binds specifically to CD3 comprising a LCVR comprising an amino acid sequence of SEQ ID NO: 70; and a HCVR comprising an amino acid sequence of SEQ ID NO: 216.

62. An isolated bispecific antigen-binding protein comprising:
(a) an antigen-binding domain that binds specifically to SLC3A2-APIS comprising a LCVR comprising an amino acid sequence of SEQ ID NO: 70; and a HCVR comprising an amino acid sequence of SEQ ID NO: 62; and
(b) an antigen-binding domain that binds specifically to CD3 comprising a LCVR comprising an amino acid sequence of SEQ ID NO: 70; and a HCVR comprising an amino acid sequence of SEQ ID NO: 224.

63. The isolated bispecific antigen-binding protein of any one of claims 48-62 that is a bispecific antibody comprising:
a first heavy chain and a paired light chain interconnected by disulfide bonds, wherein the first heavy chain comprises a HCVR and a heavy chain constant region comprising CH1, CH2, and CH3 domains, and the paired light chain comprises a LCVR and a light chain constant region, wherein the first heavy chain and paired light chain comprise the first antigen-binding domain; and
a second heavy chain and a paired light chain interconnected by disulfide bonds, wherein the second heavy chain comprises a HCVR and a heavy chain constant region comprising CH1, CH2, and CH3 domains, and the light chain comprises a LCVR and a light chain constant region, wherein the second heavy chain and paired light chain comprise the second antigen-binding domain.

64. The isolated bispecific antigen-binding protein of claim 63, wherein the first heavy chain or the second heavy chain, but not both, comprises a CH3 domain comprising a H435R (EU numbering) modification and a Y436F (EU numbering) modification.

65. The isolated bispecific antigen-binding protein of claim 63 or 64, wherein the heavy chain constant region of the first heavy chain and the heavy chain constant region of the second heavy chain are of isotype IgG1.

66. The isolated bispecific antigen-binding protein of claim 63 or 64, wherein the heavy chain constant region of the first heavy chain and the heavy chain constant region of the second heavy chain are of isotype IgG4.

67. The isolated bispecific antigen-binding protein of any one of claims 63-66, wherein the first heavy chain and the second heavy chain comprise a chimeric hinge that reduces Fcγ receptor binding relative to a wild-type hinge of the same isotype.

68. The isolated bispecific antigen-binding protein of claim 63, wherein the first heavy chain comprises an amino acid sequence selected from SEQ ID NOs: 248, 250, 252, 254, 256, 258, 260, 262, and 264; and the second heavy chain comprises an amino acid sequence selected from SEQ ID NO: 240 and 242.

69. The isolated bispecific antigen-binding protein of claim 63 or 68, wherein the paired light chain comprises an amino acid sequence selected from SEQ ID NO: 244 and 246.

70. The isolated bispecific antigen-binding protein of claim 63, which is a bispecific antibody wherein the first heavy chain comprises the amino acid sequence of SEQ ID NO: 258, the second heavy chain comprises the amino acid sequence of SEQ ID NO: 240, and the paired light chain comprises the amino acid sequence of SEQ ID NO: 246.

71. The isolated bispecific antigen-binding protein of claim 63, which is a bispecific antibody wherein the first heavy chain comprises the amino acid sequence of SEQ ID NO: 258, the second heavy chain comprises the amino acid sequence of SEQ ID NO: 242, and the paired light chain comprises the amino acid sequence of SEQ ID NO: 246.

72. A pharmaceutical composition comprising the isolated antigen-binding protein of any one of claims 1-47, and a pharmaceutically acceptable carrier or diluent.

73. A pharmaceutical composition comprising the isolated bispecific antigen-binding protein of any one of claims 48-71, and a pharmaceutically acceptable carrier or diluent.

74. A method for making the isolated antigen-binding protein of any one of claims 1-47 comprising: (a) introducing one or more nucleic acid molecules comprising polynucleotide sequences that encode the antigen-binding protein into a host cell, and (b) culturing the host cell under conditions favorable to expression of the one or more nucleic acid molecules.

75. The method of claim 74, further comprising (c) isolating the antigen-binding protein from the host cell and/or medium in which the host cell is grown.

76. The method of claim 75, wherein the host cell is a Chinese hamster ovary (CHO) cell.

77. A nucleic acid molecule comprising a nucleotide sequence encoding the antigen-binding protein of any one of claims 1-47, or a group of nucleic acid molecules comprising nucleotide sequences, respectively, encoding the HCVR and the LCVR of the antigen-binding protein of any one of claims 1-47.

78. An expression vector comprising the nucleic acid molecule of claim 77, or a group of expression vectors comprising, respectively, the group of nucleic acid molecules of claim 77.

79. A host cell comprising the antigen-binding protein of any one of claims 1-47, the nucleic acid molecule or group of nucleic acid molecules of claim 77, or the expression vector or group of expression vectors of claim 78.

80. The host cell of claim 79, wherein the host cell is a CHO cell.

81. A method of producing an antigen-binding protein that specifically binds SLC3A2-APIS comprising: (a) culturing the host cell of claim 79 under conditions favorable for production of the antigen-binding protein; and (b) optionally, isolating the antigen-binding protein from the host cell and/or medium in which the host cell is grown.

82. The method of claim 81, wherein the host is a CHO cell.

83. The method of claim 81 or 82 further comprising formulating the antigen-binding protein as a pharmaceutical composition comprising an acceptable carrier.

84. A method for making the isolated bispecific antigen-binding protein of any one of claims 44-67 comprising: (a) introducing one or more nucleic acid molecules comprising polynucleotide sequences that encode the bispecific antigen-binding protein into a host cell, and (b) culturing the host cell under conditions favorable to expression of the one or more nucleic acid molecules.

85. The method of claim 84, further comprising (c) isolating the bispecific antigen-binding protein from the host cell and/or medium in which the host cell is grown.

86. The method of claim 85, wherein the host cell is a CHO cell.

87. A nucleic acid molecule comprising a nucleotide sequence encoding a bispecific antigen-binding protein of any one of claims 48-71, or a group of nucleic acid molecules comprising nucleotide sequences, respectively, encoding the HCVR of the first antigen-binding domain, the HCVR of the second antigen-binding domain, and the LCVR of the first and second antigen-binding domains of a bispecific antigen-binding protein of any one of claims 44-67.

88. An expression vector comprising the nucleic acid molecule of claim 87, or a group of expression vectors comprising, respectively, the group of nucleic acid molecules of claim 87.

89. A host cell comprising the bispecific antigen-binding protein of any one of claims 48-71, the nucleic acid molecule or group of nucleic acid molecules of claim 85, or the expression vector or group of expression vectors of claim 88.

90. A host cell comprising:
(a) an expression vector comprising a nucleic acid molecule encoding a first immunoglobulin heavy chain comprising the amino acid sequence of SEQ ID NO: 258;
(b) an expression vector comprising a nucleic acid molecule encoding a second immunoglobulin heavy chain comprising the amino acid sequence of SEQ ID NO: 240; and
(c) an expression vector comprising a nucleic acid molecule encoding an immunoglobulin light chain comprising the amino acid sequence of SEQ ID NO: 246.

91. A host cell comprising:
(a) an expression vector comprising a nucleic acid molecule encoding a first immunoglobulin heavy chain comprising the amino acid sequence of SEQ ID NO: 258;
(b) an expression vector comprising a nucleic acid molecule encoding a second immunoglobulin heavy chain comprising the amino acid sequence of SEQ ID NO: 242; and
(c) an expression vector comprising a nucleic acid molecule encoding an immunoglobulin light chain comprising the amino acid sequence of SEQ ID NO: 246.

92. A method of producing a bispecific antigen-binding protein that specifically binds SLC3A2-APIS and CD3 comprising: (a) culturing the host cell of any one of claims 89-91 under conditions favorable for production of the bispecific antigen-binding protein; and (b) optionally, isolating the bispecific antigen-binding protein from the host cell and/or medium in which the host cell is grown.

93. The method of claim 92, wherein the host is a CHO cell.

94. The method of claim 92 or 93 further comprising formulating the bispecific antigen-binding protein as a pharmaceutical composition comprising an acceptable carrier.

95. A method of inhibiting growth of a tumor in a subject, comprising administering the isolated antigen-binding protein of any one of claims 1-47, the bispecific antigen-binding protein of any one of claims 48-71, or the pharmaceutical composition of claim 72 or 73 to the subject.

96. The method of claim 95, wherein the tumor is anal cancer, angiosarcoma, acute myeloid leukemia, acute lymphocytic leukemia, a B cell cancer, basal cell carcinoma, bladder cancer, bone cancer, brain cancer, breast cancer, cervical cancer, cholangiocarcinoma, chondrosarcoma, chronic lymphocytic leukemia colon cancer, colorectal cancer, cutaneous squamous cell carcinoma, endometrial cancer, esophageal cancer, glioblastoma multiforme, head and neck squamous cell cancer, hepatocellular carcinoma, kidney cancer, liver cancer, lung cancer, lymphoma, leukemia, Merkel cell carcinoma, melanoma, myeloma, myeloproliferative neoplasm, non-small cell lung cancer, ovarian cancer, pancreatic cancer, prostate cancer, salivary gland cancer, skin cancer, soft tissue sarcoma, stomach cancer, a T cell cancer, testicular cancer, uterine cancer, or uveal melanoma.

97. The method of claim 95 or 96, wherein the tumor comprises SLC3A2-APIS-expressing tumor cells.

98. A method of treating or ameliorating one or more symptoms of a myelodysplastic syndrome in a subject, comprising administering the antigen-binding protein of any one of claims 1-47, the bispecific antigen-binding protein of any one of claims 48-71, or the pharmaceutical composition of claim 72 or 73 to the subject.

99. A method of reducing the risk of progression to a cancer, comprising administering to a subject in need thereof the antigen-binding protein of any one of claims 1-47, or the bispecific antigen-binding protein of any one of claims 48-71, wherein the subject has been diagnosed with a premalignant condition, and wherein the risk of progression is reduced relative to a control subject not administered the antigen-binding protein or bispecific antigen-binding protein.

100. The method of claim 99, wherein the premalignant condition is clonal hematopoiesis of indeterminate potential (CHIP), clonal cytopenia of undetermined significance (CCUS), or age-related clonal hematopoiesis (ARCH).

101. The method of any one of claims 95-100, further comprising administering a second therapeutic agent or therapeutic regimen.

102. The method of claim 101, wherein the second therapeutic agent or therapeutic regimen comprises a chemotherapeutic drug, a DNA alkylator, an immunomodulator, a proteasome inhibitor, a histone deacetylase inhibitor, radiotherapy, surgery, a stem cell transplant, leukapheresis, an antibody drug conjugate, a cell containing a chimeric antigen receptor, an oncolytic virus, a bispecific antibody conjugated to an anti-tumor agent, a VEGF inhibitor, a checkpoint inhibitor, a GITR agonist, a CD27 agonist, a CD28 agonist, a 4-1BB activator, a CD38 inhibitor, a PD-1 inhibitor, a PD-L1 inhibitor, a LAG3 inhibitor, a CTLA-4 inhibitor, an EGFR inhibitor, an Ang2 inhibitor, a MUC16 inhibitor, a CD20 inhibitor, a BCMA inhibitor, a bispecific antibody comprising a CD28-binding arm, a bispecific antibody comprising a 4-1BB-binding arm, a cancer vaccine, a cytokine, a modified IL2, a modified IL12, an IL1 inhibitor, an IL4 inhibitor, an IL6 inhibitor, a corticosteroid, or combinations thereof.

103. An isolated antigen-binding protein that comprises an antigen-binding domain that binds specifically to SLC3A2-APIS in a tumor cell for use in a method of treating, inhibiting or preventing the growth of a tumor.

104. The isolated antigen-binding protein of claim 103, wherein the antigen-binding protein further comprises an antigen-binding domain that binds specifically to CD3.

105. A kit comprising an isolated antigen-binding protein that comprises an antigen-binding domain that binds specifically to SLC3A2-APIS in a tumor cell in combination with written instructions for use of a therapeutically effective amount of the antigen-binding protein for inhibiting the growth of a tumor in a subject.

106. The kit of claim 105, wherein the antigen-binding protein further comprises an antigen-binding domain that binds specifically to CD3.
